(19) United States
(12) Patent Application Publication
(10) Pub. No.: US 2026/0355120 A1
(43) Pub. Date: Nov. 12, 2026

(54) "GOOD" BUFFER-BASED CATIONIC LIPIDS FOR NUCLEIC ACID DELIVERY

(71) Applicant: SANOFI VACCINES US INC., Swiftwater, PA (US)

(72) Inventors: Saswata KARMAKAR, Cambridge, MA (US); Ryan LANDIS, Cambridge, MA (US)

(21) Appl. No.: 19/553,069

(22) Filed: Feb. 27, 2026

Related U.S. Application Data
(63) Continuation of application No. PCT/EP2024/076467, filed on Sep. 20, 2024.

(30) Foreign Application Priority Data
Sep. 20, 2023 (EP) 23306558.0

(51) Int. Cl. A61K 9/51; A61K 9/00; A61K 31/7105; A61K 38/16; A61K 47/22; A61P 31/16; C07D 241/04

(52) U.S. Cl. CPC A61K 9/5123; A61K 9/0019; A61K 31/7105; A61K 38/16; A61K 47/22; A61P 31/16; C07D 241/04

(57) ABSTRACT

The present invention provides, in part, asymmetric “good” buffer-based cationic lipids of Formula (I′), and sub-formulas thereof:

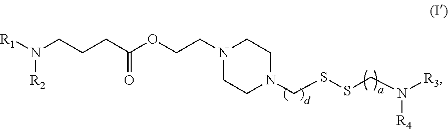

or a pharmaceutically acceptable salt thereof. The compounds provided herein can be useful for delivery and expression of mRNA and encoded protein, e.g., as a component of liposomal delivery vehicle, and accordingly can be useful for treating various diseases, disorders and conditions, such as those caused by viral or bacterial infection.

### RELATED APPLICATIONS

This application is a continuation of International Patent Application No. PCT/EP2024/076467, filed Sep. 20, 2024, which claims priority to European Patent Application No. 23306558.0 filed Sep. 20, 2023, the entire disclosures of which are hereby incorporated herein by reference.

### BACKGROUND

Delivery of nucleic acids has been explored extensively as a potential therapeutic option for certain disease states. In particular, messenger RNA (mRNA) therapy has become an increasingly important option for the prevention and treatment of various diseases (e.g. in the use of vaccines).

Efficient delivery of liposome-encapsulated nucleic acids remains an active area of research. Liposome-encapsulated nucleic acids can be administered intramuscularly (IM).

The cationic lipid component of a liposome plays an important role in facilitating effective encapsulation of the nucleic acid during the loading of liposomes. In addition, cationic lipids may play an important role in the efficient release of the nucleic acid cargo from the liposome into the cytoplasm of a target cell. Various cationic lipids suitable for in vivo use have been discovered. However, there remains a need to identify cationic lipids that are effective for e.g., intramuscular delivery of mRNA (e.g., in vaccines). There also remains a need to identify cationic lipids that can be synthesized efficiently and cheaply without the formation of potentially toxic by-products.

“Good” buffers (or Good's buffers) are buffering agents for biochemical and biological research that were first selected and described by Norman Good and his colleagues (Good, N. E., et al. (1966) Hydrogen Ion Buffers for Biological Research. Biochemistry 5 (2), 467-477). Most biological reactions take place near-neutral pH between 6 and 8. Good therefore reasoned that an ideal buffer for biochemical or biological applications would have a pKa value in this region to provide maximum buffering capacity. Additional selection criteria included high solubility, lack of toxicity, limited interference with biochemical reactions, very low absorbance between 240 nm and 700 nm, enzymatic and hydrolytic stability, minimal changes due to temperature and concentration, limited effects due to ionic or salt composition of the solution, limited interaction with mineral cations, and limited permeability of biological membranes.

The foregoing characteristics make “Good” buffers exceptionally good starting points for the synthesis of cationic lipids for use in in vivo settings. Many “Good” buffers remain crucial tools in modern laboratories and are therefore readily available at low cost.

### SUMMARY OF THE INVENTION

The present invention provides, among other things, a novel class of cationic lipid compounds for in vivo delivery of therapeutic agents, such as nucleic acids. The “Good” HEPES, HEPPS, and HEPBS buffers form the cores of the cationic lipids of the invention and were used to synthesize unique ionizable lipids containing different degradable moieties and carbon tails. The core structure with a hydroxyl and sulfonic acid group on either side allows for the ionizable lipids to contain both ester and disulfide degradable moieties. The inventors of the present invention have surprisingly found that certain lipid nanoparticles comprising a cationic lipid derived from HEPES, HEPPS, and HEPBS buffers, for example which possesses at least one pair of asymmetric lipid tails on either arm of the lipid molecule, exhibit improved properties relative to lipid nanoparticles comprising other cationic lipids derived from “Good” buffers, such as those in WO 2022/221688 A1, incorporated herein by reference and are very effective for the intramuscular delivery of mRNA encapsulated in said lipid nanoparticles. Indeed, lipid nanoparticles comprising the cationic lipids of the present invention have demonstrated high levels of peptide or protein expression when delivering mRNA encoding for said peptide or protein by intramuscular delivery. For example, lipid nanoparticles comprising cationic lipids of the present invention and encapsulating human erythropoietin (hEPO) mRNA achieved improved expression of hEPO mRNA when administered to mice by intramuscular delivery to lipid nanoparticles comprising MC3, which is currently used for in vivo delivery of e.g. siRNA (see WO2010/144740).

The cationic lipids of the present invention are also more straightforward to synthesize than other cationic lipids, such as MC3. Indeed, the synthesis of MC3 involves a six-step process and requires handling of a Grignard reagent. In contrast, the cationic lipids of the present invention can be synthesized from readily available starting reagents, such as “Good's” buffers (see Table A).

The cationic lipids of the present invention also comprise cleavable groups (e.g., esters, thioesters, disulphides, carbonates, carbamates and thiocarbamates) that are contemplated to improve biodegradability and thus contribute to their favorable safety profile.

It is contemplated that these compounds are capable of highly effective in vivo intramuscular delivery of various therapeutic agents and vaccines (e.g., therapeutic agents and/or vaccines for use in Flu, acne, chlamydia, or a disease or disorder induced by infection with P. gingivalis). It is also contemplated that lipid nanoparticles comprising these cationic lipid compounds are capable of highly effective in vivo delivery while maintaining a favorable safety profile. It is also contemplated that lipid nanoparticles comprising these cationic lipid compounds may exhibit improved degradation in vivo.

In an aspect, provided herein are cationic lipids having a structure according to Formula (I′), optionally Formula (I):

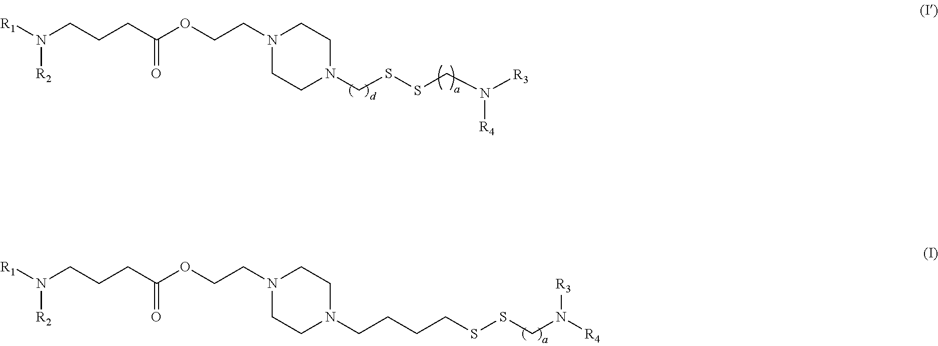

or a pharmaceutically acceptable salt thereof, wherein:
   a is 3 or 4;
   each of R₁, R₂, R₃, and R₄ is independently selected from:
      (i) optionally substituted C₅-C₃₀ alkyl, and optionally substituted C₅-C₃₀ alkenyl;
      (ii) optionally substituted C₁₋₅ alkyl which is substituted with an optionally substituted heterocyclyl moiety;
      (iii)

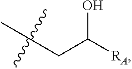

wherein each R_{A} is independently selected from optionally substituted C₃-C₂₈ alkyl, optionally substituted C₃-C₂₈ alkenyl, and —W¹—C(O)—O—X¹, wherein each W¹ is independently selected from optionally substituted C₃-C₅ alkylene and optionally substituted C₃-C₅ alkenylene, and each X¹ is independently selected from optionally substituted C₅-C₃₀ alkyl and optionally substituted C₅-C₃₀ alkenyl;
(iv)

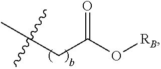

wherein each R_{B} is independently selected from optionally substituted C₅-C₃₀ alkyl, optionally substituted C₅-C₃₀ alkenyl; and
(v)

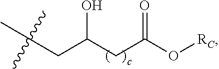

wherein each R_{C} independently selected from optionally substituted C₅-C₃₀ alkyl, optionally substituted C₅-C₃₀ alkenyl;
each b is independently selected from 5, 6, and 7;
each c is independently selected from 3, 4, and 5;
d is 2, 3, or 4;
and wherein
R₁ and R₂ are not identical to one another and/or R₃ and R₄ are not identical to one another; and wherein
at least one of R₁, R₂, R₃, and R₄, is independently selected from
option (iv)

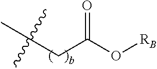

or option (v)

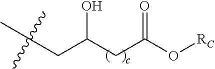

above.

In an aspect, provided herein are cationic lipids having a structure according to Formula (VI′), optionally Formula (VI):

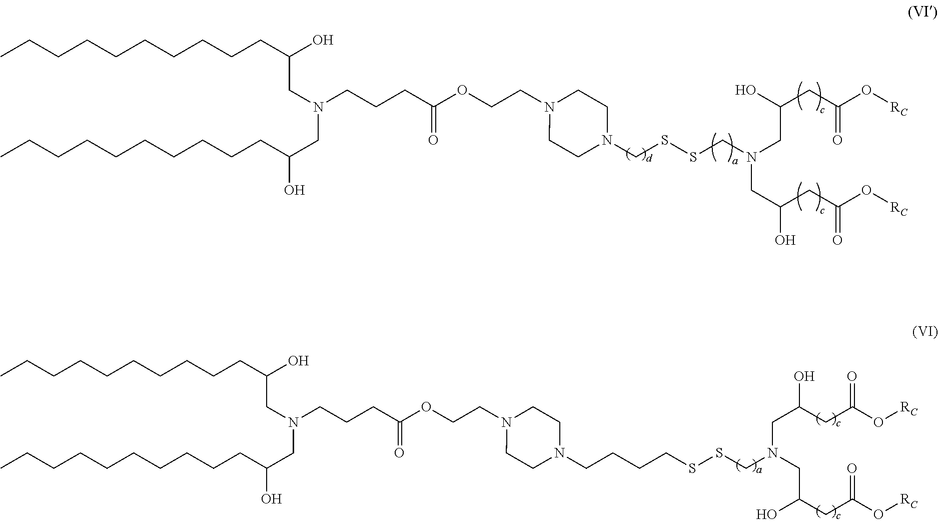

or a pharmaceutically acceptable salt thereof, wherein:
a is 3 or 4;
d is 2, 3, or 4;
each c is independently selected from 3 and 4; and
each R_{C} is independently selected from

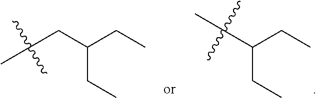

In another aspect provided herein is a compound selected from those listed in Tables 1 or 2, or a pharmaceutically acceptable salt thereof.

In another aspect provided herein is a composition comprising one or more cationic lipids of the present invention or pharmaceutically acceptable salts thereof, one or more non-cationic lipids, one or more cholesterol-based lipids and one or more PEG-modified lipids.

Also provided herein are compositions of the invention for use in methods of treating or preventing a disease or disorder amenable to treatment or prevention by a peptide or protein encoded by mRNA that is encapsulated within said composition. Methods of treating or preventing a disease or disorder wherein said method comprises administering to a subject in need thereof a composition of the invention and wherein the disease is amenable to treatment or prevention by a peptide or protein encoded by mRNA that is encapsulated within said composition are also provided.

### BRIEF DESCRIPTION OF DRAWINGS

FIG. 1 shows HAI Titers after 35 days of post intramuscular injection (0.4 μg/50 μL) in mice of mRNA encoded influenza antigen encapsulated within lipid nanoparticles comprising cationic lipids of the present invention.

### DETAILED DESCRIPTION OF CERTAIN EMBODIMENTS

### Definitions

In order for the present invention to be more readily understood, certain terms are first defined below. Additional definitions for the following terms and other terms are set forth throughout the specification. The publications and other reference materials cited herein to describe the background of the invention and to provide additional detail regarding its practice are hereby incorporated by reference.

Amino acid: As used herein, the term “amino acid,” in its broadest sense, refers to any compound and/or substance that can be incorporated into a polypeptide chain. In some embodiments, an amino acid has the general structure H₂N—C(H)(R)—COOH. In some embodiments, an amino acid is a naturally occurring amino acid. In some embodiments, an amino acid is a synthetic amino acid; in some embodiments, an amino acid is a d-amino acid; in some embodiments, an amino acid is an I-amino acid. “Standard amino acid” refers to any of the twenty standard I-amino acids commonly found in naturally occurring peptides. “Nonstandard amino acid” refers to any amino acid, other than the standard amino acids, regardless of whether it is prepared synthetically or obtained from a natural source. As used herein, “synthetic amino acid” encompasses chemically modified amino acids, including but not limited to salts, amino acid derivatives (such as amides), and/or substitutions. Amino acids, including carboxy- and/or amino-terminal amino acids in peptides, can be modified by methylation, amidation, acetylation, protecting groups, and/or substitution with other chemical groups that can change the peptide's circulating half-life without adversely affecting their activity. Amino acids may participate in a disulfide bond. Amino acids may comprise one or post-translational modifications, such as association with one or more chemical entities (e.g., methyl groups, acetate groups, acetyl groups, phosphate groups, formyl moieties, isoprenoid groups, sulfate groups, polyethylene glycol moieties, lipid moieties, carbohydrate moieties, biotin moieties, etc.). The term “amino acid” is used interchangeably with “amino acid residue,” and may refer to a free amino acid and/or to an amino acid residue of a peptide. It will be apparent from the context in which the term is used whether it refers to a free amino acid or a residue of a peptide.

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, a bovine, a primate, and/or a pig). In some embodiments, animals include, but are not limited to, mammals, birds, reptiles, amphibians, fish, insects, and/or worms. In some embodiments, an animal may be a transgenic animal, genetically engineered animal, and/or a clone.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Biologically active: As used herein, the term “biologically active” refers to a characteristic of any agent that has activity in a biological system, and particularly in an organism. For instance, an agent that, when administered to an organism, has a biological effect on that organism, is considered to be biologically active.

Delivery: As used herein, the term “delivery” encompasses both local and systemic delivery. For example, delivery of mRNA encompasses situations in which an mRNA is delivered to a target tissue and the encoded protein is expressed and retained within the target tissue (also referred to as “local distribution” or “local delivery”), and situations in which an mRNA is delivered to a target tissue and the encoded protein is expressed and secreted into patient's circulation system (e.g., serum) and systematically distributed and taken up by other tissues (also referred to as “systemic distribution” or “systemic delivery”).

Expression: As used herein, “expression” of a nucleic acid sequence refers to translation of an mRNA into a polypeptide, assemble multiple polypeptides into an intact protein (e.g., enzyme) and/or post-translational modification of a polypeptide or fully assembled protein (e.g., enzyme). In this application, the terms “expression” and “production,” and grammatical equivalents thereof, are used interchangeably.

Functional: As used herein, a “functional” biological molecule is a biological molecule in a form in which it exhibits a property and/or activity by which it is characterized.

Half-life: As used herein, the term “half-life” is the time required for a quantity such as nucleic acid or protein concentration or activity to fall to half of its value as measured at the beginning of a time period.

Helper lipid: The term “helper lipid” as used herein refers to any neutral or zwitterionic lipid material including cholesterol. Without wishing to be held to a particular theory, helper lipids may add stability, rigidity, and/or fluidity within lipid bilayers/nanoparticles.

Improve, increase, or reduce: As used herein, the terms “improve,” “increase,” or “reduce,” or grammatical equivalents, indicate values that are relative to a baseline measurement, such as a measurement in the same individual prior to initiation of the treatment described herein, or a measurement in a control subject (or multiple control subject) in the absence of the treatment described herein. A “control subject” is a subject afflicted with the same form of disease as the subject being treated, who is about the same age as the subject being treated.

In Vitro: As used herein, the term “in vitro” refers to events that occur in an artificial environment, e.g., in a test tube or reaction vessel, in cell culture, etc., rather than within a multi-cellular organism.

In Vivo: As used herein, the term “in vivo” refers to events that occur within a multi-cellular organism, such as a human and a non-human animal. In the context of cell-based systems, the term may be used to refer to events that occur within a living cell (as opposed to, for example, in vitro systems).

Liposome: As used herein, the term “liposome” refers to any lamellar, multilamellar, or solid nanoparticle vesicle. Typically, a liposome as used herein can be formed by mixing one or more lipids or by mixing one or more lipids and polymer(s). In some embodiments, a liposome suitable for the present invention contains a cationic lipid(s) and optionally further comprises:
(i) non-cationic lipid(s),
(ii) cholesterol-based lipid(s), and/or
(iii) PEG-modified lipid(s).

messenger RNA (mRNA): As used herein, the term “messenger RNA (mRNA)” or “mRNA” refers to a polynucleotide that encodes at least one polypeptide. mRNA as used herein encompasses both modified and unmodified RNA. The term “modified mRNA” related to mRNA comprising at least one chemically modified nucleotide. mRNA may contain one or more coding and non-coding regions. mRNA can be purified from natural sources, produced using recombinant expression systems and optionally purified, chemically synthesized, etc. Where appropriate, e.g., in the case of chemically synthesized molecules, mRNA can comprise nucleoside analogs such as analogs having chemically modified bases or sugars, backbone modifications, etc. An mRNA sequence is presented in the 5′ to 3′ direction unless otherwise indicated. In some embodiments, an mRNA is or comprises natural nucleosides (e.g., adenosine, guanosine, cytidine, uridine); nucleoside analogs (e.g., 2-aminoadenosine, 2-thiothymidine, inosine, pyrrolo-pyrimidine, 3-methyl adenosine, 5-methylcytidine, C5-propynyl-cytidine, C5-propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

Nucleic acid: As used herein, the term “nucleic acid,” in its broadest sense, refers to any compound and/or substance that is or can be incorporated into a polynucleotide chain. In some embodiments, a nucleic acid is a compound and/or substance that is or can be incorporated into a polynucleotide chain via a phosphodiester linkage. In some embodiments, “nucleic acid” refers to individual nucleic acid residues (e.g., nucleotides and/or nucleosides). In some embodiments, “nucleic acid” refers to a polynucleotide chain comprising individual nucleic acid residues. In some embodiments, “nucleic acid” encompasses RNA as well as single and/or double-stranded DNA and/or cDNA. In some embodiments, “nucleic acid” encompasses ribonucleic acids (RNA), including but not limited to any one or more of interference RNAs (RNAi), small interfering RNA (siRNA), short hairpin RNA (shRNA), antisense RNA (aRNA), messenger RNA (mRNA), modified messenger RNA (mmRNA), long non-coding RNA (lncRNA), micro-RNA (miRNA) multimeric coding nucleic acid (MCNA), polymeric coding nucleic acid (PCNA), guide RNA (gRNA) and CRISPR RNA (crRNA). In some embodiments, “nucleic acid” encompasses deoxyribonucleic acid (DNA), including but not limited to any one or more of single-stranded DNA (ssDNA), double-stranded DNA (dsDNA) and complementary DNA (cDNA). In some embodiments, “nucleic acid” encompasses both RNA and DNA. In embodiments, DNA may be in the form of antisense DNA, plasmid DNA, parts of a plasmid DNA, pre-condensed DNA, a product of a polymerase chain reaction (PCR), vectors (e.g., P1, PAC, BAC, YAC, artificial chromosomes), expression cassettes, chimeric sequences, chromosomal DNA, or derivatives of these groups. In embodiments, RNA may be in the form of messenger RNA (mRNA), ribosomal RNA (rRNA), signal recognition particle RNA (7 SL RNA or SRP RNA), transfer RNA (tRNA), transfer-messenger RNA (tmRNA), small nuclear RNA (snRNA), small nucleolar RNA (snoRNA), SmY RNA, small Cajal body-specific RNA (scaRNA), guide RNA (gRNA), ribonuclease P (RNase P), Y RNA, telomerase RNA component (TERC), spliced leader RNA (SL RNA), antisense RNA (aRNA or asRNA), cis-natural antisense transcript (cis-NAT), CRISPR RNA (crRNA), long noncoding RNA (lncRNA), micro-RNA (miRNA), piwi-interacting RNA (piRNA), small interfering RNA (siRNA), transacting siRNA (tasiRNA), repeat associated siRNA (rasiRNA), 73K RNA, retrotransposons, a viral genome, a viroid, satellite RNA, or derivatives of these groups. In some embodiments, a nucleic acid is a mRNA encoding a protein such as an enzyme.

Patient: As used herein, the term “patient” or “subject” refers to any organism to which a provided composition may be administered, e.g., for experimental, diagnostic, prophylactic, cosmetic, and/or therapeutic purposes. Typical patients include animals (e.g., mammals such as mice, rats, rabbits, non-human primates, and/or humans). In some embodiments, a patient is a human. A human includes pre- and post-natal forms.

Pharmaceutically acceptable: The term “pharmaceutically acceptable,” as used herein, refers to substances that, within the scope of sound medical judgment, are suitable for use in contact with the tissues of human beings and animals without excessive toxicity, irritation, allergic response, or other problem or complication, commensurate with a reasonable benefit/risk ratio.

Pharmaceutically acceptable salt: Pharmaceutically acceptable salts are well known in the art. For example, S. M. Berge et al., describes pharmaceutically acceptable salts in detail in J. Pharmaceutical Sciences (1977) 66:1-19. Pharmaceutically acceptable salts of the compounds of this invention include those derived from suitable inorganic and organic acids and bases. Examples of pharmaceutically acceptable, non-toxic acid addition salts are salts of an amino group formed with inorganic acids such as hydrochloric acid, hydrobromic acid, phosphoric acid, sulfuric acid, and perchloric acid or with organic acids such as acetic acid, oxalic acid, maleic acid, tartaric acid, citric acid, succinic acid, or malonic acid, or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄ alkyl) 4 salts. Representative alkali or alkaline earth metal salts include sodium, lithium, potassium, calcium, magnesium, and the like. Further pharmaceutically acceptable salts include, when appropriate, nontoxic ammonium. Quaternary ammonium, and amine cations formed using counterions such as halide, hydroxide, carboxylate, sulfate, phosphate, nitrate, sulfonate, and aryl sulfonate. Further pharmaceutically acceptable salts include salts formed from the quaternization of an amine using an appropriate electrophile, e.g., an alkyl halide, to form a quarternized alkylated amino salt.

Systemic distribution or delivery: As used herein, the terms “systemic distribution” or “systemic delivery,” or grammatical equivalents thereof, refer to a delivery or distribution mechanism or approach that affect the entire body or an entire organism. Typically, systemic distribution or delivery is accomplished via body's circulation system, e.g., blood stream. Compared to the definition of “local distribution or delivery.”

Subject: As used herein, the term “subject” refers to a human or any non-human animal (e.g., mouse, rat, rabbit, dog, cat, cattle, swine, sheep, horse or primate). A human includes pre- and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Target tissues: As used herein, the term “target tissues” refers to any tissue that is affected by a disease to be treated. In some embodiments, target tissues include those tissues that display disease-associated pathology, symptom, or feature.

Therapeutically effective amount: As used herein, the term “therapeutically effective amount” of a therapeutic agent means an amount that is sufficient, when administered to a subject suffering from or susceptible to a disease, disorder, and/or condition, to treat, diagnose, prevent, and/or delay the onset of the symptom(s) of the disease, disorder, and/or condition. It will be appreciated by those of ordinary skill in the art that a therapeutically effective amount is typically administered via a dosing regimen comprising at least one unit dose.

Treating: As used herein, the term “treat,” “treatment,” or “treating” refers to any method used to partially or completely alleviate, ameliorate, relieve, inhibit, prevent, delay onset of, reduce severity of and/or reduce incidence of one or more symptoms or features of a particular disease, disorder, and/or condition. Treatment may be administered to a subject who does not exhibit signs of a disease and/or exhibits only early signs of the disease for the purpose of decreasing the risk of developing pathology associated with the disease.

### Chemical Definitions

Acyl: As used herein, the term “acyl” refers to R^{Z}—(C═O)—, wherein R_{Z} is, for example, any alkyl, alkenyl, alkynyl, heteroalkyl or heteroalkylene.

Aliphatic: As used herein, the term aliphatic refers to (C₁-C₅₀) hydrocarbons and includes both saturated and unsaturated hydrocarbons. An aliphatic may be linear, branched, or cyclic. For example, (C₁-C₂₀) aliphatics can include (C₁-C₂₀) alkyls (e.g., linear or branched (C₁-C₂₀) saturated alkyls), (C₂-C₂₀) alkenyls (e.g., linear or branched (C₄-C₂₀) dienyls, linear or branched (C₆-C₂₀) trienyls, and the like), and (C₂-C₂₀) alkynyls (e.g., linear or branched (C₂-C₂₀) alkynyls). (C₁-C₂₀) aliphatics can include (C₃-C₂₀) cyclic aliphatics (e.g., (C₃-C₂₀) cycloalkyls, (C₄-C₂₀) cycloalkenyls, or (C₈-C₂₀) cycloalkynyls). In certain embodiments, the aliphatic may comprise one or more cyclic aliphatic and/or one or more heteroatoms such as oxygen, nitrogen, or sulfur and may optionally be substituted with one or more substituents such as alkyl, halo, alkoxyl, hydroxy, amino, aryl, ether, ester or amide. An aliphatic group is unsubstituted or substituted with one or more substituent groups as described herein. For example, an aliphatic may be substituted with one or more (e.g., 1, 2, 3, 4, 5, or 6 independently selected substituents) of halogen, —COR″, —CO₂H, —CO₂R″, —CN, —OH, —OR″, —OCOR″, —OCO₂R″, —NH₂, —NHR″, —N(R^{cc})₂, —SR″ or —SO₂R″, wherein each instance of R″ independently is (C₁-C₂₀) aliphatic (e.g., (C₁-C₂₀) alkyl, (C₁-C₁₅) alkyl, (C₁-C₁₀) alkyl, or (C₁-C₃)alkyl). In embodiments, R″ independently is an unsubstituted alkyl (e.g., unsubstituted (C₁-C₂₀) alkyl, (C₁-C₁₅) alkyl, (C₁-C₁₀) alkyl, or (C₁-C₃)alkyl). In embodiments, R″ independently is unsubstituted (C₁-C₃)alkyl. In embodiments, the aliphatic is unsubstituted. In embodiments, the aliphatic does not include any heteroatoms.

Alkyl: As used herein, the term “alkyl” means acyclic linear and branched hydrocarbon groups, e.g. “(C₁-C₃₀) alkyl” refers to alkyl groups having 1-30 carbons. An alkyl group may be linear or branched. Examples of alkyl groups include, but are not limited to, methyl, ethyl, n-propyl, isopropyl, butyl, isobutyl, sec-butyl, tert-butyl, pentyl, isopentyl tert-pentylhexyl, isohexyl, etc. The term “lower alkyl” means an alkyl group straight chain or branched alkyl having 1 to 6 carbon atoms. Other alkyl groups will be readily apparent to those of skill in the art given the benefit of the present disclosure. An alkyl group may be unsubstituted or substituted with one or more substituent groups as described herein. For example, an alkyl group may be substituted with one or more (e.g., 1, 2, 3, 4, 5, or 6 independently selected substituents) of halogen, —COR″, —CO₂H, —CO₂R″, —CN, —OH, —OR″, —OCOR″, —OCO₂R″, —NH₂, —NHR″, —N(R^{cc})₂, —SR″ or —SO₂R″, wherein each instance of R″ independently is (C₁-C₂₀) aliphatic (e.g., (C₁-C₂₀) alkyl, (C₁-C₁₅) alkyl, (C₁-C₁₀) alkyl, or (C₁-C₃)alkyl). In embodiments, R″ independently is an unsubstituted alkyl (e.g., unsubstituted (C₁-C₂₀) alkyl, (C₁-C₁₅) alkyl, (C₁-C₁₀) alkyl, or (C₁-C₃)alkyl). In embodiments, R″ independently is unsubstituted (C₁-C₃)alkyl. In embodiments, the alkyl is substituted (e.g., with 1, 2, 3, 4, 5, or 6 substituent groups as described herein). In embodiments, an alkyl group is substituted with a —OH group and may also be referred to herein as a “hydroxyalkyl” group, where the prefix denotes the —OH group and “alkyl” is as described herein.

As used herein, “alkyl” also refers to a radical of a straight-chain or branched saturated hydrocarbon group having from 1 to 50 carbon atoms (“(C₁-C₅₀) alkyl”). In some embodiments, an alkyl group has 1 to 40 carbon atoms (“(C₁-C₄₀) alkyl”). In some embodiments, an alkyl group has 1 to 30 carbon atoms (“(C₁-C₃₀) alkyl”). In some embodiments, an alkyl group has 1 to 20 carbon atoms (“(C₁-C₂₀) alkyl”). In some embodiments, an alkyl group has 1 to 10 carbon atoms (“(C₁-C₁₀) alkyl”). In some embodiments, an alkyl group has 1 to 9 carbon atoms (“(C₁-C₉)alkyl”). In some embodiments, an alkyl group has 1 to 8 carbon atoms (“(C₁-C₈)alkyl”). In some embodiments, an alkyl group has 1 to 7 carbon atoms (“(C₁-C₇)alkyl”). In some embodiments, an alkyl group has 1 to 6 carbon atoms (“(C₁-C₆)alkyl”). In some embodiments, an alkyl group has 1 to 5 carbon atoms (“(C₁-C₅)alkyl”). In some embodiments, an alkyl group has 1 to 4 carbon atoms (“(C₁-C₄)alkyl”). In some embodiments, an alkyl group has 1 to 3 carbon atoms (“(C₁-C₃)alkyl”). In some embodiments, an alkyl group has 1 to 2 carbon atoms (“(C₁-C₂)alkyl”). In some embodiments, an alkyl group has 1 carbon atom (“C₁ alkyl”). In some embodiments, an alkyl group has 2 to 6 carbon atoms (“(C₂-C₆)alkyl”). Examples of (C₁-C₆)alkyl groups include, without limitation, methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₅) and the like. Unless otherwise specified, each instance of an alkyl group is independently unsubstituted (an “unsubstituted alkyl”) or substituted (a “substituted alkyl”) with one or more substituents. In certain embodiments, the alkyl group is an unsubstituted (C₁-C₅₀) alkyl. In certain embodiments, the alkyl group is a substituted (C₁-C₅₀) alkyl.

Affixing the suffix “-ene” to a group indicates the group is a divalent moiety, e.g., arylene is the divalent moiety of aryl, and heteroarylene is the divalent moiety of heteroaryl.

Alkylene: The term “alkylene,” as used herein, represents a saturated divalent straight or branched chain hydrocarbon group and is exemplified by methylene, ethylene, isopropylene and the like. Likewise, the term “alkenylene” as used herein represents an unsaturated divalent straight or branched chain hydrocarbon group having one or more unsaturated carbon-carbon double bonds that may occur in any stable point along the chain, and the term “alkynylene” herein represents an unsaturated divalent straight or branched chain hydrocarbon group having one or more unsaturated carbon-carbon triple bonds that may occur in any stable point along the chain. In certain embodiments, an alkylene, alkenylene, or alkynylene group may comprise one or more cyclic aliphatic and/or one or more heteroatoms such as oxygen, nitrogen, or sulfur and may optionally be substituted with one or more substituents such as alkyl, halo, alkoxyl, hydroxy, amino, aryl, ether, ester or amide. For example, an alkylene, alkenylene, or alkynylene may be substituted with one or more (e.g., 1, 2, 3, 4, 5, or 6 independently selected substituents) of halogen, —COR″, —CO₂H, —CO₂R″, —CN, —OH, —OR″, —OCOR″, —OCO₂R″, —NH₂, —NHR″, —N(R^{cc})₂, —SR″ or —SO₂R″, wherein each instance of R″ independently is (C₁-C₂₀) aliphatic (e.g., (C₁-C₂₀) alkyl, (C₁-C₁₅) alkyl, (C₁-C₁₀) alkyl, or (C₁-C₃)alkyl). In embodiments, R″ independently is an unsubstituted alkyl (e.g., unsubstituted (C₁-C₂₀) alkyl, (C₁-C₁₅) alkyl, (C₁-C₁₀) alkyl, or (C₁-C₃)alkyl). In embodiments, R″ independently is unsubstituted (C₁-C₃)alkyl. In certain embodiments, an alkylene, alkenylene, or alkynylene is unsubstituted. In certain embodiments, an alkylene, alkenylene, or alkynylene does not include any heteroatoms.

Alkenyl: As used herein, “alkenyl” means any linear or branched hydrocarbon chains having one or more unsaturated carbon-carbon double bonds that may occur in any stable point along the chain, e.g. “(C₂-C₃₀) alkenyl” refers to an alkenyl group having 2-30 carbons. For example, an alkenyl group includes prop-2-enyl, but-2-enyl, but-3-enyl, 2-methylprop-2-enyl, hex-2-enyl, hex-5-enyl, 2,3-dimethylbut-2-enyl, and the like. In embodiments, the alkenyl comprises 1, 2, or 3 carbon-carbon double bond. In embodiments, the alkenyl comprises a single carbon-carbon double bond. In embodiments, multiple double bonds (e.g., 2 or 3) are conjugated. An alkenyl group may be unsubstituted or substituted with one or more substituent groups as described herein. For example, an alkenyl group may be substituted with one or more (e.g., 1, 2, 3, 4, 5, or 6 independently selected substituents) of halogen, —COR″, —CO₂H, —CO₂R″, —CN, —OH, —OR″, —OCOR″, —OCO₂R″, —NH₂, —NHR″, —N(R^{cc})₂, —SR″ or —SO₂R″, wherein each instance of R″ independently is (C₁-C₂₀) aliphatic (e.g., (C₁-C₂₀) alkyl, (C₁-C₁₅) alkyl, (C₁-C₁₀) alkyl, or (C₁-C₃)alkyl). In embodiments, R″ independently is an unsubstituted alkyl (e.g., unsubstituted (C₁-C₂₀) alkyl, (C₁-C₁₅) alkyl, (C₁-C₁₀) alkyl, or (C₁-C₃)alkyl). In embodiments, R″ independently is unsubstituted (C₁-C₃)alkyl. In embodiments, the alkenyl is unsubstituted. In embodiments, the alkenyl is substituted (e.g., with 1, 2, 3, 4, 5, or 6 substituent groups as described herein). In embodiments, an alkenyl group is substituted with a —OH group and may also be referred to herein as a “hydroxyalkenyl” group, where the prefix denotes the —OH group and “alkenyl” is as described herein.

As used herein, “alkenyl” also refers to a radical of a straight-chain or branched hydrocarbon group having from 2 to 50 carbon atoms and one or more carbon-carbon double bonds (e.g., 1, 2, 3, or 4 double bonds) (“(C₂-C₅₀) alkenyl”). In some embodiments, an alkenyl group has 2 to 40 carbon atoms (“(C₂-C₄₀) alkenyl”). In some embodiments, an alkenyl group has 2 to 30 carbon atoms (“(C₂-C₃₀) alkenyl”). In some embodiments, an alkenyl group has 2 to 20 carbon atoms (“(C₂-C₂₀) alkenyl”). In some embodiments, an alkenyl group has 2 to 10 carbon atoms (“(C₂-C₁₀) alkenyl”). In some embodiments, an alkenyl group has 2 to 9 carbon atoms (“(C₂-C₉)alkenyl”). In some embodiments, an alkenyl group has 2 to 8 carbon atoms (“(C₂-C₈)alkenyl”). In some embodiments, an alkenyl group has 2 to 7 carbon atoms (“(C₂-C₇)alkenyl”). In some embodiments, an alkenyl group has 2 to 6 carbon atoms (“(C₂-C₆)alkenyl”). In some embodiments, an alkenyl group has 2 to 5 carbon atoms (“(C₂-C₅)alkenyl”). In some embodiments, an alkenyl group has 2 to 4 carbon atoms (“(C₂-C₄) alkenyl”). In some embodiments, an alkenyl group has 2 to 3 carbon atoms (“(C₂-C₃)alkenyl”). In some embodiments, an alkenyl group has 2 carbon atoms (“(C₂)alkenyl”). The one or more carbon-carbon double bonds can be internal (such as in 2-butenyl) or terminal (such as in 1-butenyl). Examples of (C₂-C₄)alkenyl groups include, without limitation, ethenyl (C₂), 1-propenyl (C₃), 2-propenyl (C₃), 1-butenyl (C₄), 2-butenyl (C₄), butadienyl (C₄), and the like. Examples of (C₂-C₆)alkenyl groups include the aforementioned (C₂-C₄)alkenyl groups as well as pentenyl (C₅), pentadienyl (C₅), hexenyl (C₆), and the like. Additional examples of alkenyl include heptenyl (C₇), octenyl (C₅), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted (C₂-C₅₀) alkenyl. In certain embodiments, the alkenyl group is a substituted (C₂-C₅₀) alkenyl.

Alkynyl: As used herein, “alkynyl” means any hydrocarbon chain of either linear or branched configuration, having one or more carbon-carbon triple bonds occurring in any stable point along the chain, e.g., “(C₂-C₃₀) alkynyl”, refers to an alkynyl group having 2-30 carbons. Examples of an alkynyl group include prop-2-ynyl, but-2-ynyl, but-3-ynyl, pent-2-ynyl, 3-methylpent-4-ynyl, hex-2-ynyl, hex-5-ynyl, etc. In embodiments, an alkynyl comprises one carbon-carbon triple bond. An alkynyl group may be unsubstituted or substituted with one or more substituent groups as described herein. For example, an alkynyl group may be substituted with one or more (e.g., 1, 2, 3, 4, 5, or 6 independently selected substituents) of halogen, —COR″, —CO₂H, —CO₂R″, —CN, —OH, —OR″, —OCOR″, —OCO₂R″, —NH₂, —NHR″, —N(R^{cc})₂, —SR″ or —SO₂R″, wherein each instance of R″ independently is (C₁-C₂₀) aliphatic (e.g., (C₁-C₂₀) alkyl, (C₁-C₁₅) alkyl, (C₁-C₁₀) alkyl, or (C₁-C₃)alkyl). In embodiments, R″ independently is an unsubstituted alkyl (e.g., unsubstituted (C₁-C₂₀) alkyl, (C₁-C₁₅) alkyl, (C₁-C₁₀) alkyl, or (C₁-C₃)alkyl). In embodiments, R″ independently is unsubstituted (C₁-C₃)alkyl. In embodiments, the alkynyl is unsubstituted. In embodiments, the alkynyl is substituted (e.g., with 1, 2, 3, 4, 5, or 6 substituent groups as described herein).

As used herein, “alkynyl” also refers to a radical of a straight-chain or branched hydrocarbon group having from 2 to 50 carbon atoms and one or more carbon-carbon triple bonds (e.g., 1, 2, 3, or 4 triple bonds) and optionally one or more double bonds (e.g., 1, 2, 3, or 4 double bonds) (“(C₂-C₅₀) alkynyl”). An alkynyl group that has one or more triple bonds, and one or more double bonds is also referred to as an “ene-yne”. In some embodiments, an alkynyl group has 2 to 40 carbon atoms (“(C₂-C₄₀) alkynyl”). In some embodiments, an alkynyl group has 2 to 30 carbon atoms (“(C₂-C₃₀) alkynyl”). In some embodiments, an alkynyl group has 2 to 20 carbon atoms (“(C₂-C₂₀) alkynyl”). In some embodiments, an alkynyl group has 2 to 10 carbon atoms (“(C₂-C₁₀) alkynyl”). In some embodiments, an alkynyl group has 2 to 9 carbon atoms (“(C₂-C₉)alkynyl”). In some embodiments, an alkynyl group has 2 to 8 carbon atoms (“(C₂-C₈)alkynyl”). In some embodiments, an alkynyl group has 2 to 7 carbon atoms (“(C₂-C₇)alkynyl”). In some embodiments, an alkynyl group has 2 to 6 carbon atoms (“(C₂-C₆)alkynyl”). In some embodiments, an alkynyl group has 2 to 5 carbon atoms (“(C₂-C₅) alkynyl”). In some embodiments, an alkynyl group has 2 to 4 carbon atoms (“(C₂-C₄)alkynyl”). In some embodiments, an alkynyl group has 2 to 3 carbon atoms (“(C₂-C₃)alkynyl”). In some embodiments, an alkynyl group has 2 carbon atoms (“(C₂)alkynyl”). The one or more carbon-carbon triple bonds can be internal (such as in 2-butynyl) or terminal (such as in 1-butynyl). Examples of (C₂-C₄)alkynyl groups include, without limitation, ethynyl (C₂), 1-propynyl (C₃), 2-propynyl (C₃), 1-butynyl (C₄), 2-butynyl (C₄), and the like. Examples of (C₂-C₆)alkenyl groups include the aforementioned (C₂-C₄)alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted (C₂-C₅₀) alkynyl. In certain embodiments, the alkynyl group is a substituted (C₂-C₅₀) alkynyl.

Aryl: The term “aryl” used alone or as part of a larger moiety as in “aralkyl,” refers to a monocyclic, bicyclic, or tricyclic carbocyclic ring system having a total of six to fourteen ring members, wherein said ring system has a single point of attachment to the rest of the molecule, at least one ring in the system is aromatic and wherein each ring in the system contains 4 to 7 ring members. In embodiments, an aryl group has 6 ring carbon atoms (“(C₆) aryl,” e.g., phenyl). In some embodiments, an aryl group has 10 ring carbon atoms (“(C₁₀) aryl,” e.g., naphthyl such as 1-naphthyl and 2-naphthyl). In some embodiments, an aryl group has 14 ring carbon atoms (“(C₁₄) aryl,” e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Exemplary aryls include phenyl, naphthyl, and anthracene.

As used herein, “aryl” also refers to a radical of a monocyclic or polycyclic (e.g., bicyclic or tricyclic) 4n+2 aromatic ring system (e.g., having 6, 10, or 14 It electrons shared in a cyclic array) having 6-14 ring carbon atoms and zero heteroatoms provided in the aromatic ring system (“(C₆-C₁₄) aryl”). In some embodiments, an aryl group has 6 ring carbon atoms (“(C₆) aryl”; e.g., phenyl). In some embodiments, an aryl group has 10 ring carbon atoms (“(C₁₀) aryl”; e.g., naphthyl such as 1-naphthyl and 2-naphthyl). In some embodiments, an aryl group has 14 ring carbon atoms (“(C₁₄) aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted (C₆-C₁₄) aryl. In certain embodiments, the aryl group is a substituted (C₆-C₁₄) aryl.

Arylene: The term “arylene” as used herein refers to an aryl group that is divalent (that is, having two points of attachment to the molecule). Exemplary arylenes include phenylene (e.g., unsubstituted phenylene or substituted phenylene).

Carbocyclyl: As used herein, “carbocyclyl” or “carbocyclic” refers to a radical of a non-aromatic cyclic hydrocarbon group having from 3 to 10 ring carbon atoms (“(C₃-C₁₀) carbocyclyl”) and zero heteroatoms in the non-aromatic ring system. In some embodiments, a carbocyclyl group has 3 to 8 ring carbon atoms (“(C₃-C₈) carbocyclyl”). In some embodiments, a carbocyclyl group has 3 to 7 ring carbon atoms (“(C₃-C₇) carbocyclyl”). In some embodiments, a carbocyclyl group has 3 to 6 ring carbon atoms (“(C₃-C₆) carbocyclyl”). In some embodiments, a carbocyclyl group has 4 to 6 ring carbon atoms (“(C₄-C₆) carbocyclyl”). In some embodiments, a carbocyclyl group has 5 to 6 ring carbon atoms (“(C₅-C₆) carbocyclyl”). In some embodiments, a carbocyclyl group has 5 to 10 ring carbon atoms (“(C₅-C₁₀) carbocyclyl”). Exemplary (C₃-C₆) carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary (C₃-C₈) carbocyclyl groups include, without limitation, the aforementioned (C₃-C₆) carbocyclyl groups as well as cycloheptyl (C₇), cycloheptenyl (C₇), cycloheptadienyl (C₇), cycloheptatrienyl (C₇), cyclooctyl (C₅), cyclooctenyl (C₈), bicyclo[2.2.1]heptanyl (C₇), bicyclo[2.2.2]octanyl (C₈), and the like. Exemplary (C₃-C₁₀) carbocyclyl groups include, without limitation, the aforementioned (C₃-C₈) carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃-C₁₀ carbocyclyl. In certain embodiments, the carbocyclyl group is a substituted (C₃-C₁₀) carbocyclyl.

In some embodiments, “carbocyclyl” or “carbocyclic” is referred to as a “cycloalkyl”, i.e., a monocyclic, saturated carbocyclyl group having from 3 to 10 ring carbon atoms (“(C₃-C₁₀) cycloalkyl”). In some embodiments, a cycloalkyl group has 3 to 8 ring carbon atoms (“(C₃-C₈) cycloalkyl”). In some embodiments, a cycloalkyl group has 3 to 6 ring carbon atoms (“(C₃-C₆), cycloalkyl”). In some embodiments, a cycloalkyl group has 4 to 6 ring carbon atoms (“(C₄-C₆) cycloalkyl”). In some embodiments, a cycloalkyl group has 5 to 6 ring carbon atoms (“(C₅-C₆) cycloalkyl”). In some embodiments, a cycloalkyl group has 5 to 10 ring carbon atoms (“(C₅-C₁₀) cycloalkyl”). Examples of (C₅-C₆) cycloalkyl groups include cyclopentyl (C₅) and cyclohexyl (C₅). Examples of (C₃-C₆) cycloalkyl groups include the aforementioned (C₅-C₆) cycloalkyl groups as well as cyclopropyl (C₃) and cyclobutyl (C₄). Examples of (C₃-C₈) cycloalkyl groups include the aforementioned (C₃-C₆) cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted (C₃-C₁₀) cycloalkyl. In certain embodiments, the cycloalkyl group is a substituted (C₃-C₁₀) cycloalkyl.

Heteroalkyl: The term “heteroalkyl” is meant a branched or unbranched alkyl, alkenyl, or alkynyl group having from 1 to 14 carbon atoms in addition to 1, 2, 3 or 4 heteroatoms independently selected from the group consisting of N, O, S, and P. Heteroalkyls include tertiary amines, secondary amines, ethers, thioethers, amides, thioamides, carbamates, thiocarbamates, hydrazones, imines, phosphodiesters, phosphoramidates, sulfonamides, and disulfides. A heteroalkyl group may optionally include monocyclic, bicyclic, or tricyclic rings, in which each ring desirably has three to six members. Examples of heteroalkyls include polyethers, such as methoxymethyl and ethoxyethyl.

Heteroalkylene: The term “heteroalkylene,” as used herein, represents a divalent form of a heteroalkyl group as described herein.

Heteroaryl: The term “heteroaryl,” as used herein, represents a fully unsaturated heteroatom-containing ring wherein at least one ring atom is a heteroatom such as, but not limited to, nitrogen and oxygen.

As used herein, “heteroaryl” also refers to a radical of a 5-14 membered monocyclic or polycyclic (e.g., bicyclic or tricyclic) 4n+2 aromatic ring system (e.g., having 6, 10, or 14 It electrons shared in a cyclic array) having ring carbon atoms and 1 or more (e.g., 1, 2, 3, or 4 ring heteroatoms) ring heteroatoms provided in the aromatic ring system, wherein each heteroatom is independently selected from oxygen, sulfur, nitrogen, boron, silicon, and phosphorus (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system.

“Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1 or more (e.g., 1, 2, 3, or 4) ring heteroatoms provided in the aromatic ring system, wherein each heteroatom is independently selected from oxygen, sulfur, nitrogen, boron, silicon, and phosphorus (“5-10 membered heteroaryl”). In some embodiments, a heteroaryl group is a 5-8 membered aromatic ring system having ring carbon atoms and 1 or more (e.g., 1, 2, 3, or 4) ring heteroatoms provided in the aromatic ring system, wherein each heteroatom is independently selected from oxygen, sulfur, nitrogen, boron, silicon, and phosphorus (“5-8 membered heteroaryl”). In some embodiments, a heteroaryl group is a 5-6 membered aromatic ring system having ring carbon atoms and 1 or more (e.g., 1, 2, 3, or 4) ring heteroatoms provided in the aromatic ring system, wherein each heteroatom is independently selected from oxygen, sulfur, nitrogen, boron, silicon, and phosphorus (“5-6 membered heteroaryl”). In some embodiments, the 5-6 membered heteroaryl has 1 or more (e.g., 1, 2, or 3) ring heteroatoms selected from oxygen, sulfur, nitrogen, boron, silicon, and phosphorus. In some embodiments, the 5-6 membered heteroaryl has 1 or 2 ring heteroatoms selected from oxygen, sulfur, nitrogen, boron, silicon, and phosphorus. In some embodiments, the 5-6 membered heteroaryl has 1 ring heteroatom selected from oxygen, sulfur, nitrogen, boron, silicon, and phosphorus. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include, without limitation, pyrrolyl, furanyl and thiophenyl. Exemplary 5-membered heteroaryl groups containing 2 heteroatoms include, without limitation, imidazolyl, pyrazolyl, oxazolyl, isoxazolyl, thiazolyl, and isothiazolyl. Exemplary 5-membered heteroaryl groups containing 3 heteroatoms include, without limitation, triazolyl, oxadiazolyl, and thiadiazolyl. Exemplary 5-membered heteroaryl groups containing 4 heteroatoms include, without limitation, tetrazolyl. Exemplary 6-membered heteroaryl groups containing 1 heteroatom include, without limitation, pyridinyl. Exemplary 6-membered heteroaryl groups containing 2 heteroatoms include, without limitation, pyridazinyl, pyrimidinyl, and pyrazinyl. Exemplary 6-membered heteroaryl groups containing 3 or 4 heteroatoms include, without limitation, triazinyl and tetrazinyl, respectively. Exemplary 7-membered heteroaryl groups containing 1 heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary tricyclic heteroaryl groups include, without limitation, phenanthridinyl, dibenzofuranyl, carbazolyl, acridinyl, phenothiazinyl, phenoxazinyl and phenazinyl.

As used herein, “heterocyclyl” or “heterocyclic” refers to a radical of a 3- to 14-membered non-aromatic ring system having ring carbon atoms and 1 or more (e.g., 1, 2, 3, or 4) ring heteroatoms, wherein each heteroatom is independently selected from oxygen, sulfur, nitrogen, boron, silicon, and phosphorus (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)) and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl.

In some embodiments, a heterocyclyl group is a 5-10 membered non-aromatic ring system having ring carbon atoms and 1 or more (e.g., 1, 2, 3, or 4) ring heteroatoms, wherein each heteroatom is independently selected from oxygen, sulfur, nitrogen, boron, silicon, and phosphorus (“5-10 membered heterocyclyl”). In some embodiments, a heterocyclyl group is a 5-8 membered non-aromatic ring system having ring carbon atoms and 1 or more (e.g., 1, 2, 3, or 4) ring heteroatoms, wherein each heteroatom is independently selected from oxygen, sulfur, nitrogen, boron, silicon, and phosphorus (“5-8 membered heterocyclyl”). In some embodiments, a heterocyclyl group is a 5-6 membered non-aromatic ring system having ring carbon atoms and 1 or more (e.g., 1, 2, 3, or 4) ring heteroatoms, wherein each heteroatom is independently selected from oxygen, sulfur, nitrogen, boron, silicon, and phosphorus (“5-6 membered heterocyclyl”). In some embodiments, the 5-6 membered heterocyclyl has 1 or more (e.g., 1, 2, or 3) ring heteroatoms selected from oxygen, sulfur, nitrogen, boron, silicon, and phosphorus. In some embodiments, the 5-6 membered heterocyclyl has 1 or 2 ring heteroatoms selected from oxygen, sulfur, nitrogen, boron, silicon, and phosphorus. In some embodiments, the 5-6 membered heterocyclyl has 1 ring heteroatom selected from oxygen, sulfur, nitrogen, boron, silicon, and phosphorus.

Exemplary 3-membered heterocyclyl groups containing 1 heteroatom include, without limitation, azirdinyl, oxiranyl, thiorenyl. Exemplary 4-membered heterocyclyl groups containing 1 heteroatom include, without limitation, azetidinyl, oxetanyl and thietanyl. Exemplary 5-membered heterocyclyl groups containing 1 heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing 2 heteroatoms include, without limitation, dioxolanyl, oxathiolanyl and dithiolanyl. Exemplary 5-membered heterocyclyl groups containing 3 heteroatoms include, without limitation, triazolinyl, oxadiazolinyl, and thiadiazolinyl. Exemplary 6-membered heterocyclyl groups containing 1 heteroatom include, without limitation, piperidinyl, tetrahydropyranyl, dihydropyridinyl, and thianyl. Exemplary 6-membered heterocyclyl groups containing 2 heteroatoms include, without limitation, piperazinyl, morpholinyl, dithianyl, dioxanyl. Exemplary 6-membered heterocyclyl groups containing 2 heteroatoms include, without limitation, triazinanyl. Exemplary 7-membered heterocyclyl groups containing 1 heteroatom include, without limitation, azepanyl, oxepanyl and thiepanyl. Exemplary 8-membered heterocyclyl groups containing 1 heteroatom include, without limitation, azocanyl, oxecanyl and thiocanyl. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo-[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

Heterocycloalkyl: The term “heterocycloalkyl,” as used herein, is a non-aromatic ring wherein at least one atom is a heteroatom such as, but not limited to, nitrogen, oxygen, sulfur, or phosphorus, and the remaining atoms are carbon. The heterocycloalkyl group can be substituted or unsubstituted.

As understood from the above, alkyl, alkenyl, alkynyl, acyl, carbocyclyl, heterocyclyl, aryl, and heteroaryl groups, as defined herein, are, in certain embodiments, optionally substituted. Optionally substituted refers to a group which may be substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or ‘unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group. In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valences of the heteroatoms and results in the formation of a stable moiety.

Exemplary carbon atom substituents include, but are not limited to, halogen, —CN, —NO₂, —N₃, —SO₂, —SO₃H, —OH, —OR^{aa}, —ON(R^{bb})₂, —N(R^{bb})₂, —N(R^{bb})₃+X⁻, —N(OR^{cc})R^{bb}, —SeH, —SeR^{aa}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃—Osi (R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)₂R^{aa}, —OP(═O)₂R^{aa}, —P(═O) (R^{aa})₂, —OP(═O) (R^{aa})₂, —OP(═O)(OR^{cc})₂, —P(═O)₂N(R^{bb})₂, —OP(═O)₂N(R^{bb})₂, —P(═O)(NR^{bb})₂, —OP(═O)(NR^{bb})₂, —NR^{bb}P(═O)(OR^{cc})₂, —NR^{bb}P(═O)(NR^{bb})₂, —P(R^{cc})₂, —P(R^{cc})₃, —OP(R^{cc})₂, —OP(R^{cc})₃, —B(R^{aa})₂, —B(OR^{cc})₂, —BR^{aa}(OR^{cc}), (C₁-C₅₀) alkyl, (C₂-C₅₀) alkenyl, (C₂-C₅₀) alkynyl, (C₃-C₁₄) carbocyclyl, 3-14 membered heterocyclyl, (C₆-C₁₄) aryl, and 5-14 membered heteroaryl, wherein each alkyl, alkenyl, alkynyl, carbocyclyl, heterocyclyl, aryl, and heteroaryl is independently substituted with 0, 1, 2, 3, 4, or 5 R^{dd} groups;
or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};
each instance of R^{aa} is, independently, selected from (C₁-C₅₀) alkyl, (C₂-C₅₀) alkenyl, (C₂-C₅₀) alkynyl, (C₃-C₁₀) carbocyclyl, 3-14 membered heterocyclyl, (C₆-C₁₄) aryl, and 5-14 membered heteroaryl, or two R^{aa} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, (C₁-C₅₀) alkyl, (C₂-C₅₀) alkenyl, (C₂-C₅₀) alkynyl, (C₃-C₁₀) carbocyclyl, 3-14 membered heterocyclyl, (C₆-C₁₄) aryl, and 5-14 membered heteroaryl, or two R^{bb} groups, together with the heteroatom to which they are attached, form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;
each instance of R^{cc} is, independently, selected from hydrogen, (C₁-C₅₀) alkyl, (C₂-C₅₀) alkenyl, (C₂-C₅₀) alkynyl, (C₃-C₁₀) carbocyclyl, 3-14 membered heterocyclyl, (C₆-C₁₄) aryl, and 5-14 membered heteroaryl, or two R^{cc} groups, together with the heteroatom to which they are attached, form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;

each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃+X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}(═O)R^{ee}, —NR^{ff}CO₂R^{ee}, —NR^{ff}C(═O)N(R^{ff})₂, —C(═NR^{ff})OR^{ee}, —OC(═NR^{ff}) R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O) R^{ee}, —Si(R^{ee})₃, —Osi(R^{ee})₃, —C(═S)N(R^{ff})₂, —C(═O)SR^{ee}, —C(═S)SR^{ee}, —SC(═S)SR^{ee}, —P(═O)₂R^{ee}, —P(═O)(R^{ee})₂, —OP(═O)(R^{ee})₂, —OP(═O)(OR^{ee})₂, (C₁-C₅₀) alkyl, (C₂-C₅₀) alkenyl, (C₂-C₅₀) alkynyl, (C₃-C₁₀) carbocyclyl, 3-10 membered heterocyclyl, (C₆-C₁₀) aryl, 5-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S;
each instance of R^{ee} is, independently, selected from (C₁-C₅₀) alkyl, (C₂-C₅₀) alkenyl, (C₂-C₅₀) alkynyl, (C₃-C₁₀) carbocyclyl, (C₆-C₁₀) aryl, 3-10 membered heterocyclyl, and 3-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups;
each instance of R^{ff} is, independently, selected from hydrogen, (C₁-C₅₀) alkyl, (C₂-C₅₀) alkenyl, (C₂-C₅₀) alkynyl, (C₃-C₁₀) carbocyclyl, 3-10 membered heterocyclyl, (C₆-C₁₀) aryl and 5-10 membered heteroaryl, or two R^{ff} groups, together with the heteroatom to which they are attached, form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{gg} groups; and
each instance of R^{gg} is, independently, halogen, —CN, —NO₂, —N₃, —SO₂H, —SO₃H, —OH, —O(C₁-C₅₀) alkyl, —ON((C₁-C₅₀) alkyl)₂, —N((C₁-C₅₀) alkyl)₂, —N((C₁-C₅₀) alkyl)₃+X⁻, —NH((C₁-C₅₀) alkyl)₂+X⁻, —NH₂ ((C₁-C₅₀) alkyl)+X⁻, —NH₃+X⁻, —N(O(C₁-C₅₀) alkyl)((C₁-C₅₀) alkyl), —N(OH)((C₁-C₅₀) alkyl), —NH(OH), —SH, —S(C₁-C₅₀) alkyl, —SS((C₁-C₅₀) alkyl), —C(═O)((C₁-C₅₀) alkyl), —CO₂H, —CO₂ ((C₁-C₅₀) alkyl), —OC(═O)((C₁-C₅₀) alkyl), —OCO₂ ((C₁-C₅₀) alkyl), —C(═O)NH₂, —C(═O)N((C₁-C₅₀) alkyl)₂, —OC(═O)NH((C₁-C₅₀) alkyl), —NHC(═O)((C₁-C₅₀) alkyl), —N((C₁-C₅₀) alkyl) C(═O)((C₁-C₅₀) alkyl), —NHCO₂ ((C₁-C₅₀) alkyl), —NHC(═O)N((C₁-C₅₀) alkyl)₂, —NHC(═O)NH((C₁-C₅₀) alkyl), —NHC(═O)NH₂, —C(═NH)O((C₁-C₅₀) alkyl), —OC(═NH)((C₁-C₅₀) alkyl), —OC(═NH) O(C₁-C₅₀) alkyl, —C(═NH)N((C₁-C₅₀) alkyl)₂, —C(═NH)NH((C₁-C₅₀) alkyl), —C(═NH)NH₂, —OC(═NH)N((C₁-C₅₀) alkyl)₂, —OC(NH)NH((C₁-C₅₀) alkyl), —OC(NH)NH₂, —NHC(NH)N((C₁-C₅₀) alkyl)₂, —NHC(═NH)NH₂, —NHSO₂((C₁-C₅₀) alkyl), —SO₂N((C₁-C₅₀) alkyl)₂, —SO₂NH((C₁-C₅₀) alkyl), —SO₂NH₂, —SO₂(C₁-C₅₀) alkyl), —SO₂O((C₁-C₅₀) alkyl), —OSO₂(C₁-C₆) alkyl), —SO((C₁-C₅)alkyl), —Si((C₁-C₅₀) alkyl)₃, —Osi((C₁-C₆)alkyl)₃, —C(═S)N((C₁-C₅₀) alkyl)₂, C(═S)NH((C₁-C₅₀) alkyl), C(═S)NH₂, —C(═O)S((C₁-C₆)alkyl), —C(═S)S((C₁-C₆)alkyl), —SC(═S)S((C₁-C₆)alkyl), —P(═O)₂((C₁-C₅₀) alkyl), —P(═O)((C₁-C₅₀) alkyl)₂, —OP(═O)((C₁-C₅₀) alkyl)₂, —OP(═O)(O(C₁-C₅₀) alkyl)₂, (C₁-C₅₀) alkyl, (C₂-C₅₀) alkenyl, (C₂-C₅₀) alkynyl, (C₃-C₁₀) carbocyclyl, (C₆-C₁₀) aryl, 3-10 membered heterocyclyl, 5-10 membered heteroaryl; or two geminal R^{gg} substituents can be joined to form ═O or ═S; wherein X is a counterion.

As used herein, the term “halo” or “halogen” refers to fluorine (fluoro, —F), chlorine (chloro, —Cl), bromine (bromo, —Br), or iodine (iodo, —I).

As used herein, a “counterion” is a negatively charged group associated with a positively charged quaternary amine in order to maintain electronic neutrality. Exemplary counterions include halide ions (e.g., F⁻, Cl⁻, Br⁻, I⁻), NO₃⁻, ClO₄⁻, OH⁻, H₂PO₄⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-I-sulfonic acid-5-sulfonate, ethan-1-sulfonic acid-2-sulfonate, and the like), and carboxylate ions (e.g., acetate, ethanoate, propanoate, benzoate, glycerate, lactate, tartrate, glycolate, and the like).

Nitrogen atoms can be substituted or unsubstituted as valency permits, and include primary, secondary, tertiary, and quaternary nitrogen atoms. Exemplary nitrogen atom substituents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}C, —C(═S)SR^{cc}C, —P(═O)₂R^{aa}, —P(═O)(R^{aa})₂, —P(═O)₂N(R^{cc})₂, —P(═O)(NR^{cc})₂, (C₁-C₅₀) alkyl, (C₂-C₅₀) alkenyl, (C₂-C₅₀) alkynyl, (C₃-C₁₀) carbocyclyl, 3-14 membered heterocyclyl, (C₆-C₁₄) aryl, and 5-14 membered heteroaryl, or two R^{cc} groups, together with the N atom to which they are attached, form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, the substituent present on a nitrogen atom is a nitrogen protecting group (also referred to as an amino protecting group). Nitrogen protecting groups are well known in the art and include those described in detail in Protecting Groups in Organic Synthesis, T. W. Greene and P. G. M. Wuts, 3rd edition, John Wiley & Sons, 1999, incorporated herein by reference.

For example, nitrogen protecting groups such as amide groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxyacylamino) acetamide, 3-(p-hydroxyphenyl) propanamide, 3-(o-nitrophenyl) propanamide, 2-methyl-2-(o-nitrophenoxy) propanamide, 2-methyl-2-(o-phenylazophenoxy) propanamide, 4-chlorobutanamide, 3-methyl-3-nitrobutanamide, o-nitrocinnamide, N-acetylmethionine derivative, o-nitrobenzamide and o-(benzoyloxymethyl)benzamide.

Nitrogen protecting groups such as carbamate groups (e.g., —C(═O)OR^{aa}) include, but are not limited to, methyl carbamate, ethyl carbamate, 9-fluorenylmethyl carbamate (Fmoc), 9-(2-sulfo) fluorenylmethyl carbamate, 9-(2,7-dibromo) fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl (o-nitrophenyl) methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido) benzyl carbamate, 1,1-dimethyl-3-(N,N-dimethylcarboxamido) propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-I-cyclopropylmethyl carbamate, 1-methyl-1 (3,5-dimethoxyphenyl)ethyl carbamate, 1-methyl-1-(p-phenylazophenyl)ethyl carbamate, 1-methyl-I-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Nitrogen protecting groups such as sulfonamide groups (e.g., —S(═O)₂R^{aa}) include, but are not limited to, p-toluenesulfonamide (Ts), benzenesulfonamide, 2,3,6, -trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), B-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl) ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl) mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-I-cyclohexenyl)amine, N-borane derivative, N-diphenylborinic acid derivative, N-[phenyl(pentaacylchromium- or tungsten) acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

In certain embodiments, the substituent present on an oxygen atom is an oxygen protecting group (also referred to as a hydroxyl protecting group). Oxygen protecting groups are well known in the art and include those described in detail in Protecting Groups in Organic Synthesis, T. W. Greene and P. G. M. Wuts, 3rd edition, John Wiley & Sons, 1999, incorporated herein by reference.

Exemplary oxygen protecting groups include, but are not limited to, methyl, methoxylmethyl (MOM), methylthiomethyl (MTM), t-butylthiomethyl, (phenyldimethylsilyl) methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl) ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-I-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, a-naphthyldiphenylmethyl, p-methoxyphenyldiphenylmethyl, di(p-methoxyphenyl)phenylmethyl, tri (p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl) xanthenyl, 9-(9-phenyl-10-oxo) anthryl, 1,3-benzodisulfuran-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio) pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl)ethyl carbonate (Psec), 2-(triphenylphosphonio)ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy) butyrate, 2-(methylthiomethoxymethyl)benzoate, 2,6-dichloro-4-methylphenoxyacetate, 2,6-dichloro-4-(1,1,3,3-tetramethylbutyl) phenoxyacetate, 2,4-bis(1,1-dimethylpropyl) phenoxyacetate, chlorodiphenylacetate, isobutyrate, monosuccinoate, (E)-2-methyl-2-butenoate, o-(methoxyacyl)benzoate, a-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, the substituent present on a sulfur atom is a sulfur protecting group (also referred to as a thiol protecting group). Sulfur protecting groups are well known in the art and include those described in detail in Protecting Groups in Organic Synthesis, T. W. Greene and P. G. M. Wuts, 3rd edition, John Wiley & Sons, 1999, incorporated herein by reference.

Exemplary sulfur protecting groups include, but are not limited to, alkyl, benzyl, p-methoxybenzyl, 2,4,6-trimethylbenzyl, 2,4,6-trimethoxybenzyl, o-hydroxybenzyl, p-hydroxybenzyl, o-acetoxybenzyl, p-acetoxybenzyl, p-nitrobenzyl, 4-picolyl, 2-quinolinylmethyl, 2-picolyl N-oxido, 9-anthrylmethyl, 9-fluorenylmethyl, xanthenyl, ferrocenylmethyl, diphenylmethyl, bis(4-methoxyphenyl)methyl, 5-dibenzosuberyl, triphenylmethyl, diphenyl-4-pyridylmethyl, phenyl, 2,4-dinitrophenyl, t-butyl, 1-adamantyl, methoxymethyl (MOM), isobutoxymethyl, benzyloxymethyl, 2-tetrahydropyranyl, benzylthiomethyl, phenylthiomethyl, thiazolidino, acetamidomethyl, trimethylacetamidomethyl, benzamidomethyl, allyloxycarbonylaminomethyl, phenylacetamidomethyl, phthalimidomethyl, acetylmethyl, carboxymethyl, cyanomethyl, (2-nitro-1-phenyl)ethyl, 2-(2,4-dinitrophenyl)ethyl, 2-cyanoethyl, 2-(Trimethylsilyl)ethyl, 2,2-bis(carboethoxy)ethyl, (1-m-nitrophenyl-2-benzoyl)othyl, 2-phenylsulfonylethyl, 2-(4-methylphenylsulfonyl)-2-methylprop-2-yl, acetyl, benzoyl, trifluoroacetyl, N-[[(p-biphenylyl)isopropoxy]carbonyl]-N-methyl]-γ-aminothiobutyrate, 2,2,2-trichloroethoxycarbonyl, t-butoxycarbonyl, benzyloxycarbonyl, p-methoxybenzyloxycarbonyl, N-ethyl, N-methoxymethyl, sulfonate, sulfenylthiocarbonate, 3-nitro-2-pyridinesulfenyl sulfide, oxathiolone.

### Compounds of the Invention

Liposomal-based vehicles are considered as an attractive carrier for therapeutic agents and remain subject to continued development efforts. While liposomal-based vehicles that comprise certain lipid components have shown promising results with regard to encapsulation, stability and site localization, there remains a great need for improvement of liposomal-based delivery systems. For example, a significant drawback of liposomal delivery systems relates to the construction of liposomes that have sufficient cell culture or in vivo stability to reach desired target cells and/or intracellular compartments, and the ability of such liposomal delivery systems to efficiently release their encapsulated materials to such target cells.

In particular, there remains a need for cationic lipids that are effective for intramuscular delivery of mRNA (e.g., for preventing or treating Flu, acne, chlamydia, or a disease or disorder induced by infection with P. gingivalis). There also remains a need for improved lipids compounds that demonstrate improved pharmacokinetic properties, and which are capable of delivering macromolecules, such as nucleic acids, to a wide variety cell types and tissues with enhanced efficiency. Importantly, there also remains a particular need for novel lipid compounds that are characterized as having improved safety profiles and are capable of efficiently delivering encapsulated nucleic acids and polynucleotides to targeted cells, tissues and organs.

Described herein is a novel class of cationic lipid compounds for improved in vivo delivery of therapeutic agents, such as nucleic acids (e.g., for treating or preventing Flu, acne, chlamydia, or a disease or disorder induced by infection with P. gingivalis). In particular, a cationic lipid described herein may be used, optionally with other lipids, to formulate a lipid-based nanoparticle (e.g., liposome) for encapsulating therapeutic agents, such as nucleic acids (e.g., DNA, siRNA, mRNA, microRNA) for therapeutic use, such as disease treatment and prevention (e.g. for use as a vaccine e.g., for Flu, or for the prevention or treatment of acne, chlamydia, or a disease or disorder induced by infection with P. gingivalis) purposes.

In embodiments, compounds of the invention as described herein can provide one or more desired characteristics or properties. That is, in certain embodiments, compounds of the invention as described herein can be characterized as having one or more properties that afford such compounds advantages relative to other similarly classified lipids. For example, compounds disclosed herein can allow for the control and tailoring of the properties of liposomal compositions (e.g., lipid nanoparticles) of which they are a component. In particular, compounds disclosed herein can be characterized by enhanced transfection efficiencies and their ability to provoke specific biological outcomes. Such outcomes can include, for example enhanced cellular uptake, endosomal/lysosomal disruption capabilities and/or promoting the release of encapsulated materials (e.g., polynucleotides) intracellularly. The compounds disclosed herein can also be characterized by achieving high levels of peptide or protein expression when delivering mRNA encoding for said peptide or protein by intravenous, intrathecal, intramuscular, intranasal, sublingual, or by pulmonary delivery, optionally through nebulization. The compounds disclosed herein can also be characterized by achieving high Hemagglutination Inhibition (HAI) when delivering mRNA encoding an influenza antigen by intramuscular delivery. Additionally, the compounds disclosed herein have advantageous pharmacokinetic properties, biodistribution, and efficiency.

Additionally, the cationic lipids of the present invention have cleavable groups such as ester groups. These cleavable groups (e.g. esters, thioesters, disulphides, carbonates, carbamates and thiocarbamates) are contemplated to improve biodegradability and thus contribute to the lipids' favorable safety profiles.

It is contemplated that the cationic lipids of the present invention are capable of highly effective in vivo intramuscular delivery of therapeutic agents and vaccines (e.g., for treating or preventing Flu, acne, chlamydia, or a disease or disorder induced by infection with P. gingivalis). It is also contemplated that lipid nanoparticles comprising the cationic lipids of the present invention are capable of highly effective in vivo delivery while maintaining a favorable safety profile. It is also contemplated that lipid nanoparticles comprising the cationic lipids of the present invention may exhibit improved degradation in vivo.

Provided herein are compounds which are cationic lipids. In embodiments, the cationic lipids include compounds having a structure according to Formula (I′):

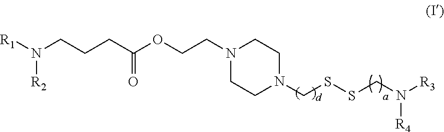

or a pharmaceutically acceptable salt thereof, wherein:
a is 3 or 4;
each of R₁, R₂, R₃, and R₄ is independently selected from:
   (i) optionally substituted C₅-C₃₀ alkyl, and optionally substituted C₅-C₃₀ alkenyl;
   (ii) optionally substituted C₁₋₅ alkyl which is substituted with an optionally substituted heterocyclyl moiety;
   (iii)

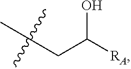

wherein each R_{A} is independently selected from optionally substituted C₃-C₂₈ alkyl, optionally substituted C₃-C₂₈ alkenyl, and —W¹—C(O)—O—X¹, wherein each W¹ is independently selected from optionally substituted C₃-C₅ alkylene and optionally substituted C₃-C₅ alkenylene, and each X¹ is independently selected from optionally substituted C₅-C₃₀ alkyl and optionally substituted C₅-C₃₀ alkenyl;
(iv)

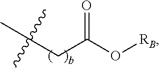

wherein each R_{B} is independently selected from optionally substituted C₅-C₃₀ alkyl, optionally substituted C₅-C₃₀ alkenyl; and
(v)

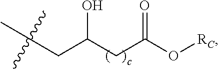

wherein each R_{C} independently selected from optionally substituted C₅-C₃₀ alkyl, optionally substituted C₅-C₃₀ alkenyl;
each b is independently selected from 5, 6, and 7;
each c is independently selected from 3, 4, and 5;
d is 2, 3, or 4;
and wherein
R₁ and R₂ are not identical to one another and/or R₃ and R₄ are not identical to one another; and wherein
at least one of R₁, R₂, R₃, and R₄, is independently selected from
option (iv)

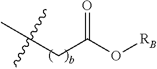

or option (v)

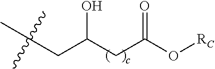

above.

In embodiments, the cationic lipids include compounds having a structure according to Formula (I):

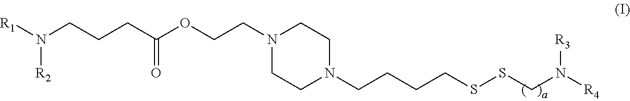

or a pharmaceutically acceptable salt thereof, wherein:
a is 3 or 4;
each of R₁, R₂, R₃, and R₄ is independently selected from:
   (i) optionally substituted C₅-C₃₀ alkyl, and optionally substituted C₅-C₃₀ alkenyl;
   (ii) optionally substituted C₁₋₅ alkyl which is substituted with an optionally substituted heterocyclyl moiety;
   (iii)

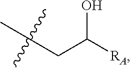

wherein each R_{A} is independently selected from optionally substituted C₃-C₂₈ alkyl, optionally substituted C₃-C₂₈ alkenyl, and —W¹—C(O)—O—X¹, wherein each W¹ is independently selected from optionally substituted C₃-C₅ alkylene and optionally substituted C₃-C₅ alkenylene, and each X¹ is independently selected from optionally substituted C₅-C₃₀ alkyl and optionally substituted C₅-C₃₀ alkenyl;
(iv)

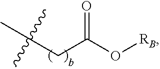

wherein each R_{B} is independently selected from optionally substituted C₅-C₃₀ alkyl, optionally substituted C₅-C₃₀ alkenyl; and
(v)

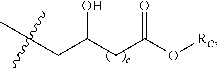

wherein each R_{C} independently selected from optionally substituted C₅-C₃₀ alkyl, optionally substituted C₅-C₃₀ alkenyl;
each b is independently selected from 5, 6, and 7;
each c is independently selected from 3, 4, and 5; and wherein
R₁ and R₂ are not identical to one another and/or R₃ and R₄ are not identical to one another; and wherein
at least one of R₁, R₂, R₃, and R₄, is independently selected from
option (iv)

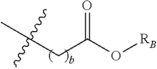

or option (v)

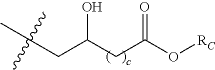

above.

In embodiments, if R₁ and R₂ are both

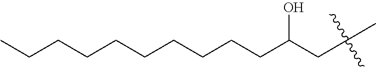 [SMILES: *CC(O)CCCCCCCCCC]

and one of R₃ and R₄ is

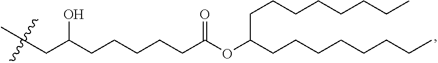

then the other of R₃ and R₄ is not selected from

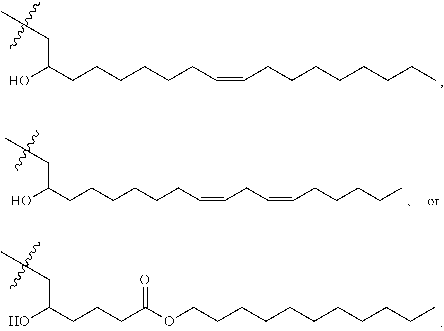 [SMILES: *CC(O)CCCCCC/C=C\CCCCCCCC ; *CC(O)CCCCCC/C=C\C/C=C\CCCCC ; *CC(O)CCCC(=O)OCCCCCCCCCCC]

In embodiments, if R₁ and R₂ are both

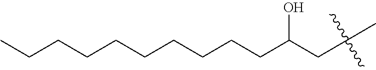 [SMILES: *CC(O)CCCCCCCCCC]

and one of R₃ and R₄ is

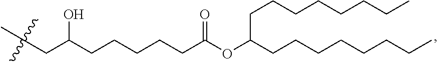

and a is 4 then the other of R₃ and R₄ is not selected from

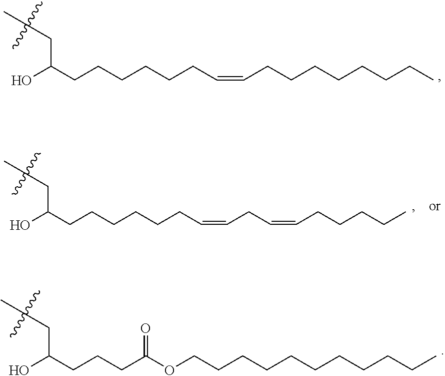

In embodiments, the cationic lipids do not include the following compound:

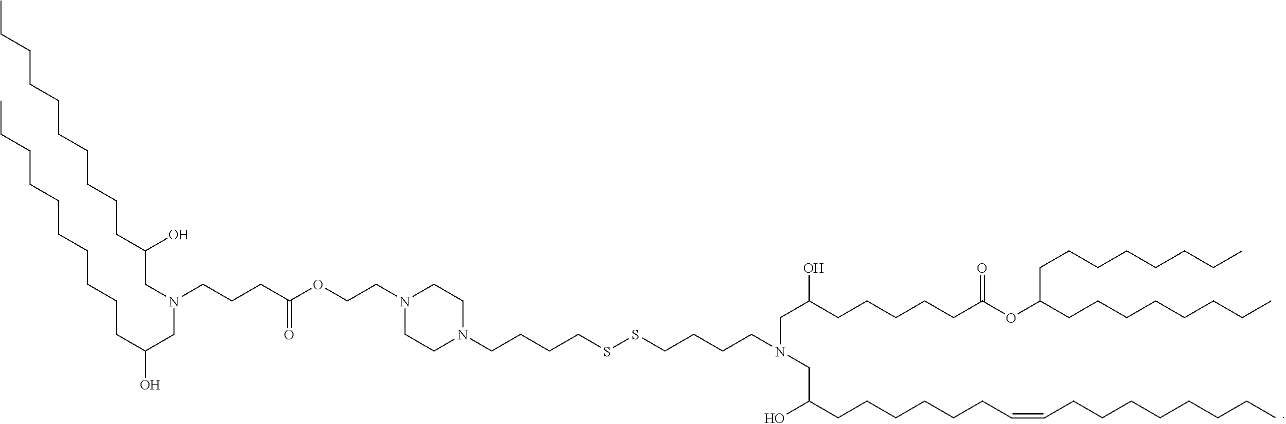

In embodiments, the cationic lipids do not include the following compound:

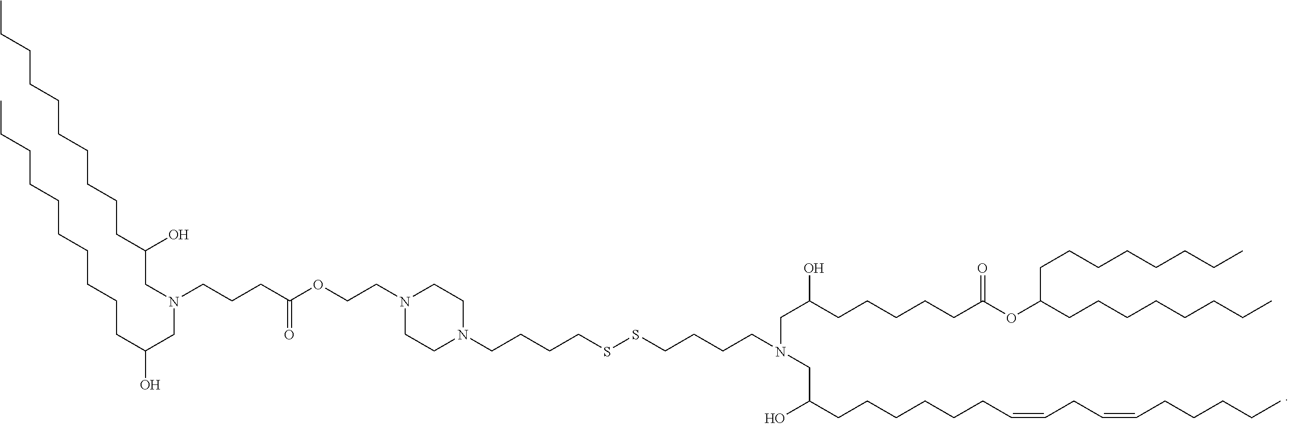

In embodiments, the cationic lipids do not include the following compound:

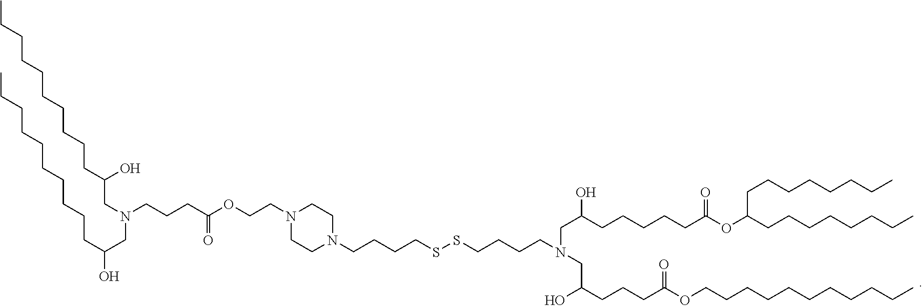

In embodiments, the cationic lipids do not include the following compounds:

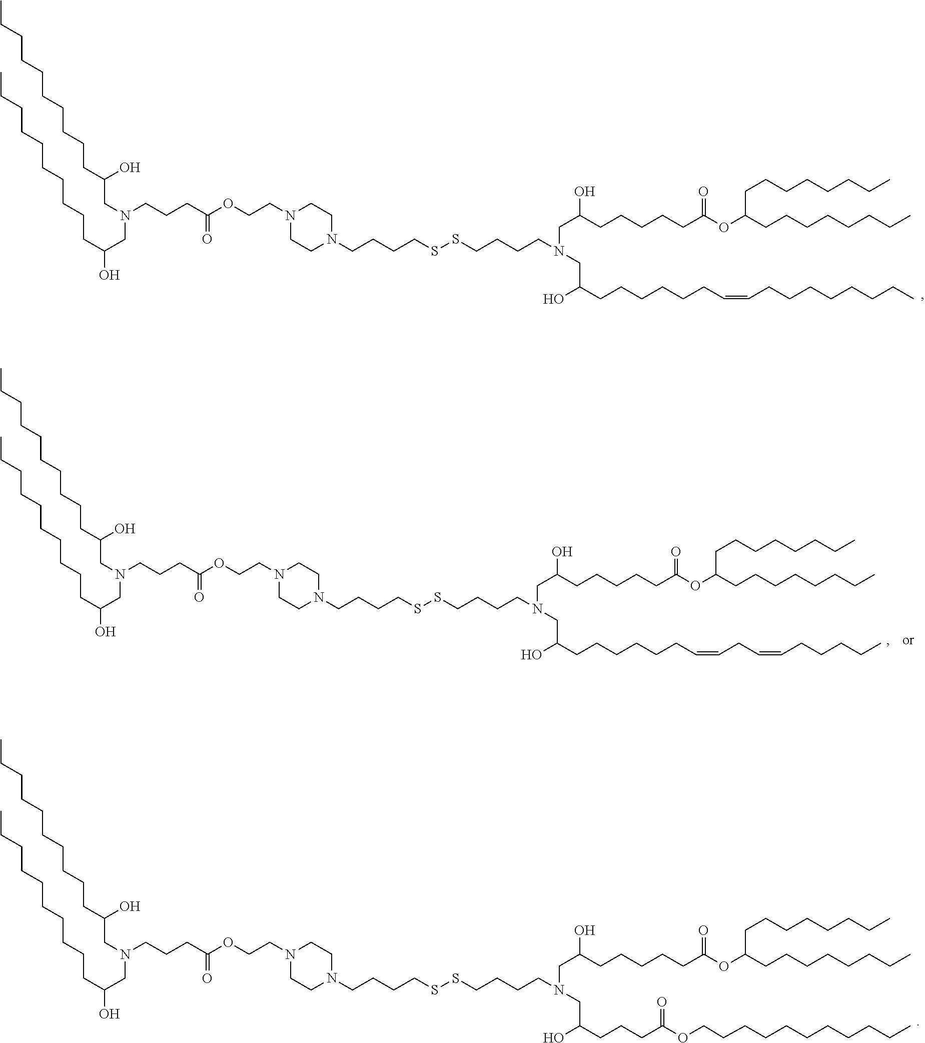

In embodiments, a is 3. In embodiments, a is 4.

In embodiments, b is 5. In embodiments, b is 6. In embodiments, b is 7. In embodiments, each b is independently selected from 5 or 6. In embodiments, each b is independently selected from 6 or 7. In one embodiment, each b is independently selected from 5 or 7.

In embodiments, d is 2. In embodiments, d is 3. In embodiments, d is 4.

In embodiments, one of R₁, R₂, R₃, and R₄ is selected from option (iv)

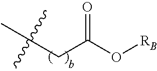

or option (v)

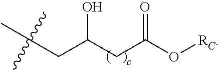

In embodiments, two of R₁, R₂, R₃, and R₄ are independently selected from option (iv)

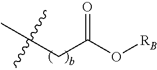

In and/or option (v)

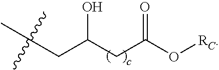

In embodiments, three of R₁, R₂, R₃, and R₄ are independently selected from option (iv)

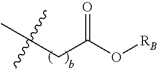

and/or option (v)

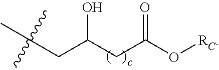

In embodiments, all of R₁, R₂, R₃, and R₄ are independently selected from option (iv)

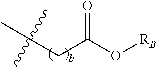

and/or option (v)

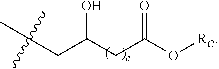

In embodiments, R₁ and R₂ are independently selected from option (iv)

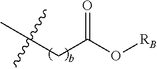

and/or option (v)

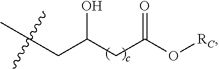

and R₃ and R₄ are not selected from option (iv)

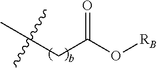

and/or option (v)

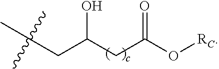

In embodiments, R₃ and R₄ are independently selected from option (iv)

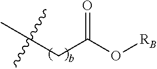

and/or option (v)

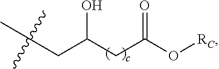

and R₁ and R₂ are not selected from option (iv)

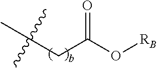

and/or option (v)

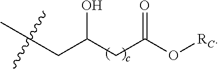

In embodiments, R₁ and R₂ are not identical to one another and R₃ and R₄ are identical to one another.

In embodiments, R₁ and R₂ are identical to one another and R₃ and R₄ are not identical to one another.

In embodiments, R₁ and R₂ are not identical to one another and R₃ and R₄ are not identical to one another.

In embodiments, none of R₁, R₂, R₃, and R₄ are identical.

In embodiments, one of R₁, R₂, R₃, and R₄ is selected from optionally substituted C₅-C₃₀ alkyl. In embodiments, two of R₁, R₂, R₃, and R₄ are independently selected from optionally substituted C₅-C₃₀ alkyl. In embodiments, three of R₁, R₂, R₃, and R₄ are independently selected from optionally substituted C₅-C₃₀ alkyl. In embodiments, R₁ and R₂ are independently selected from optionally substituted C₅-C₃₀ alkyl.

In embodiments, one of R₁, R₂, R₃, and R₄ is selected from optionally substituted C₅-C₁₂ alkyl. In embodiments, two of R₁, R₂, R₃, and R₄ are independently selected from optionally substituted C₅-C₁₂ alkyl. In embodiments, three of R₁, R₂, R₃, and R₄ are independently selected from optionally substituted C₅-C₁₂ alkyl. In embodiments, R₁ and R₂ are independently selected from optionally substituted C₅-C₁₂ alkyl.

In embodiments, one of R₁, R₂, R₃, and R₄ is

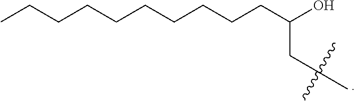 [SMILES: *CC(O)CCCCCCCCCC]

In embodiments, two of R₁, R₂, R₃, and R₄ are

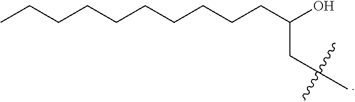 [SMILES: *CC(O)CCCCCCCCCC]

In embodiments, three of R₁, R₂, R₃, and R₄ are

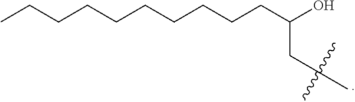 [SMILES: *CC(O)CCCCCCCCCC]

In embodiments, R₁ and R₂ are

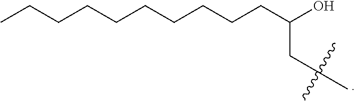 [SMILES: *CC(O)CCCCCCCCCC]

In embodiments, the cationic lipids include compounds having a structure according to Formula (II′):

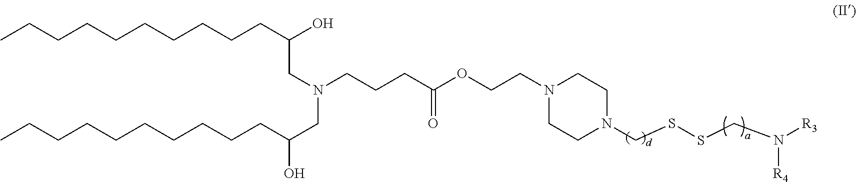

or a pharmaceutically acceptable salt thereof, wherein a, d, R₃ and R₄ are as defined herein, for example wherein a is 4.

In embodiments, the cationic lipids of the present invention include compounds having a structure according to Formula (II):

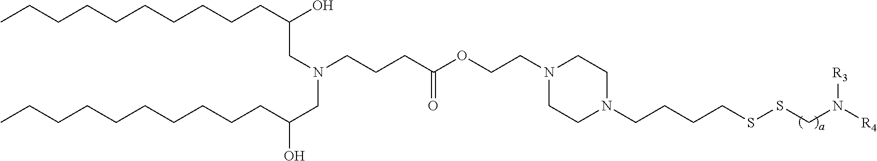

or a pharmaceutically acceptable salt thereof, wherein a, R₃ and R₄ are as defined herein, for example wherein a is 4.

In embodiments, one of R₁, R₂, R₃, and R₄ is selected from optionally substituted C₅-C₃₀ alkenyl. In embodiments, two of R₁, R₂, R₃, and R₄ are independently selected from optionally substituted C₅-C₃₀ alkenyl. In embodiments, three of R₁, R₂, R₃, and R₄ are independently selected from optionally substituted C₅-C₃₀ alkenyl.

In embodiments, one of R₃ and R₄ is selected from optionally substituted C₅-C₃₀ alkenyl. In embodiments, one of R₃ and R₄ is selected from optionally substituted C₅-C₃₀ alkenyl and the other of R₃ and R₄ is selected from option (iv)

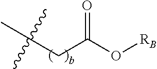

and/or option (v)

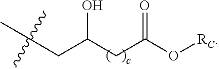

In embodiments, one of R₁, R₂, R₃, and R₄ is optionally substituted C₅-C₁₈ alkenyl. In embodiments, two of R₁, R₂, R₃, and R₄ are independently selected from optionally substituted C₅-C₁₈ alkenyl. In embodiments, three of R₁, R₂, R₃, and R₄ are independently selected from optionally substituted C₅-C₁₈ alkenyl. In embodiments, one of R₃ and R₄ is optionally substituted C₅-C₃₀ alkenyl. In embodiments, one of R₃ and R₄ is optionally substituted C₅-C₁₈ alkenyl and the other of R₃ and R₄ is selected from option (iv)

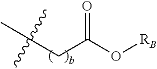

and/or option (v)

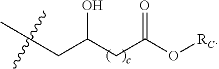

In embodiments, one of R₁, R₂, R₃, and R₄ is

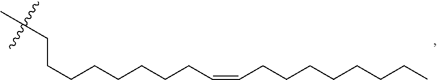 [SMILES: *CCCCCCCC/C=C\CCCCCCCC]

In embodiments, two of R₁, R₂, R₃, and R₄ are

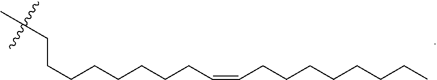 [SMILES: *CCCCCCCC/C=C\CCCCCCCC]

In embodiments, three of R₁, R₂, R₃, and R₄ are

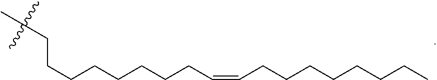 [SMILES: *CCCCCCCC/C=C\CCCCCCCC]

In embodiments, one of R₃ and R₄ is

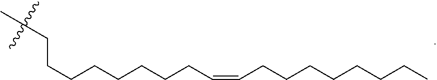 [SMILES: *CCCCCCCC/C=C\CCCCCCCC]

In embodiments, one of R₃ and R₄ is

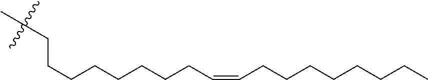 [SMILES: *CCCCCCCC/C=C\CCCCCCCC]

and the other of R₃ and R₄ is selected from option (iv)

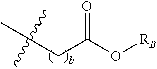

and/or option (v)

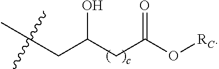

In embodiments, one of R₁, R₂, R₃, and R₄ is

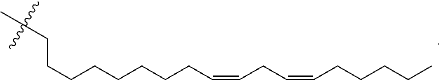 [SMILES: *CCCCCCCC/C=C\C/C=C\CCCCC]

In embodiments, two of R₁, R₂, R₃, are

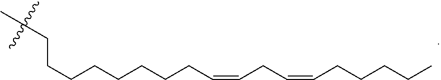 [SMILES: *CCCCCCCC/C=C\C/C=C\CCCCC]

In embodiments, three of R₁, R₂, R₃, and R₄ are

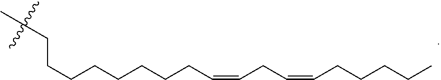 [SMILES: *CCCCCCCC/C=C\C/C=C\CCCCC]

In embodiments, one of R₃ and R₄ is

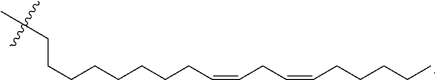 [SMILES: *CCCCCCCC/C=C\C/C=C\CCCCC]

In embodiments, one of R₃ and R₄ is

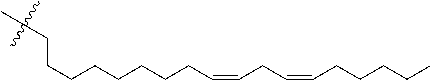 [SMILES: *CCCCCCCC/C=C\C/C=C\CCCCC]

and the other of R₃ and R₄ is selected from option (iv)

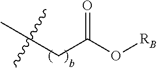

and/or option (v)

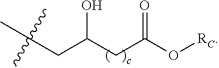

In embodiments, two of R₁, R₂, R₃, and R₄ are independently selected from optionally substituted C₅-C₃₀ alkyl and one of R₁, R₂, R₃, and R₄ is selected from optionally substituted C₅-C₃₀ alkenyl. In embodiments, R₁ and R₂ are independently selected from optionally substituted C₅-C₃₀ alkyl and one of R₃ and R₄ is selected from optionally substituted C₅-C₃₀ alkenyl. In embodiments, two of R₁, R₂, R₃, and R₄ are independently selected from optionally substituted C₅-C₁₂ alkyl and one of R₁, R₂, R₃, and R₄ is selected from optionally substituted C₅-C₁₈ alkenyl. In embodiments, R₁ and R₂ are independently selected from optionally substituted C₅-C₁₂ alkyl and one of R₃ and R₄ is selected from optionally substituted C₅-C₁₈ alkenyl.

In embodiments, R₁ and R₂ are each

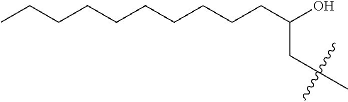 [SMILES: *CC(O)CCCCCCCCCC]

and one of R₃ and R₄ is selected from optionally substituted C₅-C₁₈ alkenyl. In embodiments, R₁ and R₂ are each

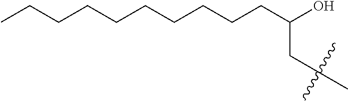 [SMILES: *CC(O)CCCCCCCCCC]

and one of R₃ and R₄ is

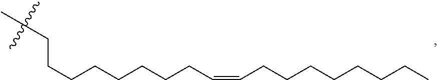 [SMILES: *CCCCCCCC/C=C\CCCCCCCC]

In embodiments, R₁ and R₂ are each

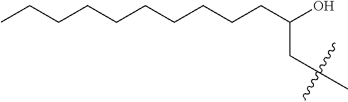 [SMILES: *CC(O)CCCCCCCCCC]

and one of R₃ and R₄ is

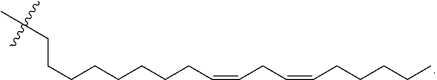 [SMILES: *CCCCCCCC/C=C\C/C=C\CCCCC]

In embodiments, one or more of R₁, R₂, R₃, and R₄ are independently selected from

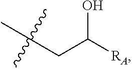

wherein each R_{A} is independently selected from optionally substituted C₃-C₂₈ alkyl, optionally substituted C₃-C₂₈ alkenyl, and —W¹—C(O)—O—X¹, wherein each W¹ is independently selected from optionally substituted C₃-C₅ alkylene and optionally substituted C₃-C₅ alkenylene, and each X¹ is independently selected from optionally substituted C₅-C₃₀ alkyl and optionally substituted C₅-C₃₀ alkenyl.

In embodiments, one of R₁, R₂, R₃, and R₄ is selected from

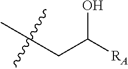

as defined above. In embodiments, two of R₁, R₂, R₃, and R₄ are independently selected from

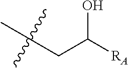

as defined above. In embodiments, three of R₁, R₂, R₃, and R₄ are independently selected from

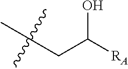

as defined above. In embodiments, R₁ and R₂ are independently selected from

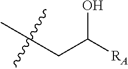

as defined above. In embodiments, R₁ and R₂ are independently selected from

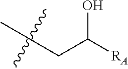

as defined above and one of R₃ and R₄ is selected from optionally substituted C₅-C₃₀ alkenyl. In embodiments, R₁ and R₂ are independently selected from

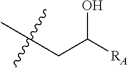

as defined above and one of R₃ and R₄ is selected from optionally substituted C₅-C₁₈ alkenyl. In embodiments, R₁ and R₂ are independently selected from

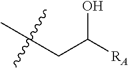

as defined above and one of R₃ and R₄ is

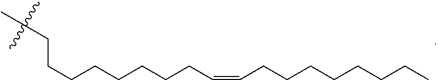 [SMILES: *CCCCCCCC/C=C\CCCCCCCC]

In embodiments, R₁ and R₂ are independently selected from

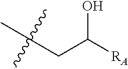

as defined above and one of R₃ and R₄ is

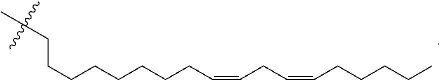 [SMILES: *CCCCCCCC/C=C\C/C=C\CCCCC]

In embodiments, R₃ and R₄ are independently selected from

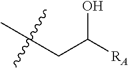

as defined above.

In embodiments, one R_{A} is selected from optionally substituted C₃-C₂₈ alkyl. In embodiments, two R_{A} are independently selected from optionally substituted C₃-C₂₈ alkyl. In embodiments, each R_{A} is independently selected from optionally substituted C₃-C₂₈ alkyl. In embodiments, one R_{A} is selected from optionally substituted C₃-C₁₀ alkyl. In embodiments, two R_{A} are independently selected from optionally substituted C₃-C₁₀ alkyl. In embodiments, each R_{A} is independently selected from optionally substituted C₃-C₁₀ alkyl. In embodiments, one R_{A} is

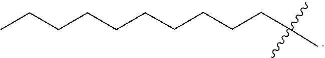 [SMILES: *CCCCCCCCCC]

In embodiments, two R_{A} are

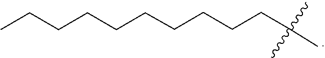 [SMILES: *CCCCCCCCCC]

In embodiments, each R_{A} is

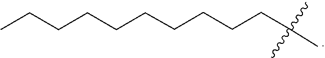 [SMILES: *CCCCCCCCCC]

In embodiments, one R_{A} is selected from optionally substituted C₃-C₂₈ alkenyl. In embodiments, two R_{A} are independently selected from optionally substituted C₃-C₂₈ alkenyl. In embodiments, each R_{A} is independently selected from optionally substituted C₃-C₂₈ alkenyl. In embodiments, one R_{A} is selected from optionally substituted C₃-C₁₆ alkenyl. In embodiments, two R_{A} are independently selected from optionally substituted C₃-C₁₆ alkyl. In embodiments, each R_{A} is independently selected from optionally substituted C₃-C₁₆ alkyl. In embodiments, one R_{A} is

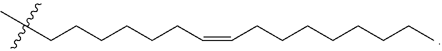 [SMILES: *CCCCCC/C=C\CCCCCCCC]

In embodiments, two R_{A} are

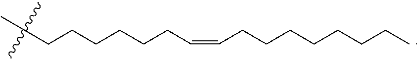 [SMILES: *CCCCCC/C=C\CCCCCCCC]

In embodiments, each R_{A} is

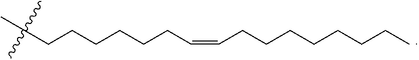 [SMILES: *CCCCCC/C=C\CCCCCCCC]

In embodiments, one R_{A} is

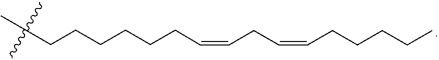 [SMILES: *CCCCCC/C=C\C/C=C\CCCCC]

In embodiments, two R_{A} are

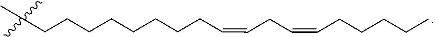 [SMILES: *CCCCCCCC/C=C\C/C=C\CCCCC]

In embodiments, each R_{A} is

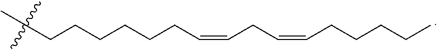 [SMILES: *CCCCCC/C=C\C/C=C\CCCCC]

In embodiments, one R_{A} is selected from —W¹—C(O)—O—X¹. In embodiments, two R_{A} are independently selected from —W¹—C(O)—O—X¹. In embodiments, each R_{A} is independently selected from —W¹—C(O)—O—X¹.

In embodiments, one W¹ is selected from optionally substituted C₃-C₅ alkylene. In embodiments, two W¹ are independently selected from optionally substituted C₃-C₅ alkylene. In embodiments, each W¹ is independently selected from optionally substituted C₃-C₅ alkylene. In embodiments, one W¹ is selected from optionally substituted C₃-C₅ alkenylene. In embodiments, two W¹ are independently selected from optionally substituted C₃-C₅ alkenylene. In embodiments, each W¹ is independently selected from optionally substituted C₃-C₅ alkenylene.

In embodiments, one X¹ is selected from optionally substituted C₅-C₃₀ alkyl. In embodiments, two X¹ are independently selected from optionally substituted C₅-C₃₀ alkyl. In embodiments, each X¹ is independently selected from optionally substituted C₅-C₃₀ alkyl. In embodiments, one X¹ is selected from optionally substituted C₆-C₁₇ alkyl. In embodiments, two X¹ are independently selected from optionally substituted C₆-C₁₇ alkyl. In embodiments, each X¹ is independently selected from optionally substituted C₆-C₁₇ alkyl. In embodiments, one X¹ is selected from optionally substituted branched C₆-C₁₇ alkyl. In embodiments, two X¹ are independently selected from optionally substituted branched C₆-C₁₇ alkyl. In embodiments, each X¹ is independently selected from optionally substituted branched C₆-C₁₇ alkyl.

In embodiments, one X¹ is

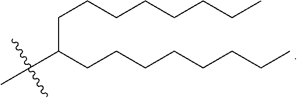 [SMILES: *C(CCCCCCCC)CCCCCCCC]

In embodiments, two X¹ are

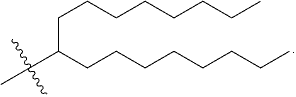 [SMILES: *C(CCCCCCCC)CCCCCCCC]

In embodiments, each X¹ is

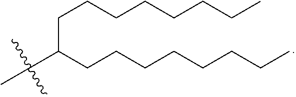 [SMILES: *C(CCCCCCCC)CCCCCCCC]

In embodiments, one X¹ is

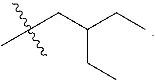 [SMILES: *CC(CC)CC]

In embodiments, two X¹ are

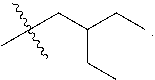 [SMILES: *CC(CC)CC]

In embodiments, each X¹ is

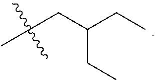 [SMILES: *CC(CC)CC]

In embodiments, one X¹ is

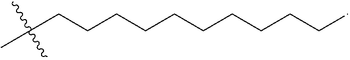

In embodiments, two X¹ are

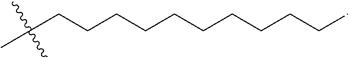

In embodiments, each X¹ is

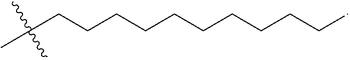

In embodiments, one X¹ is

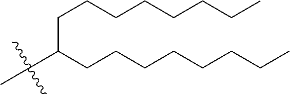 [SMILES: *C(CCCCCCCC)CCCCCCCC]

### and one X¹ is

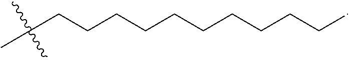

In embodiments, two X¹ are

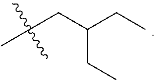 [SMILES: *CC(CC)CC]

In embodiments, two X¹ are

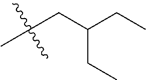 [SMILES: *CC(CC)CC]

### and one X¹ is

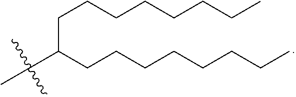 [SMILES: *C(CCCCCCCC)CCCCCCCC]

In embodiments, two X¹ are

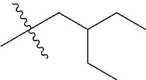 [SMILES: *CC(CC)CC]

### and one X¹ is

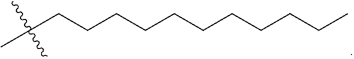 [SMILES: *CCCCCCCCCCC]

In embodiments, two X¹ are

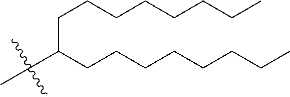 [SMILES: *C(CCCCCCCC)CCCCCCCC]

### and one X¹ is

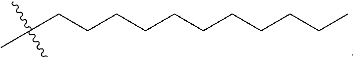 [SMILES: *CCCCCCCCCCC]

In embodiments, two X¹ are

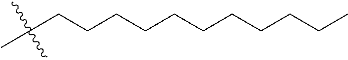 [SMILES: *CCCCCCCCCCC]

### and one X¹ is

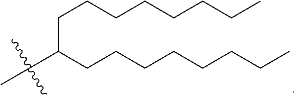 [SMILES: *C(CCCCCCCC)CCCCCCCC]

In embodiments, one X¹ is selected from optionally substituted C₅-C₃₀ alkenyl. In embodiments, two X¹ are independently selected from optionally substituted C₅-C₃₀ alkenyl. In embodiments, each X¹ is independently selected from optionally substituted C₅-C₃₀ alkenyl. In embodiments, one X¹ is selected from optionally substituted C₅-C₉ alkenyl. In embodiments, two X¹ are independently selected from optionally substituted C₅-C₉ alkenyl. In embodiments, each X¹ is independently selected from optionally substituted C₅-C₉ alkenyl. In embodiments, one X¹ is

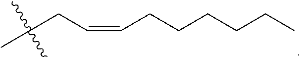 [SMILES: *C/C=C\CCCCCC]

In embodiments, two X¹ are

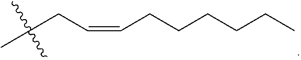 [SMILES: *C/C=C\CCCCCC]

In embodiments, each X¹ is

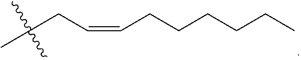 [SMILES: *C/C=C\CCCCCC]

In embodiments, one or more of R₁, R₂, R₃, and R₄ are independently selected from

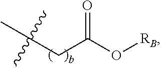

wherein each R_{B} is independently selected from optionally substituted C₅-C₃₀ alkyl and optionally substituted C₅-C₃₀ alkenyl. In embodiments, one of R₁, R₂, R₃, and R₄ is selected from

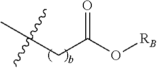

as defined above. In embodiments, two of R₁, R₂, R₃, and R₄ are independently selected from

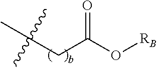

as defined above. In embodiments, three of R₁, R₂, R₃, and R₄ are independently selected from

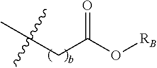

as defined above. In embodiments, each of R₁, R₂, R₃, and R₄ are independently selected from

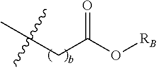

as defined above. In embodiments, R₁ and R₂ are independently selected from

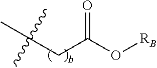

as defined above. In embodiments, one of R₃ and R₄ is selected from

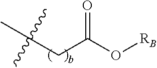

as defined above. In embodiments, R₃ and R₄ are independently selected from

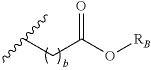

as defined above.

In embodiments, one R_{B} is selected from optionally substituted C₅-C₃₀ alkyl. In embodiments, two R_{B} are independently selected from optionally substituted C₅-C₃₀ alkyl. In embodiments, three R_{B} are independently selected from optionally substituted C₅-C₃₀ alkyl. In embodiments, each R_{B} is independently selected from optionally substituted C₅-C₃₀ alkyl.

In embodiments, one R_{B} is selected from optionally substituted C₆-C₁₇ alkyl. In embodiments, two R_{B} are independently selected from optionally substituted C₆-C₁₇ alkyl. In embodiments, three R_{B} are independently selected from optionally substituted C₆-C₁₇ alkyl. In embodiments, each R_{B} is independently selected from optionally substituted C₆-C₁₇ alkyl.

In embodiments, one R_{B} is selected from optionally substituted branched C₆-C₁₇ alkyl. In embodiments, two R_{B} are independently selected from optionally substituted branched C₆-C₁₇ alkyl. In embodiments, three R_{B} are independently selected from optionally substituted branched C₆-C₁₇ alkyl. In embodiments, each R_{B} is independently selected from optionally substituted branched C₆-C₁₇ alkyl.

In embodiments, one R_{B} is

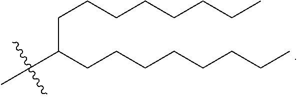 [SMILES: *C(CCCCCCCC)CCCCCCCC]

In embodiments, two R_{B} are

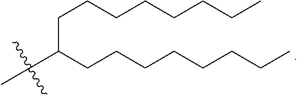 [SMILES: *C(CCCCCCCC)CCCCCCCC]

In embodiments, three R_{B} are

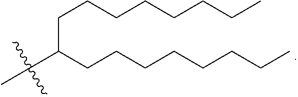 [SMILES: *C(CCCCCCCC)CCCCCCCC]

In embodiments, each R_{B} is

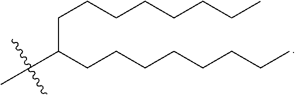 [SMILES: *C(CCCCCCCC)CCCCCCCC]

In embodiments, one R_{B} is

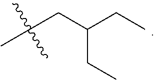 [SMILES: *CC(CC)CC]

In embodiments, two R_{B} are

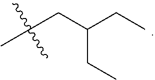 [SMILES: *CC(CC)CC]

In embodiments, three R_{B} are

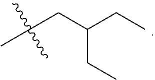 [SMILES: *CC(CC)CC]

In embodiments, each R_{B} is

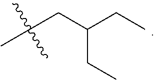 [SMILES: *CC(CC)CC]

In embodiments, one R_{B} is

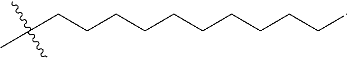

In embodiments, two R_{B} are

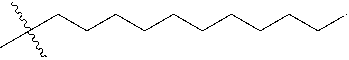

In embodiments, three R_{B} are

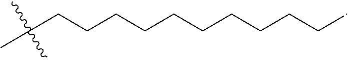

In embodiments, each R_{B} is

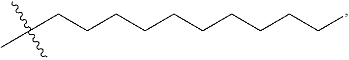

In embodiments, the cationic lipids include compounds having a structure according to Formula (III′):

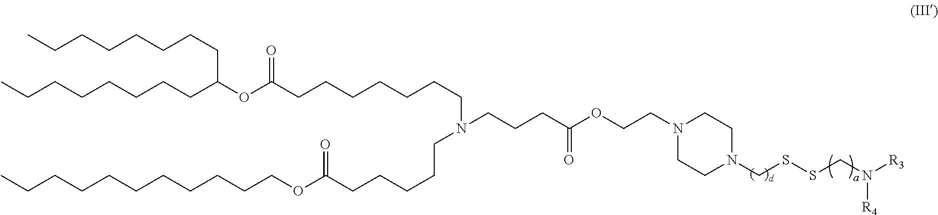

or a pharmaceutically acceptable salt thereof, wherein a, d, R₃ and R₄ are as defined herein.

In embodiments, the cationic lipids include compounds having a structure according to Formula (III):

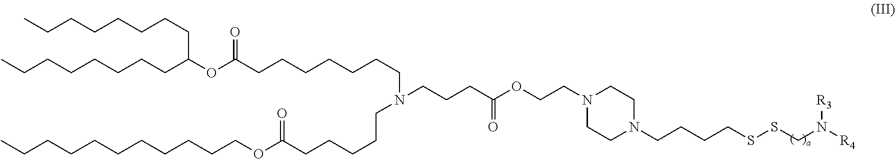

or a pharmaceutically acceptable salt thereof, wherein a, R₃ and R₄ are as defined herein.

In embodiments, one R_{B} is selected from optionally substituted C₅-C₃₀ alkenyl. In embodiments, two R_{B} are independently selected from optionally substituted C₅-C₃₀ alkenyl. In embodiments, three R_{B} are independently selected from optionally substituted C₅-C₃₀ alkenyl. In embodiments, each R_{B} is independently selected from optionally substituted C₅-C₃₀ alkenyl.

In embodiments, one R_{B} is selected from optionally substituted C₅-C₉ alkenyl. In embodiments, two R_{B} are independently selected from optionally substituted C₅-C₉ alkenyl. In embodiments, three R_{B} are independently selected from optionally substituted C₅-C₉ alkenyl. In embodiments, each R_{B} is independently selected from optionally substituted C₅-C₉ alkenyl. In embodiments, each R_{B} is

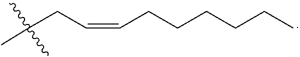 [SMILES: *C/C=C\CCCCCC]

In embodiments, two R_{B} are

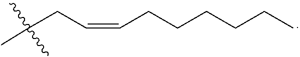 [SMILES: *C/C=C\CCCCCC]

In embodiments, three R_{B} are

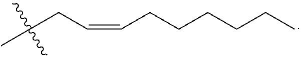 [SMILES: *C/C=C\CCCCCC]

In embodiments, each R_{B} is

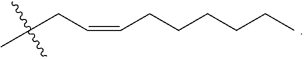 [SMILES: *C/C=C\CCCCCC]

In embodiments, one or more of R₁, R₂, R₃, and R₄ are independently selected from

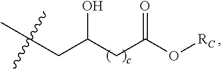

wherein each R_{C} independently selected from optionally substituted C₅-C₃₀ alkyl and optionally substituted C₅-C₃₀ alkenyl. In embodiments, one of R₁, R₂, R₃, and R₄ is selected from

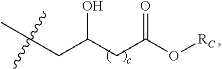

as defined above. In embodiments, two of R₁, R₂, R₃, and R₄ are independently selected from

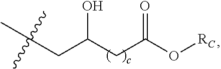

as defined above. In embodiments, three of R₁, R₂, R₃, and R₄ are independently selected from

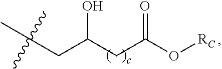

as defined above. In embodiments, each of R₁, R₂, R₃, and R₄ are independently selected from

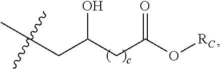

as defined above.

In embodiments, R₁ and R₂ are independently selected from

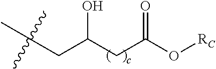

as defined above.

In embodiments, one of R₃ and R₄ is selected from

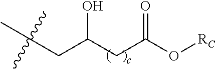

as defined above. In embodiments, R₃ and R₄ are independently selected from

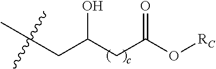

as defined above.

In embodiments, one or more c is selected from 3 or 5. In embodiments, one c is selected from 3 or 5. In embodiments, two c are selected from 3 or 5. In embodiments, three c are selected from 3 or 5. In embodiments, each c is selected from 3 or 5. In embodiments, each c is 3. In embodiments, each c is 5.

In embodiments, one or more c is 4. In embodiments, one c is 4. In embodiments, two c are 4. In embodiments, three care 4. In embodiments, each c is 4.

In embodiments, one R_{C} is selected from optionally substituted C₅-C₃₀ alkyl. In embodiments, two R_{C} are independently selected from optionally substituted C₅-C₃₀ alkyl. In embodiments, three R_{C} are independently selected from optionally substituted C₅-C₃₀ alkyl. In embodiments, each R_{C} is independently selected from optionally substituted C₅-C₃₀ alkyl.

In embodiments, one R_{C} is selected from optionally substituted C₆-C₁₇ alkyl. In embodiments, two R_{C} are independently selected from optionally substituted C₆-C₁₇ alkyl. In embodiments, three R_{C} are independently selected from optionally substituted C₆-C₁₇ alkyl. In embodiments, each R_{C} is independently selected from optionally substituted C₆-C₁₇ alkyl.

In embodiments, one R_{C} is selected from optionally substituted branched C₆-C₁₇ alkyl. In embodiments, two R_{C} are independently selected from optionally substituted branched C₆-C₁₇ alkyl. In embodiments, three R_{C} are independently selected from optionally substituted branched C₆-C₁₇ alkyl. In embodiments, each R_{C} is independently selected from optionally substituted branched C₆-C₁₇ alkyl.

In embodiments, one R_{C} is

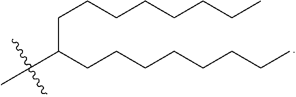 [SMILES: *C(CCCCCCCC)CCCCCCCC]

In embodiments, two R_{C} are

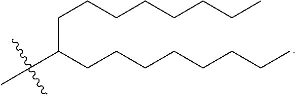 [SMILES: *C(CCCCCCCC)CCCCCCCC]

In embodiments, three R_{C} are

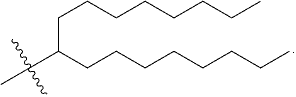 [SMILES: *C(CCCCCCCC)CCCCCCCC]

In embodiments, each R_{C} are

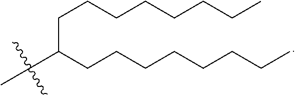 [SMILES: *C(CCCCCCCC)CCCCCCCC]

In embodiments, one R_{C} is

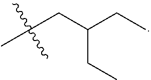 [SMILES: *CC(CC)CC]

In embodiments, two R_{C} are

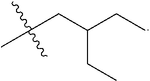 [SMILES: *CC(CC)CC]

In embodiments, three R_{C} are

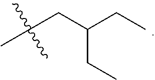 [SMILES: *CC(CC)CC]

In embodiments, each R_{C} are

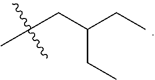 [SMILES: *CC(CC)CC]

In embodiments, one R_{C} is

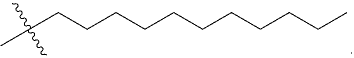 [SMILES: *CCCCCCCCCCC]

In embodiments, two R_{C} are

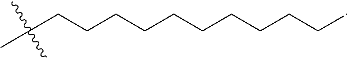

In embodiments, three R_{C} are

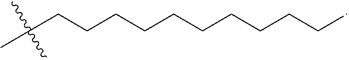

In embodiments, each R_{C} is

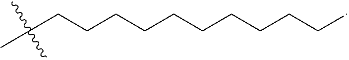

In embodiments, the cationic lipids include compounds having a structure according to Formula (IV′):

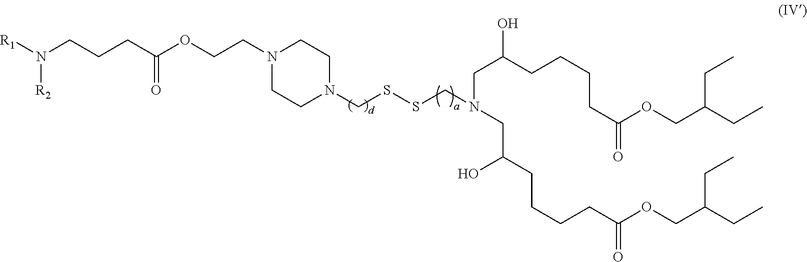

or a pharmaceutically acceptable salt thereof, wherein a, d, R₁ and R₂ are as defined herein, for example wherein a is 3.

In embodiments, the cationic lipids of the present invention include compounds having a structure according to Formula (IV):

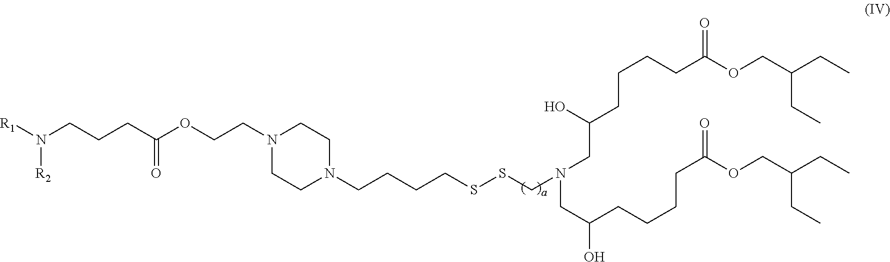

or a pharmaceutically acceptable salt thereof, wherein a, R₁ and R₂ are as defined herein, for example wherein a is 3.

In embodiments, the cationic lipids of the present invention include compounds having a structure according to Formula (V′):

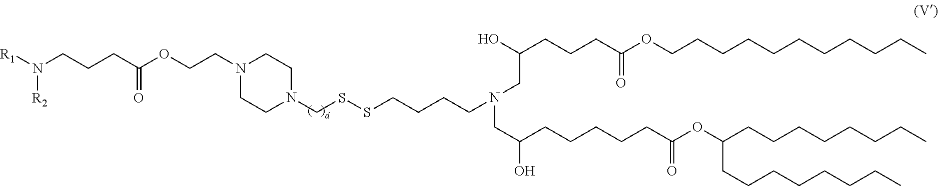

or a pharmaceutically acceptable salt thereof, wherein d, R₁ and R₂ are as defined herein.

In embodiments, the cationic lipids of the present invention include compounds having a structure according to Formula (V):

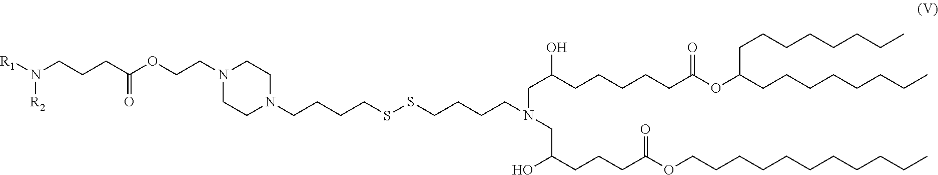

or a pharmaceutically acceptable salt thereof, wherein R₁ and R₂ are as defined herein.

In embodiments, one R_{C} is selected from optionally substituted C₅-C₃₀ alkenyl. In embodiments, two R_{C} are independently selected from optionally substituted C₅-C₃₀ alkenyl. In embodiments, three R_{C} are independently selected from optionally substituted C₅-C₃₀ alkenyl. In embodiments, each R_{C} is independently selected from optionally substituted C₅-C₃₀ alkenyl.

In embodiments, one R_{C} is selected from optionally substituted C₅-C₉ alkenyl. In embodiments, two R_{C} are independently selected from optionally substituted C₅-C₉ alkenyl. In embodiments, three R_{C} are independently selected from optionally substituted C₅-C₉ alkenyl. In embodiments, each R_{C} is independently selected from optionally substituted C₅-C₉ alkenyl.

In embodiments, one R_{C} is

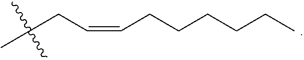 [SMILES: *C/C=C\CCCCCC]

In embodiments, two R_{C} are

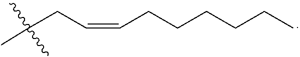 [SMILES: *C/C=C\CCCCCC]

In embodiments, three R_{C} are

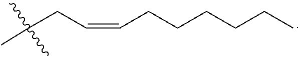 [SMILES: *C/C=C\CCCCCC]

In embodiments, each R_{C} is

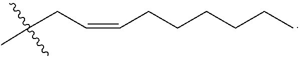 [SMILES: *C/C=C\CCCCCC]

In embodiments one or more of R₁, R₂, R₃, and R₄ is independently selected from optionally substituted C₁-C₅ alkyl which is substituted with an optionally substituted heterocyclyl moiety. In embodiments, one of R₁, R₂, R₃, and R₄ is selected from optionally substituted C₁-C₅ alkyl which is substituted with an optionally substituted heterocyclyl moiety. In embodiments, two of R₁, R₂, R₃, and R₄ are independently selected from optionally substituted C₁-C₅ alkyl which is substituted with an optionally substituted heterocyclyl moiety. In embodiments, three of R₁, R₂, R₃, and R₄ are independently selected from optionally substituted C₁-C₅ alkyl which is substituted with an optionally substituted heterocyclyl moiety. In embodiments, the heterocyclyl moiety is substituted. In one embodiment, said optionally substituted C₁-C₅ alkyl which is substituted with an optionally substituted heterocyclyl moiety is

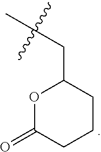 [SMILES: *CC1CCCC(=O)O1]

In embodiments, the cationic lipids include compounds having a structure according to Formula (VI′):

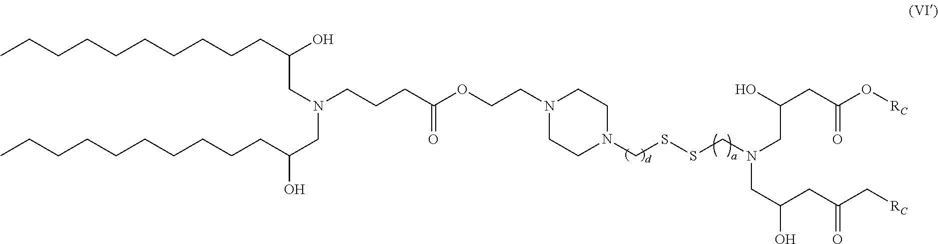

or a pharmaceutically acceptable salt thereof, wherein:
a is 3 or 4;
d is 2, 3, or 4;
each c is independently selected from 3 and 4; and
each R_{C} is independently selected from

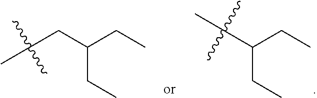

In embodiments, the cationic lipids of the present invention include compounds having a structure according to Formula (VI):

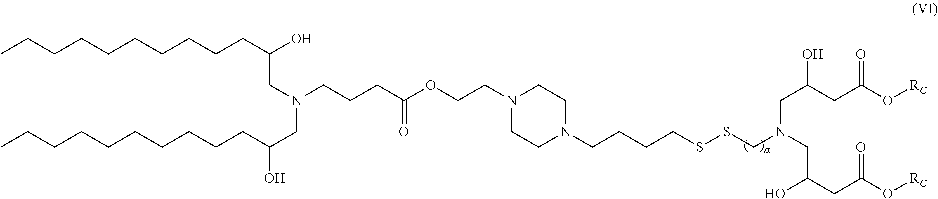

or a pharmaceutically acceptable salt thereof, wherein:
a is 3 or 4;
each c is independently selected from 3 and 4; and
each R_{C} is independently selected from

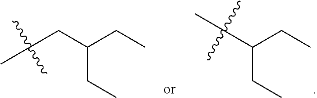

In embodiments, a is 3. In embodiments, a is 4.

In embodiments, each c is 3. In embodiments, one c is 3 and one c is 4. In embodiments, each c is 4.

In embodiments, each R_{C} is

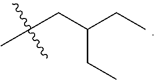 [SMILES: *CC(CC)CC]

In embodiments, each R_{C} is

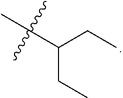 [SMILES: *C(CC)CC]

In embodiments, one R_{C} is

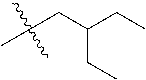 [SMILES: *CC(CC)CC]

and the other R_{C} is

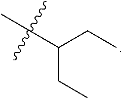 [SMILES: *C(CC)CC]

In embodiments, the substituents are not optionally substituted. In embodiments, the R₁ and R₂ substituents are optionally substituted and the R₃ and R₄ substituents are not optionally substituted. In embodiments, the R₃ and R₄ substituents are optionally substituted and the R₁ and R₂ substituents are not optionally substituted.

In embodiments, the cationic lipids of the present invention have any one of the structures in Table 1 or Table 2, or a pharmaceutically acceptable salt thereof.

In embodiments, provided herein is a composition comprising a cationic lipid of the present invention, and further comprising:
(i) one or more non-cationic lipids,
(ii) one or more cholesterol-based lipids and
(iii) one or more PEG-modified lipids.

In embodiments, this composition is a lipid nanoparticle, optionally a liposome.

In embodiments, the one or more cationic lipid(s) constitute(s) about 30 mol %-60 mol % of the lipid nanoparticle. In embodiments, the one or more cationic lipid(s) constitute(s) about 35 mol %-55 mol % of the lipid nanoparticle. In embodiments, the one or more cationic lipid(s) constitute(s) about 40 mol %-50 mol % of the lipid nanoparticle.

In embodiments, the one or more non-cationic lipid(s) constitute(s) about 10 mol %-50 mol % of the lipid nanoparticle. In embodiments, the one or more non-cationic lipid(s) constitute(s) about 15 mol %-45 mol % of the lipid nanoparticle. In embodiments, the one or more non-cationic lipid(s) constitute(s) about 20 mol %-40 mol % of the lipid nanoparticle. In embodiments, the one or more non-cationic lipid(s) constitute(s) about 25 mol %-35 mol % of the lipid nanoparticle.

In embodiments, the one or more PEG-modified lipid(s) constitute(s) about 1 mol %-10 mol % of the lipid nanoparticle. In embodiments, the one or more PEG-modified lipid(s) constitute(s) about 2 mol %-9 mol % of the lipid nanoparticle. In embodiments, the one or more PEG-modified lipid(s) constitute(s) about 3 mol %-8 mol % of the lipid nanoparticle. In embodiments, the one or more PEG-modified lipid(s) constitute(s) about 4 mol %-7 mol % of the lipid nanoparticle. In embodiments, the one or more PEG-modified lipid(s) constitute(s) about 5 mol %-6 mol % of the lipid nanoparticle.

In embodiments, the cholesterol-based lipid constitutes about 10 mol %-50 mol % of the lipid nanoparticle. In embodiments, the cholesterol-based lipid constitutes about 15 mol %-45 mol % of the lipid nanoparticle. In embodiments, the cholesterol-based lipid constitutes about 20 mol %-40 mol % of the lipid nanoparticle. In embodiments, the cholesterol-based lipid constitutes about 25 mol %-35 mol % of the lipid nanoparticle.

In embodiments, the lipid nanoparticle encapsulates a nucleic acid, optionally an mRNA encoding a peptide or protein. In embodiments, the lipid nanoparticle encapsulates an mRNA encoding a peptide or protein, optionally for use in a vaccine. In embodiments, the peptide encoded by the mRNA is an antigen. In embodiments, the mRNA encodes an influenza antigen. In embodiments, the mRNA encodes an antigen of influenza A virus. In embodiments, the mRNA encodes an antigen of influenza B virus. In embodiments, the mRNA encodes an antigen of Chlamydia sp. bacteria. In embodiments, the mRNA encodes an antigen of C. trachomatis. In embodiments, the mRNA encodes an antigen of P. gingivalis. In embodiments, the mRNA encodes an acne antigen. In embodiments, the mRNA encodes an antigen of C. acnes.

As used herein, the phrase “encapsulation percentage” refers to the fraction of therapeutic agent (e.g. mRNA) that is effectively encapsulated within a liposomal-based vehicle (e.g. a lipid nanoparticle) relative to the initial fraction of therapeutic agent present in the lipid phase. In embodiments, the lipid nanoparticles have an encapsulation percentage for mRNA of at least 50%. In embodiments, the lipid nanoparticles have an encapsulation percentage for mRNA of at least 55%. In embodiments, the lipid nanoparticles have an encapsulation percentage for mRNA of at least 60%. In embodiments, the lipid nanoparticles have an encapsulation percentage for mRNA of at least 65%. In embodiments, the lipid nanoparticles have an encapsulation percentage for mRNA of at least 70%. In embodiments, the lipid nanoparticles have an encapsulation percentage for mRNA of at least 75%. In embodiments, the lipid nanoparticles have an encapsulation percentage for mRNA of at least 80%. In embodiments, the lipid nanoparticles have an encapsulation percentage for mRNA of at least 85%. In embodiments, the lipid nanoparticles have an encapsulation percentage for mRNA of at least 90%. In embodiments, the lipid nanoparticles have an encapsulation percentage for mRNA of at least 95%. In embodiments, the encapsulation percentage is calculated by performing the Ribogreen assay (Invitrogen) with and without the presence of 0.1% Triton-X 100.

In embodiments, the composition of the present invention is for use in therapy.

In embodiments, the composition of the present invention is for use in a method of treating or preventing a disease amenable to treatment or prevention by the peptide or protein encoded by the mRNA encapsulated within said composition.

In embodiments, the disease or disorder is caused by a viral infection. In embodiments, the disease or disorder is influenza.

In embodiments, the disease or disorder is caused by a bacterial infection. In embodiments, the disease or disorder is chlamydia.

In embodiments, the disease or disorder is induced by infection with P. gingivalis.

In embodiments, the disease or disorder is acne.

In embodiments, a method for treating or preventing a disease is provided, wherein said method comprises administering to a subject in need thereof a composition of the present invention and wherein the disease is amenable to treatment or prevention by the peptide or protein encoded by the mRNA.

In embodiments, provided herein is the use of a composition of the present invention for the manufacture of a medicament for the treatment of a disease, wherein the disease is amenable to treatment or prevention by the peptide or protein encoded by the mRNA.

In embodiments, the composition is administered intravenously, intrathecally, intramuscularly, intranasally, sublingually, or by pulmonary delivery, optionally through nebulization. In one embodiment, the composition is administered intramuscularly.

### Exemplary Compounds

In embodiments, the cationic lipids of the present invention include compounds selected from those depicted in Table 1 or Table 2, or a pharmaceutically acceptable salt thereof.

TABLE 1
Table 1

| | |
|---|---|
| Com- | |
| pound | |
| # | |
| Form- | |
| ula | Compound structure |

Formula (I) 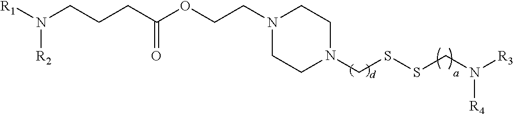
Formula (II)

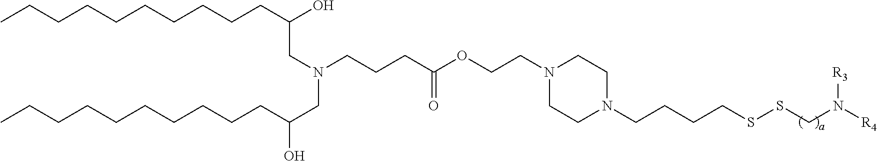

| | |
|---|---|
| 1 | 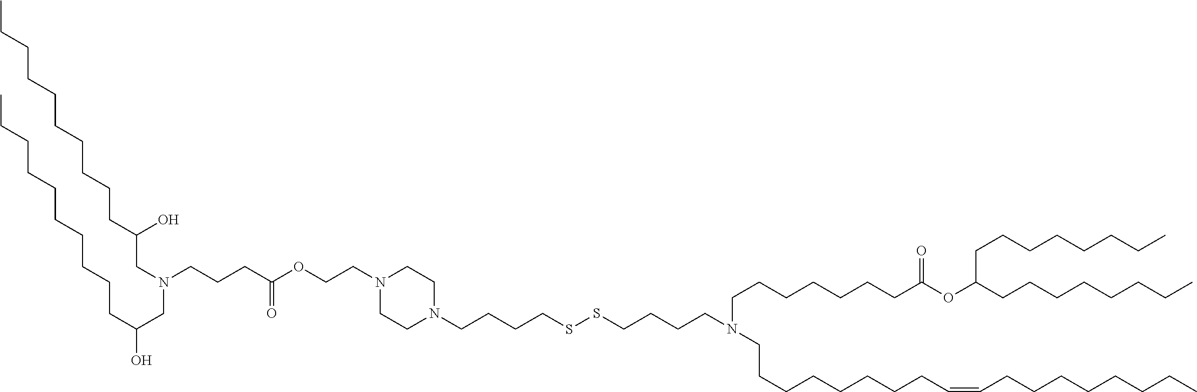 [SMILES: CCCCCCCC/C=C\CCCCCCCCN(CCCCCCCC(=O)OC(CCCCCCCC)CCCCCCCC)CCCCSSCCCCN1CCN(CCOC(=O)CCCN(CC(O)CCCCCCCCCC)CC(O)CCCCCCCCCC)CC1] |
| 2 | 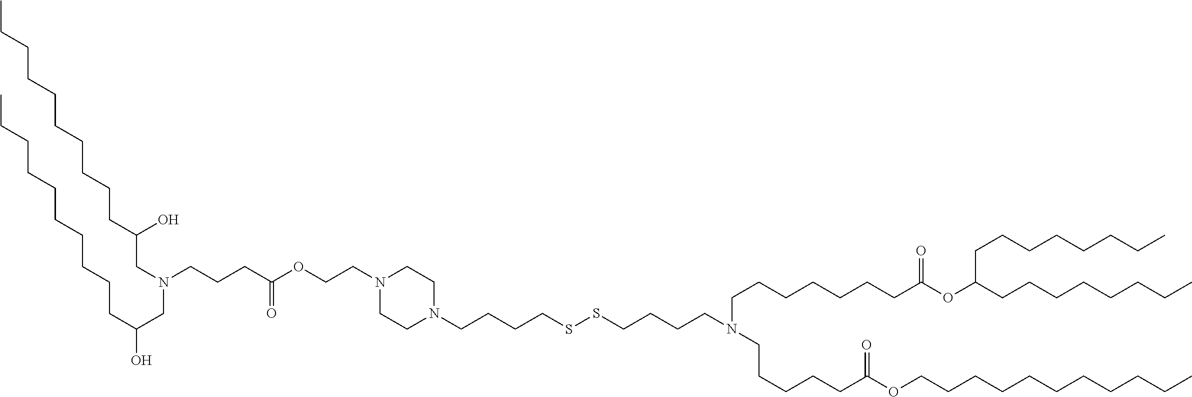 [SMILES: CCCCCCCCCCCOC(=O)CCCCCN(CCCCCCCC(=O)OC(CCCCCCCC)CCCCCCCC)CCCCSSCCCCN1CCN(CCOC(=O)CCCN(CC(O)CCCCCCCCCC)CC(O)CCCCCCCCCC)CC1] |
| 3 | 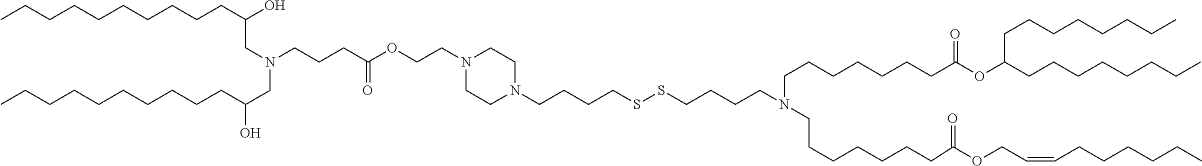 [SMILES: CCCCCC/C=C\COC(=O)CCCCCCCN(CCCCCCCC(=O)OC(CCCCCCCC)CCCCCCCC)CCCCSSCCCCN1CCN(CCOC(=O)CCCN(CC(O)CCCCCCCCCC)CC(O)CCCCCCCCCC)CC1] |
| 4 | 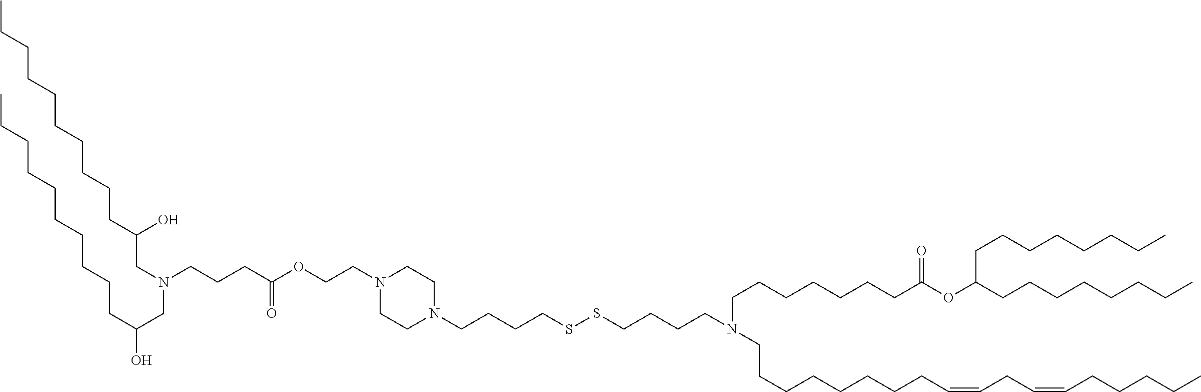 [SMILES: CCCCC/C=C\C/C=C\CCCCCCCCN(CCCCCCCC(=O)OC(CCCCCCCC)CCCCCCCC)CCCCSSCCCCN1CCN(CCOC(=O)CCCN(CC(O)CCCCCCCCCC)CC(O)CCCCCCCCCC)CC1] |
| 5 | 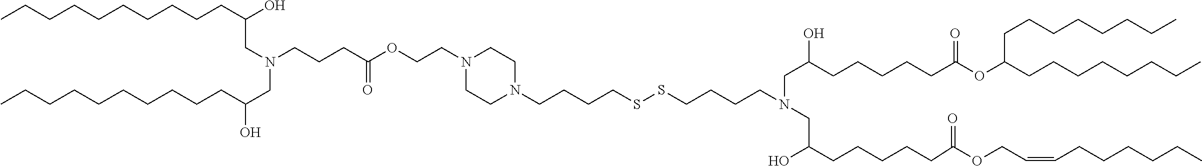 [SMILES: CCCCCC/C=C\COC(=O)CCCCCC(O)CN(CCCCSSCCCCN1CCN(CCOC(=O)CCCN(CC(O)CCCCCCCCCC)CC(O)CCCCCCCCCC)CC1)CC(O)CCCCCC(=O)OC(CCCCCCCC)CCCCCCCC] |
| 6 | 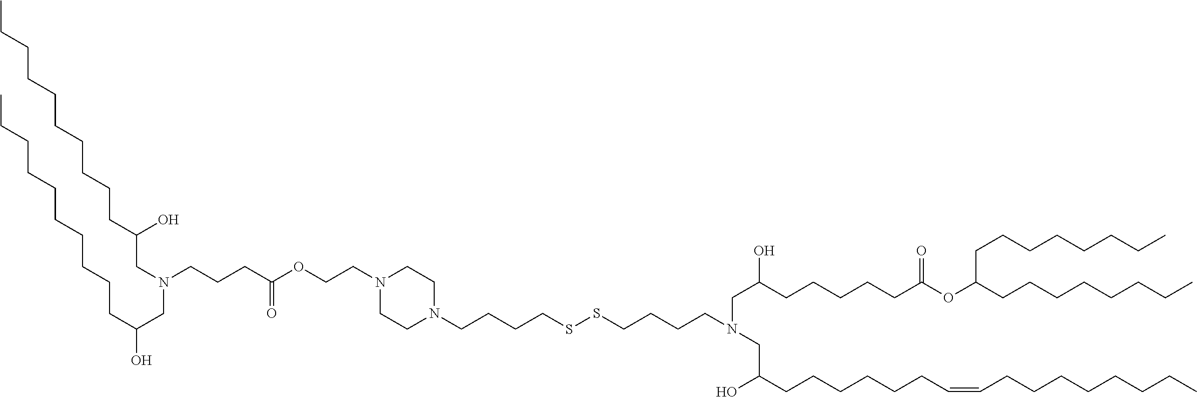 [SMILES: CCCCCCCC/C=C\CCCCCCC(O)CN(CCCCSSCCCCN1CCN(CCOC(=O)CCCN(CC(O)CCCCCCCCCC)CC(O)CCCCCCCCCC)CC1)CC(O)CCCCCC(=O)OC(CCCCCCCC)CCCCCCCC] |
| 7 | 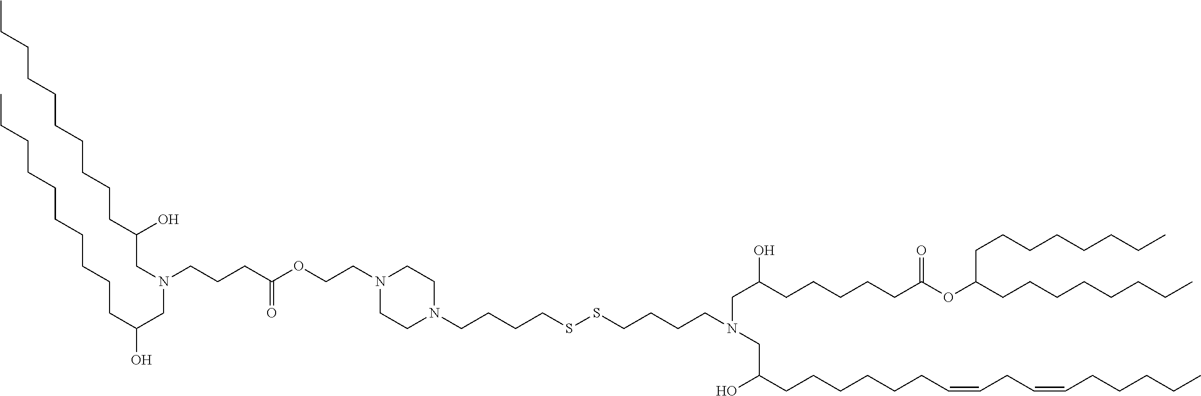 [SMILES: CCCCC/C=C\C/C=C\CCCCCCC(O)CN(CCCCSSCCCCN1CCN(CCOC(=O)CCCN(CC(O)CCCCCCCCCC)CC(O)CCCCCCCCCC)CC1)CC(O)CCCCCC(=O)OC(CCCCCCCC)CCCCCCCC] |
| 8 | 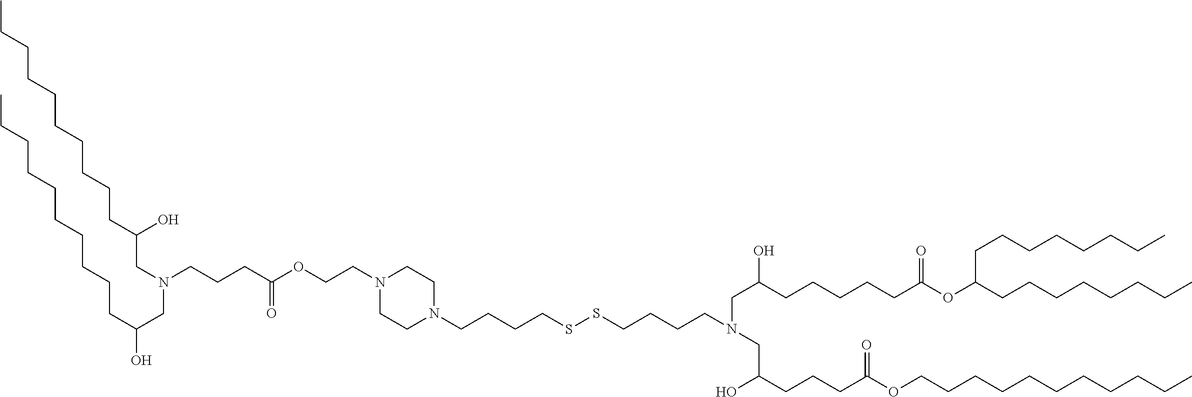 [SMILES: CCCCCCCCCCCOC(=O)CCCC(O)CN(CCCCSSCCCCN1CCN(CCOC(=O)CCCN(CC(O)CCCCCCCCCC)CC(O)CCCCCCCCCC)CC1)CC(O)CCCCCC(=O)OC(CCCCCCCC)CCCCCCCC] |

Formula (III)

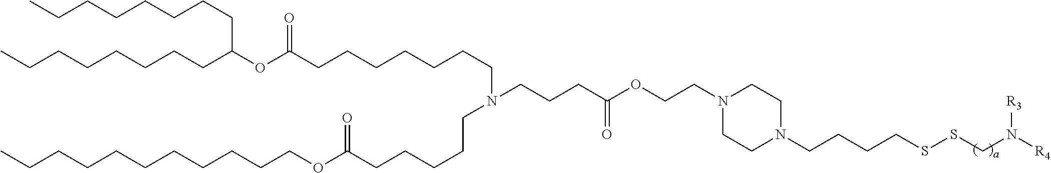

| | |
|---|---|
| 9 | 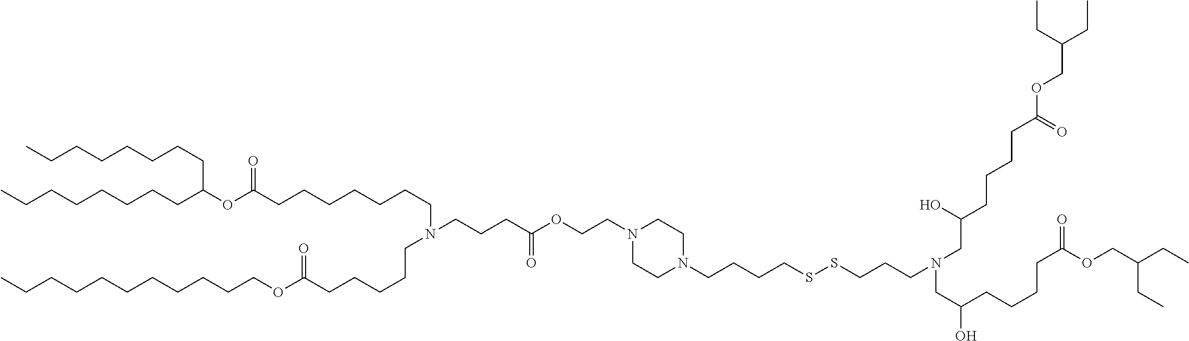 [SMILES: CCCCCCCCCCCOC(=O)CCCCCN(CCCCCCCC(=O)OC(CCCCCCCC)CCCCCCCC)CCCC(=O)OCCN1CCN(CCCCSSCCCN(CC(O)CCCCC(=O)OCC(CC)CC)CC(O)CCCCC(=O)OCC(CC)CC)CC1] |
| 10 | 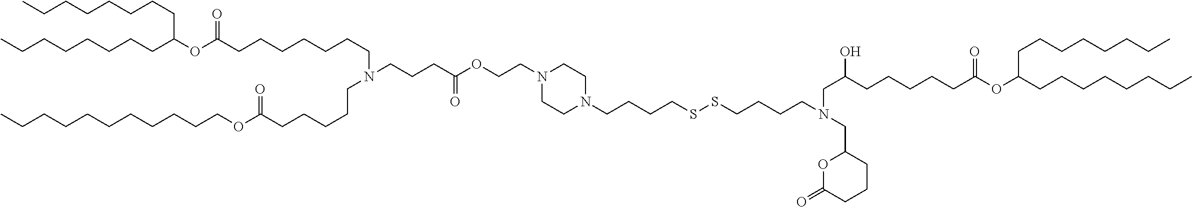 [SMILES: CCCCCCCCCCCOC(=O)CCCCCN(CCCCCCCC(=O)OC(CCCCCCCC)CCCCCCCC)CCCC(=O)OCCN1CCN(CCCCSSCCCCN(CC(O)CCCCCC(=O)OC(CCCCCCCC)CCCCCCCC)CC2CCCC(=O)O2)CC1] |

Formula (IV)

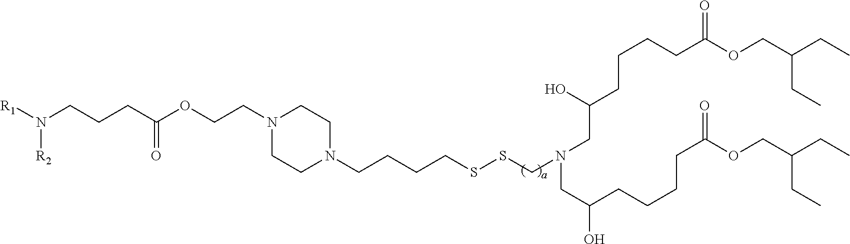

| | |
|---|---|
| 11 | 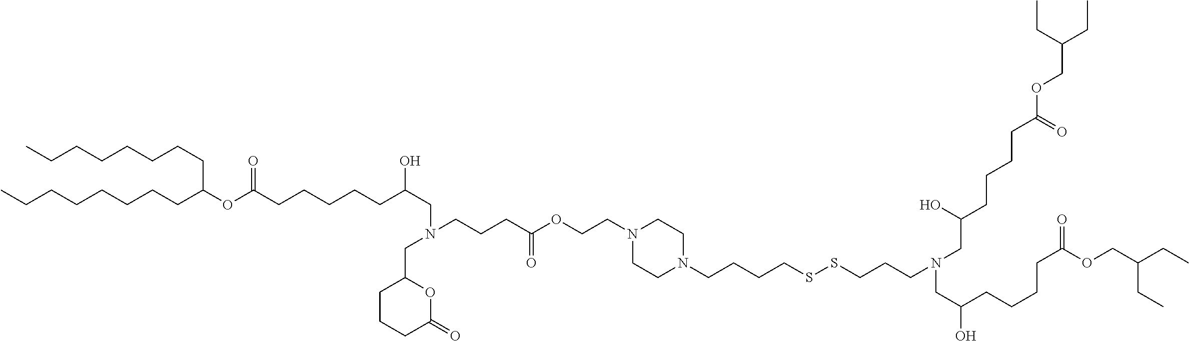 [SMILES: CCCCCCCCC(CCCCCCCC)OC(=O)CCCCCC(O)CN(CCCC(=O)OCCN1CCN(CCCCSSCCCN(CC(O)CCCCC(=O)OCC(CC)CC)CC(O)CCCCC(=O)OCC(CC)CC)CC1)CC1CCCC(=O)O1] |
| 12 | 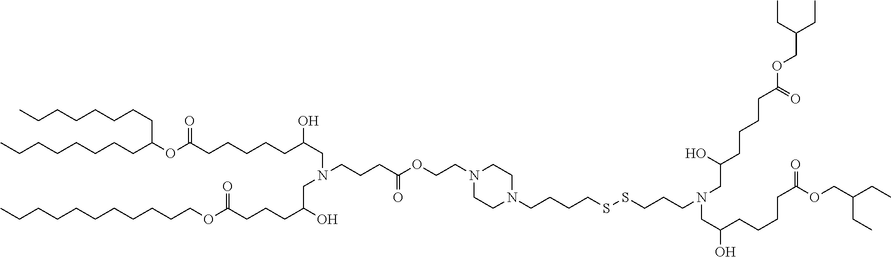 [SMILES: CCCCCCCCCCCOC(=O)CCCC(O)CN(CCCC(=O)OCCN1CCN(CCCCSSCCCN(CC(O)CCCCC(=O)OCC(CC)CC)CC(O)CCCCC(=O)OCC(CC)CC)CC1)CC(O)CCCCCC(=O)OC(CCCCCCCC)CCCCCCCC] |

Formula (V)

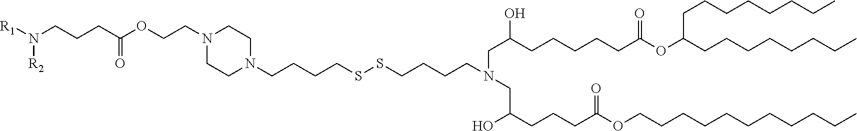

| | |
|---|---|
| 13 | 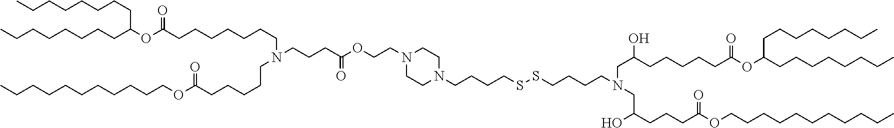 [SMILES: CCCCCCCCCCCOC(=O)CCCCCN(CCCCCCCC(=O)OC(CCCCCCCC)CCCCCCCC)CCCC(=O)OCCN1CCN(CCCCSSCCCCN(CC(O)CCCCCC(=O)OC(CCCCCCCC)CCCCCCCC)CC(O)CCCC(=O)OCCCCCCCCCCC)CC1] |
| 14 | 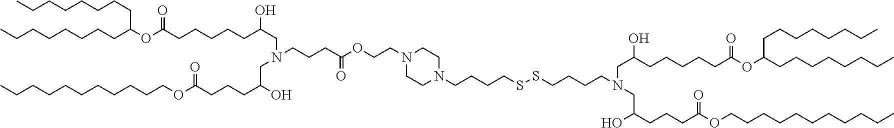 [SMILES: CCCCCCCCCCCOC(=O)CCCC(O)CN(CCCCSSCCCCN1CCN(CCOC(=O)CCCN(CC(O)CCCCCC(=O)OC(CCCCCCCC)CCCCCCCC)CC(O)CCCC(=O)OCCCCCCCCCCC)CC1)CC(O)CCCCCC(=O)OC(CCCCCCCC)CCCCCCCC] |

TABLE 2
Table 2
Formula (VI) (symmetric compounds)

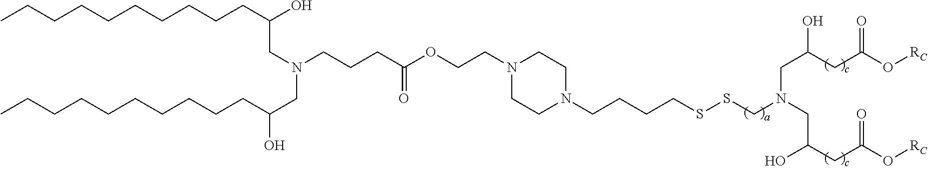

| | |
|---|---|
| 15 | 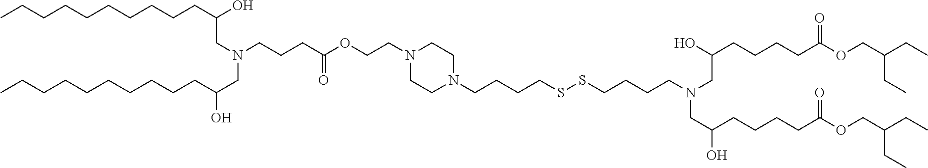 [SMILES: CCCCCCCCCCC(O)CN(CCCC(=O)OCCN1CCN(CCCCSSCCCCN(CC(O)CCCCC(=O)OCC(CC)CC)CC(O)CCCCC(=O)OCC(CC)CC)CC1)CC(O)CCCCCCCCCC] |
| 16 | 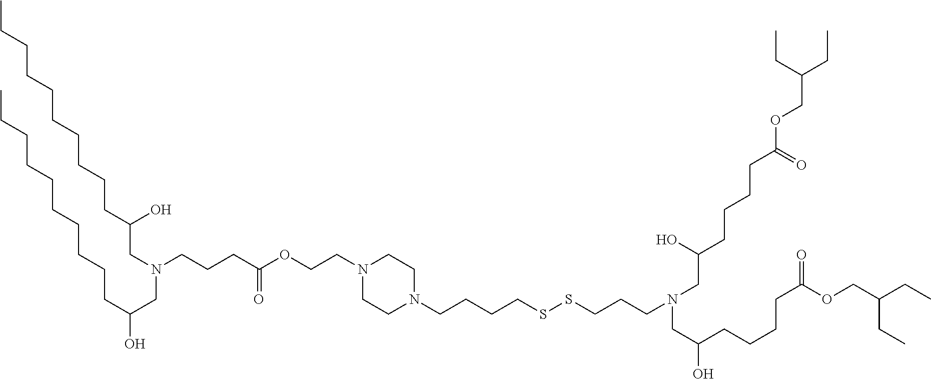 [SMILES: CCCCCCCCCCC(O)CN(CCCC(=O)OCCN1CCN(CCCCSSCCCN(CC(O)CCCCC(=O)OCC(CC)CC)CC(O)CCCCC(=O)OCC(CC)CC)CC1)CC(O)CCCCCCCCCC] |
| 17 | 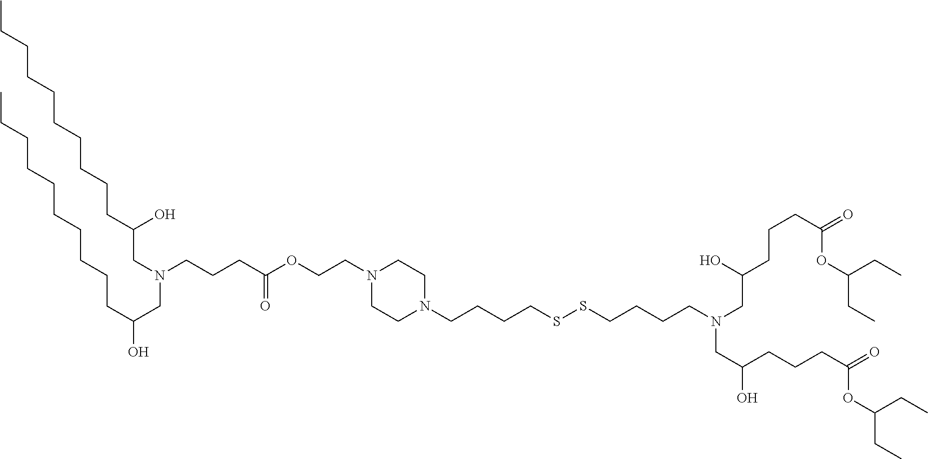 [SMILES: CCCCCCCCCCC(O)CN(CCCC(=O)OCCN1CCN(CCCCSSCCCCN(CC(O)CCCC(=O)OC(CC)CC)CC(O)CCCC(=O)OC(CC)CC)CC1)CC(O)CCCCCCCCCC] |
| 18 | 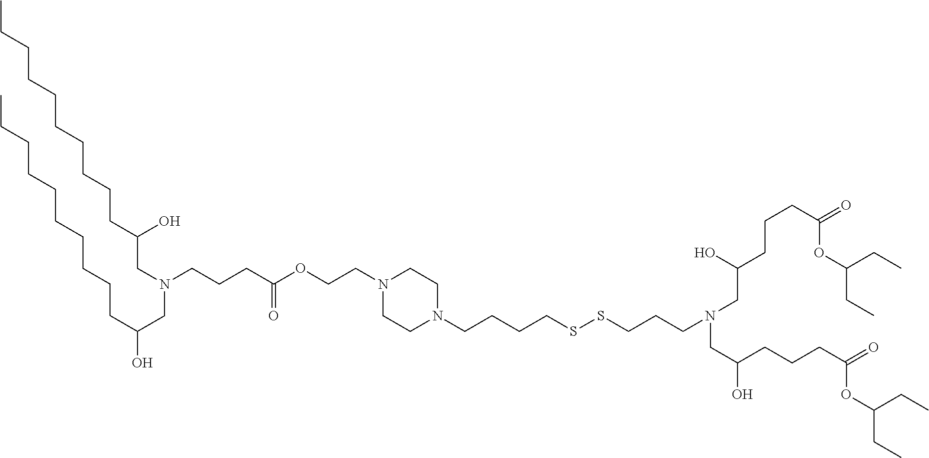 [SMILES: CCCCCCCCCCC(O)CN(CCCC(=O)OCCN1CCN(CCCCSSCCCN(CC(O)CCCC(=O)OC(CC)CC)CC(O)CCCC(=O)OC(CC)CC)CC1)CC(O)CCCCCCCCCC] |

Any of the compounds (1-18) identified in Table 1 and Table 2 above may be provided in the form of a pharmaceutically acceptable salt and such salts are intended to be encompassed by the present invention.

The compounds of the invention as described herein can be prepared according to methods known in the art, including the exemplary syntheses of the Examples provided herein.

### Nucleic Acids

The compounds of the invention as described herein can be used to prepare compositions useful for the delivery of nucleic acids.

### Synthesis of Nucleic Acids

Nucleic acids according to the present invention may be synthesized according to any known methods. For example, mRNAs according to the present invention may be synthesized via in vitro transcription (IVT). Briefly, IVT is typically performed with a linear or circular DNA template containing a promoter, a pool of ribonucleotide triphosphates, a buffer system that may include DTT and magnesium ions, and an appropriate RNA polymerase (e.g., T3, T7, mutated T7 or SP6 RNA polymerase), DNAse I, pyrophosphatase, and/or RNAse inhibitor. The exact conditions will vary according to the specific application.

In some embodiments, for the preparation of mRNA according to the invention, a DNA template is transcribed in vitro. A suitable DNA template typically has a promoter, for example a T3, T7, mutated T7 or SP6 promoter, for in vitro transcription, followed by desired nucleotide sequence for desired mRNA and a termination signal.

Desired mRNA sequence(s) according to the invention may be determined and incorporated into a DNA template using standard methods. For example, starting from a desired amino acid sequence (e.g., an enzyme sequence), a virtual reverse translation is carried out based on the degenerated genetic code. Optimization algorithms may then be used for selection of suitable codons. Typically, the G/C content can be optimized to achieve the highest possible G/C content on one hand, taking into the best possible account the frequency of the tRNAs according to codon usage on the other hand. The optimized RNA sequence can be established and displayed, for example, with the aid of an appropriate display device and compared with the original (wild-type) sequence. A secondary structure can also be analyzed to calculate stabilizing and destabilizing properties or, respectively, regions of the RNA.

Modified mRNA

In some embodiments, mRNA according to the present invention may be synthesized as unmodified or modified mRNA. Modified mRNA comprises nucleotide modifications in the RNA. A modified mRNA according to the invention can thus include nucleotide modification that are, for example, backbone modifications, sugar modifications or base modifications. In some embodiments, mRNAs may be synthesized from naturally occurring nucleotides and/or nucleotide analogues (modified nucleotides) including, but not limited to, purines (adenine (A), guanine (G)) or pyrimidines (thymine (T), cytosine (C), uracil (U)), and as modified nucleotides analogues or derivatives of purines and pyrimidines, such as e.g., 1-methyl-adenine, 2-methyl-adenine, 2-methylthio-N-6-isopentenyl-adenine, N-6-methyl-adenine, N-6-isopentenyl-adenine, 2-thio-cytosine, 3-methyl-cytosine, 4-acetyl-cytosine, 5-methyl-cytosine, 2,6-diaminopurine, 1-methyl-guanine, 2-methyl-guanine, 2,2-dimethyl-guanine, 7-methyl-guanine, inosine, 1-methyl-inosine, pseudouracil (5-uracil), dihydro-uracil, 2-thio-uracil, 4-thio-uracil, 5-carboxymethylaminomethyl-2-thio-uracil, 5-(carboxyhydroxymethyl)-uracil, 5-fluoro-uracil, 5-bromo-uracil, 5-carboxymethylaminomethyl-uracil, 5-methyl-2-thio-uracil, 5-methyl-uracil, N-uracil-5-oxyacetic acid methyl ester, 5-methylaminomethyl-uracil, 5-methoxyaminomethyl-2-thio-uracil, 5′-methoxycarbonylmethyl-uracil, 5-methoxy-uracil, uracil-5-oxyacetic acid methyl ester, uracil-5-oxyacetic acid (v), 1-methyl-pseudouracil, queuosine, beta-D-mannosyl-queuosine, wybutoxosine, and phosphoramidates, phosphorothioates, peptide nucleotides, methylphosphonates, 7-deazaguanosine, 5-methylcytosine and inosine. The preparation of such analogues is known to a person skilled in the art e.g., from the U.S. Pat. Nos. 4,373,071, 4,401,796, 4,415,732, 4,458,066, 4,500,707, 4,668,777, 4,973,679, 5,047,524, 5,132,418, 5,153,319, 5,262,530 and 5,700,642, the disclosures of which are incorporated by reference in their entirety.

### Pharmaceutical Formulations of Cationic Lipids and Nucleic Acids

In certain embodiments, the compounds of the invention as described herein, as well as pharmaceutical and liposomal compositions comprising such lipids, can be used in formulations to facilitate the delivery of encapsulated materials (e.g., one or more polynucleotides such as mRNA) to, and subsequent transfection of one or more target cells. For example, in certain embodiments cationic lipids described herein (and compositions such as liposomal compositions comprising such lipids) are characterized as resulting in one or more of receptor-mediated endocytosis, clathrin-mediated and caveolae-mediated endocytosis, phagocytosis and macropinocytosis, fusogenicity, endosomal or lysosomal disruption and/or releasable properties that afford such compounds advantages relative other similarly classified lipids.

According to the present invention, a nucleic acid, e.g., mRNA encoding a protein (e.g., a full length, fragment or portion of a protein) as described herein may be delivered via a delivery vehicle comprising a compound of the invention as described herein.

As used herein, the terms “delivery vehicle,” “transfer vehicle,” “nanoparticle,” or grammatical equivalents thereof, are used interchangeably.

For example, the present invention provides a composition (e.g., a pharmaceutical composition) comprising a cationic lipids as described herein and one or more polynucleotides. A composition (e.g., a pharmaceutical composition) may further comprise
(i) one or more additional cationic lipids,
(ii) one or more non-cationic lipids,
(iii) one or more cholesterol-based lipids and/or
(iv) one or more PEG-modified lipids.

In certain embodiments a composition exhibits an enhanced (e.g., increased) ability to transfect one or more target cells. Accordingly, also provided herein are methods of transfecting one or more target cells. Such methods generally comprise the step of contacting the one or more target cells with the cationic lipids and/or pharmaceutical compositions disclosed herein (e.g., a liposomal formulation comprising a compound described herein encapsulating one or more polynucleotides) such that the one or more target cells are transfected with the materials encapsulated therein (e.g., one or more polynucleotides). As used herein, the terms “transfect” or “transfection” refer to the intracellular introduction of one or more encapsulated materials (e.g., nucleic acids and/or polynucleotides) into a cell (e.g., into a target cell). The introduced polynucleotide may be stably or transiently maintained in the target cell. The term “transfection efficiency” refers to the relative amount of such encapsulated material (e.g., polynucleotides) up-taken by, introduced into, and/or expressed by the target cell which is subject to transfection. In practice, transfection efficiency may be estimated by the amount of a reporter polynucleotide product produced by the target cells following transfection. In certain embodiments, the compounds and pharmaceutical compositions described herein demonstrate high transfection efficiencies thereby improving the likelihood that appropriate dosages of the encapsulated materials (e.g., one or more polynucleotides) will be delivered to the site of pathology and subsequently expressed, while at the same time minimizing potential systemic adverse effects or toxicity associated with the compound or their encapsulated contents.

Following transfection of one or more target cells by, for example, the polynucleotides encapsulated in the one or more lipid nanoparticles comprising the pharmaceutical or liposomal compositions disclosed herein, the production of the product (e.g., a polypeptide or protein) encoded by such polynucleotide may be stimulated and the capability of such target cells to express the polynucleotide and produce, for example, a polypeptide or protein of interest is enhanced. For example, transfection of a target cell by one or more compounds or pharmaceutical compositions encapsulating mRNA will enhance (i.e., increase) the production of the protein or enzyme encoded by such mRNA.

Further, delivery vehicles described herein (e.g., liposomal delivery vehicles) may be prepared to preferentially distribute to other target tissues, cells or organs, such as the heart, lungs, kidneys, spleen. In embodiments, the delivery vehicles described herein (e.g., liposomal delivery vehicles) may be prepared to preferentially distribute to the lungs. In embodiments, the lipid nanoparticles of the present invention may be prepared to achieve enhanced delivery to the target cells and tissues. For example, polynucleotides (e.g., mRNA) encapsulated in one or more of the compounds or pharmaceutical and liposomal compositions described herein can be delivered to and/or transfect targeted cells or tissues. In some embodiments, the encapsulated polynucleotides (e.g., mRNA) are capable of being expressed and functional polypeptide products produced (and in some instances excreted) by the target cell, thereby conferring a beneficial property to, for example the target cells or tissues. Such encapsulated polynucleotides (e.g., mRNA) may encode, for example, an antigen hormone, enzyme, receptor, polypeptide, peptide or other protein of interest.

### Liposomal Delivery Vehicles

In some embodiments, a composition is a suitable delivery vehicle. In embodiments, a composition is a liposomal delivery vehicle, e.g., a lipid nanoparticle.

The terms “liposomal delivery vehicle” and “liposomal composition” are used interchangeably.

Enriching liposomal compositions with one or more of the cationic lipids disclosed herein may be used as a means of improving the safety profile or otherwise conferring one or more desired properties to such enriched liposomal composition (e.g., improved delivery of the encapsulated polynucleotides to one or more target cells and/or reduced in vivo toxicity of a liposomal composition). Accordingly, also contemplated are pharmaceutical compositions, and in particular liposomal compositions, that comprise one or more of the cationic lipids disclosed herein.

Thus, in certain embodiments, the compounds of the invention as described herein may be used as a component of a liposomal composition to facilitate or enhance the delivery and release of encapsulated materials (e.g., one or more therapeutic agents) to one or more target cells (e.g., by permeating or fusing with the lipid membranes of such target cells).

As used herein, liposomal delivery vehicles, e.g., lipid nanoparticles, are usually characterized as microscopic vesicles having an interior aqua space sequestered from an outer medium by a membrane of one or more bilayers. Bilayer membranes of liposomes are typically formed by amphiphilic molecules, such as lipids of synthetic or natural origin that comprise spatially separated hydrophilic and hydrophobic domains (Lasic, Trends Biotechnol., 16:307-321, 1998). Bilayer membranes of the liposomes can also be formed by amphophilic polymers and surfactants (e.g., polymerosomes, niosomes, etc.). In the context of the present invention, a liposomal delivery vehicle typically serves to transport a desired mRNA to a target cell or tissue.

In certain embodiments, such compositions (e.g., liposomal compositions) are loaded with or otherwise encapsulate materials, such as for example, one or more biologically-active polynucleotides (e.g., mRNA).

In embodiments, a composition (e.g., a pharmaceutical composition) comprises an mRNA encoding a peptide or protein, encapsulated within a liposome. In embodiments, a liposome comprises:
(i) one or more cationic lipids,
(ii) one or more non-cationic lipids,
(iii) one or more cholesterol-based lipids and
(iv) one or more PEG-modified lipids, wherein at least one cationic lipid is a compound of the invention as described herein.

In embodiments, a composition comprises an mRNA encoding for a peptide or protein (e.g., any peptide or protein described herein). In embodiments, a composition comprises an mRNA encoding for a peptide (e.g., any peptide described herein). In embodiments, a composition comprises an mRNA encoding for a protein (e.g., any protein described herein).

In embodiments, a composition (e.g., a pharmaceutical composition) comprises a nucleic acid encapsulated within a liposome, wherein the liposome comprises a compound described herein.

In embodiments, a nucleic acid is an mRNA encoding a peptide or protein. In embodiments, an mRNA encodes a peptide or protein for use in the delivery to or treatment of the human body. Exemplary mRNAs are described herein.

In embodiments, a liposomal delivery vehicle (e.g., a lipid nanoparticle) can have a net positive charge.

In embodiments, a liposomal delivery vehicle (e.g., a lipid nanoparticle) can have a net negative charge.

In embodiments, a liposomal delivery vehicle (e.g., a lipid nanoparticle) can have a net neutral charge.

In embodiments, a lipid nanoparticle that encapsulates a nucleic acid (e.g., mRNA encoding a peptide or protein) comprises one or more compounds of the invention as described herein.

For example, the amount of a compound of the invention as described herein in a composition can be described as a percentage (“wt %”) of the combined dry weight of all lipids of a composition (e.g., the combined dry weight of all lipids present in a liposomal composition).

In embodiments of the pharmaceutical compositions described herein, a compound of the invention as described herein is present in an amount that is about 0.5 wt % to about 30 wt % (e.g., about 0.5 wt % to about 20 wt %) of the combined dry weight of all lipids present in a composition (e.g., a liposomal composition).

In embodiments, a compound of the invention as described herein is present in an amount that is about 1 wt % to about 30 wt %, about 1 wt % to about 20 wt %, about 1 wt % to about 15 wt %, about 1 wt % to about 10 wt %, or about 5 wt % to about 25 wt % of the combined dry weight of all lipids present in a composition (e.g., a liposomal composition). In embodiments, a compound of the invention as described herein is present in an amount that is about 0.5 wt % to about 5 wt %, about 1 wt % to about 10 wt %, about 5 wt % to about 20 wt %, or about 10 wt % to about 20 wt % of the combined dry weight of all lipids present in a composition such as a liposomal delivery vehicle.

In embodiments, the amount of a compound of the invention as described herein is present in an amount that is at least about 5 wt %, about 10 wt %, about 15 wt %, about 20 wt %, about 25 wt %, about 30 wt %, about 35 wt %, about 40 wt %, about 45 wt %, about 50 wt %, about 55 wt %, about 60 wt %, about 65 wt %, about 70 wt %, about 75 wt %, about 80 wt %, about 85 wt %, about 90 wt %, about 95 wt %, about 96 wt %, about 97 wt %, about 98 wt %, or about 99 wt % of the combined dry weight of total lipids in a composition (e.g., a liposomal composition).

In embodiments, the amount of a compound of the invention as described herein is present in an amount that is no more than about 5 wt %, about 10 wt %, about 15 wt %, about 20 wt %, about 25 wt %, about 30 wt %, about 35 wt %, about 40 wt %, about 45 wt %, about 50 wt %, about 55 wt %, about 60 wt %, about 65 wt %, about 70 wt %, about 75 wt %, about 80 wt %, about 85 wt %, about 90 wt %, about 95 wt %, about 96 wt %, about 97 wt %, about 98 wt %, or about 99 wt % of the combined dry weight of total lipids in a composition (e.g., a liposomal composition).

In embodiments, a composition (e.g., a liposomal delivery vehicle such as a lipid nanoparticle) comprises about 0.1 wt % to about 20 wt % (e.g., about 0.1 wt % to about 15 wt %) of a compound described herein. In embodiments, a delivery vehicle (e.g., a liposomal delivery vehicle such as a lipid nanoparticle) comprises about 0.5 wt %, about 1 wt %, about 3 wt %, about 5 wt %, or about 10 wt % of a compound described herein. In embodiments, a delivery vehicle (e.g., a liposomal delivery vehicle such as a lipid nanoparticle) comprises up to about 0.5 wt %, about 1 wt %, about 3 wt %, about 5 wt %, about 10 wt %, about 15 wt %, or about 20 wt % of a compound described herein. In embodiments, the percentage results in an improved beneficial effect (e.g., improved delivery to targeted tissues).

The amount of a compound of the invention as described herein in a composition also can be described as a percentage (“mol %”) of the combined molar amounts of total lipids of a composition (e.g., the combined molar amounts of all lipids present in a liposomal delivery vehicle).

In embodiments of pharmaceutical compositions described herein, a compound of the invention as described herein is present in an amount that is about 0.5 mol % to about 50 mol % (e.g., about 0.5 mol % to about 20 mol %) of the combined molar amounts of all lipids present in a composition such as a liposomal delivery vehicle.

In embodiments, a compound of the invention as described herein is present in an amount that is about 0.5 mol % to about 5 mol %, about 1 mol % to about 10 mol %, about 5 mol % to about 20 mol %, about 10 mol % to about 20 mol %, about 15 mol % to about 30 mol %, about 20 mol % to about 35 mol %, about 25 mol % to about 40 mol %, about 30 mol % to about 45 mol %, about 35 mol % to about 50 mol %, about 40 mol % to about 55 mol %, or about 45 mol % to about 60 mol % of the combined molar amounts of all lipids present in a composition such as a liposomal delivery vehicle. In embodiments, a compound of the invention as described herein is present in an amount that is about 1 mol % to about 60 mol %, 1 mol % to about 50 mol %, 1 mol % to about 40 mol %, 1 mol % to about 30 mol %, about 1 mol % to about 20 mol %, about 1 mol % to about 15 mol %, about 1 mol % to about 10 mol %, about 5 mol % to about 55 mol %, about 5 mol % to about 45 mol %, about 5 mol % to about 35 mol % or about 5 mol % to about 25 mol % of the combined molar amounts of all lipids present in a composition such as a liposomal delivery vehicle.

In certain embodiments, a compound of the invention as described herein can comprise from about 0.1 mol % to about 50 mol %, or from 0.5 mol % to about 50 mol %, or from about 1 mol % to about 50 mol %, or from about 5 mol % to about 50 mol %, or from about 10 mol % to about 50 mol %, or from about 15 mol % to about 50 mol %, or from about 20 mol % to about 50 mol %, or from about 25 mol % to about 50 mol %, or from about 30 mol % to about 50 mol %, of the total amount of lipids in a composition (e.g., a liposomal delivery vehicle).

In certain embodiments, a compound of the invention as described herein can comprise greater than about 0.1 mol %, or greater than about 0.5 mol %, or greater than about 1 mol %, greater than about 5 mol %, greater than about 10 mol %, greater than about 20 mol %, greater than about 30 mol %, or greater than about 40 mol % of the total amount of lipids in the lipid nanoparticle.

In certain embodiments, a compound as described can comprise less than about 60 mol %, or less than about 55 mol %, or less than about 50 mol %, or less than about 45 mol %, or less than about 40 mol %, or less than about 35 mol %, less than about 30 mol %, or less than about 25 mol %, or less than about 10 mol %, or less than about 5 mol %, or less than about 1 mol % of the total amount of lipids in a composition (e.g., a liposomal delivery vehicle).

In embodiments, the amount of a compound of the invention as described herein is present in an amount that is at least about 5 mol %, about 10 mol %, about 15 mol %, about 20 mol %, about 25 mol %, about 30 mol %, about 35 mol %, about 40 mol %, about 45 mol %, about 50 mol %, about 55 mol %, about 60 mol %, about 65 mol %, about 70 mol %, about 75 mol %, about 80 mol %, about 85 mol %, about 90 mol %, about 95 mol %, about 96 mol %, about 97 mol %, about 98 mol %, or about 99 mol % of the combined molar amounts of total lipids in a composition (e.g., a liposomal composition).

In embodiments, the amount of a compound of the invention as described herein is present in an amount that is no more than about 5 mol %, about 10 mol %, about 15 mol %, about 20 mol %, about 25 mol %, about 30 mol %, about 35 mol %, about 40 mol %, about 45 mol %, about 50 mol %, about 55 mol %, about 60 mol %, about 65 mol %, about 70 mol %, about 75 mol %, about 80 mol %, about 85 mol %, about 90 mol %, about 95 mol %, about 96 mol %, about 97 mol %, about 98 mol %, or about 99 mol % of the combined molar amounts of total lipids in a composition (e.g., a liposomal composition).

In embodiments, the percentage results in an improved beneficial effect (e.g., improved delivery to targeted tissues).

In a typical embodiment, a composition of the invention (e.g., a liposomal composition) comprises:
(i) one or more cationic lipids,
(ii) one or more non-cationic lipids,
(iii) one or more cholesterol-based lipids, and
(iv) one or more PEG-modified lipids, wherein at least one cationic lipid is a compound of the invention as described herein.

For example, a composition suitable for practicing the invention has four lipid components comprising a compound of the invention as described herein as the cationic lipid component, and further comprising:
(i) a non-cationic lipid,
(ii) a cholesterol-based lipid and
(iii) a PEG-modified lipid.

The non-cationic lipid may be DOPE or DEPE. The cholesterol-based lipid may be cholesterol. The PEG-modified lipid may be DMG-PEG2K.

In further embodiments, pharmaceutical (e.g., liposomal) compositions comprise one or more of a PEG-modified lipid, a non-cationic lipid and a cholesterol lipid. In other embodiments, such pharmaceutical (e.g., liposomal) compositions comprise: one or more PEG-modified lipids; one or more non-cationic lipids; and one or more cholesterol lipids. In yet further embodiments, such pharmaceutical (e.g., liposomal) compositions comprise: one or more PEG-modified lipids and one or more cholesterol lipids.

In embodiments, a composition (e.g., lipid nanoparticle) that encapsulates a nucleic acid (e.g., mRNA encoding a peptide or protein) comprises one or more compounds of the invention as described herein, and one or more lipids selected from the group consisting of a cationic lipid, a non-cationic lipid, and a PEGylated lipid.

In embodiments, a composition (e.g., lipid nanoparticle) that encapsulates a nucleic acid (e.g., mRNA encoding a peptide or protein) comprises one or more compound of the invention as described herein; one or more lipids selected from the group consisting of a cationic lipid, a non-cationic lipid, and a PEGylated lipid; and further comprises a cholesterol-based lipid. Typically, such a composition has four lipid components comprising a compound of the invention as described herein as the cationic lipid component, and further comprising:
(i) a non-cationic lipid (e.g., DOPE),
(ii) a cholesterol-based lipid (e.g., cholesterol) and
(iii) a PEG-modified lipid (e.g., DMG-PEG2K).

In embodiments, a lipid nanoparticle that encapsulates a nucleic acid (e.g., mRNA encoding a peptide or protein) comprises one or more compounds of the invention as described herein, as well as one or more lipids selected from the group consisting of:
(i) a cationic lipid,
(ii) a non-cationic lipid,
(iii) a PEGylated lipid, and
(iv) a cholesterol-based lipid.

According to various embodiments, the selection of cationic lipids, non-cationic lipids and/or PEG-modified lipids which comprise the lipid nanoparticle, as well as the relative molar ratio of such lipids to each other, is based upon the characteristics of the selected lipid(s), the nature of the intended target cells, the characteristics of the mRNA to be delivered. Additional considerations include, for example, the saturation of the alkyl chain, as well as the size, charge, pH, pKa, fusogenicity and toxicity of the selected lipid(s). Thus, the molar ratios may be adjusted accordingly.

### Cationic Lipids

In addition to any of the compounds of the invention as described herein, a composition may comprise one or more additional cationic lipids.

In some embodiments, liposomes may comprise one or more additional cationic lipids. As used herein, the phrase “cationic lipid” refers to any of a number of lipid species that have a net positive charge at a selected pH, such as physiological pH. Several cationic lipids have been described in the literature, many of which are commercially available.

Suitable additional cationic lipids for use in the compositions include the cationic lipids as described in the literature.

### Helper Lipids

Compositions (e.g., liposomal compositions) may also comprise one or more helper lipids. Such helper lipids include non-cationic lipids. As used herein, the phrase “non-cationic lipid” refers to any neutral, zwitterionic or anionic lipid. As used herein, the phrase “anionic lipid” refers to any of a number of lipid species that carry a net negative charge at a selected pH, such as physiological pH. Non-cationic lipids include, but are not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), 1,2-Dierucoyl-sn-glycero-3-phosphoethanolamine (DEPE), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, 1-stearoyl-2-oleoyl-phosphatidyethanolamine (SOPE), or a mixture thereof. A non-cationic or helper lipid suitable for practicing the invention is dioleoylphosphatidylethanolamine (DOPE). Alternatively, 1,2-Dierucoyl-sn-glycero-3-phosphoethanolamine (DEPE) can be used as a non-cationic or helper lipid.

In some embodiments, a non-cationic lipid is a neutral lipid, i.e., a lipid that does not carry a net charge in the conditions under which the composition is formulated and/or administered.

In some embodiments, a non-cationic lipid may be present in a molar ratio (mol %) of about 5% to about 90%, about 5% to about 70%, about 5% to about 50%, about 5% to about 40%, about 5% to about 30%, about 10% to about 70%, about 10% to about 50%, or about 10% to about 40% of the total lipids present in a composition. In some embodiments, total non-cationic lipids may be present in a molar ratio (mol %) of about 5% to about 90%, about 5% to about 70%, about 5% to about 50%, about 5% to about 40%, about 5% to about 30%, about 10% to about 70%, about 10% to about 50%, or about 10% to about 40% of the total lipids present in a composition. In some embodiments, the percentage of non-cationic lipid in a liposome may be greater than about 5 mol %, greater than about 10 mol %, greater than about 20 mol %, greater than about 30 mol %, or greater than about 40 mol %. In some embodiments, the percentage total non-cationic lipids in a liposome may be greater than about 5 mol %, greater than about 10 mol %, greater than about 20 mol %, greater than about 30 mol %, or greater than about 40 mol %. In some embodiments, the percentage of non-cationic lipid in a liposome is no more than about 5 mol %, no more than about 10 mol %, no more than about 20 mol %, no more than about 30 mol %, or no more than about 40 mol %. In some embodiments, the percentage total non-cationic lipids in a liposome may be no more than about 5 mol %, no more than about 10 mol %, no more than about 20 mol %, no more than about 30 mol %, or no more than about 40 mol %.

In some embodiments, a non-cationic lipid may be present in a weight ratio (wt %) of about 5% to about 90%, about 5% to about 70%, about 5% to about 50%, about 5% to about 40%, about 5% to about 30%, about 10% to about 70%, about 10% to about 50%, or about 10% to about 40% of the total lipids present in a composition. In some embodiments, total non-cationic lipids may be present in a weight ratio (wt %) of about 5% to about 90%, about 5% to about 70%, about 5% to about 50%, about 5% to about 40%, about 5% to about 30%, about 10% to about 70%, about 10% to about 50%, or about 10% to about 40% of the total lipids present in a composition. In some embodiments, the percentage of non-cationic lipid in a liposome may be greater than about 5 wt %, greater than about 10 wt %, greater than about 20 wt %, greater than about 30 wt %, or greater than about 40 wt %. In some embodiments, the percentage total non-cationic lipids in a liposome may be greater than about 5 wt %, greater than about 10 wt %, greater than about 20 wt %, greater than about 30 wt %, or greater than about 40 wt %. In some embodiments, the percentage of non-cationic lipid in a liposome is no more than about 5 wt %, no more than about 10 wt %, no more than about 20 wt %, no more than about 30 wt %, or no more than about 40 wt %. In some embodiments, the percentage total non-cationic lipids in a liposome may be no more than about 5 wt %, no more than about 10 wt %, no more than about 20 wt %, no more than about 30 wt %, or no more than about 40 wt %.

### Cholesterol-Based Lipids

In some embodiments, a composition (e.g., a liposomal composition) comprising a cationic lipid of the present invention further comprises one or more cholesterol-based lipids. For example, a suitable cholesterol-based lipid for practicing the invention is cholesterol. Other suitable cholesterol-based lipids include, for example, DC-Chol (N,N-dimethyl-N-ethylcarboxamidocholesterol), 1,4-bis(3-N-oleylamino-propyl) piperazine (Gao, et al. Biochem. Biophys. Res. Comm. 179, 280 (1991); Wolf et al. BioTechniques 23, 139 (1997); U.S. Pat. No. 5,744,335), beta-sitosterol, or imidazole cholesterol ester (ICE), which has the following structure,

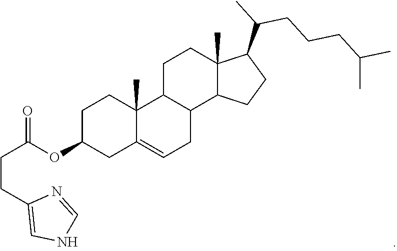 [SMILES: CC(C)CCCC(C)[C@H]1CCC2C3CC=C4C[C@@H](OC(=O)CCc5c[nH]cn5)CC[C@]4(C)C3CC[C@@]21C]

In some embodiments, a cholesterol-based lipid may be present in a molar ratio (mol %) of about 1% to about 30%, or about 5% to about 20% of the total lipids present in a liposome. In some embodiments, the percentage of cholesterol-based lipid in the lipid nanoparticle may be greater than about 5 mol %, greater than about 10 mol %, greater than about 20 mol %, greater than about 30 mol %, or greater than about 40 mol %. In some embodiments, the percentage of cholesterol-based lipid in the lipid nanoparticle may be no more than about 5 mol %, no more than about 10 mol %, no more than about 20 mol %, no more than about 30 mol %, or no more than about 40 mol %.

In some embodiments, a cholesterol-based lipid may be present in a weight ratio (wt %) of about 1% to about 30%, or about 5% to about 20% of the total lipids present in a liposome. In some embodiments, the percentage of cholesterol-based lipid in the lipid nanoparticle may be greater than about 5 wt %, greater than about 10 wt %, greater than about 20 wt %, greater than about 30 wt %, or greater than about 40 wt %. In some embodiments, the percentage of cholesterol-based lipid in the lipid nanoparticle may be no more than about 5 wt %, no more than about 10 wt %, no more than about 20 wt %, no more than about 30 wt %, or no more than about 40 wt %.

### PEGylated Lipids

In some embodiments, a composition (e.g., a liposomal composition) comprises one or more further PEGylated lipids. A suitable PEG-modified or PEGylated lipid for practicing the invention is 1,2-dimyristoyl-rac-glycero-3-methoxypolyethylene glycol-2000 (DMG-PEG2K).

For example, the use of polyethylene glycol (PEG)-modified phospholipids and derivatized lipids such as derivatized ceramides (PEG-CER), including N-octanoyl-sphingosine-1-[succinyl(methoxy polyethylene glycol)-2000] (C8 PEG-2000 ceramide) is also contemplated by the present invention in combination with one or more of compounds of the invention as described herein and, in some embodiments, other lipids together which comprise the liposome. In some embodiments, particularly useful exchangeable lipids are PEG-ceramides having shorter acyl chains (e.g., (C₁₄) or (C₁₈)).

Contemplated further PEG-modified lipids (also referred to herein as a PEGylated lipid, which term is interchangeable with PEG-modified lipid) include, but are not limited to, a polyethylene glycol chain of up to 5 kDa in length covalently attached to a lipid with alkyl chain(s) of (C₆-C₂₀) length. In some embodiments, a PEG-modified or PEGylated lipid is PEGylated cholesterol or PEG-2K. The addition of such components may prevent complex aggregation and may also provide a means for increasing circulation lifetime and increasing the delivery of the lipid-nucleic acid composition to the target cell, (Klibanov et al. (1990) FEBS Letters, 268 (1): 235-237), or they may be selected to rapidly exchange out of the formulation in vivo (see U.S. Pat. No. 5,885,613).

Further PEG-modified phospholipid and derivatized lipids of the present invention may be present in a molar ratio (mol %) from about 0% to about 10%, about 0.5% to about 10%, about 1% to about 10%, about 2% to about 10%, about 3% to about 5%, about 1% to about 5%, or about 1.5% to about 3% of the total lipid present in the composition (e.g., a liposomal composition).

### Pharmaceutical Formulations and Therapeutic Uses

Compounds of the invention as described herein may be used in the preparation of compositions (e.g., to construct liposomal compositions) that facilitate or enhance the delivery and release of encapsulated materials (e.g., one or more therapeutic polynucleotides) to one or more target cells (e.g., by permeating or fusing with the lipid membranes of such target cells).

For example, when a liposomal composition (e.g., a lipid nanoparticle) comprises or is otherwise enriched with one or more of the compounds disclosed herein, the phase transition in the lipid bilayer of the one or more target cells may facilitate the delivery of the encapsulated materials (e.g., one or more therapeutic polynucleotides encapsulated in a lipid nanoparticle) into the one or more target cells.

Similarly, in certain embodiments compounds of the invention as described herein may be used to prepare liposomal vehicles that are characterized by their reduced toxicity in vivo. In certain embodiments, the reduced toxicity is a function of the high transfection efficiencies associated with the compositions disclosed herein, such that a reduced quantity of such composition may be administered to the subject to achieve a desired therapeutic response or outcome.

In certain embodiments, compounds of the invention as described herein may be used to prepare liposomal vehicles that are characterized by effective intranasal delivery of mRNA. In certain embodiments, compounds of the invention as described herein may be used to prepare liposomal vehicles that are characterized by effective pulmonary delivery of mRNA. In certain embodiments, compounds of the invention as described herein may be used to prepare liposomal vehicles that are characterized by achieving high levels of peptide or protein expression when delivering mRNA encoding for said peptide or protein by intravenous, intrathecal, intramuscular, intranasal, sublingual, or by pulmonary delivery, optionally through nebulization. In certain embodiments, compounds of the invention as described herein may be used to prepare liposomal vehicles that are characterized by achieving high levels of peptide or protein expression when delivering mRNA encoding for said peptide or protein by intramuscular delivery.

Thus, pharmaceutical formulations comprising a compound described and nucleic acids provided by the present invention may be used for various therapeutic disease and/or disease prevention purposes. To facilitate delivery of nucleic acids in vivo, a compound described herein and nucleic acids can be formulated in combination with one or more additional pharmaceutical carriers, targeting ligands or stabilizing reagents. In some embodiments, a compound described herein can be formulated via pre-mixed lipid solution. In other embodiments, a composition comprising a compound described herein can be formulated using post-insertion techniques into the lipid membrane of the nanoparticles. Techniques for formulation and administration of drugs may be found in “Remington's Pharmaceutical Sciences,” Mack Publishing Co., Easton, Pa., latest edition.

Suitable routes of administration include, for example, oral, rectal, vaginal, transmucosal, pulmonary including intratracheal or inhaled, or intestinal administration; parenteral delivery, including intradermal, transdermal (topical), intramuscular, subcutaneous, intramedullary injections, as well as intrathecal, direct intraventricular, intravenous, intraperitoneal, or intranasal. In embodiments, the route of administration is selected from intravenous, intrathecal, intramuscular, intranasal, sublingual, or by pulmonary delivery, optionally through nebulization. In embodiments, the route of administration can be intramuscular. In particular embodiments, the intramuscular administration is to a muscle selected from the group consisting of skeletal muscle, smooth muscle and cardiac muscle. In some embodiments the administration results in delivery of the nucleic acids to a muscle cell. In some embodiments the administration results in delivery of the nucleic acids to a hepatocyte (i.e., liver cell).

One route for administering a liposomal composition of the invention may be intravenous delivery, in particular when treating metabolic disorders, especially those affecting the liver (e.g., ornithine transcarbamylase (OTC) deficiency). Alternatively, depending on the disease or disorder to be treated, the liposomal composition may be administered via pulmonary delivery (e.g., for the treatment of cystic fibrosis). For vaccination, a liposomal composition of the invention is typically administered intramuscularly. Alternatively, a liposomal composition of the invention may be administered intranasally for vaccination. Diseases or disorders affecting the eye may be treated by administering a liposomal composition of the invention intravitreally.

Alternatively or additionally, pharmaceutical formulations of the invention may be administered in a local rather than systemic manner, for example, via injection of the pharmaceutical formulation directly into a targeted tissue (e.g., in a sustained release formulation). Local delivery can be affected in various ways, depending on the tissue to be targeted. Exemplary tissues in which mRNA may be delivered and/or expressed include, but are not limited to the liver, kidney, heart, spleen, serum, brain, skeletal muscle, lymph nodes, skin, and/or cerebrospinal fluid. For example, aerosols containing compositions of the present invention can be inhaled (for nasal, tracheal, or bronchial delivery); compositions of the present invention can be injected into the site of injury, disease manifestation, or pain, for example; compositions can be provided in lozenges for oral, tracheal, or esophageal application; can be supplied in liquid, tablet or capsule form for administration to the stomach or intestines, can be supplied in suppository form for rectal or vaginal application; or can even be delivered to the eye by use of creams, drops, or even injection.

Alternatively or additionally, pharmaceutical formulations of the invention may be administered intranasally. For example, the pharmaceutical formulations of the invention may be administered via nasal spray. Exemplary tissues in which mRNA may be delivered and/or expressed include, but are not limited to the lungs, heart, liver, spleen and muscle. In embodiments, the tissue to be targeted is in the lungs. In embodiments, the tissue to be targeted is in muscle.

Alternatively or additionally, pharmaceutical formulations of the invention may be administered by pulmonary delivery, optionally through nebulization or dry powder inhalation. In embodiments, the pharmaceutical formulations of the invention are administered by pulmonary delivery through nebulization. In embodiments, the pharmaceutical formulations of the invention are administered by pulmonary delivery through dry powder inhalation. Exemplary tissues in which mRNA may be delivered and/or expressed include, but are not limited to the lungs, heart, liver, spleen and muscle. In embodiments, the tissue to be targeted is in the lungs. In embodiments, the tissue to be targeted is in muscle.

Compositions described herein can comprise mRNA encoding peptides including those described herein (e.g., a polypeptide such as a protein).

In embodiments, a mRNA encodes a polypeptide. In embodiments, a mRNA encodes a peptide. In embodiments, the peptide is an antigen. In embodiments, a mRNA encodes a peptide for treating or preventing Flu. In embodiments, a mRNA encodes a peptide for treating or preventing influenza A virus. In embodiments, a mRNA encodes a peptide for treating or preventing influenza B virus. In embodiments, a mRNA encodes a peptide for treating or preventing chlamydia. In embodiments, a mRNA encodes a peptide for treating or preventing a disease or disorder induced by infection with P. gingivalis. In embodiments, a mRNA encodes a peptide for treating or preventing acne.

In embodiments, a mRNA encodes a protein. In embodiments, a mRNA encodes a protein for treating or preventing Flu. In embodiments, a mRNA encodes a protein for treating or preventing influenza A virus. In embodiments, a mRNA encodes a protein for treating or preventing influenza B virus. In embodiments, a mRNA encodes a protein for treating or preventing chlamydia. In embodiments, a mRNA encodes a protein for treating or preventing a disease or disorder induced by infection with P. gingivalis. In embodiments, a mRNA encodes a protein for treating or preventing acne.

In embodiments, the mRNA encodes an influenza antigen. In embodiments, the mRNA encodes an antigen of influenza A virus. In embodiments, the mRNA encodes an antigen of influenza B virus. In embodiments, the mRNA encodes an antigen of Chlamydia sp. bacteria. In embodiments, the mRNA encodes an antigen of C. trachomatis. In embodiments, the mRNA encodes an antigen of P. gingivalis. In embodiments, the mRNA encodes an acne antigen. In embodiments, the mRNA encodes an antigen of C. acnes.

The present invention provides methods for delivering a composition having full-length mRNA molecules encoding a peptide or protein of interest for use in the treatment of a subject, e.g., a human subject or a cell of a human subject or a cell that is treated and delivered to a human subject.

In embodiments, compositions comprising a cationic lipid described herein are provided for use in treating or preventing Flu in a subject (e.g. human). In embodiments, compositions comprising a cationic lipid described herein are provided for use in treating or preventing a disease or disorder caused by influenza A virus in a subject (e.g. human). In embodiments, compositions comprising a cationic lipid described herein are provided for use in treating or preventing a disease or disorder caused by influenza B virus in a subject (e.g. human). In embodiments, compositions comprising a cationic lipid described herein are provided for use in treating or preventing a chlamydia in a subject (e.g. human). In embodiments, compositions comprising a cationic lipid described herein are provided for use in treating or preventing C. trachomatis infection in a subject (e.g. human). In embodiments, compositions comprising a cationic lipid described herein are provided for use in treating or preventing acne in a subject (e.g. human). In embodiments, compositions comprising a cationic lipid described herein are provided for use in treating or preventing C. acnes infection in a subject (e.g. human). In embodiments, compositions comprising a cationic lipid described herein are provided for use in treating or preventing a disease or disorder induced by infection with P. gingivalis. In any of these embodiments, said composition further comprises mRNA encoding a polypeptide, peptide or antigen suitable for the treatment or prevention of said disease or disorder or infection, such as those described herein.

Provided herein is a method of treating or preventing Flu in a subject (e.g. human), the method comprising administering a composition of the invention comprising a cationic lipid described herein to the subject. Provided herein is a method of treating or preventing a disease or disorder caused by influenza A virus in a subject (e.g. human), the method comprising administering a composition of the invention comprising a cationic lipid described herein to the subject. Provided herein is a method of treating or preventing a disease or disorder caused by influenza B virus in a subject (e.g. human), the method comprising administering a composition of the invention comprising a cationic lipid described herein to the subject. Provided herein is a method of treating or preventing chlamydia in a subject (e.g. human), the method comprising administering a composition of the invention comprising a cationic lipid described herein to the subject. Provided herein is a method of treating or preventing C. trachomatis infection in a subject (e.g. human), the method comprising administering a composition of the invention comprising a cationic lipid described herein to the subject. Provided herein is a method of treating or preventing acne in a subject (e.g. human), the method comprising administering a composition of the invention comprising a cationic lipid described herein to the subject. Provided herein is a method of treating or preventing C. acnes infection in a subject (e.g. human), the method comprising administering a composition of the invention comprising a cationic lipid described herein to the subject. Provided herein is a method of treating or preventing a disease or disorder induced by infection with P. gingivalis in a subject (e.g. human), the method comprising administering a composition of the invention comprising a cationic lipid described herein to the subject. In any of these embodiments, said composition further comprises mRNA encoding a polypeptide, peptide or antigen suitable for the treatment or prevention of said disease or disorder or infection, such as those described herein.

### Delivery Methods

The route of delivery used in the methods of the invention allows for non-invasive, self-administration of the compounds of the invention. In some embodiments, the methods involve intranasal, intratracheal or pulmonary administration by aerosolization, nebulization, or instillation of a composition comprising mRNA encoding a therapeutic peptide or protein in a suitable transfection or lipid carrier vehicles as described above. In some embodiments, the methods involve intranasal, intratracheal or pulmonary administration by intravenous, intrathecal, intramuscular, intranasal, sublingual, or by pulmonary delivery, optionally through nebulization of a composition comprising mRNA encoding a therapeutic peptide or protein in a suitable transfection or lipid carrier vehicles as described above. In some embodiments, the administration is intramuscular. In some embodiments, the peptide or protein is encapsulated with a liposome. In some embodiments, the liposome comprises a lipid, which is a compound of the invention. As used herein below, administration of a compound of the invention includes administration of a composition comprising a compound of the invention.

In certain embodiments compositions of the invention are provided for use in skin injection, e.g., in the epidermis, the dermis or the hypodermis of the skin. In some embodiments, the compositions are provided in a device suitable for skin injection, such as a needle (e.g., an epidermic, dermic or hypodermic needle), a needle free device, a microneedle device or a microprojection array device. Examples of microneedle or microprojection array devices suitable for the skin injection as described in US20230270842A1, US20220339416A1, US20210085598A1, US20200246450A1, US20220143376A1, US20180264244A1, US20180263641A1, US20110245776A1.

Following administration of the composition to the subject, the peptide or protein product encoded by the mRNA (e.g., a functional protein or enzyme) is detectable in the peripheral target tissues for at least about one to seven days or longer following administration of the compound to the subject. The amount of peptide or protein product necessary to achieve a therapeutic effect will vary depending on the condition being treated, the peptide or protein encoded, and the condition of the patient. For example, the peptide or protein product may be detectable in the peripheral target tissues at a concentration (e.g., a therapeutic concentration) of at least 0.025-1.5 μg/ml (e.g., at least 0.050 μg/ml, at least 0.075 μg/ml, at least 0.1 μg/ml, at least 0.2 μg/ml, at least 0.3 μg/ml, at least 0.4 μg/ml, at least 0.5 μg/ml, at least 0.6 μg/ml, at least 0.7 μg/ml, at least 0.8 μg/ml, at least 0.9 μg/ml, at least 1.0 μg/ml, at least 1.1 μg/ml, at least 1.2 μg/ml, at least 1.3 μg/ml, at least 1.4 ug/ml, or at least 1.5 μg/ml), for at least about 1, 2, 3, 4, 5, 6, 7, 8, 9, 10, 11,12, 13, 14, 15, 16, 17, 18, 19, 20, 21, 22, 23, 24, 25, 26, 27, 28, 29, 30, 35, 40, 45 days or longer following administration of the compound to the subject.

It has been demonstrated that nucleic acids can be delivered to the lungs by intratracheal administration of a liquid suspension of the compound and inhalation of an aerosol mist produced by a liquid nebulizer or the use of a dry powder apparatus such as that described in U.S. Pat. No. 5,780,014, incorporated herein by reference.

In certain embodiments, the compounds of the invention may be formulated such that they may be aerosolized or otherwise delivered as a particulate liquid or solid prior to or upon administration to the subject. Such compounds may be administered with the assistance of one or more suitable devices for administering such solid or liquid particulate compositions (such as, e.g., an aerosolized aqueous solution or suspension) to generate particles that are easily respirable or inhalable by the subject. In some embodiments, such devices (e.g., a metered dose inhaler, jet-nebulizer, ultrasonic nebulizer, dry-powder-inhalers, propellant-based inhaler or an insufflator) facilitate the administration of a predetermined mass, volume or dose of the compositions (e.g., about 0.5 mg/kg of mRNA per dose) to the subject. For example, in certain embodiments, the compounds of the invention are administered to a subject using a metered dose inhaler containing a suspension or solution comprising the compound and a suitable propellant. In certain embodiments, the compounds of the invention may be formulated as a particulate powder (e.g., respirable dry particles) intended for inhalation. In certain embodiments, compositions of the invention formulated as respirable particles are appropriately sized such that they may be respirable by the subject or delivered using a suitable device (e.g., a mean D50 or D90 particle size less than about 500 μm, 400 μm, 300 μm, 250 μm, 200 μm, 150 μm, 100 μm, 75 μm, 50 μm, 25 μm, 20 μm, 15 μm, 12.5 μm, 10 μm, 5 μm, 2.5 μm or smaller). In yet other embodiments, the compounds of the invention are formulated to include one or more pulmonary surfactants (e.g., lamellar bodies). In some embodiments, the compounds of the invention are administered to a subject such that a concentration of at least 0.05 mg/kg, at least 0.1 mg/kg, at least 0.5 mg/kg, at least 1.0 mg/kg, at least 2.0 mg/kg, at least 3.0 mg/kg, at least 4.0 mg/kg, at least 5.0 mg/kg, at least 6.0 mg/kg, at least 7.0 mg/kg, at least 8.0 mg/kg, at least 9.0 mg/kg, at least 10 mg/kg, at least 15 mg/kg, at least 20 mg/kg, at least 25 mg/kg, at least 30 mg/kg, at least 35 mg/kg, at least 40 mg/kg, at least 45 mg/kg, at least 50 mg/kg, at least 55 mg/kg, at least 60 mg/kg, at least 65 mg/kg, at least 70 mg/kg, at least 75 mg/kg, at least 80 mg/kg, at least 85 mg/kg, at least 90 mg/kg, at least 95 mg/kg, or at least 100 mg/kg body weight is administered in a single dose. In some embodiments, the compounds of the invention are administered to a subject such that a total amount of at least 0.1 mg, at least 0.5 mg, at least 1.0 mg, at least 2.0 mg, at least 3.0 mg, at least 4.0 mg, at least 5.0 mg, at least 6.0 mg, at least 7.0 mg, at least 8.0 mg, at least 9.0 mg, at least 10 mg, at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg or at least 100 mg mRNA is administered in one or more doses.

### EXAMPLES

While certain compounds, compositions and methods of the present invention have been described with specificity in accordance with certain embodiments, the following examples serve only to illustrate the compounds of the invention and are not intended to limit the same.

### LIST OF ABBREVIATIONS

DCM: Dichloromethane
DIPEA: N,N-Diisopropylethylamine
DMAP: 4-Dimethylaminopyridine
EDC: 1-Ethyl-3-(3-dimethylaminopropyl) carbodiimide
EtOAc: Ethyl acetate
NaHCO₃: Sodium hydrogencarbonate
Py: Pyridine
Na₂SO₄: Sodium Sulfate
TEA: Triethylamine
TFA: Trifluoroacetic Acid
MS: Mass spectrometry
ESI-MS: Electrospray ionization mass spectrometry
TLC: Thin Layer Chromatography

### Example 1: Synthesis of Compounds of the Invention

The cationic lipid MC3 is the currently used for in vivo delivery of e.g. siRNA (see WO2010/144740). However, the synthesis of this lipid involves a six-step process and requires handling of a Grignard reagent. In contrast, the present invention provides cationic lipids that can be prepared from readily available starting reagents, such as “Good's” buffers (see Table A below). These starting reagents can be coupled to cationic headgroups and lipid tails using coupling reactions, such as sulfonylation, acetylation and alkylation (see for example, Tables B1 and B2 below).

TABLE A
Examples of “Good” buffers

| “Good” Buffer name | Structure |
|---|---|
| HEPES | 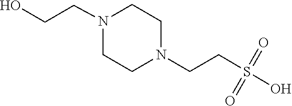 [SMILES: O=S(=O)(O)CCN1CCN(CCO)CC1] |
| HEPPS | 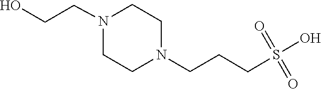 [SMILES: O=S(=O)(O)CCCN1CCN(CCO)CC1] |
| HEPBS | 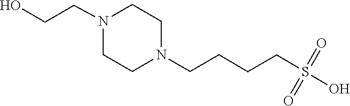 [SMILES: O=S(=O)(O)CCCCN1CCN(CCO)CC1] |

TABLE B1
Examples of lipid chains that are suitable for the present invention at positions R₁ and R₂
Example tails suitable for positions R₁ and R₂

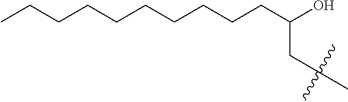 [SMILES: *CC(O)CCCCCCCCCC]

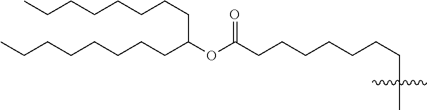 [SMILES: *CCCCCCCC(=O)OC(CCCCCCCC)CCCCCCCC]

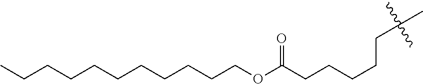 [SMILES: *CCCCCC(=O)OCCCCCCCCCCC]

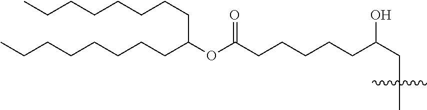 [SMILES: *CC(O)CCCCCC(=O)OC(CCCCCCCC)CCCCCCCC]

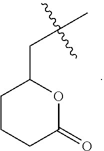 [SMILES: *CC1CCCC(=O)O1]

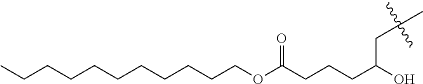 [SMILES: *CC(O)CCCC(=O)OCCCCCCCCCCC]

TABLE B2
Examples of lipid chains that are suitable for the present invention at positions R₃ and R₄
Example tails suitable for positions R₃ and R₄

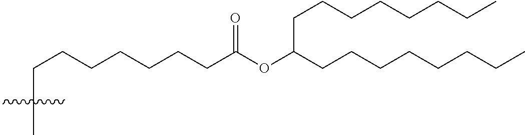 [SMILES: *CCCCCCCC(=O)OC(CCCCCCCC)CCCCCCCC]

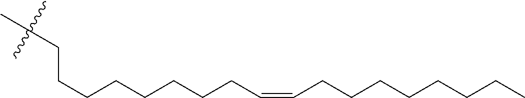 [SMILES: *CCCCCCCC/C=C\CCCCCCCC]

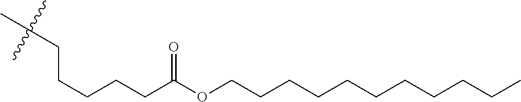 [SMILES: *CCCCCC(=O)OCCCCCCCCCCC]

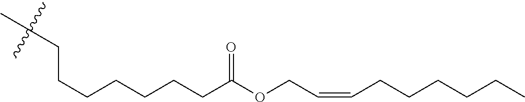 [SMILES: *CCCCCCCC(=O)OC/C=C\CCCCCC]

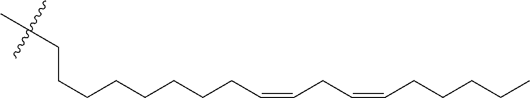 [SMILES: *CCCCCCCC/C=C\C/C=C\CCCCC]

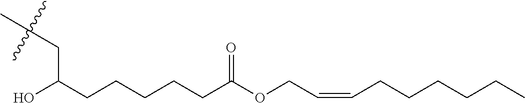 [SMILES: *CC(O)CCCCCC(=O)OC/C=C\CCCCCC]

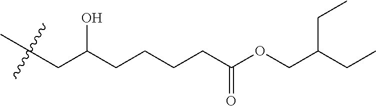 [SMILES: *CC(O)CCCCC(=O)OCC(CC)CC]

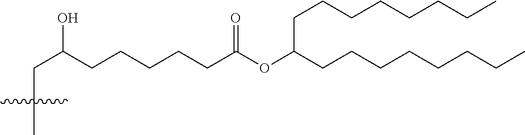 [SMILES: *CC(O)CCCCCC(=O)OC(CCCCCCCC)CCCCCCCC]

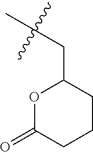 [SMILES: *CC1CCCC(=O)O1]

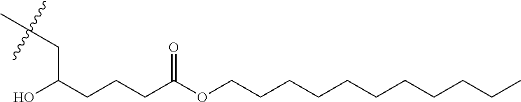 [SMILES: *CC(O)CCCC(=O)OCCCCCCCCCCC]

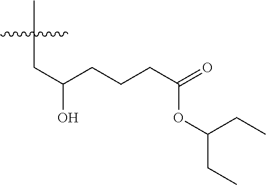 [SMILES: *CC(O)CCCC(=O)OC(CC)CC]

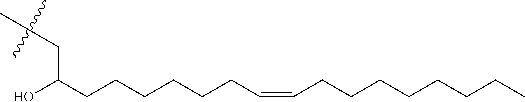 [SMILES: *CC(O)CCCCCC/C=C\CCCCCCCC]

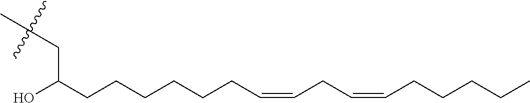 [SMILES: *CC(O)CCCCCC/C=C\C/C=C\CCCCC]

### Synthetic Protocol for Compound 1

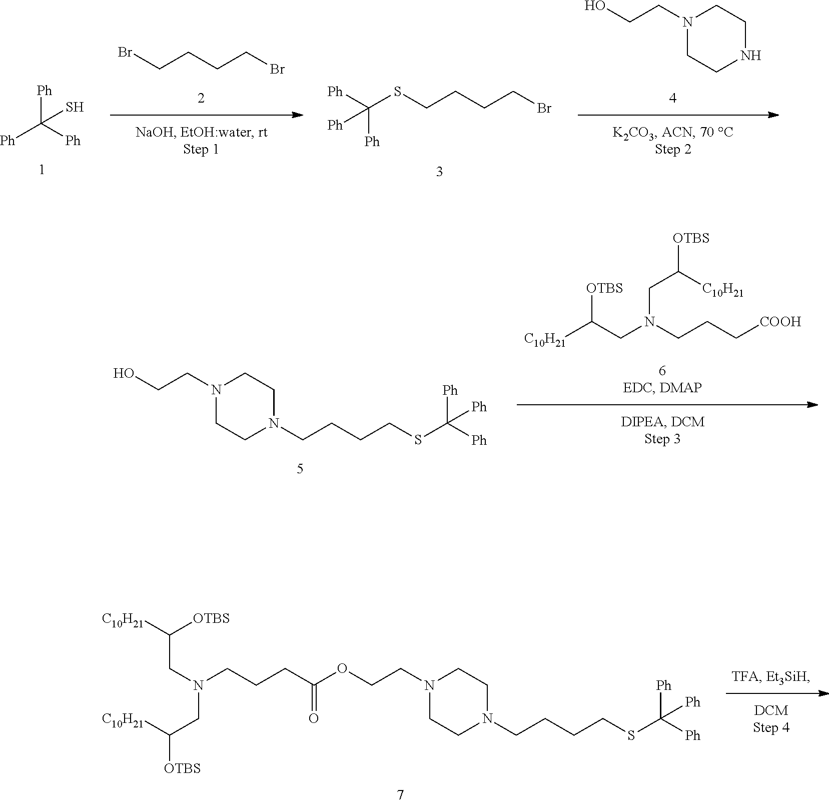

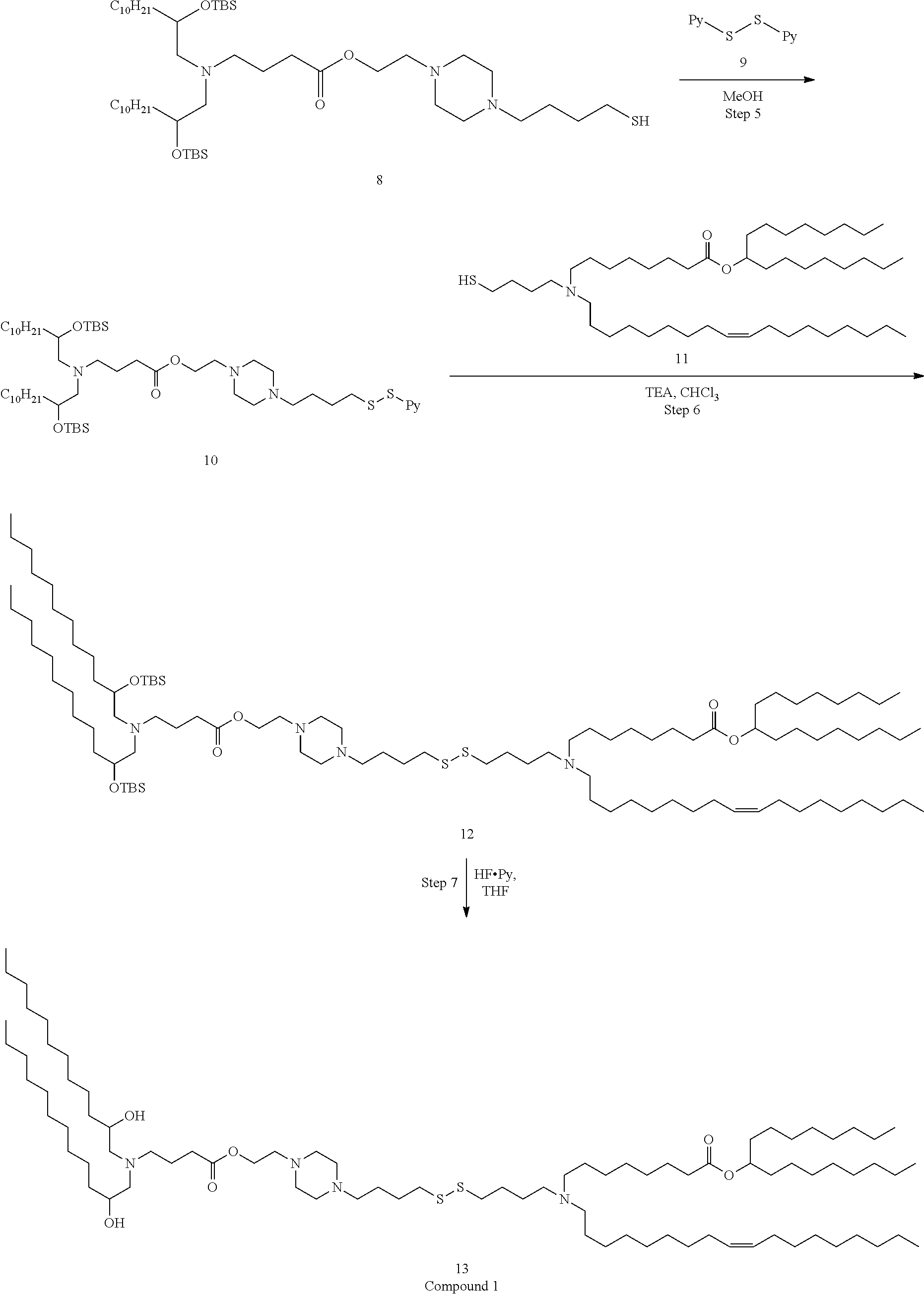

### Intermediate [3]:

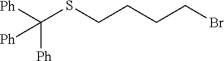 [SMILES: BrCCCCSC(c1ccccc1)(c1ccccc1)c1ccccc1]

To a solution of triphenylmethanethiol (5.0 g, 18.08 mmol) in EtOH (40 mL) and water (40 mL) was added a solution (in 40 ml water) of NaOH (1.44 g, 36.16 mmol). The reaction mixture was stirred for 10 min and added a solution (in 40 ml EtOH) of 1,4-dibromobutane (3.65 g, 18.08 mmol) to reaction mixture. The reaction mixture was stirred for 4 hours at room temperature. The progress of reaction was monitored by TLC (5% EtOAc/hexanes). The reaction mixture was diluted with DCM and aqueous sodium bicarbonate solution, the organic layer was washed with brine. The organic layer was dried over sodium sulphate, concentrated under vacuum to give crude compound. To the crude was added MeOH (15 mL) and stirred for 15 min at 0-10 °C, the solid compound was filtered and dried under vacuum to give [3] (5.1 g, 69%) as a white solid.

### Results:

¹H NMR (400 MHz, CDCl₃): δ 7.42-7.39 (m, 6H), 7.30-7.26 (m, 6H), 7.23-7.19 (m, 3H), 3.24 (t, 2H), 2.17 (t, 2H), 1.82-1.77 (m, 2H), 1.55-1.50 (m, 2H). LCMS: Purity 84.99% (low ionization)

### Intermediate [5]:

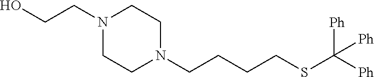 [SMILES: OCCN1CCN(CCCCSC(c2ccccc2)(c2ccccc2)c2ccccc2)CC1]

To a solution of [3] (5.0 g, 12.16 mmol) and [4] (3.16 g, 24.32 mmol) in ACN (75 mL) was added K₂CO₃ (6.72 g, 48.62 mmol). The reaction mixture was heated at 40 °C for 48 hours. The reaction progress was monitored by TLC (2.5% MeOH in DCM)). The reaction mixture was cooled to room temperature and filtered. The filtrate was concentrated under vacuum to give crude product. The crude was purified by flash chromatography (0 to 2.5% MeOH in DCM) to give [5] (2.6 g, 46%) as a white solid.

### Results:

¹H NMR (400 MHz, DMSO-d6): δ 7.41 (d, 6H), 7.28 (d, 6H), 7.20 (t, 3H), 3.59 (t, 2H), 2.73 (brs, 1H), 2.53-2.39 (m, 10H), 2.20-2.14 (m, 4H), 1.41 (brs, 4H). LCMS: Purity 98% ESI-MS analysis: Calculated C₂₉H₃₇N₂OS, [M+H]=461.26, Observed=461.29

### Intermediate [7]:

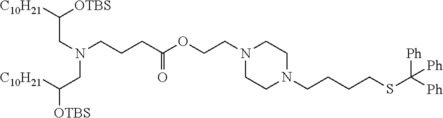

To a solution of [5] (0.5 g, 1.09 mmol) in DCM (8 mL) were added [6] (0.84 g, 1.19 mmol) in DCM (7 mL), EDC (0.31 g, 1.63 mmol), DMAP (27 mg, 0.22 mmol), DIPEA (0.38 mL, 2.17 mmol) and stirred at room temperature for 14 hours. After completion of the reaction as monitored by MS. The reaction mixture was diluted with DCM washed with NaHCO₃ solution, water and brine. The organic layer was dried over anhydrous Na₂SO₄, concentrated, and the crude compound was purified (eluent: 20% EtOAc in hexanes) to obtain pure compound [7] as a color less oil (0.75 g, 60%). It was confirmed by MS analysis.

### Results:

ESI-MS analysis: Calculated C₆₉H₁₂₀N₃O₄SSi₂, [M+H]=1142.85, Observed=1142.8

### Intermediate [8]:

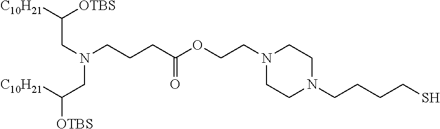

To a solution of [7] (0.75 g, 0.66 mmol) in DCM (6 mL) was slowly added TFA (6 mL) at room temperature and stirred at room temperature for 0.5 hour. To that triethylsilane (0.13 mL, 0.82 mmol) was added slowly and stirred for 1 hour. After completion of the reaction as monitored by MS. The reaction mixture was concentrated to obtain crude product [8] (>0.59 g, quantitative). It was confirmed by MS analysis.

### Results:

ESI-MS analysis: Calculated C₅₀H₁₀₆N₃O₄SSi₂, [M+H]=900.74, Observed=900.7

### Intermediate [10]:

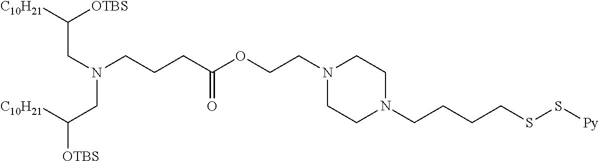

To a solution of [8] (0.59 g, 0.65 mmol) in MeOH (10 mL) was added [9] (0.22 g, 0.98 mmol) at room temperature and stirred for 2 hours. After completion of the reaction as monitored by MS. The reaction mixture was concentrated, and the crude compound was purified (eluent: 8% MeOH in DCM) to obtain pure product (0.45 g, 68%). It was confirmed by MS analysis.

### Results:

ESI-MS analysis: Calculated for C₅₅H₁₀₉N₄₀₄S₂Si₂, [M+H]=1009.74; Observed=1009.70

### Intermediate [12]:

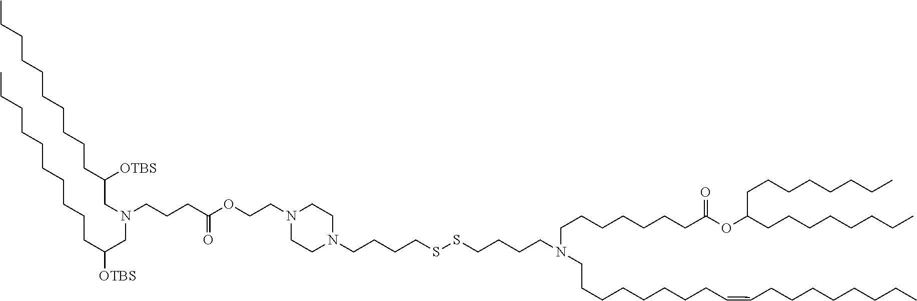 [SMILES: CCCCCCCC/C=C\CCCCCCCCN(CCCCCCCC(=O)OC(CCCCCCCC)CCCCCCCC)CCCCSSCCCCN1CCN(CCOC(=O)CCCN(CC(CCCCCCCCCC)O[Si](C)(C)C(C)(C)C)CC(CCCCCCCCCC)O[Si](C)(C)C(C)(C)C)CC1]

To a solution of (0.500 g, 0.495 mmol) and (0.583 g, 0.792 mmol) in chloroform was added triethylamine (0.35 ml, 2.47 mmol) and allowed to react at room temperature for 2.5 hours. After completion of the reaction, the reaction mixture was concentrated and purified to obtain as colorless oil (0.355 g, 44% yield).

ESI-MS analysis: Calculated for C₉₇H₁₉₆N₄₀₆S₂Si₂, [M+H]=1634.95; Observed=1634.10

### Compound 1 [13]:

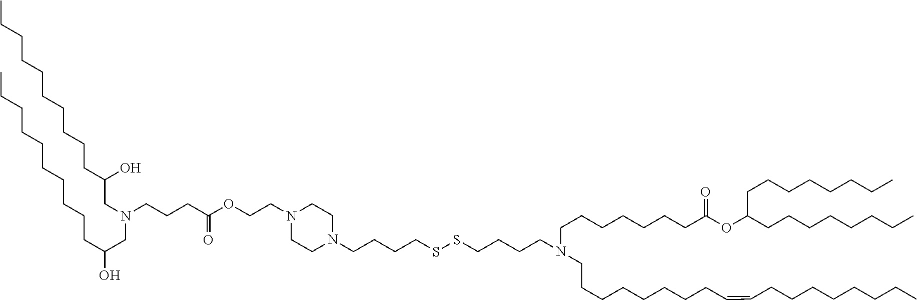 [SMILES: CCCCCCCC/C=C\CCCCCCCCN(CCCCCCCC(=O)OC(CCCCCCCC)CCCCCCCC)CCCCSSCCCCN1CCN(CCOC(=O)CCCN(CC(O)CCCCCCCCCC)CC(O)CCCCCCCCCC)CC1]

To a 20 ml polypropylene scintillation vial was added (0.355 g, 0.217 mmol, 1.0 eq) along with 4 mL of dry tetrahydrofuran. The vial was cooled to 0-5 °C and HF/pyridine (1.22 mL, 42.78 mmol) was added dropwise. After addition, the reaction vial was allowed to warm to room temperature and stirred for 18 hours. Afterwards, the reaction mixture was cooled back to 0 °C and neutralized with solid sodium bicarbonate solid, diluted with ethyl acetate, washed with NaHCO₃ solution, water and brine. The organic layer was dried over anhydrous Na₂SO₄ and concentrated. The crude product was purified to obtain compound (0.20 g, 65%). It was confirmed by ¹H NMR and MS analysis.

### Results:

¹H NMR (400 MHz, CDCl₃) 5.39-5.30 (m, 2H), 4.85 (quin, 1H), 4.19 (t, 2H), 3.62 (br, 2H), 3.03-2.18 (m, 29H), 2.07-1.89 (m, 4H), 1.88-1.54 (m, 16H), 1.53-1.40 (m, 8H), 1.39-1.14 (m, 90H), 0.86 (t, 15H).

ESI-MS analysis: Calculated for C₈₅H₁₆₈N₄₀₆S₂, [M+H]=1406.42; Observed=1406.94

### Synthetic Protocol for Compound 2

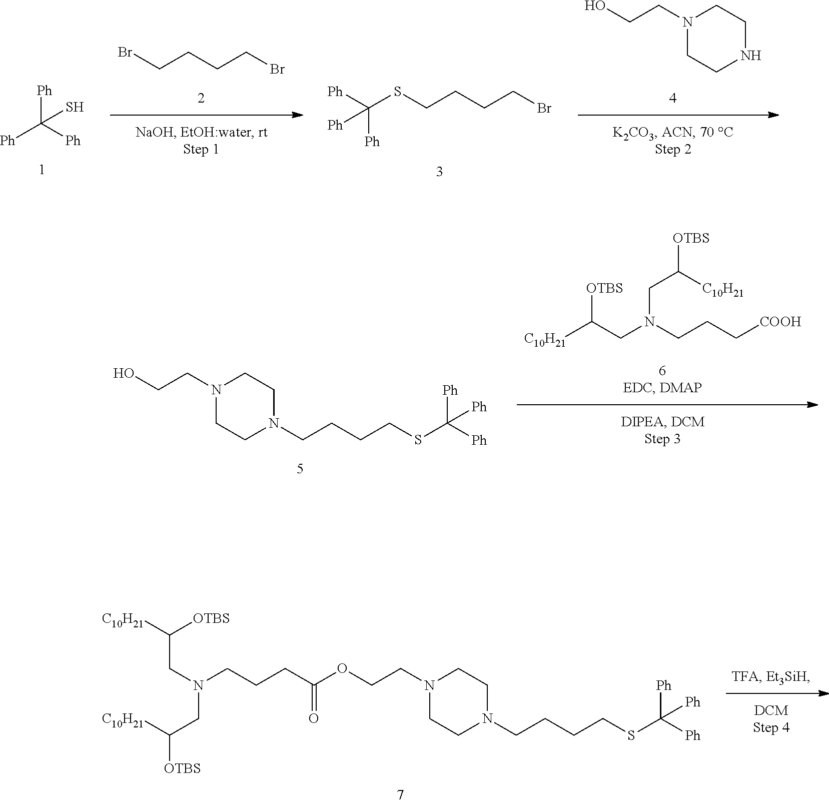

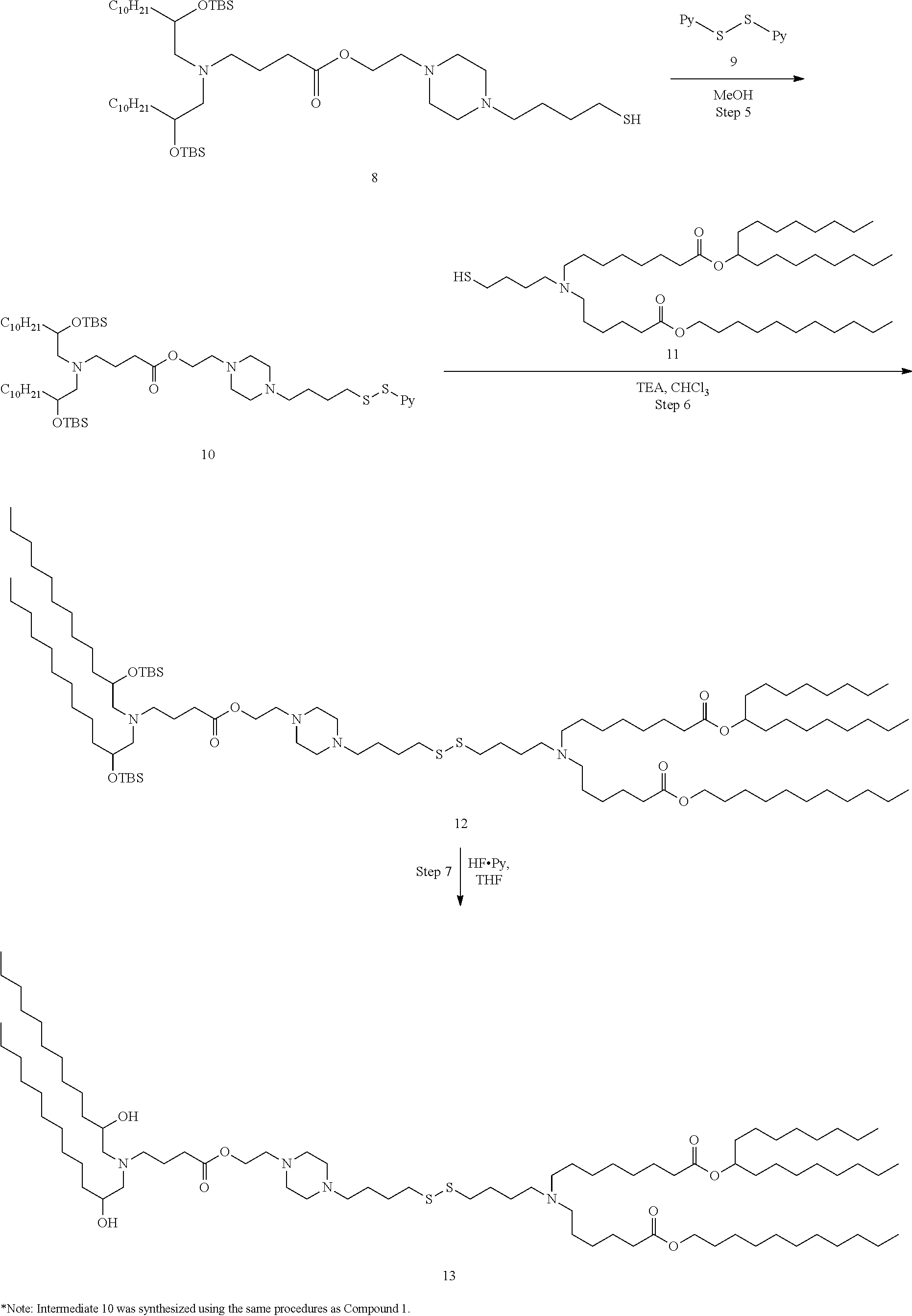

### Intermediate [12]:

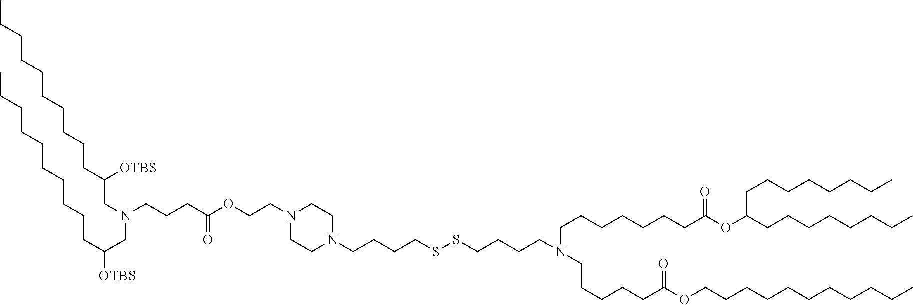 [SMILES: CCCCCCCCCCCOC(=O)CCCCCN(CCCCCCCC(=O)OC(CCCCCCCC)CCCCCCCC)CCCCSSCCCCN1CCN(CCOC(=O)CCCN(CC(CCCCCCCCCC)O[Si](C)(C)C(C)(C)C)CC(CCCCCCCCCC)O[Si](C)(C)C(C)(C)C)CC1]

To a solution of (0.500 g, 0.495 mmol) and (0.596 g, 0.792 mmol) in chloroform was added triethylamine (0.35 ml, 2.47 mmol) and allowed to react at room temperature for 2.5 hours. After completion of the reaction, the reaction mixture was concentrated and purified to obtain as colorless oil (0.500 g, 61% yield).

ESI-MS analysis: Calculated for C₉₆H₁₉₄N₄₀₈S₂Si₂, [M+H]=1652.92; Observed=1653.

### Compound 2 [13]:

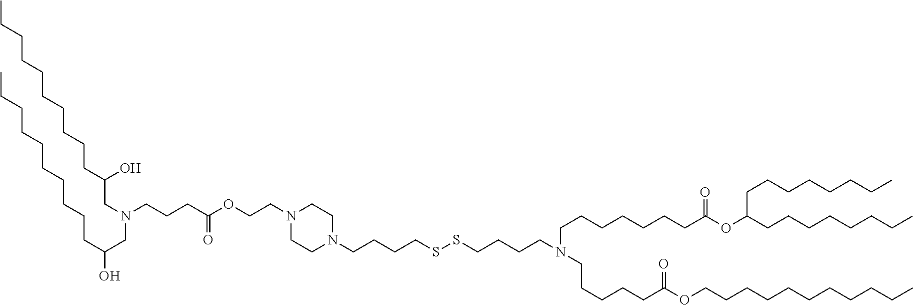 [SMILES: CCCCCCCCCCCOC(=O)CCCCCN(CCCCCCCC(=O)OC(CCCCCCCC)CCCCCCCC)CCCCSSCCCCN1CCN(CCOC(=O)CCCN(CC(O)CCCCCCCCCC)CC(O)CCCCCCCCCC)CC1]

To a 20 ml polypropylene scintillation vial was added (0.500 g, 0.302 mmol, 1.0 eq) along with 4 mL of dry tetrahydrofuran. The vial was cooled to 0-5 °C and HF/pyridine (1.55 mL, 59.59 mmol) was added dropwise. After addition, the reaction vial was allowed to warm to room temperature and stirred for 18 hours. Afterwards, the reaction mixture was cooled back to 0 °C and neutralized with solid sodium bicarbonate solid, diluted with ethyl acetate, washed with NaHCO₃ solution, water and brine. The organic layer was dried over anhydrous Na₂SO₄ and concentrated. The crude product was purified to obtain compound (0.30 g, 69%). It was confirmed by ¹H NMR and MS analysis.

### Results:

¹H NMR (400 MHz, CDCl₃) 4.85 (quin, 1H), 4.20 (t, 2H), 4.04 (t, 2H), 3.63 (m, 2H), 3.04-2.49 (m, 20H), 2.48-2.18 (m, 12H), 1.95-1.55 (m, 18H), 1.54-1.40 (m, 8H), 1.39-1.16 (m, 86H), 0.86 (t, 15H).

ESI-MS analysis: Calculated for C₈₄H₁₆₆N₄₀₈S₂, [M+H]=1424.39; Observed=1424.

### Synthetic Protocol for Compound 3

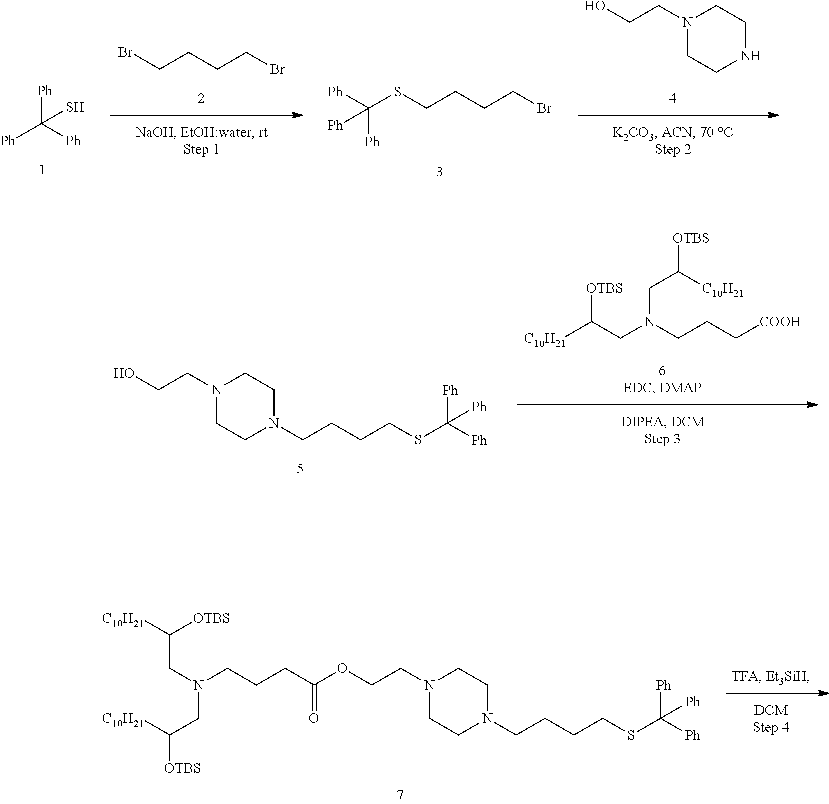

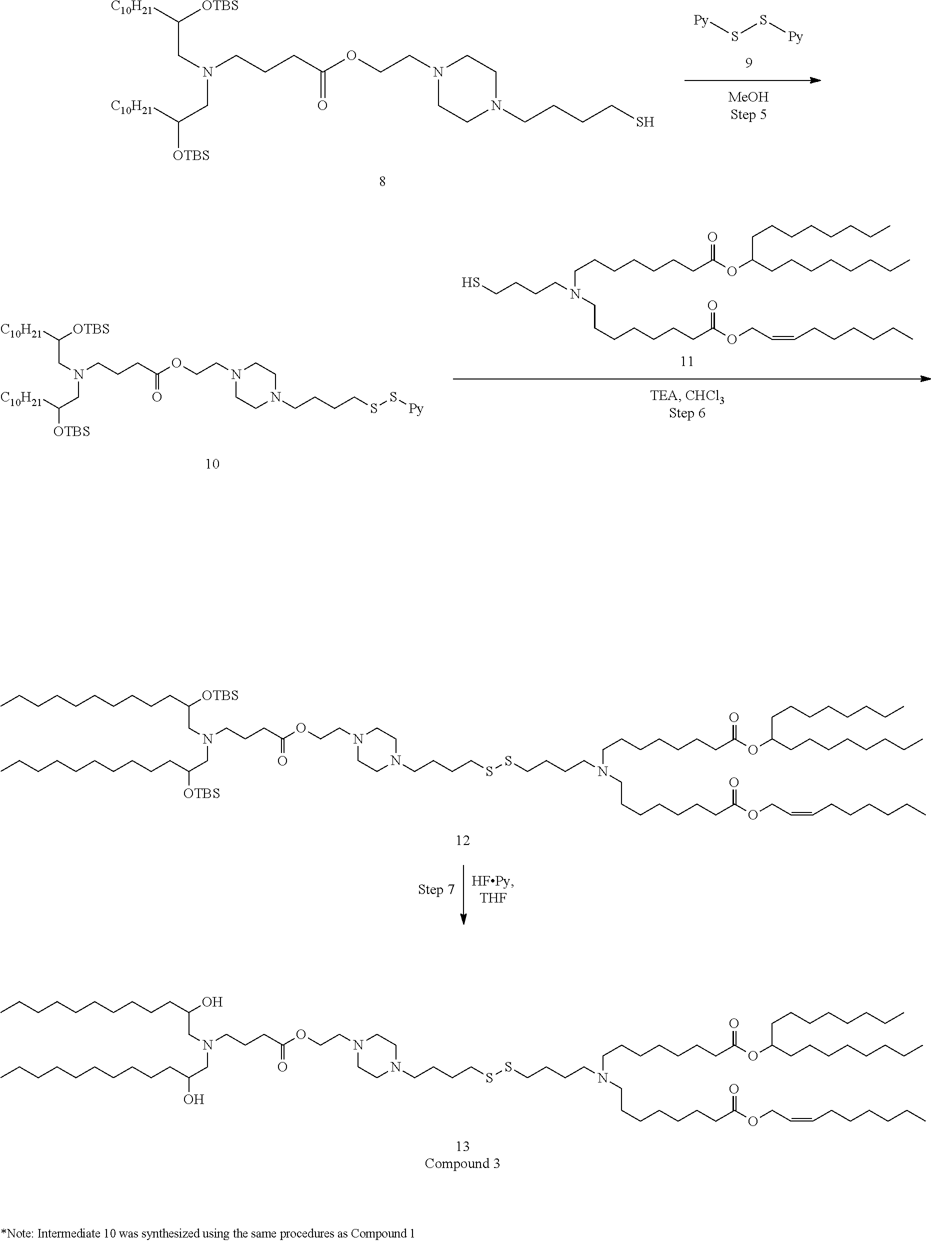

### Intermediate [12]:

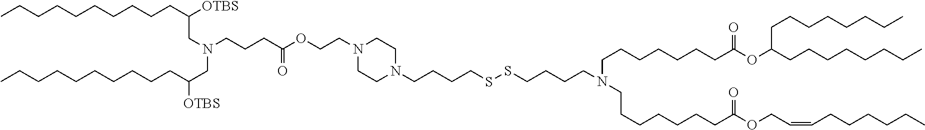 [SMILES: CCCCCC/C=C\COC(=O)CCCCCCCN(CCCCCCCC(=O)OC(CCCCCCCC)CCCCCCCC)CCCCSSCCCCN1CCN(CCOC(=O)CCCN(CC(CCCCCCCCCC)O[Si](C)(C)C(C)(C)C)CC(CCCCCCCCCC)O[Si](C)(C)C(C)(C)C)CC1]

To a solution of (0.500 g, 0.495 mmol) and (0.558 g, 0.743 mmol) in chloroform was added triethylamine (0.35 ml, 2.47 mmol) and allowed to react at room temperature for 2.5 hours. After completion of the reaction, the reaction mixture was concentrated and purified to obtain as colorless oil (0.400 g, 49% yield).

ESI-MS analysis: Calculated for C₉₆H₁₉₂N₄O₈S₂Si₂, [M+H]=1650.90; Observed=1650.91

### Compound 3 [13]:

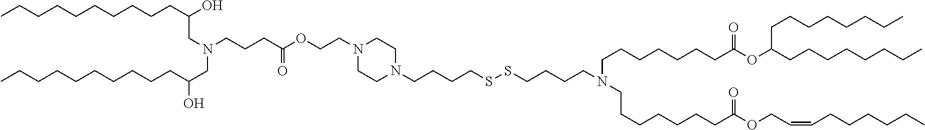 [SMILES: CCCCCC/C=C\COC(=O)CCCCCCCN(CCCCCCCC(=O)OC(CCCCCCCC)CCCCCCCC)CCCCSSCCCCN1CCN(CCOC(=O)CCCN(CC(O)CCCCCCCCCC)CC(O)CCCCCCCCCC)CC1]

To a 20 ml polypropylene scintillation vial was added (0.400 g, 0.242 mmol, 1.0 eq) along with 4 mL of dry tetrahydrofuran. The vial was cooled to 0-5 °C and HF/pyridine (1.24 mL, 47.73 mmol) was added dropwise. After addition, the reaction vial was allowed to warm to room temperature and stirred for 18 hours. Afterwards, the reaction mixture was cooled back to 0 °C and neutralized with solid sodium bicarbonate solid, diluted with ethyl acetate, washed with NaHCO₃ solution, water and brine. The organic layer was dried over anhydrous Na₂SO₄ and concentrated. The crude product was purified to obtain compound (0.220 g, 64%). It was confirmed by ¹H NMR and MS analysis.

### Results:

¹H NMR (400 MHz, CDCl₃) 5.67-5.46 (m, 2H), 4.84 (quin, 1H), 4.60 (d, 2H), 4.20 (t, 2H), 3.65 (br, 2H), 3.05-2.23 (m, 34H), 2.13-2.04 (m, 2H), 1.94-1.54 (m, 10H), 1.53-1.40 (m, 6H), 1.39-1.14 (m, 90H), 0.87 (t, 15H).

ESI-MS analysis: Calculated for C₈₄H₁₆₆N₄O₈S₂, [M+H]=1422.38; Observed=1422.42

### Synthetic Protocol for Compound 4

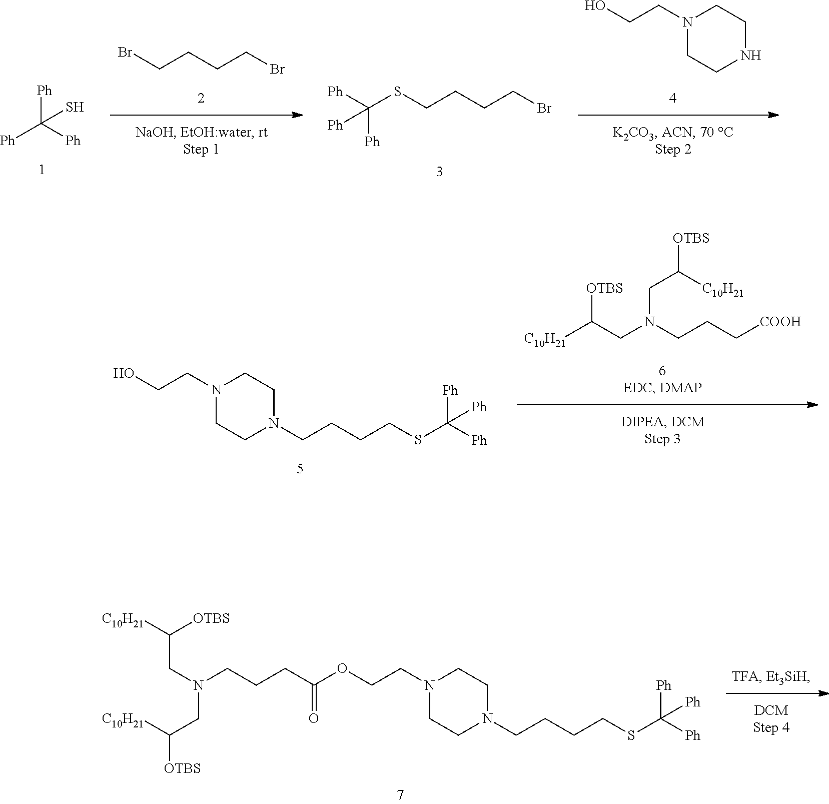

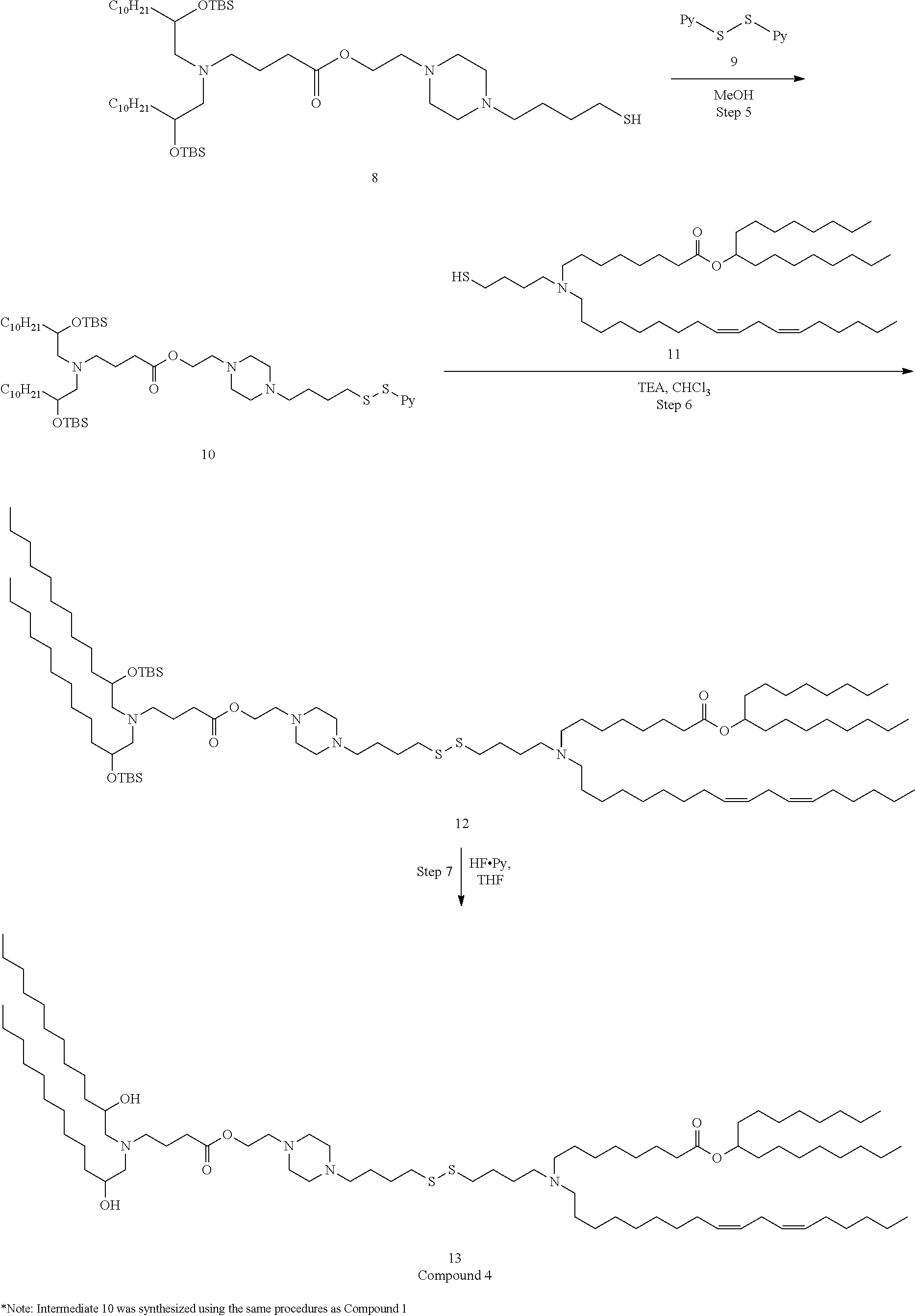

### Intermediate [12]:

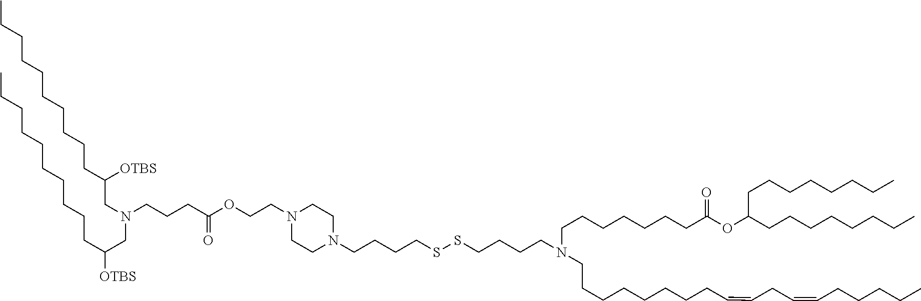 [SMILES: CCCCC/C=C\C/C=C\CCCCCCCCN(CCCCCCCC(=O)OC(CCCCCCCC)CCCCCCCC)CCCCSSCCCCN1CCN(CCOC(=O)CCCN(CC(CCCCCCCCCC)O[Si](C)(C)C(C)(C)C)CC(CCCCCCCCCC)O[Si](C)(C)C(C)(C)C)CC1]

To a solution of (0.350 g, 0.346 mmol) and (0.646 g, 0.420 mmol) in chloroform was added triethylamine (0.24 ml, 1.73 mmol) and allowed to react at room temperature for 2.5 hours. After completion of the reaction, the reaction mixture was concentrated and purified to obtain as colorless oil (0.350 g, 62% yield).

ESI-MS analysis: Calculated for C₉₇H₁₉₄N₄O₆S₂Si₂, [M+H]=1632.93; Observed=1632.01

### Compound 4 [13]:

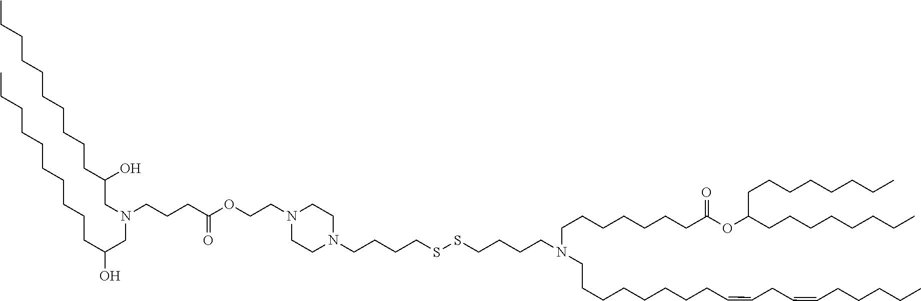 [SMILES: CCCCC/C=C\C/C=C\CCCCCCCCN(CCCCCCCC(=O)OC(CCCCCCCC)CCCCCCCC)CCCCSSCCCCN1CCN(CCOC(=O)CCCN(CC(O)CCCCCCCCCC)CC(O)CCCCCCCCCC)CC1]

To a 20 ml polypropylene scintillation vial was added (0.350 g, 0.214 mmol, 1.0 eq) along with 4 mL of dry tetrahydrofuran. The vial was cooled to 0-5 °C and HF/pyridine (1.1 mL, 42.22 mmol) was added dropwise. After addition, the reaction vial was allowed to warm to room temperature and stirred for 18 hours. Afterwards, the reaction mixture was cooled back to 0 °C and neutralized with solid sodium bicarbonate solid, diluted with ethyl acetate, washed with NaHCO₃ solution, water and brine. The organic layer was dried over anhydrous Na₂SO₄ and concentrated. The crude product was purified to obtain compound (0.219 g, 72%). It was confirmed by ¹H NMR and MS analysis.

¹H NMR (400 MHz, CDCl₃) 5.43-5.29 (m, 4H), 4.85 (quin, 1H), 4.19 (t, 2H), 3.61 (m, 2H), 2.96-2.18 (m, 32H), 2.12-1.99 (m, 4H), 1.90-1.41 (m, 20H), 1.40-1.13 (m, 86H), 0.86 (t, 15H).

ESI-MS analysis: Calculated for C₈₅H₁₆₆N₄O₆S₂, [M+H]=1404.41; Observed=1404.40

### Synthetic Protocol for Compound 5

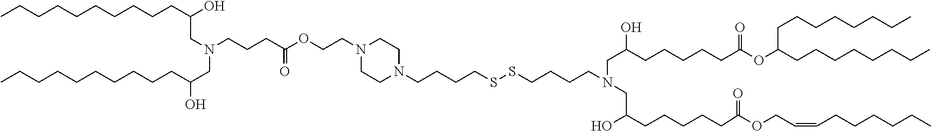 [SMILES: CCCCCC/C=C\COC(=O)CCCCCC(O)CN(CCCCSSCCCCN1CCN(CCOC(=O)CCCN(CC(O)CCCCCCCCCC)CC(O)CCCCCCCCCC)CC1)CC(O)CCCCCC(=O)OC(CCCCCCCC)CCCCCCCC]

Following the very similar synthetic route described before, Compound 5 was obtained as a light-yellow oil (0.34 g, 30% over two steps). It was confirmed by ¹H NMR and MS analysis.

### Results:

¹H NMR (400 MHz, CDCl₃) 5.67-5.46 (m, 2H), 4.84 (m, 1H), 4.51-4.48 (m, 2H), 4.20 (m, 2H), 3.65 (m, 4H), 2.67-2.23 (m, 32H), 2.13-1.81 (m, 2H), 1.80-1.51 (m, 14H), 1.50-1.13 (m, 90H), 0.87-0.75 (m, 15H).

ESI-MS analysis: Calculated for C₈₄H₁₆₄N₄O₁₀S₂, [M+H]=1454.0; Observed=1454.1

### Synthetic Protocol for Compound 6

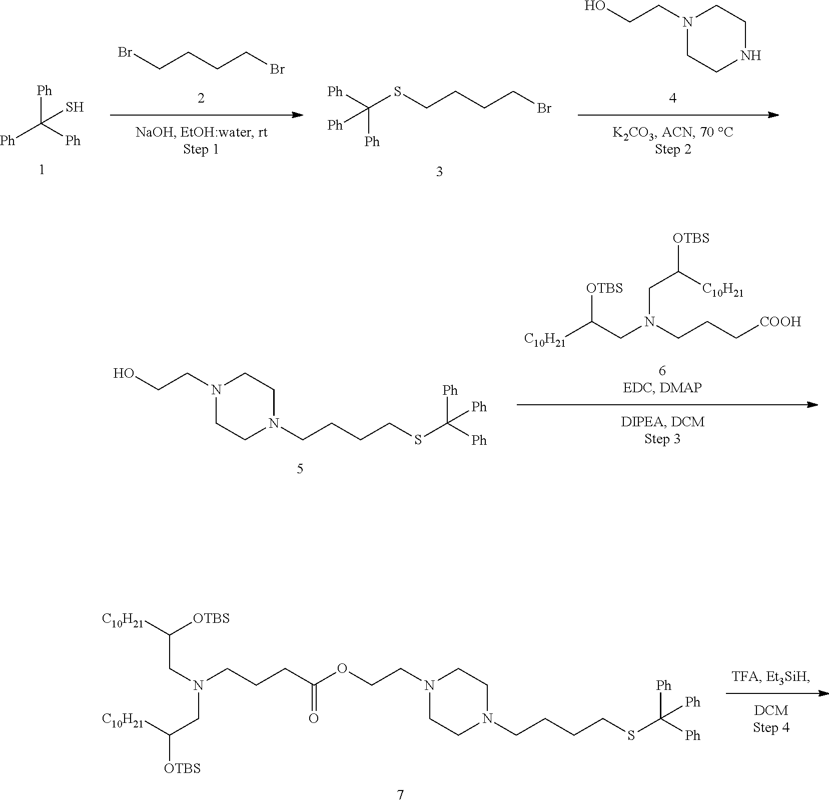

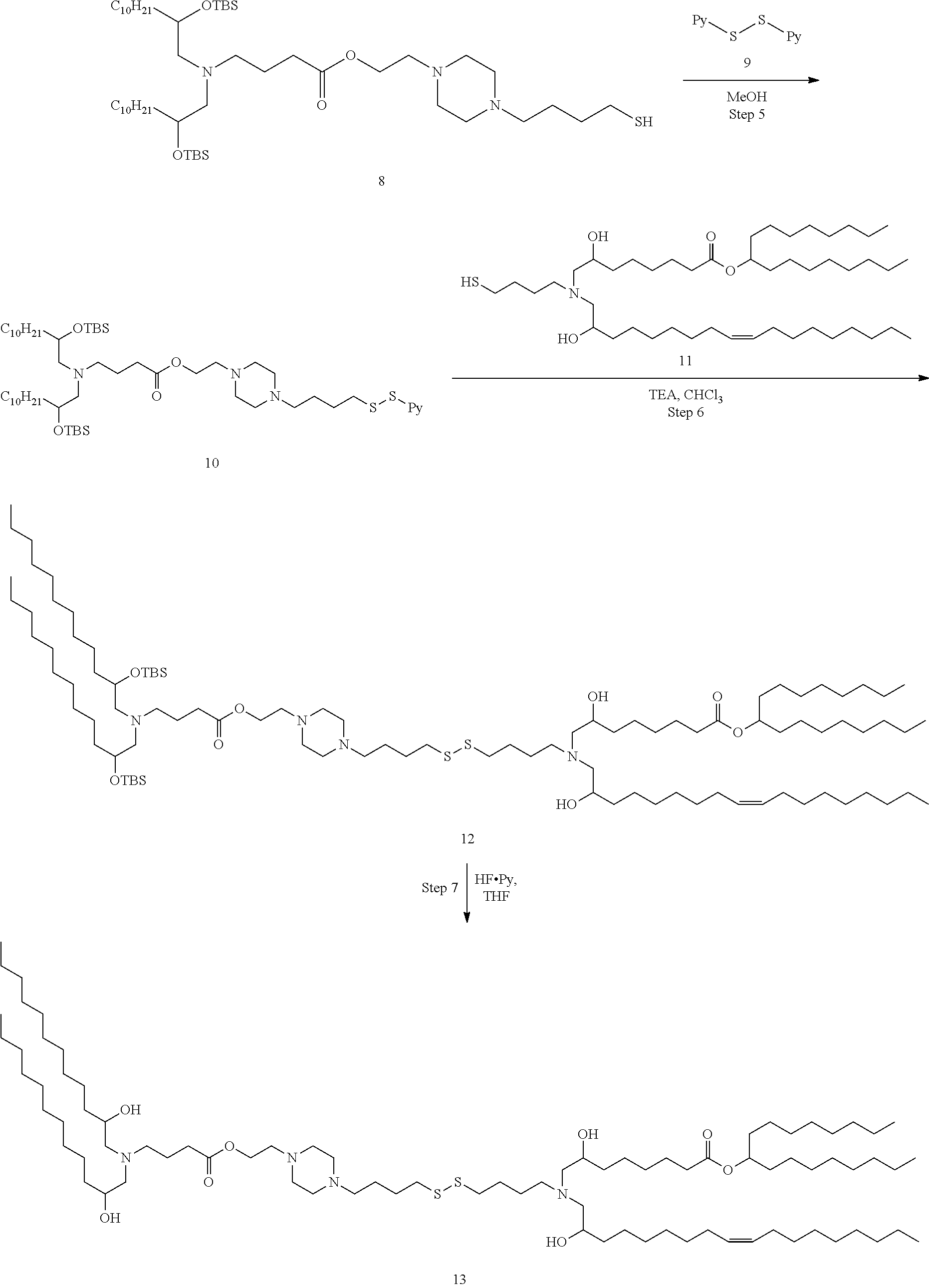

Intermediate 10 was synthesized using the same procedures as for Compound 1.

### Intermediate [12]:

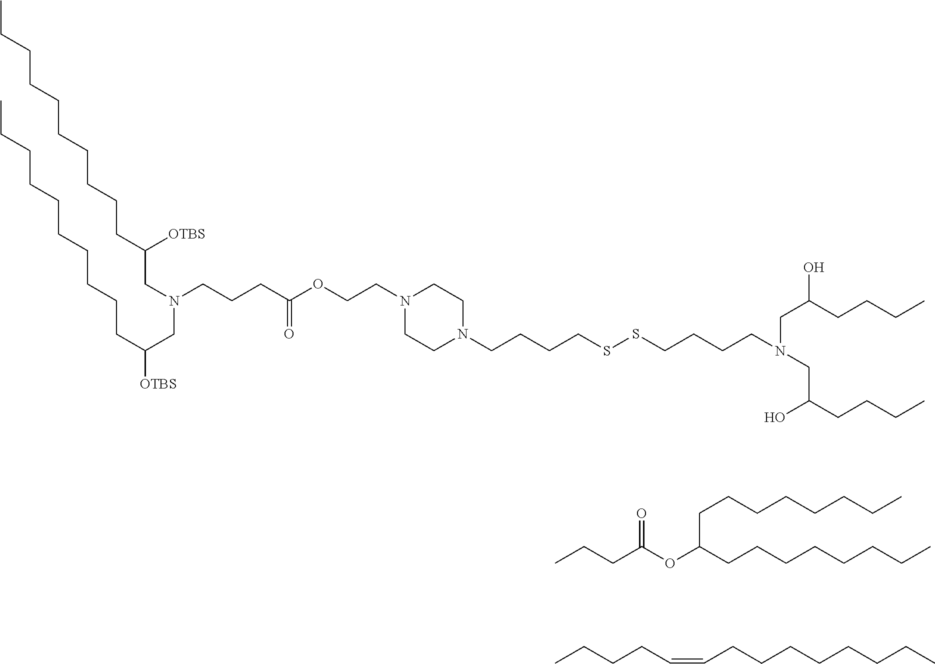

To a solution of (0.500 g, 0.495 mmol) and (0.646 g, 0.841 mmol) in chloroform was added triethylamine (0.35 ml, 2.47 mmol) and allowed to react at room temperature for 2.5 hours. After completion of the reaction, the reaction mixture was concentrated and purified to obtain as colorless oil (0.370 g, 45% yield).

ESI-MS analysis: Calculated for C₉₇H₁₉₆N₄O₈S₂Si₂, [M+H]=1666.95; Observed=1666.

### Compound 6:

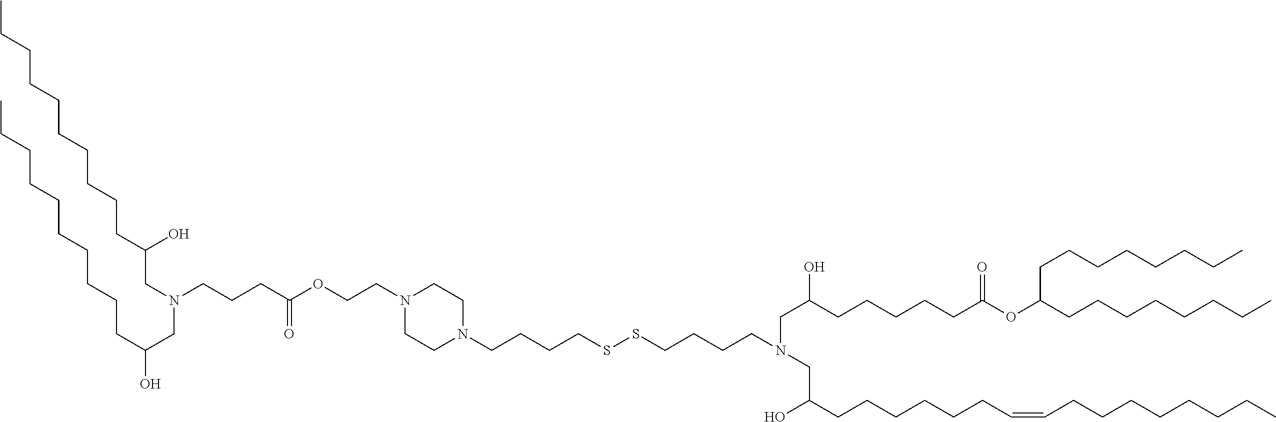 [SMILES: CCCCCCCC/C=C\CCCCCCC(O)CN(CCCCSSCCCCN1CCN(CCOC(=O)CCCN(CC(O)CCCCCCCCCC)CC(O)CCCCCCCCCC)CC1)CC(O)CCCCCC(=O)OC(CCCCCCCC)CCCCCCCC]

To a 20 ml polypropylene scintillation vial was added (0.370 g, 0.222 mmol, 1.0 eq) along with 4 mL of dry tetrahydrofuran. The vial was cooled to 0-5 °C and HF/pyridine (1.14 mL, 43.73 mmol) was added dropwise. After addition, the reaction vial was allowed to warm to room temperature and stirred for 18 hours. Afterwards, the reaction mixture was cooled back to 0 °C and neutralized with solid sodium bicarbonate solid, diluted with ethyl acetate, washed with NaHCO₃ solution, water and brine. The organic layer was dried over anhydrous Na₂SO₄ and concentrated. The crude product was purified to obtain compound (0.213 g, 66%). It was confirmed by ¹H NMR and MS analysis.

¹H NMR (400 MHz, CDCl₃) 5.42-5.29 (m, 2H), 4.84 (quin, 1H), 4.20 (t, 2H), 3.68 (br, 3H), 2.92-2.31 (m, 22H), 2.30-2.23 (t, 2H), 2.03-1.91 (m, 4H), 1.88-1.76 (m, 2H), 1.75-1.55 (m, 12H), 1.54-1.11 (m, 102H), 0.86 (t, 15H).

ESI-MS analysis: Calculated for C₈₅H₁₆₈N₄O₈S₂, [M+H]=1438.42; Observed=1438

### Synthetic Protocol for Compound 7

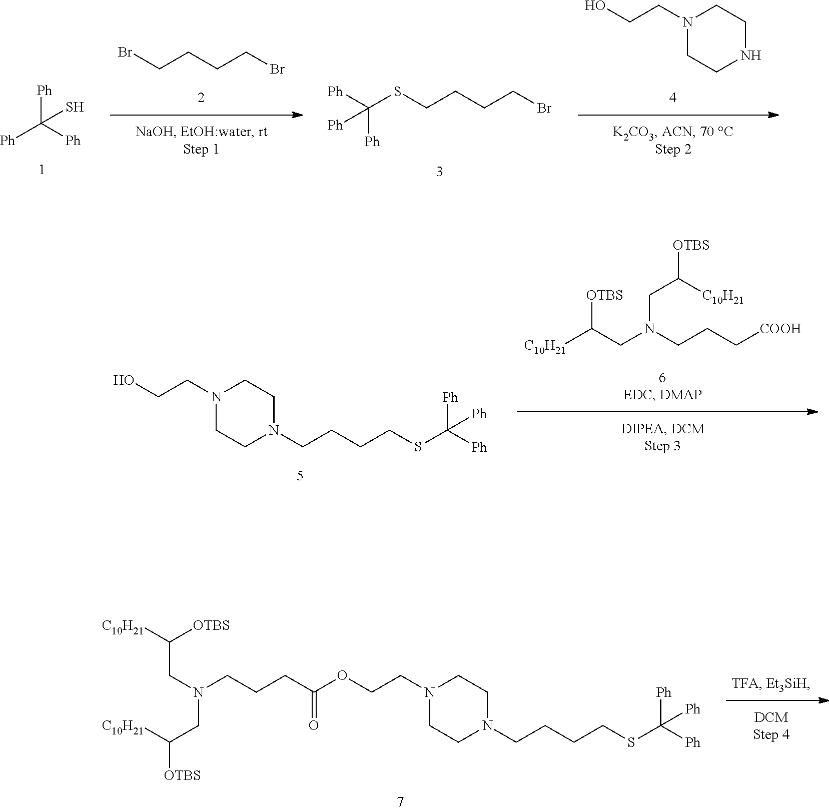

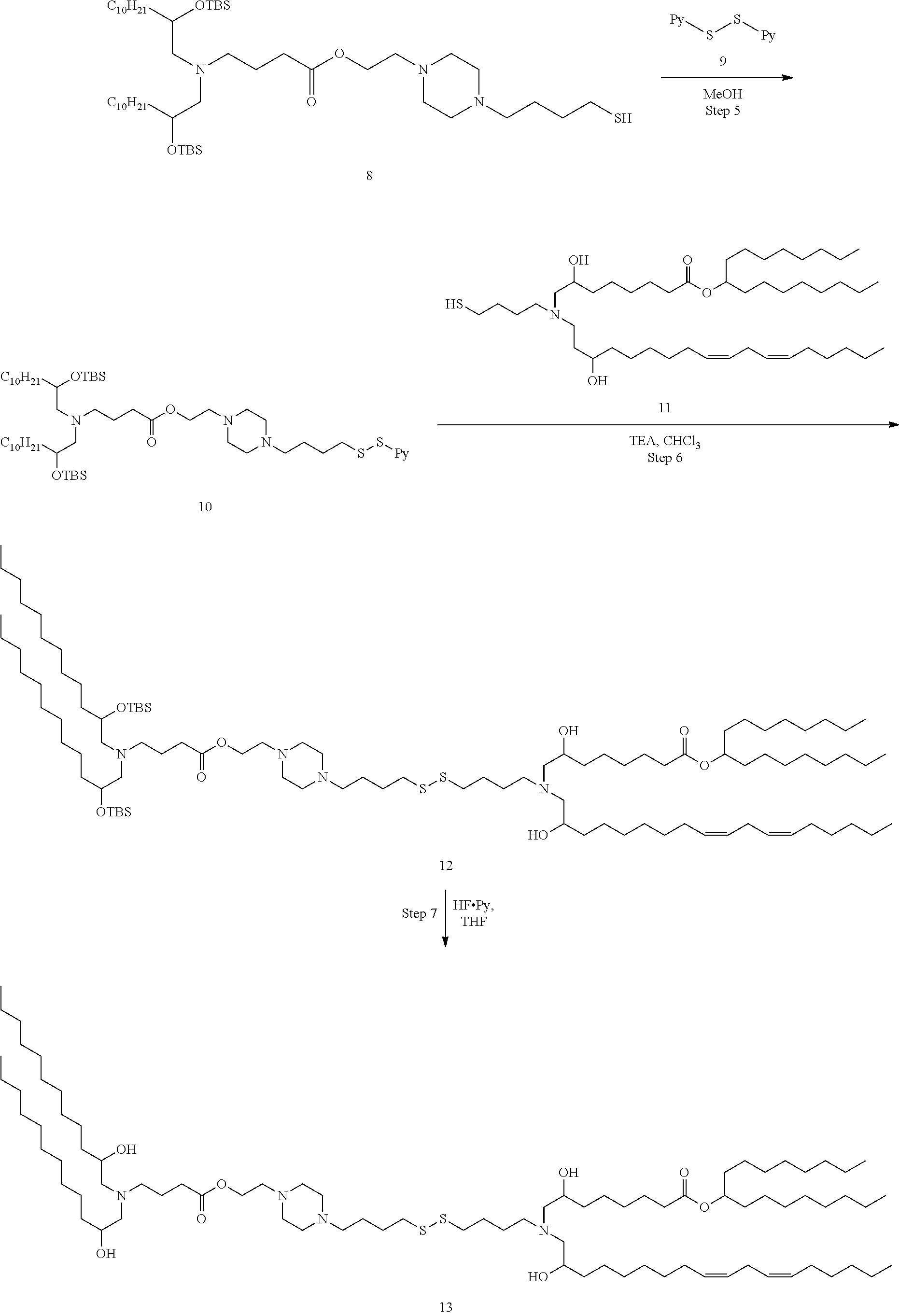

Intermediate 10 was prepared as for Compound 1.

### Intermediate [12]:

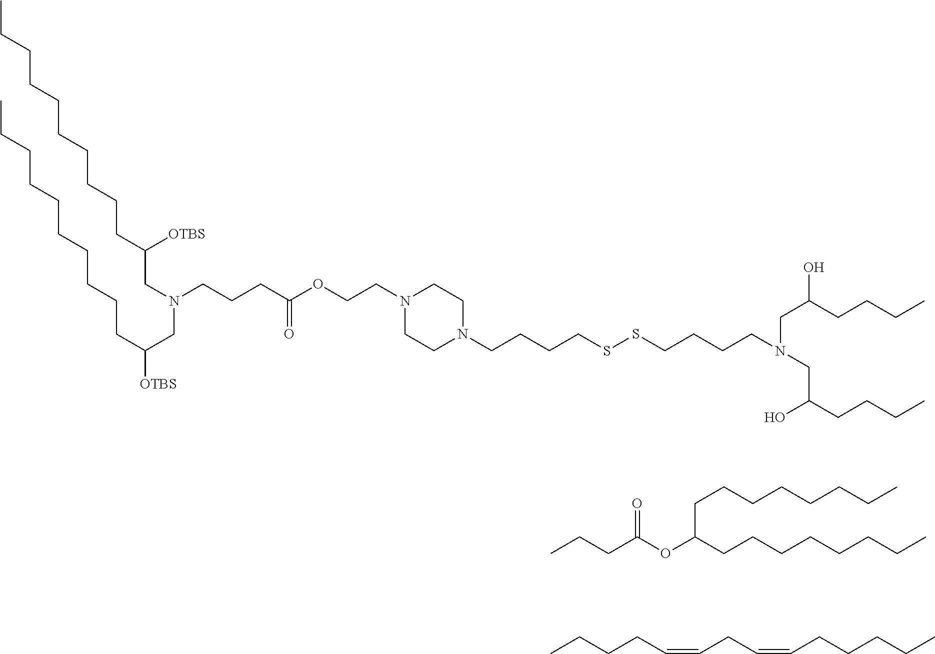

To a solution of (0.390 g, 0.386 mmol) and (0.518 g, 0.675 mmol) in chloroform was added triethylamine (0.322 ml, 2.32 mmol) and allowed to react at room temperature for 2.5 hours. After completion of the reaction, the reaction mixture was concentrated and purified to obtain as colorless oil (0.320 g, 50% yield).

ESI-MS analysis: Calculated for C₉₇H₁₉₄N₄O₈S₂Si₂, [M+H]=1664.93; Observed=1664.

### Compound 7:

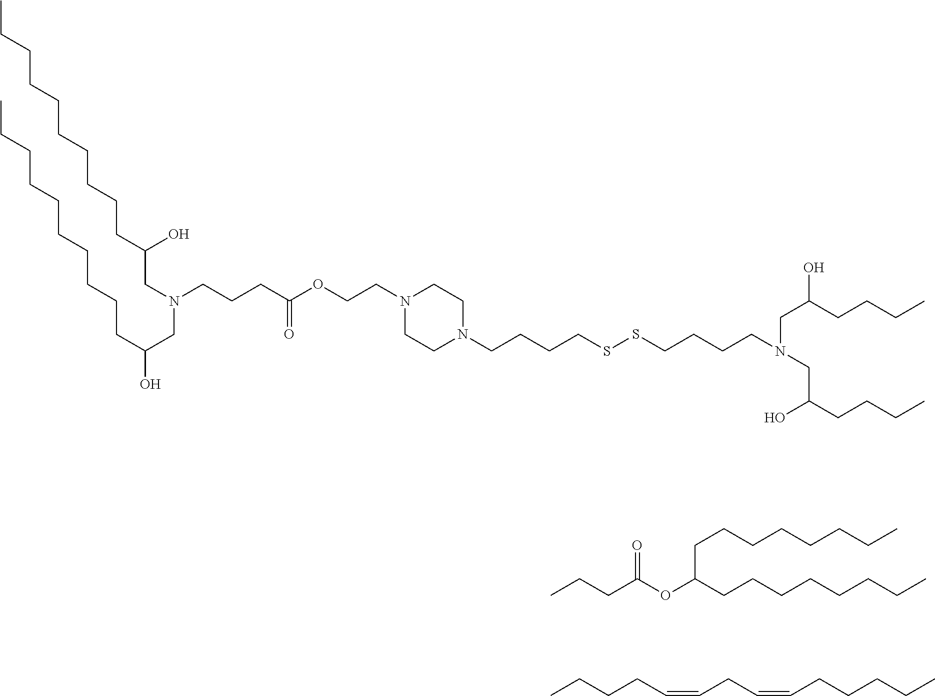

To a 20 ml polypropylene scintillation vial was added (0.320 g, 0.192 mmol, 1.0 eq) along with 4 mL of dry tetrahydrofuran. The vial was cooled to 0-5 °C and HF/pyridine (0.983 mL, 37.86 mmol) was added dropwise. After addition, the reaction vial was allowed to warm to room temperature and stirred for 18 hours. Afterwards, the reaction mixture was cooled back to 0 °C and neutralized with solid sodium bicarbonate solid, diluted with ethyl acetate, washed with NaHCO₃ solution, water and brine. The organic layer was dried over anhydrous Na₂SO₄ and concentrated. The crude product was purified to obtain compound (0.176 g, 64%). It was confirmed by ¹H NMR and MS analysis.

¹H NMR (400 MHz, CDCl₃) 5.44-5.26 (m, 4H), 4.84 (quin, 1H), 4.20 (t, 2H), 3.70 (br, 4H), 2.99-2.33 (m, 32H), 2.32-2.24 (t, 2H), 2.09-1.92 (m, 4H), 1.91-1.56 (m, 12H), 1.55-1.14 (m, 90H), 0.87 (t, 15H).

ESI-MS analysis: Calculated for C₈₅H₁₆₆N₄O₈S₂, [M+H]=1436.40; Observed=1436.

### Synthetic Protocol for Compound 8

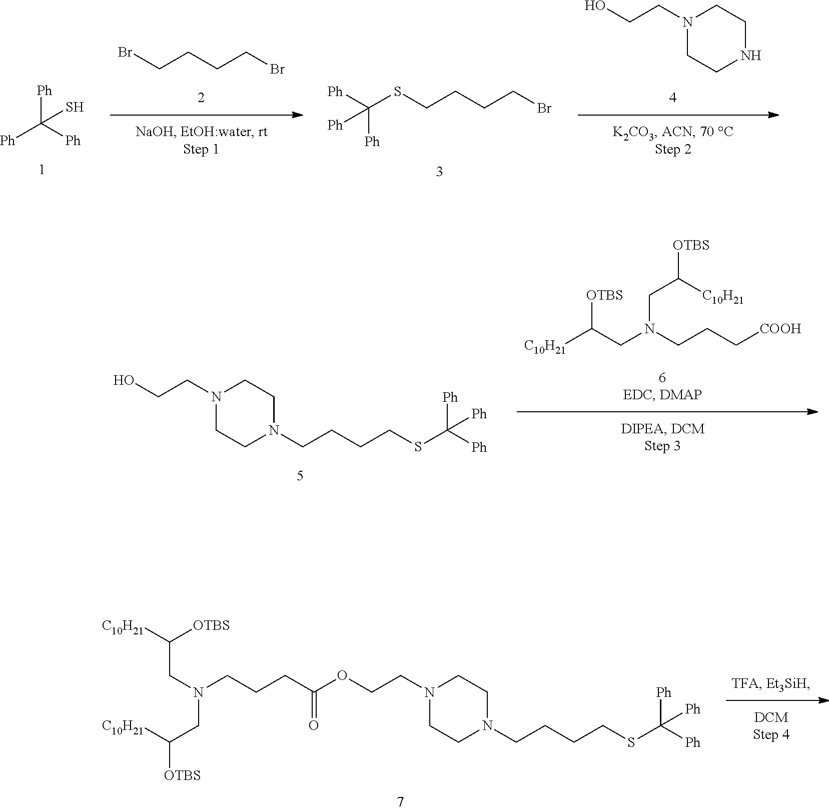

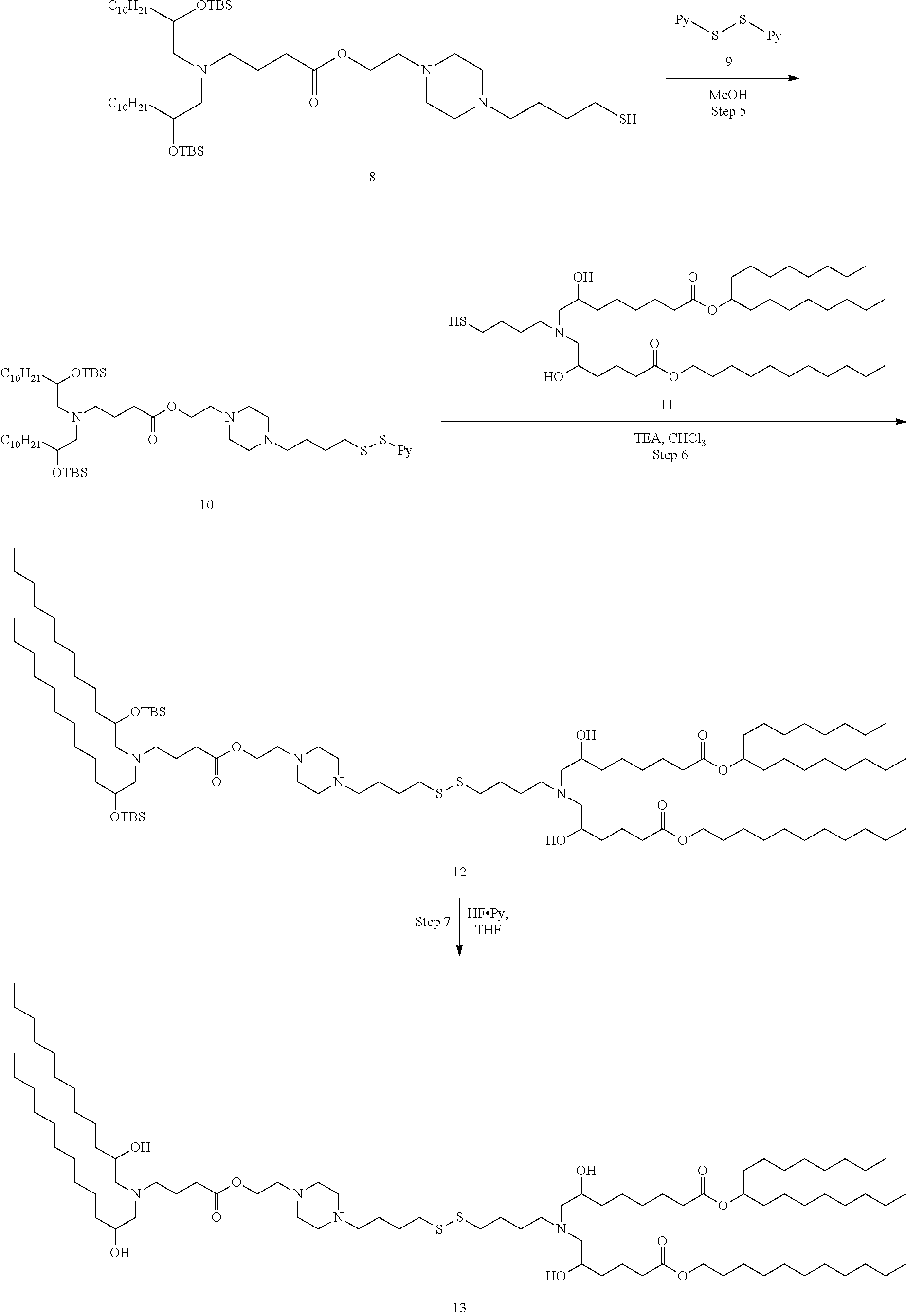

Intermediate 10 was prepared as for Compound 1.

### Intermediate [12]:

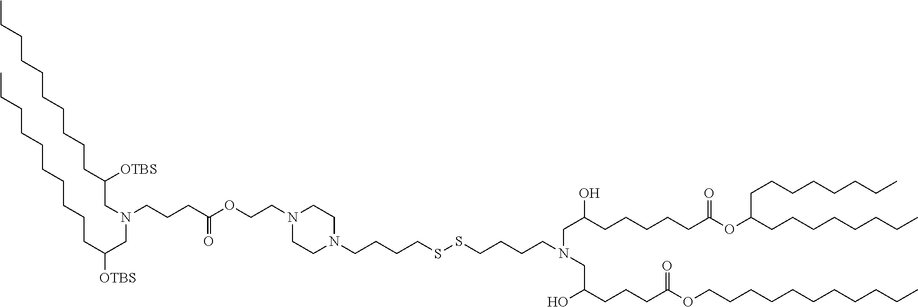 [SMILES: CCCCCCCCCCCOC(=O)CCCC(O)CN(CCCCSSCCCCN1CCN(CCOC(=O)CCCN(CC(CCCCCCCCCC)O[Si](C)(C)C(C)(C)C)CC(CCCCCCCCCC)O[Si](C)(C)C(C)(C)C)CC1)CC(O)CCCCCC(=O)OC(CCCCCCCC)CCCCCCCC]

To a solution of (0.190 g, 0.188 mmol) and (0.259 g, 0.329 mmol) in chloroform was added triethylamine (0.157 ml, 1.13 mmol) and allowed to react at room temperature for 2.5 hours. After completion of the reaction, the reaction mixture was concentrated and taken to the next step without purification (0.317 g Crude Material).

ESI-MS analysis: Calculated for C₉₆H₁₉₄N₄O₁₀S₂Si₂, [M+H]=1684.92; Observed=1684.

### Compound 8

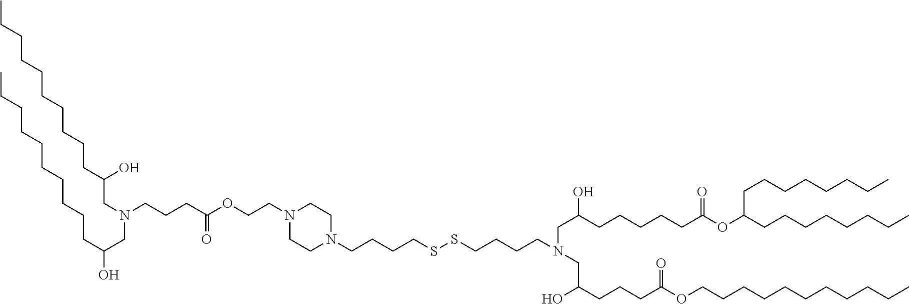 [SMILES: CCCCCCCCCCCOC(=O)CCCC(O)CN(CCCCSSCCCCN1CCN(CCOC(=O)CCCN(CC(O)CCCCCCCCCC)CC(O)CCCCCCCCCC)CC1)CC(O)CCCCCC(=O)OC(CCCCCCCC)CCCCCCCC]

To a 20 ml polypropylene scintillation vial was added (Crude Material, 0.317 g) along with 4 mL of dry tetrahydrofuran. The vial was cooled to 0-5 °C and HF/pyridine (1.0 mL, 37.06 mmol) was added dropwise. After addition, the reaction vial was allowed to warm to room temperature and stirred for 18 hours. Afterwards, the reaction mixture was cooled back to 0 °C and neutralized with solid sodium bicarbonate solid, diluted with ethyl acetate, washed with NaHCO₃ solution, water and brine. The organic layer was dried over anhydrous Na₂SO₄ and concentrated. The crude product was purified to obtain compound (0.090 g, 33% over two steps). It was confirmed by ¹H NMR and MS analysis.

### Results:

¹H NMR (400 MHz, CDCl₃) 4.84 (quin, 1H), 4.20 (t, 2H), 4.04 (t, 2H), 3.98-3.65 (br, 3H), 3.20-2.47 (m, 22H), 2.46-2.19 (m, 6H), 1.99-1.66 (m, 10H), 1.65-1.56 (m, 5H), 1.55-1.13 (m, 80H), 0.87 (t, 15H).

ESI-MS analysis: Calculated for C₈₄H₁₆₆N₄O₁₀S₂, [M+H]=1456.39; Observed=1456.

### Synthetic Protocol for Compound 9

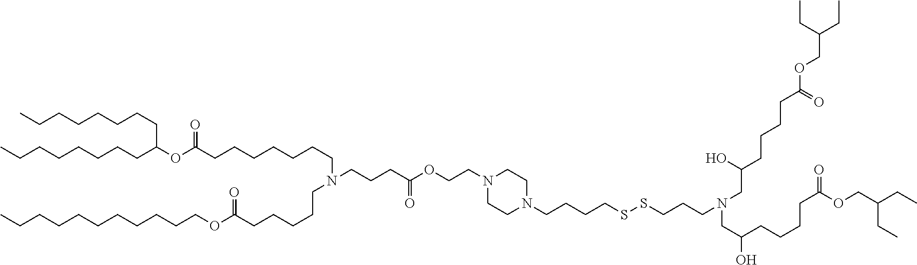 [SMILES: CCCCCCCCCCCOC(=O)CCCCCN(CCCCCCCC(=O)OC(CCCCCCCC)CCCCCCCC)CCCC(=O)OCCN1CCN(CCCCSSCCCN(CC(O)CCCCC(=O)OCC(CC)CC)CC(O)CCCCC(=O)OCC(CC)CC)CC1]

Following the very similar synthetic route described before, Compound 9 was obtained as a dark-yellow oil 0.295 (64% yield). It was confirmed by the MS analysis.

### Results:

ESI-MS analysis: Calculated for C₈₅H₁₆₅N₄O₁₂S₂, [M+H]=1498.19; Observed=1498.1

### Synthetic Protocol for Compound 10

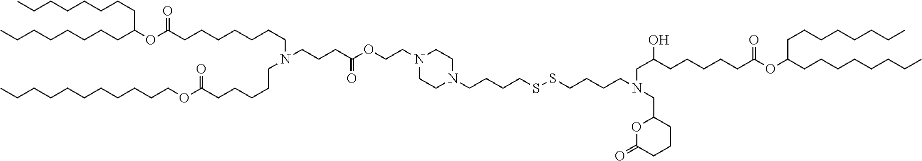 [SMILES: CCCCCCCCCCCOC(=O)CCCCCN(CCCCCCCC(=O)OC(CCCCCCCC)CCCCCCCC)CCCC(=O)OCCN1CCN(CCCCSSCCCCN(CC(O)CCCCCC(=O)OC(CCCCCCCC)CCCCCCCC)CC2CCCC(=O)O2)CC1]

Following the very similar synthetic route described before, Compound 10 was obtained as a light-brown oil (0.085 mg, 21%). It was confirmed by the MS analysis.

### Results:

ESI-MS analysis: Calculated for C₉₁H₁₇₅N₄O₁₁S₂, [M+H]=1565.27; Observed=1565.1

### Synthetic Protocol for Compound 11

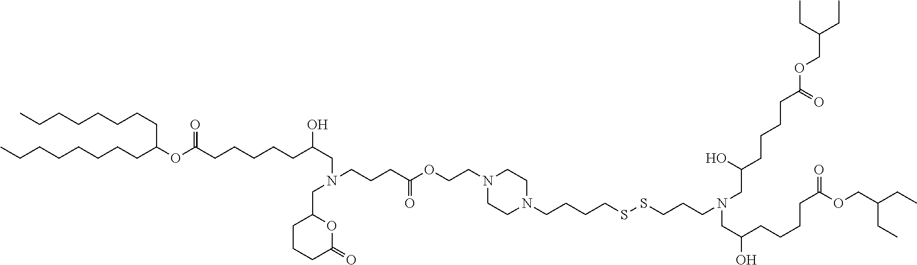 [SMILES: CCCCCCCCC(CCCCCCCC)OC(=O)CCCCCC(O)CN(CCCC(=O)OCCN1CCN(CCCCSSCCCN(CC(O)CCCCC(=O)OCC(CC)CC)CC(O)CCCCC(=O)OCC(CC)CC)CC1)CC1CCCC(=O)O1]

Following the very similar synthetic route described before, Compound 11 was obtained as a yellow oil (0.050 g, 13%). It was confirmed by the MS analysis.

ESI-MS analysis: Calculated for C₇₄H₁₄₁N₄O₁₃S₂, [M+H]=1358.07; Observed=1358.1

### Synthetic Protocol for Compound 12

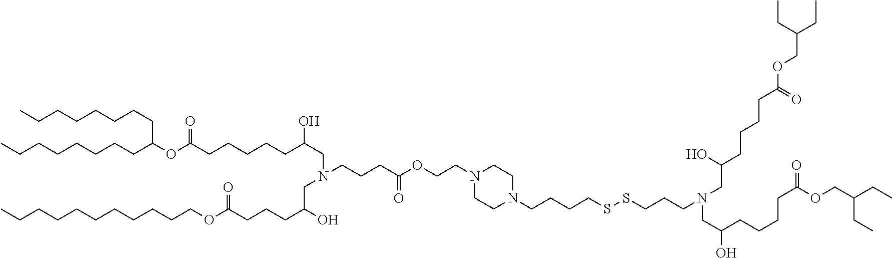 [SMILES: CCCCCCCCCCCOC(=O)CCCC(O)CN(CCCC(=O)OCCN1CCN(CCCCSSCCCN(CC(O)CCCCC(=O)OCC(CC)CC)CC(O)CCCCC(=O)OCC(CC)CC)CC1)CC(O)CCCCCC(=O)OC(CCCCCCCC)CCCCCCCC]

Following the very similar synthetic route described before, Asymmetric Good lipid (GL-Asymm-032) was obtained as a light-yellow oil (0.060 g, 15%). It was confirmed by the MS analysis.

### Results:

ESI-MS analysis: Calculated for C₈₅H₁₆₅N₄O₁₄S₂, [M+H]=1530.18; Observed=1530.0

### Synthetic Protocol for Compound 13

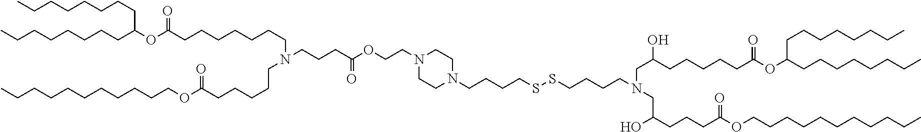 [SMILES: CCCCCCCCCCCOC(=O)CCCCCN(CCCCCCCC(=O)OC(CCCCCCCC)CCCCCCCC)CCCC(=O)OCCN1CCN(CCCCSSCCCCN(CC(O)CCCCCC(=O)OC(CCCCCCCC)CCCCCCCC)CC(O)CCCC(=O)OCCCCCCCCCCC)CC1]

Following the very similar synthetic route described before, Asymmetric Good lipid (GL-Asymm-031) was obtained as a light-yellow oil (0.197 g, 50%). It was confirmed by the MS analysis.

### Results:

ESI-MS analysis: Calculated for C₁₀₂H₁₉₉N₄O₁₂S₂, [M+H]=1737.46; Observed=1736.84

### Synthetic Protocol for Compound 14

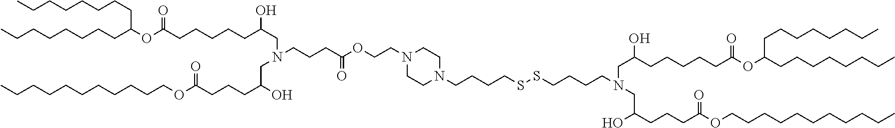 [SMILES: CCCCCCCCCCCOC(=O)CCCC(O)CN(CCCCSSCCCCN1CCN(CCOC(=O)CCCN(CC(O)CCCCCC(=O)OC(CCCCCCCC)CCCCCCCC)CC(O)CCCC(=O)OCCCCCCCCCCC)CC1)CC(O)CCCCCC(=O)OC(CCCCCCCC)CCCCCCCC]

Following the very similar synthetic route described before, Compound 14 was obtained as a pale-yellow oil (0.15 g, 15% over two steps). It was confirmed by ¹H NMR and MS analysis.

### Results:

¹H NMR (400 MHz, CDCl₃) 4.80-4.77 (m, 2H), 4.10-3.96 (m, 6H), 3.65-3.98 (m, 4H), 3.30-2.47 (m, 24H), 2.23-2.19 (m, 12H), 1.99-1.66 (m, 12H), 1.65-1.56 (m, 30H), 1.22-1.18 (m, 90H), 0.82-0.79 (m, 18H).

ESI-MS analysis: Calculated for C₁₀₂H₁₉₈N₄O₁₄S₂, [M+H]=1768.84; Observed=1768.74

### Synthetic Protocol for Compound 15

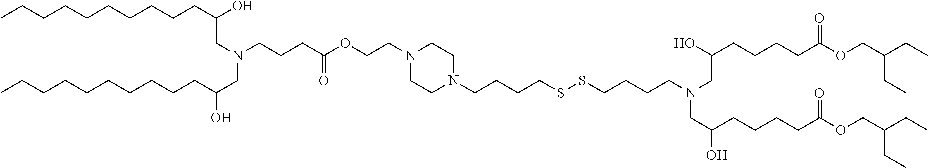 [SMILES: CCCCCCCCCCC(O)CN(CCCC(=O)OCCN1CCN(CCCCSSCCCCN(CC(O)CCCCC(=O)OCC(CC)CC)CC(O)CCCCC(=O)OCC(CC)CC)CC1)CC(O)CCCCCCCCCC]

Following the very similar synthetic route described before, Compound 15 was obtained as a light-yellow oil (0.32 g, 53%). It was confirmed by the MS analysis.

### Results:

ESI-MS analysis: Calculated for C₆₈H₁₃₅N₄O₁₀S₂, [M+H]=1231.96; Observed=1231.10

### Synthetic Protocol for Compound 16

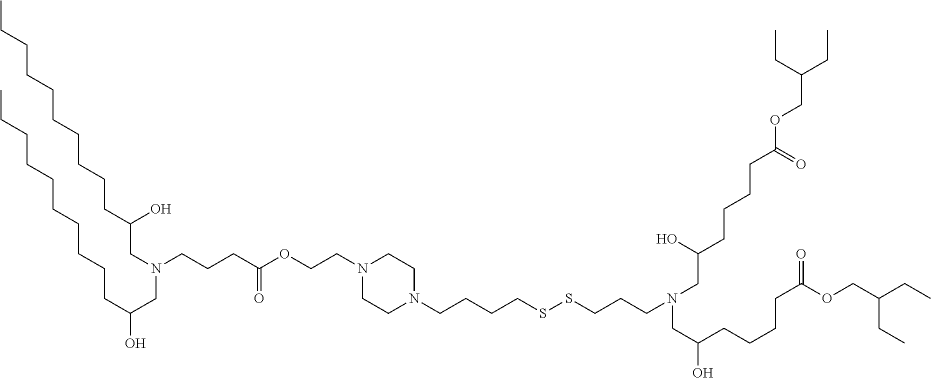 [SMILES: CCCCCCCCCCC(O)CN(CCCC(=O)OCCN1CCN(CCCCSSCCCN(CC(O)CCCCC(=O)OCC(CC)CC)CC(O)CCCCC(=O)OCC(CC)CC)CC1)CC(O)CCCCCCCCCC]

Following the very similar synthetic route described before, Compound 16 was obtained as a pale-yellow oil (0.275 g, 35%). It was confirmed by the MS analysis.

ESI-MS analysis: Calculated for C₆₇H₁₃₃N₄O₁₀S₂, [M+H]=1217.95; Observed=1217.10

### Synthetic Protocol for Compound 17

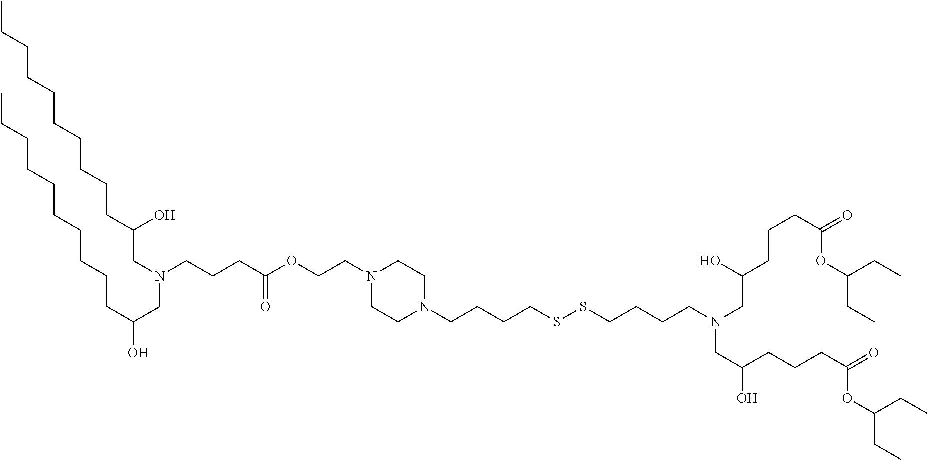 [SMILES: CCCCCCCCCCC(O)CN(CCCC(=O)OCCN1CCN(CCCCSSCCCCN(CC(O)CCCC(=O)OC(CC)CC)CC(O)CCCC(=O)OC(CC)CC)CC1)CC(O)CCCCCCCCCC]

Following the very similar synthetic route described before, Compound 17 was obtained as a light-yellow oil 0.150 g (38%). It was confirmed by the MS analysis.

### Results:

ESI-MS analysis: Calculated for C₆₄H₁₂₇N₄O₁₀S₂, [M+H]=1175.90; Observed=1175.10

### Synthetic Protocol for Compound 18

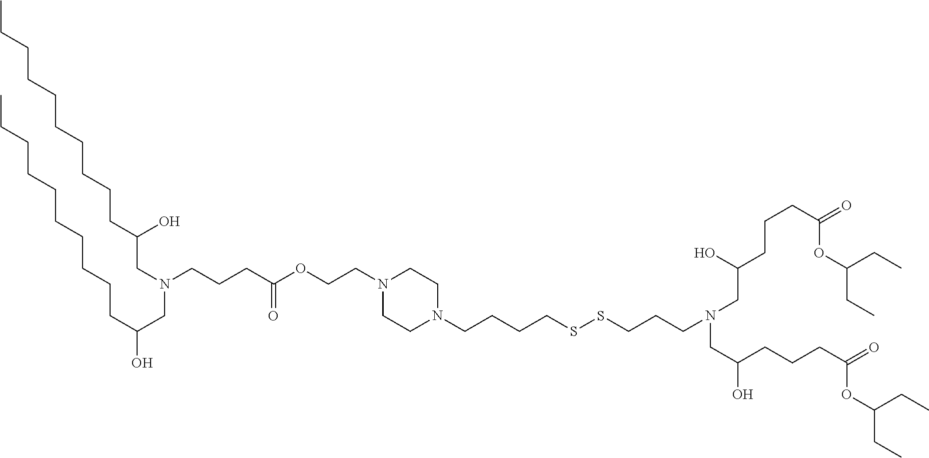 [SMILES: CCCCCCCCCCC(O)CN(CCCC(=O)OCCN1CCN(CCCCSSCCCN(CC(O)CCCC(=O)OC(CC)CC)CC(O)CCCC(=O)OC(CC)CC)CC1)CC(O)CCCCCCCCCC]

Following the very similar synthetic route described before, Asymmetric Good lipid (GL-Asymm-029) was obtained as a light-yellow oil (0.208 g, 42%). It was confirmed by the MS analysis.

ESI-MS analysis: Calculated for C₆₃H₁₂₅N₄O₁₀S₂, [M+H]=1161.88; Observed=1161.1

### Example 2: Lipid Nanoparticle Formulation

Cationic lipids described herein can be used in the preparation of lipid nanoparticles according to methods known in the art. For example, suitable methods include methods described in International Publication No. WO 2018/089801, which is hereby incorporated by reference in its entirety.

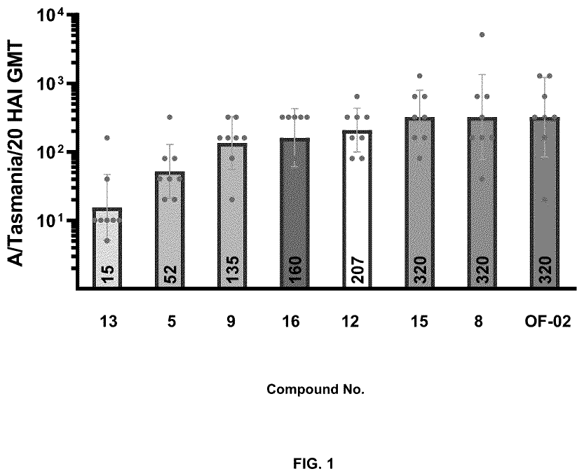

The lipid nanoparticles in the examples of the present invention were formulated using Process A of WO 2018/089801 (see, e.g., Example 1 and FIG. 1 of WO 2018/089801). Process A (“A”) relates to a conventional method of encapsulating mRNA by mixing mRNA with a mixture of lipids, without first pre-forming the lipids into lipid nanoparticles. In an exemplary process, an ethanolic solution of a mixture of lipids (cationic lipid, phosphatidylethanolamine, cholesterol, and polyethylene glycol-lipid) at a fixed lipid to mRNA ratio were combined with an aqueous buffered solution of target mRNA at an acidic pH under controlled conditions to yield a suspension of uniform LNPs. After ultrafiltration and diafiltration into a suitable diluent system, the resulting nanoparticle suspensions were diluted to final concentration, filtered, and stored frozen at −80 °C until use.

Lipid nanoparticle formulations of Table C were prepared by Process A. All of the lipid nanoparticle formulations comprised hEPO mRNA and the different lipids (Cationic Lipid: DMG-PEG2000: Cholesterol: DOPE/DSPC) in the mol % ratios specified in Table C.

TABLE C
Exemplary lipid nanoparticle characterizations

| Formulation | | Formulation Composition | | |
|---|---|---|---|---|
| number | mRNA | Cationic Lipid:DMG-PEG2000:Cholesterol:DOPE/DSPC | Process | N/P* |
| 1 | hEPO | Cationic Lipid:DMG-PEG2000:Cholesterol:DOPE: | A | 4 |
| | | 40:1.5:28.5:30 | | |
| 2 | hEPO | Cationic Lipid:DMG-PEG2000:Cholesterol:DSPC: | A | 3 |
| | | 50:1.5:38.5:10 | | |

*The N/P ratio is defined as the ratio of the number of nitrogen in cationic lipid to the number of phosphate in nucleic acid.

The cationic lipids of the present invention and lipid OF-02 were evaluated with lipid nanoparticle formulation 1. MC3 and SM-102 were evaluated with lipid nanoparticle formulation 2, which is a typical formulation for those lipids.

### Example 3: Delivery of hEPO mRNA by Intramuscular Administration

### Mouse Studies

In summary, lipid screening studies were conducted with female BALB/cJ mice 6-8 weeks of age (Jax #000651). Mice were dosed with 0.1 μg in 30 μL of LNPs by a single intramuscular (IM) injection into the gastrocnemius leg muscle. Blood samples were taken 6 and 24 hours post injection and hEPO levels were measured in the blood serum of the mice using an ELISA assay according to the manufacture's protocol (R&D systems, Cat #DEP-00). WO2022/099003 A1 also describes an in vivo assay for intramuscular administration (e.g. on page 46, paragraph [00206]). Measured serum hEPO expression data are presented in Table D below. This data demonstrates that the disclosed lipids can induce expression of delivered mRNAs.

TABLE D
Results of hEPO mRNA delivery studies - intramuscular
administration of hEPO mRNA lipid formulations
comprising the disclosed cationic lipids

| | | hEPO | hEPO | hEPO | hEPO |
|---|---|---|---|---|---|
| Cationic | | 6 Hr | 6 Hr | 24 Hr | 24 Hr |
| Lipid | Dose | (ng/mL) | (ng/mL) | (ng/mL) | (ng/mL) |
| (Cat) | (μg/An) | Mean | SD | Mean | SD |
| MC3 | 0.1 | 3.9 | 0.3 | 2.9 | 0.6 |
| SM102 | 0.1 | 12.5 | 6.4 | 13.3 | 6.0 |
| 1 | 0.1 | 11 | 2.7 | 9.5 | 3 |
| 2 | 0.1 | 35.4 | 5.2 | 29.2 | 7.3 |
| 3 | 0.1 | 20.5 | 4.6 | 19.3 | 4.9 |
| 4 | 0.1 | 18.8 | 3.1 | 12.1 | 3.5 |
| 5 | 0.1 | 35 | 3.3 | 18.7 | 2.6 |
| 6 | 0.1 | 34.7 | 3.7 | 18.4 | 2.2 |
| 7 | 0.1 | 31.4 | 3.2 | 25.8 | 4.3 |
| 8 | 0.1 | 41.8 | 6.7 | 33.3 | 11.1 |
| 9 | 0.1 | 24.3 | 7.2 | 15.7 | 5 |
| 10 | 0.1 | 19.6 | 5.7 | 7.7 | 2.5 |
| 11 | 0.1 | 18.7 | 3.2 | 10.1 | 1.6 |
| 12 | 0.1 | 37.4 | 5.1 | 21 | 5.6 |
| 13 | 0.1 | 29.5 | 4.1 | 13.8 | 2.9 |
| 14 | 0.1 | 26.4 | 4.6 | 12.8 | 3 |
| 15 | 0.1 | 36 | 7 | 22.8 | 4.7 |
| 16 | 0.1 | 32.4 | 4.1 | 18.7 | 4.4 |
| 17 | 0.1 | 19.7 | 4.4 | 10.4 | 1.4 |
| 18 | 0.1 | 18.2 | 2.8 | 9.2 | 1.7 |

### Example 4: Influenza Titer

Groups of Balb/c mice (Mus musculus) as per the treatment group were immunized under isoflurane anesthesia with a dose of 0.4 μg per mouse in 0.05 mL of Modified Tasmania H₃ mRNA-lipid nanoparticles via the IM route in the quadriceps, on day 0 in one hind leg and day 21 in the contralateral leg. Mice were evaluated for a minimum of 3 days post-administration and any animal that displayed severe clinical signs after the veterinarian's assessment was euthanized by administration of 5 mg/kg of meloxicam by subcutaneous injection.

Blood was collected via submandibular or orbital sinus bleeds (in-life bleeds were performed on day-1 and on day 20) and cardiac puncture (terminal bleed, day 35) from all animals under sedation. Mice were bled on pre-study to obtain a base-line pre-immune serum sample and for pre-screening purposes.

HAI assays were performed using the A/Tasmania/503/2020 (H₃N₂) virus stocks (BIOQUAL, Inc.). Sera were treated with receptor-destroying enzyme (RDE) by diluting one part serum with three parts enzyme and incubated overnight in a 37 °C water bath. Enzyme was inactivated by a 30-minute incubation period at 56 °C followed by addition of six parts PBS for a final dilution of 1/10. HAI assays were performed in V-bottom 96-well plates using four hemagglutinating units (HAU) of virus and 0.5% turkey RBC. The reference serum for each strain was included as a positive control on every assay plate. Each plate also included a back-titration to confirm the antigen dose (4 HAU/25 μl) as well as a negative control sample (PBS or naive control serum). The HAI titer was determined as the highest dilution of serum resulting in complete inhibition of hemagglutination. Results were only considered valid for plates with the appropriate back-titration result (verifying 4 HAU/25 μl added) and a reference serum titer within 2-fold of the expected titer. The majority of tested lipids induced HAI titers not significantly different from that of clinical lipid OF-02 (see FIG. 1). This example demonstrates that the disclosed lipids can deliver antigenic sequences that induce significant immune responses.

### NUMBERED EMBODIMENTS

1. A compound having a structure according to Formula (I′), optionally Formula (I):

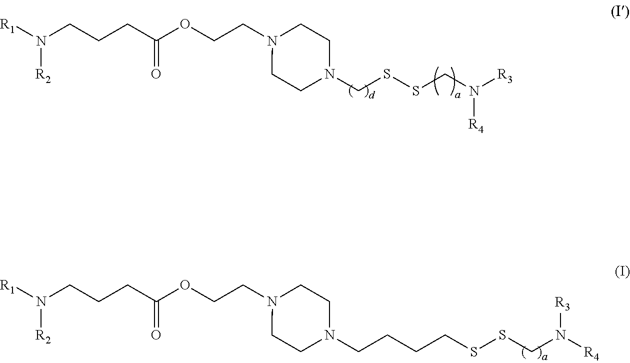

or a pharmaceutically acceptable salt thereof, wherein:
a is 3 or 4;
each of R₁, R₂, R₃, and R₄ is independently selected from:
   (i) optionally substituted C₅-C₃₀ alkyl, and optionally substituted C₅-C₃₀ alkenyl;
   (ii) optionally substituted C₁₋₅ alkyl which is substituted with a heterocycle moiety;
   (iii)

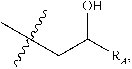

wherein each R_{A} is independently selected from optionally substituted C₃-C₂₈ alkyl, optionally substituted C₃-C₂₈ alkenyl, and —W¹—C(O)—O—X¹, wherein each W¹ is independently selected from optionally substituted C₃-C₅ alkylene and optionally substituted C₃-C₅ alkenylene, and each X¹ is independently selected from optionally substituted C₅-C₃₀ alkyl and optionally substituted C₅-C₃₀ alkenyl;
(iv)

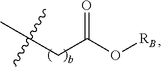

wherein each R_{B} is independently selected from optionally substituted C₅-C₃₀ alkyl, optionally substituted C₅-C₃₀ alkenyl; and
(v)

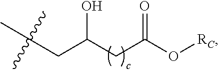

wherein each R_{C} independently selected from optionally substituted C₅-C₃₀ alkyl, optionally substituted C₅-C₃₀ alkenyl;
each b is independently selected from 5, 6, and 7;
each c is independently selected from 3, 4, and 5;
d is 2, 3, or 4;
and wherein
R₁ and R₂ are not identical to one another and/or R₃ and R₄ are not identical to one another; and wherein
at least one of R₁, R₂, R₃, and R₄, is independently selected from option (iv)

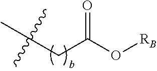

or option (v)

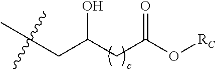

above.

2. The compound of embodiment 1, or a pharmaceutically acceptable salt thereof, wherein
(a) if R₁ and R₂ are both

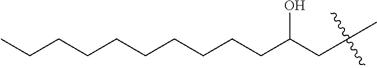 [SMILES: *CC(O)CCCCCCCCCC]

and one of R₃ and R₄ is

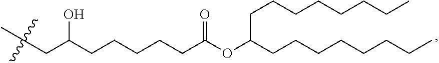 [SMILES: *CC(O)CCCCCC(=O)OC(CCCCCCCC)CCCCCCCC]

then the other of R₃ and R₄ is not selected from

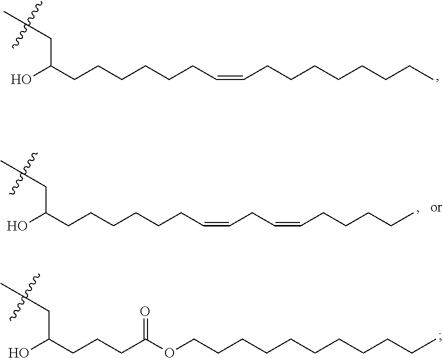 [SMILES: *CC(O)CCCCCC/C=C\CCCCCCCC ; *CC(O)CCCCCC/C=C\C/C=C\CCCCC ; *CC(O)CCCC(=O)OCCCCCCCCCCC]

or
(b) if R₁ and R₂ are both

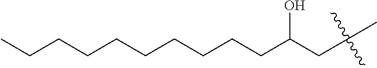 [SMILES: *CC(O)CCCCCCCCCC]

and one of R₃ and R₄ is

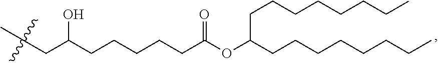 [SMILES: *CC(O)CCCCCC(=O)OC(CCCCCCCC)CCCCCCCC]

and a is 4 then the other of R₃ and R₄ is not selected from

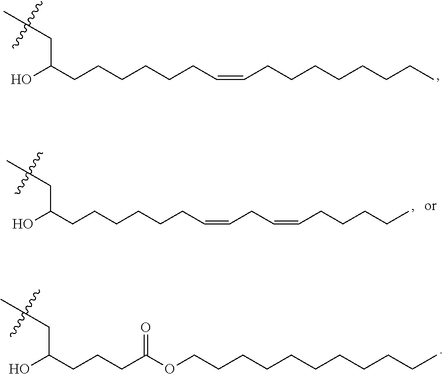 [SMILES: *CC(O)CCCCCC/C=C\CCCCCCCC ; *CC(O)CCCCCC/C=C\C/C=C\CCCCC ; *CC(O)CCCC(=O)OCCCCCCCCCCC]

3. The compound of embodiments 1 or 2, or a pharmaceutically acceptable salt thereof, wherein the compound is not

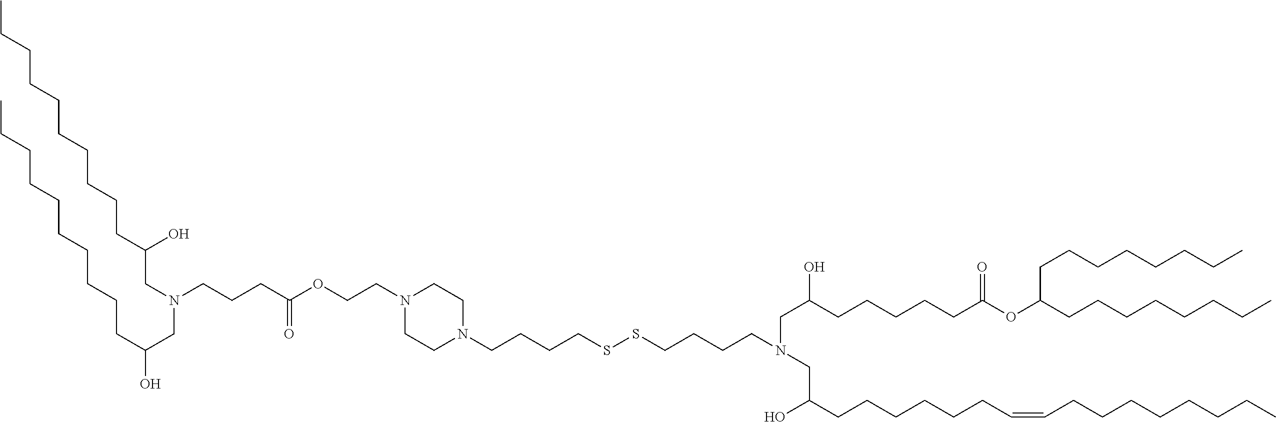 [SMILES: CCCCCCCC/C=C\CCCCCCC(O)CN(CCCCSSCCCCN1CCN(CCOC(=O)CCCN(CC(O)CCCCCCCCCC)CC(O)CCCCCCCCCC)CC1)CC(O)CCCCCC(=O)OC(CCCCCCCC)CCCCCCCC]

4. The compound of any one of embodiments 1-3, or a pharmaceutically acceptable salt thereof, wherein the compound is not

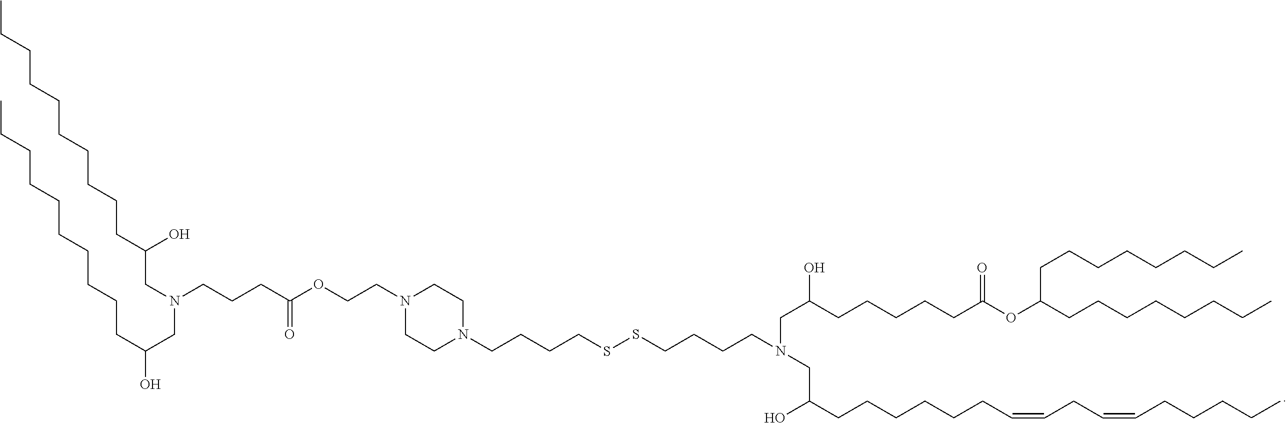 [SMILES: CCCCC/C=C\C/C=C\CCCCCCC(O)CN(CCCCSSCCCCN1CCN(CCOC(=O)CCCN(CC(O)CCCCCCCCCC)CC(O)CCCCCCCCCC)CC1)CC(O)CCCCCC(=O)OC(CCCCCCCC)CCCCCCCC]

5. The compound of any one of embodiments 1-4, or a pharmaceutically acceptable salt thereof, wherein the compound is not

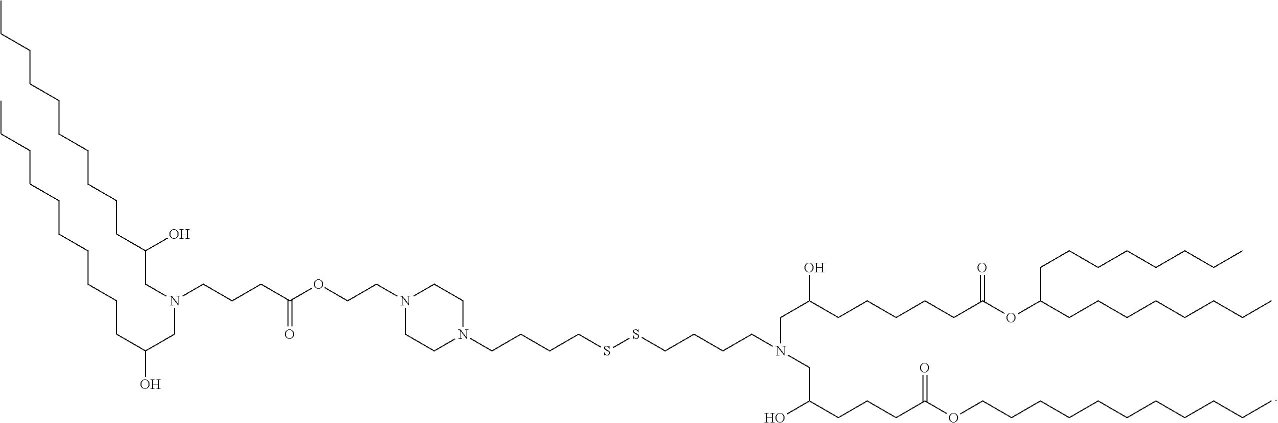 [SMILES: CCCCCCCCCCCOC(=O)CCCC(O)CN(CCCCSSCCCCN1CCN(CCOC(=O)CCCN(CC(O)CCCCCCCCCC)CC(O)CCCCCCCCCC)CC1)CC(O)CCCCCC(=O)OC(CCCCCCCC)CCCCCCCC]

6. The compound of any one of embodiments 1-5, or a pharmaceutically acceptable salt thereof, wherein:
(i) a is 3; or
(ii) a is 4.

7. The compound of any one of embodiments 1-6, or a pharmaceutically acceptable salt thereof, wherein:
(i) b is 5; or
(ii) b is 6; or
(iii) b is 7; or
(iv) each b is independently selected from 5 or 7.

8. The compound of any one of embodiments 1-7, or a pharmaceutically acceptable salt thereof, wherein R₁ and R₂ are not identical to one another and R₃ and R₄ are identical to one another.

9. The compound of any one of embodiments 1-7, or a pharmaceutically acceptable salt thereof, wherein R₁ and R₂ are identical to one another and R₃ and R₄ are not identical to one another.

10. The compound of any one of embodiments 1-7, or a pharmaceutically acceptable salt thereof, wherein:
(i) R₁ and R₂ are not identical to one another and R₃ and R₄ are not identical to one another; or
(ii) none of R₁, R₂, R₃, and R₄ are identical.

11. The compound of any one of embodiments 1-10, or a pharmaceutically acceptable salt thereof, wherein one or more of R₁, R₂, R₃, and R₄ are independently selected from optionally substituted C₅-C₃₀ alkyl or optionally substituted C₅-C₃₀ alkenyl.

12. The compound of embodiment 11, or a pharmaceutically acceptable salt thereof, wherein one or more of R₁, R₂, R₃, and R₄ are independently selected from optionally substituted C₅-C₃₀ alkyl.

13. The compound of embodiment 12, or a pharmaceutically acceptable salt thereof, wherein one or more of R₁, R₂, R₃, and R₄ are independently selected from optionally substituted C₅-C₁₂ alkyl, for example

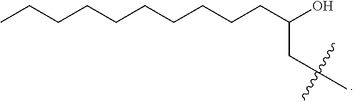 [SMILES: *CC(O)CCCCCCCCCC]

14. The compound of any one of embodiments 1-7, 9, or 11-13, wherein the compound has a structure according to Formula (II′), optionally Formula (II):

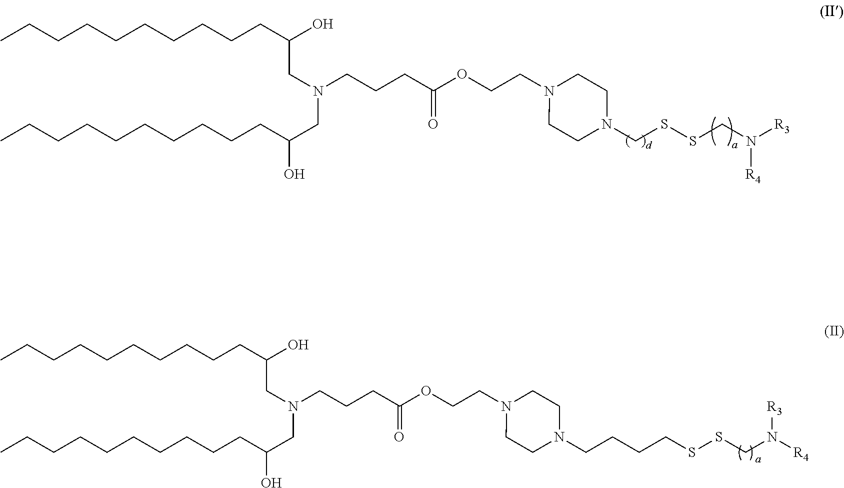

or a pharmaceutically acceptable salt thereof, optionally wherein a is 4.

15. The compound of any one of embodiments 11-14, or a pharmaceutically acceptable salt thereof, wherein one or more of R₁, R₂, R₃, and R₄ are independently selected from optionally substituted C₅-C₃₀ alkenyl.

16. The compound of embodiment 15, or a pharmaceutically acceptable salt thereof, wherein one or more of R₁, R₂, R₃, and R₄ are independently selected from optionally substituted C₅-C₁₈ alkenyl, for example

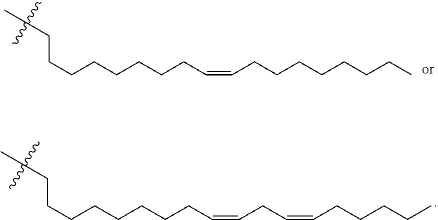 [SMILES: *CCCCCCCC/C=C\CCCCCCCC ; *CCCCCCCC/C=C\C/C=C\CCCCC]

17. The compound of any one of embodiments 11-16, or a pharmaceutically acceptable salt thereof, wherein two of R₁, R₂, R₃, and R₄ are independently selected from optionally substituted C₅-C₃₀ alkyl and one of R₁, R₂, R₃, and R₄ is selected from optionally substituted C₅-C₃₀ alkenyl.

18. The compound of any one of embodiments 1-17, or a pharmaceutically acceptable salt thereof, wherein one or more of R₁, R₂, R₃, and R₄ is independently selected from

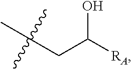

wherein each R_{A} is independently selected from optionally substituted C₃-C₂₈ alkyl, optionally substituted C₃-C₂₈ alkenyl, and —W¹—C(O)—O—X¹, wherein each W¹ is independently selected from optionally substituted C₃-C₅ alkylene and optionally substituted C₃-C₅ alkenylene, and each X¹ is independently selected from optionally substituted C₅-C₃₀ alkyl and optionally substituted C₅-C₃₀ alkenyl.

19. The compound of embodiment 18, or a pharmaceutically acceptable salt thereof, wherein one or more R_{A} is independently selected from optionally substituted C₃-C₂₈ alkyl, optionally wherein each R_{A} is independently selected from optionally substituted C₃-C₂₈ alkyl.

20. The compound of embodiment 19, or a pharmaceutically acceptable salt thereof, wherein one or more R_{A} is independently selected from optionally substituted C₃-C₁₀ alkyl, for example

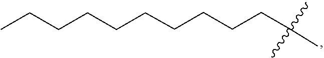 [SMILES: *CCCCCCCCCC]

optionally wherein each R_{A} is independently selected from optionally substituted C₃-C₁₀ alkyl, for example

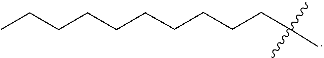 [SMILES: *CCCCCCCCCC]

21. The compound of any one of embodiments 18-20, or a pharmaceutically acceptable salt thereof, wherein one or more R_{A} is independently selected from optionally substituted C₃-C₂₈ alkenyl, for example optionally substituted C₃-C₁₆ alkenyl, optionally wherein each R_{A} is independently selected from optionally substituted C₃-C₂₈ alkenyl, for example optionally substituted C₃-C₁₆ alkenyl.

22. The compound of any one of embodiments 18-21, or a pharmaceutically acceptable salt thereof, wherein one or more R_{A} is independently selected from —W¹—C(O)—O—X¹, optionally wherein each R_{A} is independently selected from —W¹—C(O)—O—X¹.

23. The compound of embodiment 22, or a pharmaceutically acceptable salt thereof, wherein one or more W¹ is independently selected from optionally substituted C₃-C₅ alkylene, optionally wherein each W¹ is independently selected from optionally substituted C₃-C₅ alkylene.

24. The compound of embodiment 22, or a pharmaceutically acceptable salt thereof, wherein one or more W¹ is independently selected from optionally substituted C₃-C₅ alkenylene, optionally wherein each W¹ is independently selected from optionally substituted C₃-C₅ alkenylene.

25. The compound of any one of embodiments 22-24, or a pharmaceutically acceptable salt thereof, wherein one or more X¹ is independently selected from optionally substituted C₅-C₃₀ alkyl, optionally wherein each X¹ is independently selected from optionally substituted C₅-C₃₀ alkyl.

26. The compound of embodiment 25, or a pharmaceutically acceptable salt thereof, wherein one or more X¹ is independently selected from optionally substituted C₆-C₁₇ alkyl, for example optionally substituted branched C₆-C₁₇ alkyl, optionally wherein each X¹ is independently selected from optionally substituted C₆-C₁₇ alkyl, for example optionally substituted branched C₆-C₁₇ alkyl.

27. The compound of embodiment 26, or a pharmaceutically acceptable salt thereof, wherein one or more X¹ is independently selected from
(i)

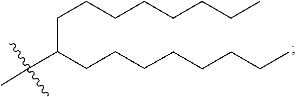 [SMILES: *C(CCCCCCCC)CCCCCCCC]

(ii)

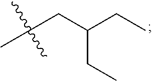 [SMILES: *CC(CC)CC]

and
(iii)

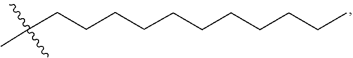 [SMILES: *CCCCCCCCCCC]

optionally wherein each X¹ is independently selected from options (i), (ii), and (iii) above.

28. The compound of any one of embodiments 22-27, or a pharmaceutically acceptable salt thereof, wherein one or more X¹ is independently selected from optionally substituted C₅-C₃₀ alkenyl, optionally wherein each X¹ is independently selected from optionally substituted C₅-C₃₀ alkenyl.

29. The compound of embodiment 28, or a pharmaceutically acceptable salt thereof, wherein one or more X¹ is independently selected from optionally substituted C₅-C₉ alkenyl, for example

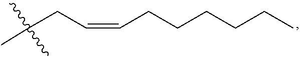 [SMILES: *C/C=C\CCCCCC]

optionally wherein each X¹ is independently selected from optionally substituted C₅-C₉ alkenyl, for example

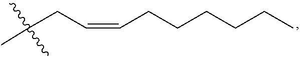 [SMILES: *C/C=C\CCCCCC]

30. The compound of any one of embodiments 1-29, or a pharmaceutically acceptable salt thereof, wherein one or more of R₁, R₂, R₃, and R₄ is independently selected from

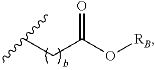

wherein each R_{B} is independently selected from optionally substituted C₅-C₃₀ alkyl and optionally substituted C₅-C₃₀ alkenyl.

31. The compound of embodiment 30, or a pharmaceutically acceptable salt thereof, wherein one or more R_{B} is independently selected from optionally substituted C₅-C₃₀ alkyl, optionally wherein each R_{B} is independently selected from optionally substituted C₅-C₃₀ alkyl.

32. The compound of embodiment 31, or a pharmaceutically acceptable salt thereof, wherein one or more R_{B} is independently selected from optionally substituted C₆-C₁₇ alkyl, for example optionally substituted branched C₆-C₁₇ alkyl, optionally wherein each R_{B} is independently selected from optionally substituted C₆-C₁₇ alkyl, for example optionally substituted branched C₆-C₁₇ alkyl.

33. The compound of embodiment 32, or a pharmaceutically acceptable salt thereof, wherein one or more R_{B} is independently selected from
(i)

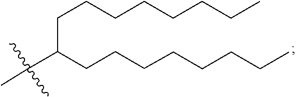 [SMILES: *C(CCCCCCCC)CCCCCCCC]

(ii)

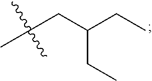 [SMILES: *CC(CC)CC]

and
(iii)

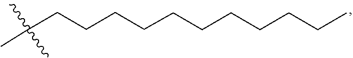 [SMILES: *CCCCCCCCCCC]

optionally wherein each R_{B} is independently selected from options (i), (ii), and (iii) above.

34. The compound of any one of embodiments 1-8, 10-13, or 15-33 wherein the compound has a structure according to Formula (III′), optionally Formula (III):

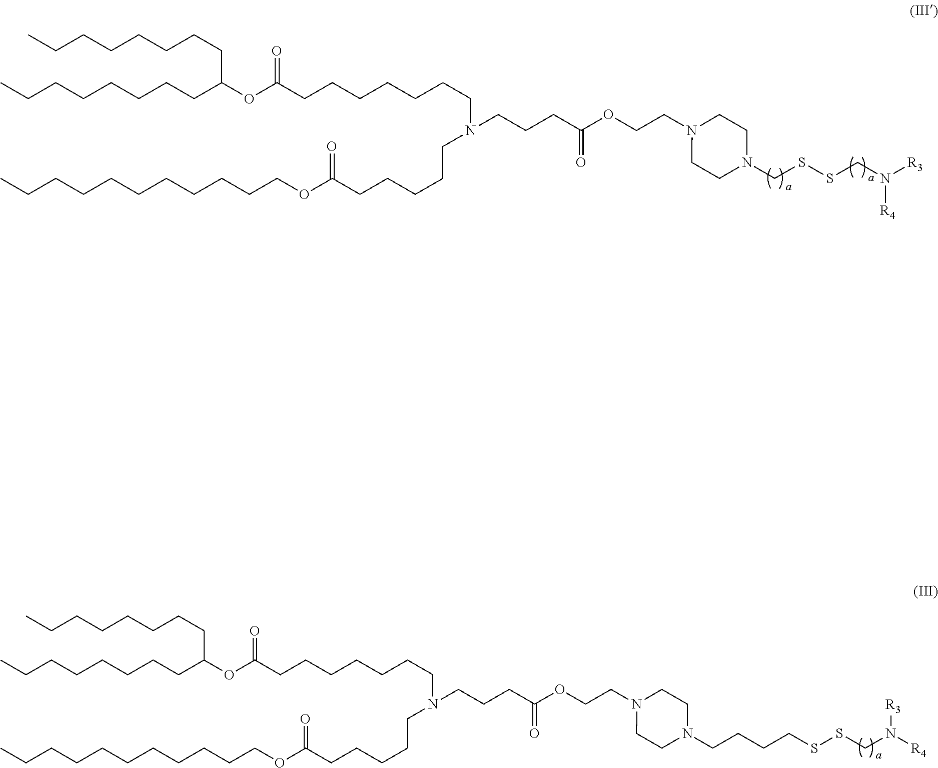

or a pharmaceutically acceptable salt thereof.

35. The compound of any one of embodiments 30-34, or a pharmaceutically acceptable salt thereof, wherein one or more R_{B} is independently selected from optionally substituted C₅-C₃₀ alkenyl, optionally wherein each R_{B} is independently selected from optionally substituted C₅-C₃₀ alkenyl.

36. The compound of embodiment 35, or a pharmaceutically acceptable salt thereof, wherein one or more R_{B} is independently selected from optionally substituted C₅-C₉ alkenyl, for example

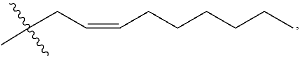 [SMILES: *C/C=C\CCCCCC]

optionally wherein each R_{B} is independently selected from optionally substituted C₅-C₉ alkenyl, for example

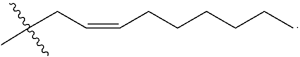 [SMILES: *C/C=C\CCCCCC]

37. The compound of any one of embodiments 1-36, or a pharmaceutically acceptable salt thereof, wherein one or more of R₁, R₂, R₃, and R₄ are independently selected from

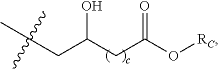

wherein each R_{C} independently selected from optionally substituted C₅-C₃₀ alkyl and optionally substituted C₅-C₃₀ alkenyl.

38. The compound of embodiment 37, or a pharmaceutically acceptable salt thereof, wherein one or more c is selected from 3 or 5, optionally wherein each c is selected from 3 or 5.

39. The compound of embodiment 37, or a pharmaceutically acceptable salt thereof, wherein one or more c is 4, optionally wherein each c is 4.

40. The compound of any one of embodiments 37 to 39, or a pharmaceutically acceptable salt thereof, wherein one or more R_{C} is independently selected from optionally substituted C₅-C₃₀ alkyl, optionally wherein each R_{C} is independently selected from optionally substituted C₅-C₃₀ alkyl.

41. The compound of embodiment 40, or a pharmaceutically acceptable salt thereof, wherein one or more R_{C} is independently selected from optionally substituted C₆-C₁₇ alkyl, for example optionally substituted branched C₆-C₁₇ alkyl, optionally wherein each R_{C} is independently selected from optionally substituted C₆-C₁₇ alkyl, for example optionally substituted branched C₆-C₁₇ alkyl.

42. The compound of embodiment 41, or a pharmaceutically acceptable salt thereof, wherein one or more R_{C} is independently selected from
(i)

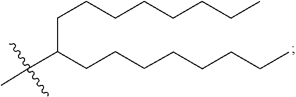 [SMILES: *C(CCCCCCCC)CCCCCCCC]

(ii)

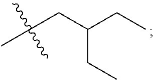 [SMILES: *CC(CC)CC]

and
(iii)

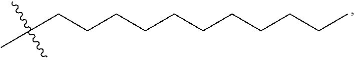 [SMILES: *CCCCCCCCCCC]

optionally wherein each R_{C} is independently selected from options (i), (ii) and (iii) above.

43. The compound of any one of embodiments 1-8, 11-13, 15-33, or 35-42 wherein the compound has a structure according to Formula (IV′), optionally Formula (IV); or Formula (V′), optionally Formula (V):

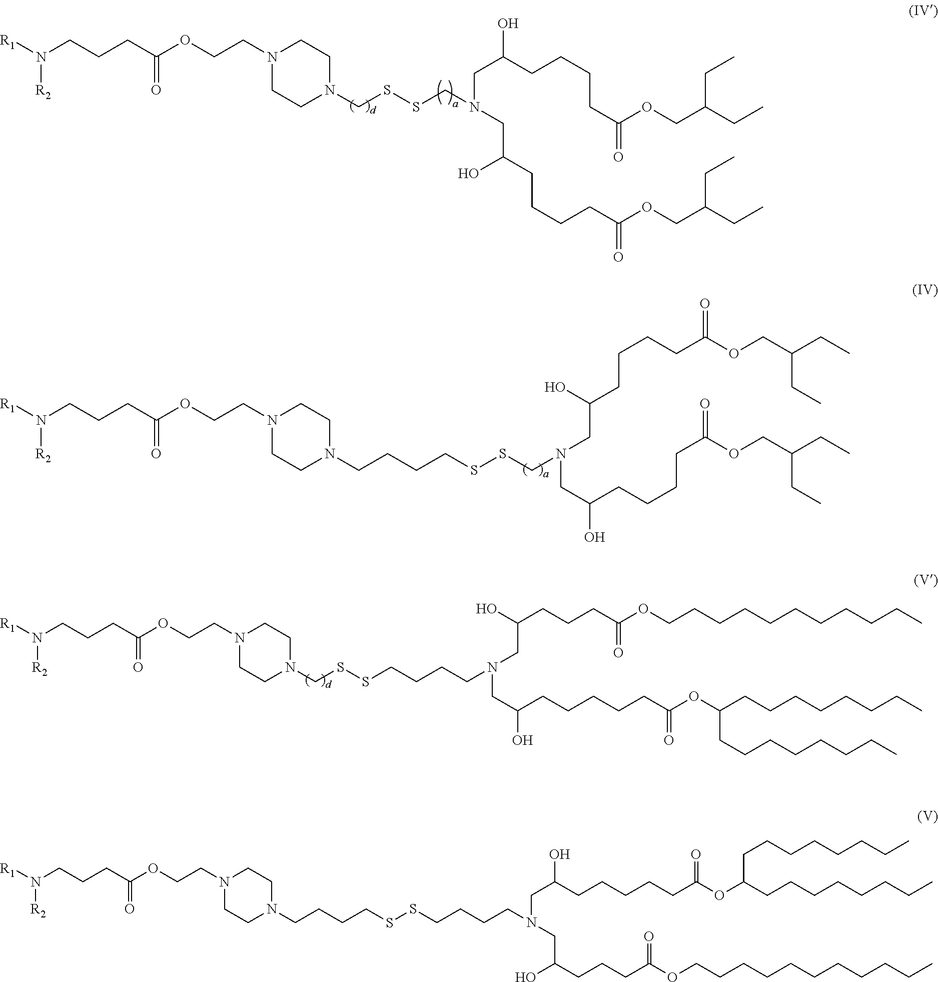

or a pharmaceutically acceptable salt thereof, optionally wherein a is 3.

44. The compound of any one of embodiments 37-43, or a pharmaceutically acceptable salt thereof, wherein one or more R_{C} is independently selected from optionally substituted C₅-C₃₀ alkenyl, optionally wherein each R_{C} is independently selected from optionally substituted C₅-C₃₀ alkenyl.

45. The compound of embodiment 44, or a pharmaceutically acceptable salt thereof, wherein one or more R_{C} are independently selected from optionally substituted C₅-C₉ alkenyl, for example

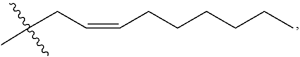 [SMILES: *C/C=C\CCCCCC]

optionally wherein each R_{C} are independently selected from optionally substituted C₅-C₉ alkenyl, for example

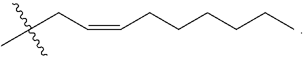 [SMILES: *C/C=C\CCCCCC]

46. The compound of any one of embodiments 1-45, or a pharmaceutically acceptable salt thereof, wherein one or more of R₁, R₂, R₃, and R₄ is independently selected from optionally substituted C₁₋₅ alkyl which is substituted with an optionally substituted heterocyclyl moiety.

47. The compound of embodiment 46, or a pharmaceutically acceptable salt thereof, wherein said optionally substituted C₁₋₅ alkyl which is substituted with an optionally substituted heterocyclyl moiety is

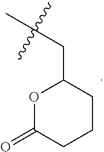 [SMILES: *CC1CCCC(=O)O1]

48. A compound having a structure according to Formula (VI′), optionally Formula (VI):

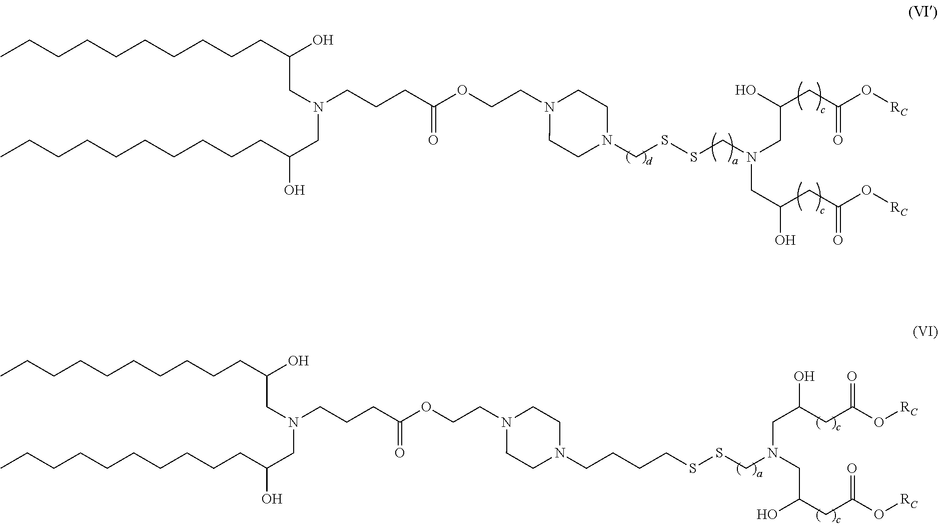

or a pharmaceutically acceptable salt thereof, wherein:
a is 3 or 4;
d is 2, 3, or 4;
each c is independently selected from 3 and 4; and
each R_{C} is independently selected from

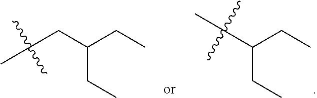

49. The compound of embodiment 48, or a pharmaceutically acceptable salt thereof, wherein a is 3.

50. The compound of embodiment 48, or a pharmaceutically acceptable salt thereof, wherein a is 4.

51. The compound of any one of embodiments 48-50, or a pharmaceutically acceptable salt thereof, wherein each c is 3.

52. The compound of any one of embodiments 48-50, or a pharmaceutically acceptable salt thereof, wherein each c is 4.

53. The compound of any one of embodiments 48-52, or a pharmaceutically acceptable salt thereof, wherein:
(i) each R_{C} is

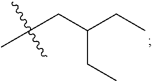 [SMILES: *CC(CC)CC]

or
(ii) each R_{C} is

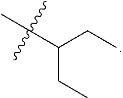 [SMILES: *C(CC)CC]

54. The compound of any one of embodiments 1-53, or a pharmaceutically acceptable salt thereof, wherein, in the compound of Formula (I′), (II′), (III′), (IV′), (V′), or (VI′):
(i) d is 2;
(ii) d is 3; or
(iii) d is 4.

55. A compound selected from those listed in Tables 1 or 2, or a pharmaceutically acceptable salt thereof.

56. A compound of embodiment 1, wherein the compound is selected from:

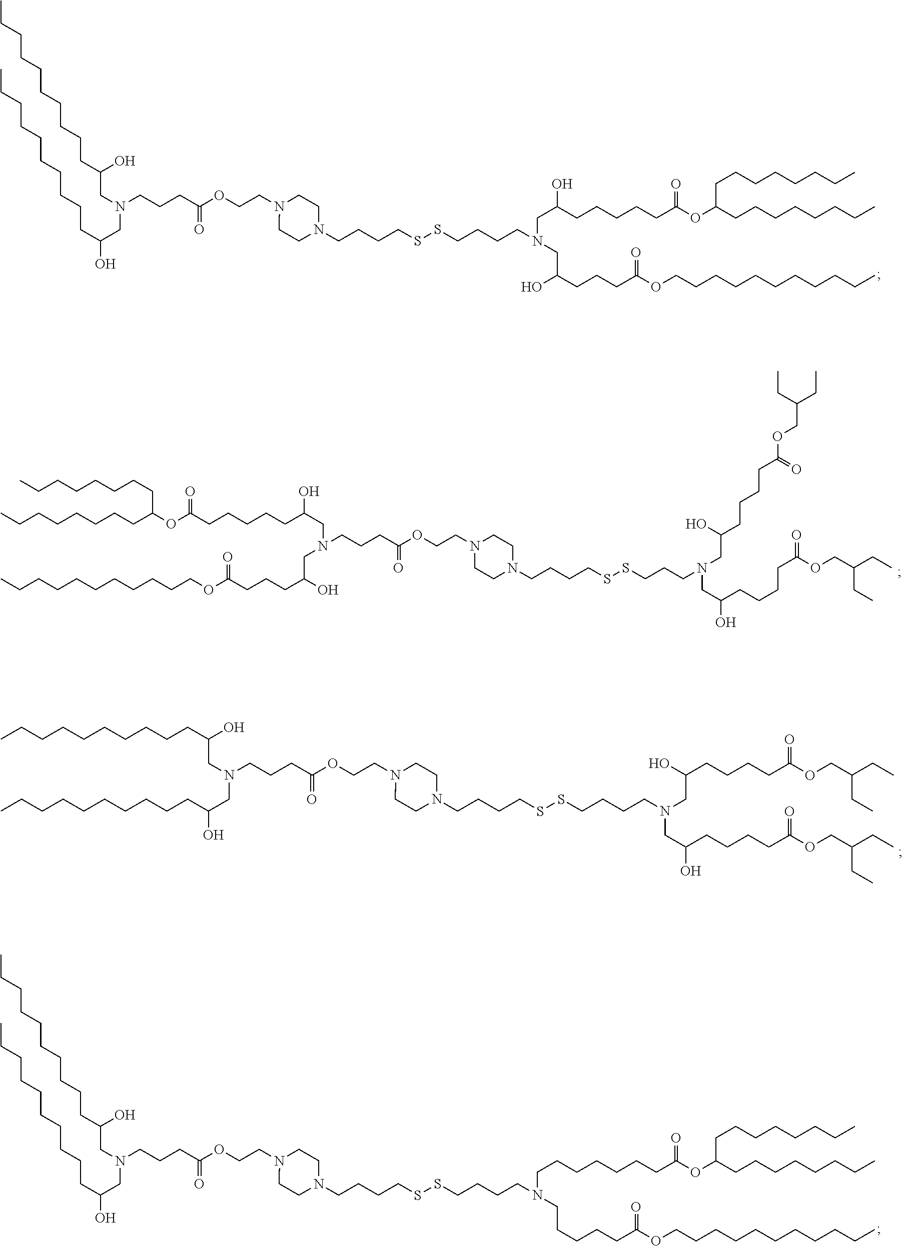

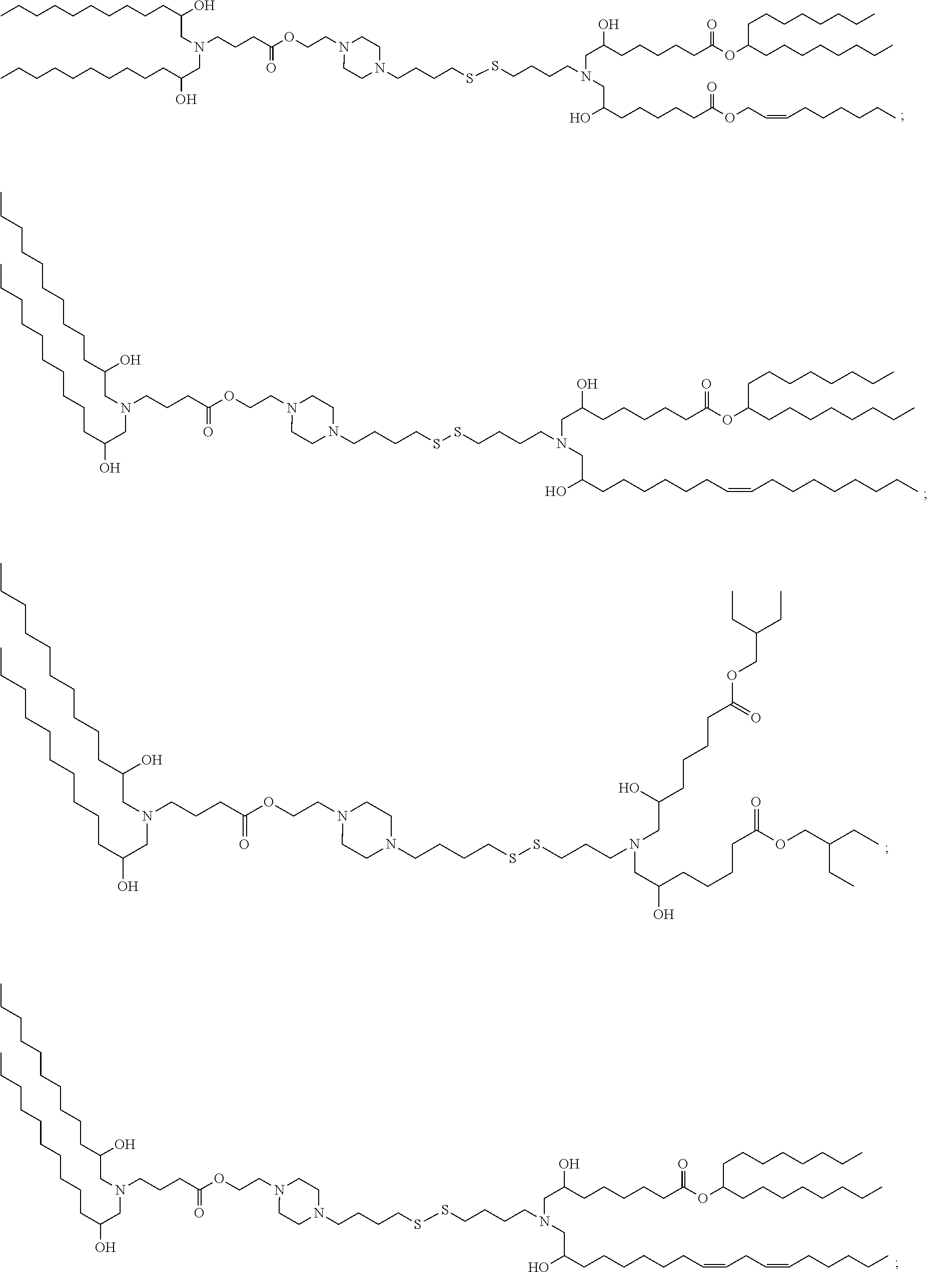

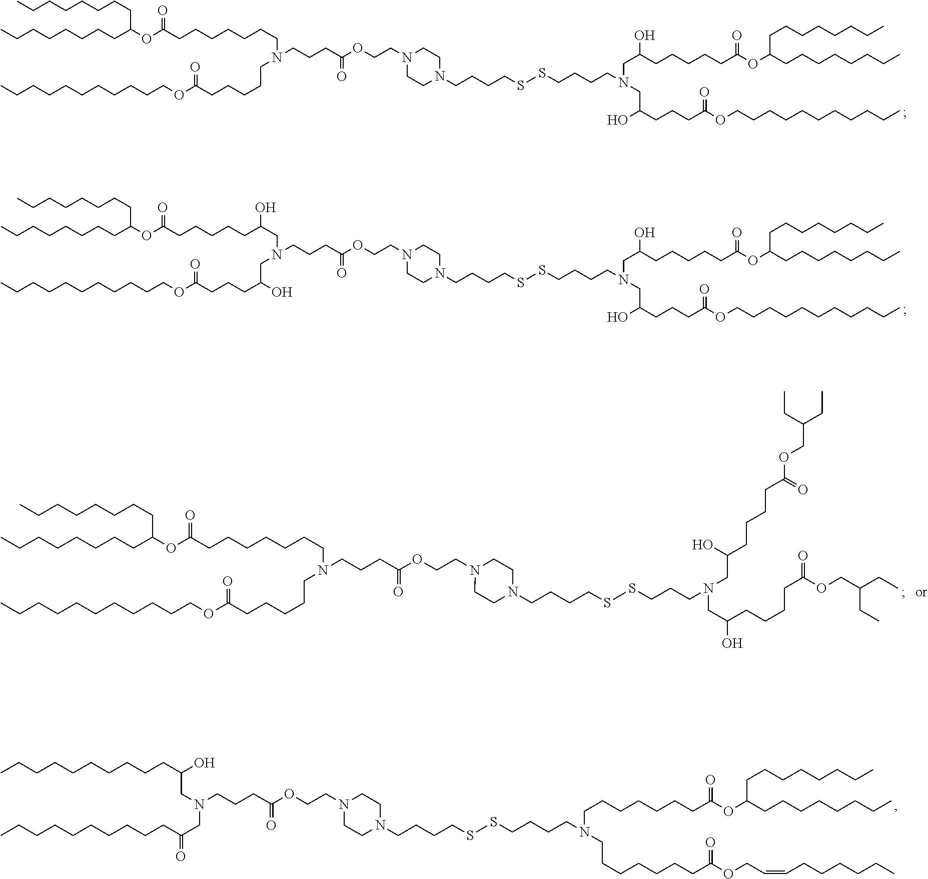

or a pharmaceutically acceptable salt thereof.

57. A compound of any one of embodiments 1-33, 35-42, 44-47, or 55-56, wherein the compound is selected from:

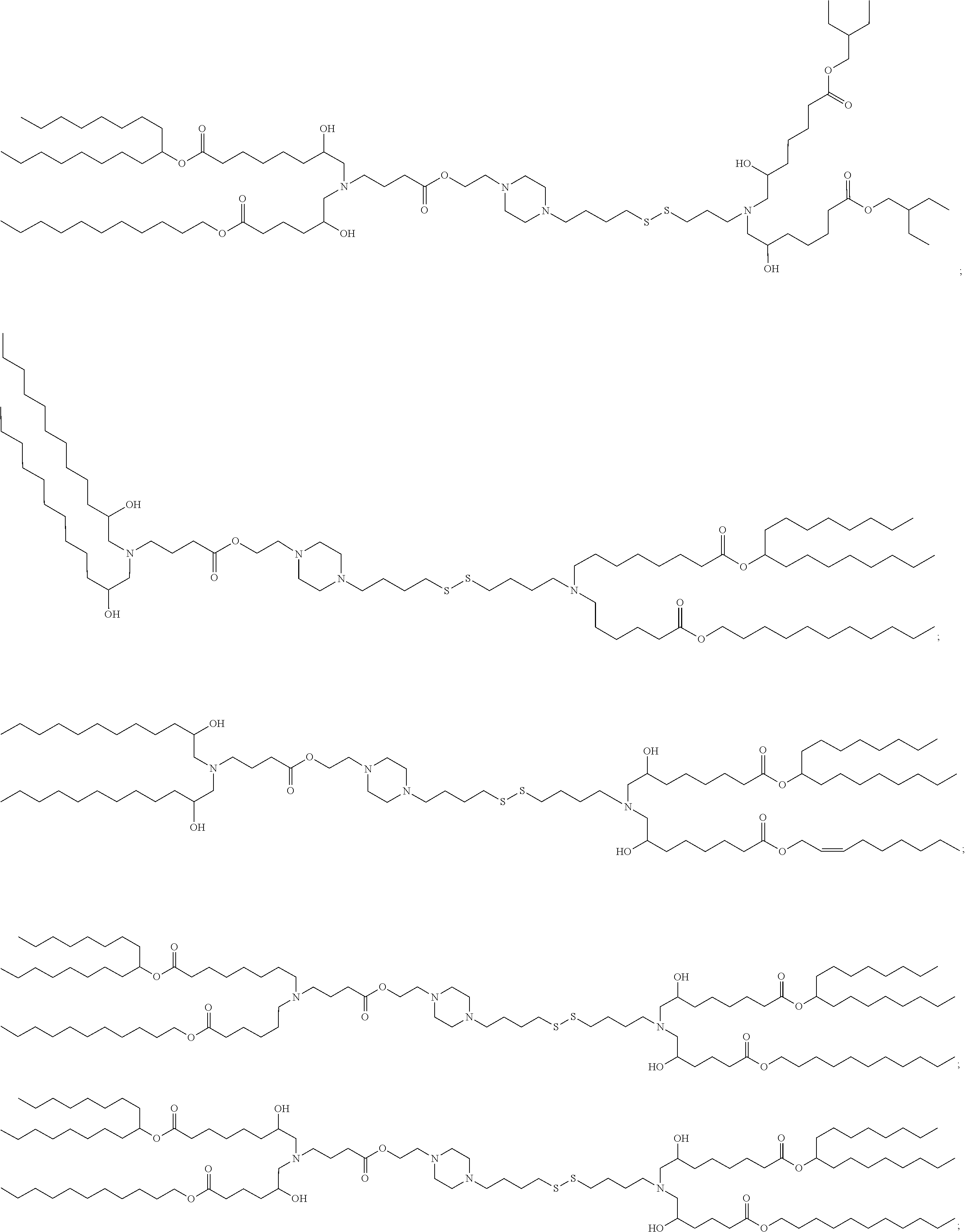

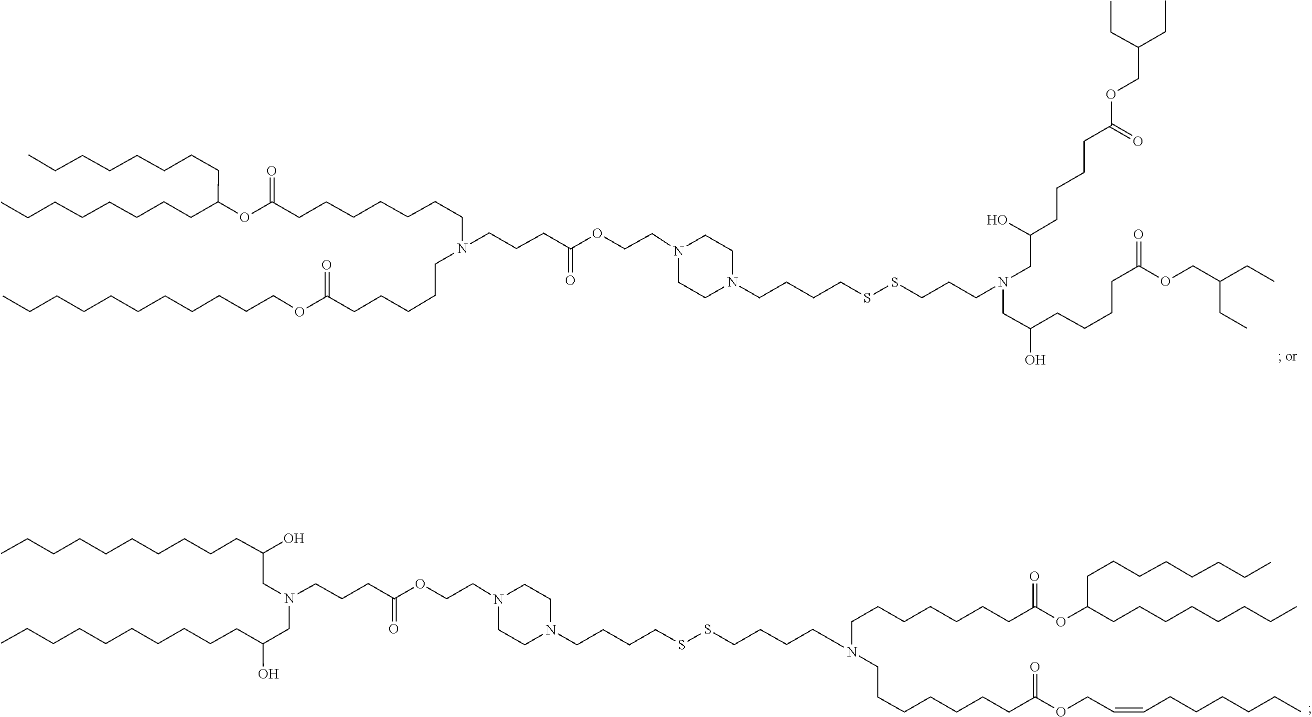

or a pharmaceutically acceptable salt thereof.

58. A compound of embodiment 1, wherein the compound is

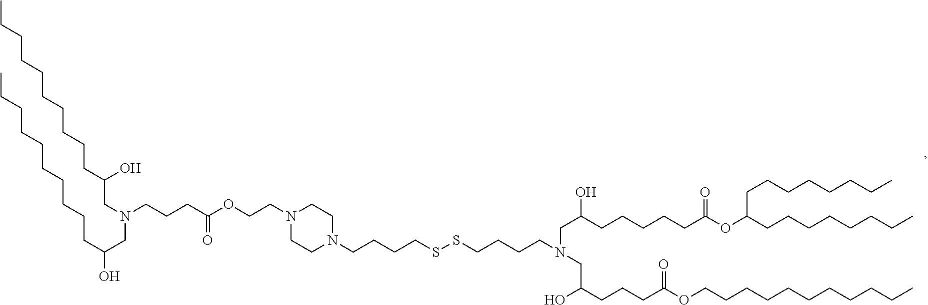 [SMILES: CCCCCCCCCCCOC(=O)CCCC(O)CN(CCCCSSCCCCN1CCN(CCOC(=O)CCCN(CC(O)CCCCCCCCCC)CC(O)CCCCCCCCCC)CC1)CC(O)CCCCCC(=O)OC(CCCCCCCC)CCCCCCCC]

or a pharmaceutically acceptable salt thereof.

59. A compound of any one of embodiments 48-55, wherein the compound is

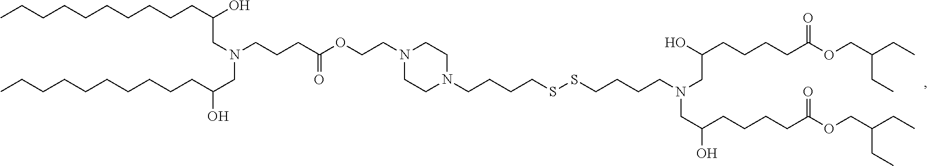 [SMILES: CCCCCCCCCCC(O)CN(CCCC(=O)OCCN1CCN(CCCCSSCCCCN(CC(O)CCCCC(=O)OCC(CC)CC)CC(O)CCCCC(=O)OCC(CC)CC)CC1)CC(O)CCCCCCCCCC]

or a pharmaceutically acceptable salt thereof.

60. A composition comprising the compound of any one of embodiments 1-59 or a pharmaceutically acceptable salt thereof, one or more non-cationic lipids, one or more cholesterol-based lipids and one or more PEG-modified lipids.

61. The composition of embodiment 60, wherein the composition is a lipid nanoparticle, optionally a liposome.

62. The composition of embodiment 61, wherein the one or more cationic lipid(s) constitute(s) about 30 mol %-60 mol % of the lipid nanoparticle.

63. The composition of any one of embodiments 61-62, wherein the one or more non-cationic lipid(s) constitute(s) 10 mol %-50 mol % of the lipid nanoparticle.

64. The composition of any one of embodiments 61-63, wherein the one or more PEG-modified lipid(s) constitute(s) 1 mol %-10 mol % of the lipid nanoparticle.

65. The composition of any one of embodiments 61-64, wherein the cholesterol-based lipid constitutes 10 mol %-50 mol % of the lipid nanoparticle.

66. The composition of any one of embodiments 61-65, wherein the lipid nanoparticle encapsulates a nucleic acid.

67. The composition of any one of embodiments 61-65, wherein the lipid nanoparticle encapsulates a mRNA encoding a peptide or protein.

68. The composition of embodiment 67, wherein the mRNA encodes an antigen.

69. The composition of embodiment 68, wherein the mRNA encodes an influenza antigen, for example an antigen of influenza A virus or an antigen of influenza B virus.

70. The composition of embodiment 68, wherein the mRNA encodes an antigen of Chlamydia sp. bacteria, for example an antigen of C. trachomatis.

71. The composition of embodiment 68, wherein the mRNA encodes an antigen of P. gingivalis.

72. The composition of embodiment 68, wherein the mRNA encodes an acne antigen, for example an antigen of C. acnes.

73. The composition of any one of embodiments 67-72, wherein the lipid nanoparticles have an encapsulation percentage for mRNA of
(i) at least 60%
(ii) at least 65%
(iii) at least 70%;
(iv) at least 75%;
(v) at least 80%;
(vi) at least 85%;
(vii) at least 90%; or
(viii) at least 95%.

74. The composition of any one of embodiments 66-73 for use in therapy.

75. The composition of any one of embodiments 67-73 for use in a method of treating or preventing a disease or disorder amenable to treatment or prevention by the peptide or protein encoded by the mRNA.

76. The composition for use according to embodiment 75, wherein the disease or disorder is caused by a viral infection.

77. The composition for use according to embodiment 76, wherein the disease or disorder is influenza.

78. The composition for use according to embodiment 75, wherein the disease or disorder is caused by a bacterial infection.

79. The composition for use according to embodiment 78, wherein the disease or disorder is chlamydia.

80. The composition for use according to embodiment 78, wherein the disease or disorder is induced by infection with P. gingivalis.

81. The composition for use according to embodiment 78 wherein the disease or disorder is acne.

82. A method of treating or preventing a disease or disorder wherein said method comprises administering to a subject in need thereof the composition of any one of embodiments 67-73 and wherein the disease is amenable to treatment or prevention by the peptide or protein encoded by the mRNA.

83. The method of embodiment 82 wherein the disease or disorder is caused by a viral infection.

84. The method of embodiment 83, wherein the disease or disorder is influenza.

85. The method of embodiment 82, wherein the disease or disorder is caused by a bacterial infection.

86. The method of embodiment 85, wherein the disease or disorder is chlamydia.

87. The method of embodiment 85, wherein the disease or disorder is a disease induced by infection with P. gingivalis.

88. The method of embodiment 85, wherein the disease or disorder is acne.

89. The composition for use according to any of embodiments 74-81, or the method of any one of embodiments 82-88, wherein the composition is administered intravenously, intrathecally or intramuscularly, or by pulmonary delivery, optionally through nebulization.

90. The composition for use according to embodiment 89, or the method of embodiment 89, wherein the composition is administered intramuscularly.

91. The composition of embodiment 69 for use in a method of treating or preventing influenza, wherein the composition is administered intramuscularly.

92. The composition of embodiment 70 for use in a method of treating or preventing chlamydia, wherein the composition is administered intramuscularly.

93. The composition of embodiment 71 for use in a method of treating or preventing a disease induced by infection with P. gingivalis, wherein the composition is administered intramuscularly.

94. The composition of embodiment 72 for use in a method of treating or preventing acne, wherein the composition is administered intramuscularly.

95. A method for treating or preventing influenza, wherein said method comprises administering to a subject in need thereof the composition of embodiment 69, wherein the composition is administered intramuscularly.

96. A method for treating or preventing chlamydia, wherein said method comprises administering to a subject in need thereof the composition of embodiment 70, wherein the composition is administered intramuscularly.

97. A method for treating or preventing a disease induced by infection with P. gingivalis, wherein said method comprises administering to a subject in need thereof the composition of embodiment 71, wherein the composition is administered intramuscularly.

98. A method for treating or preventing acne, wherein said method comprises administering to a subject in need thereof the composition of embodiment 72, wherein the composition is administered intramuscularly.

99. A compound having a structure according to Formula (I′), optionally Formula (I):

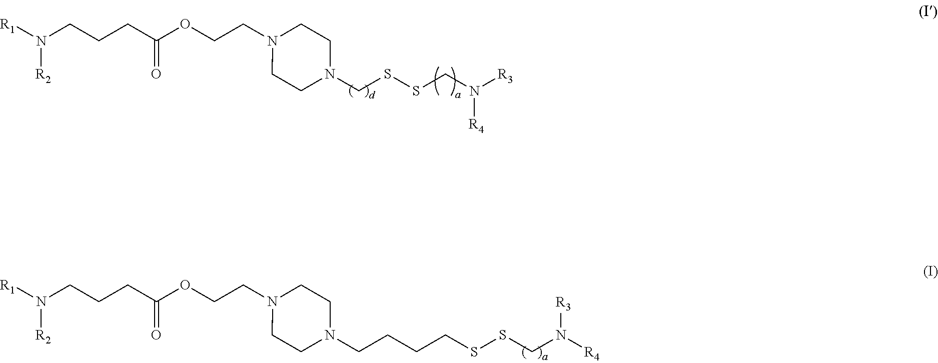

or a pharmaceutically acceptable salt thereof, wherein:
a is 3 or 4;
each of R₁, R₂, R₃, and R₄ is independently selected from:
   (i) optionally substituted C₅-C₃₀ alkyl, and optionally substituted C₅-C₃₀ alkenyl;
   (ii) optionally substituted C₁₋₅ alkyl which is substituted with an optionally substituted heterocyclyl moiety;
   (iii)

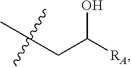

wherein each R_{A} is independently selected from optionally substituted C₃-C₂₈ alkyl, optionally substituted C₃-C₂₈ alkenyl, and —W¹—C(O)—O—X¹, wherein each W¹ is independently selected from optionally substituted C₃-C₅ alkylene and optionally substituted C₃-C₅ alkenylene, and each X¹ is independently selected from optionally substituted C₅-C₃₀ alkyl and optionally substituted C₅-C₃₀ alkenyl;
(iv)

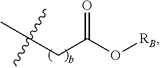

wherein each R_{B} is independently selected from optionally substituted C₅-C₃₀ alkyl, optionally substituted C₅-C₃₀ alkenyl; and
(v)

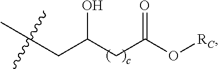

wherein each R_{C} independently selected from optionally substituted C₅-C₃₀ alkyl, optionally substituted C₅-C₃₀ alkenyl;
each b is independently selected from 5, 6, and 7;
each c is independently selected from 3, 4, and 5;
d is 2, 3, or 4;
and wherein
R₁ and R₂ are not identical to one another and/or R₃ and R₄ are not identical to one another; and wherein
at least one of R₁, R₂, R₃, and R₄, is independently selected from option (iv)

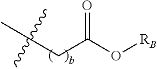

or option (v)

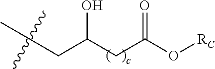

above.

100. The compound of embodiment 99, or a pharmaceutically acceptable salt thereof, wherein:
(a) if R₁ and R₂ are both

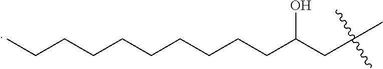 [SMILES: *CC(O)CCCCCCCCCC]

and one of R₃ and R₄ is

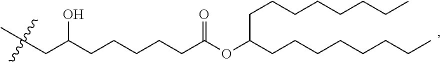 [SMILES: *CC(O)CCCCCC(=O)OC(CCCCCCCC)CCCCCCCC]

then the other of R₃ and R₄ is not selected from

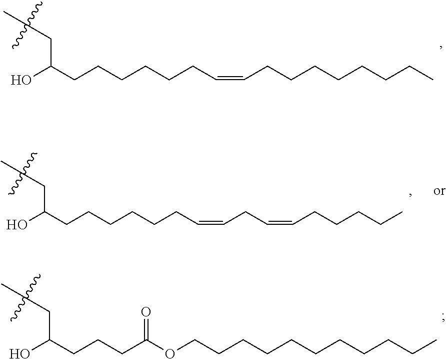 [SMILES: *CC(O)CCCCCC/C=C\CCCCCCCC ; *CC(O)CCCCCC/C=C\C/C=C\CCCCC ; *CC(O)CCCC(=O)OCCCCCCCCCCC]

or

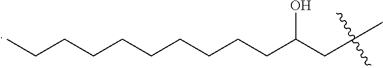 [SMILES: *CC(O)CCCCCCCCCC]

(b) if R₁ and R₂ are both and one of R₃ and R₄ is

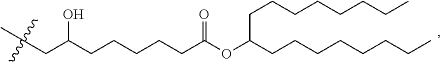 [SMILES: *CC(O)CCCCCC(=O)OC(CCCCCCCC)CCCCCCCC]

and a is 4 then the other of R₃ and R₄ is not selected from

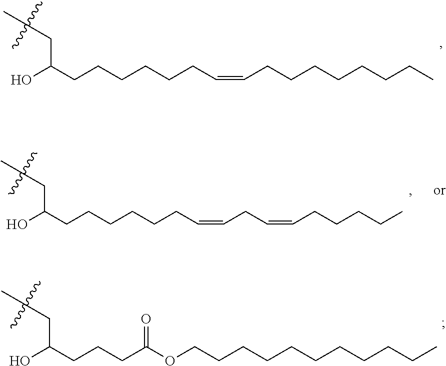 [SMILES: *CC(O)CCCCCC/C=C\CCCCCCCC ; *CC(O)CCCCCC/C=C\C/C=C\CCCCC ; *CC(O)CCCC(=O)OCCCCCCCCCCC]

or
(c) the compound is not one of the following structures:

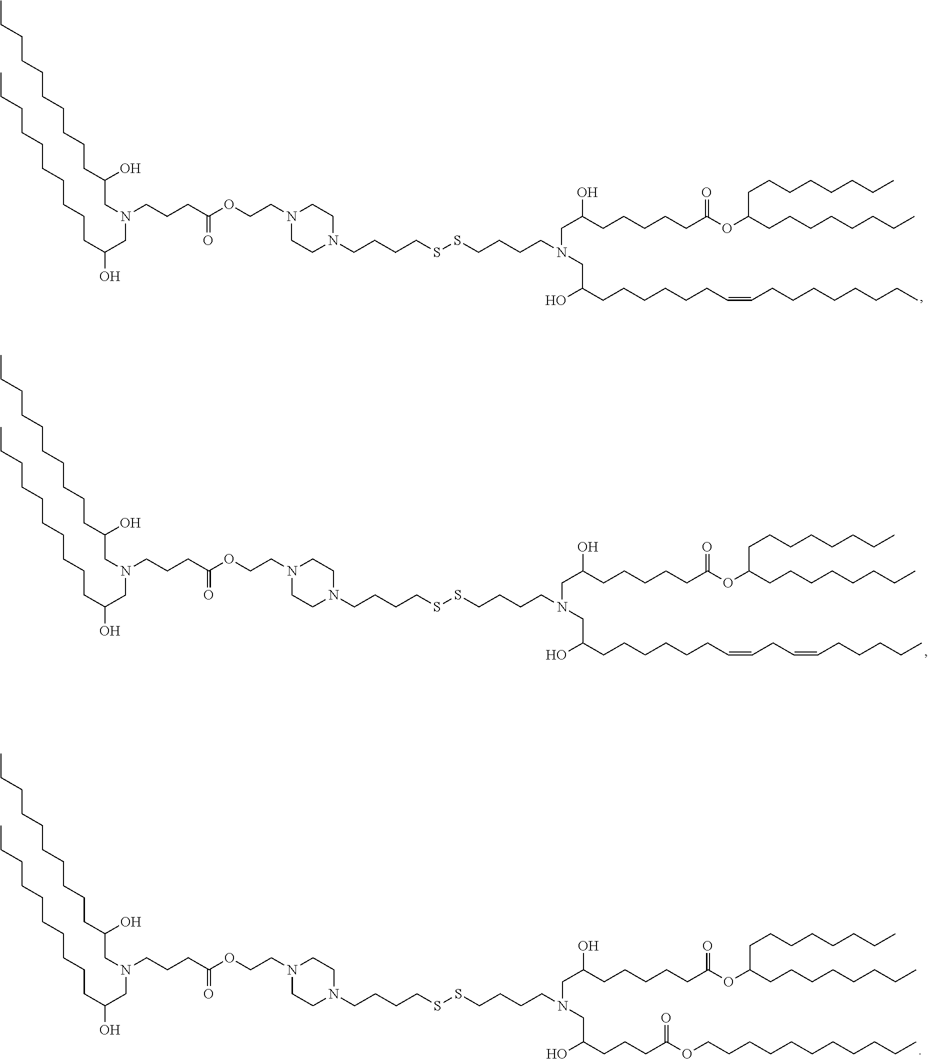 [SMILES: CCCCCCCC/C=C\CCCCCCC(O)CN(CCCCSSCCCCN1CCN(CCOC(=O)CCCN(CC(O)CCCCCCCCCC)CC(O)CCCCCCCCCC)CC1)CC(O)CCCCCC(=O)OC(CCCCCCCC)CCCCCCCC ; CCCCC/C=C\C/C=C\CCCCCCC(O)CN(CCCCSSCCCCN1CCN(CCOC(=O)CCCN(CC(O)CCCCCCCCCC)CC(O)CCCCCCCCCC)CC1)CC(O)CCCCCC(=O)OC(CCCCCCCC)CCCCCCCC ; CCCCCCCCCCCOC(=O)CCCC(O)CN(CCCCSSCCCCN1CCN(CCOC(=O)CCCN(CC(O)CCCCCCCCCC)CC(O)CCCCCCCCCC)CC1)CC(O)CCCCCC(=O)OC(CCCCCCCC)CCCCCCCC]

101. The compound of any one of embodiments 99-100, or a pharmaceutically acceptable salt thereof, wherein:
(a) R₁ and R₂ are not identical to one another and R₃ and R₄ are identical to one another;
(b) R₁ and R₂ are identical to one another and R₃ and R₄ are not identical to one another;
(c) R₁ and R₂ are not identical to one another and R₃ and R₄ are not identical to one another; or
(d) none of R₁, R₂, R₃, and R₄ are identical.

102. The compound of any one of embodiments 99-101, or a pharmaceutically acceptable salt thereof, wherein:
(a) one or more of R₁, R₂, R₃, and R₄ are independently selected from optionally substituted C₅-C₃₀ alkyl or optionally substituted C₅-C₃₀ alkenyl;
(b) one or more of R₁, R₂, R₃, and R₄ are independently selected from optionally substituted C₅-C₃₀ alkyl;
(c) one or more of R₁, R₂, R₃, and R₄ are independently selected from optionally substituted C₅-C₁₂ alkyl, for example

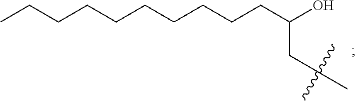 [SMILES: *CC(O)CCCCCCCCCC]

(d) one or more of R₁, R₂, R₃, and R₄ are independently selected from optionally substituted C₅-C₃₀ alkenyl;
(e) one or more of R₁, R₂, R₃, and R₄ are independently selected from optionally substituted C₅-C₁₈ alkenyl, for example

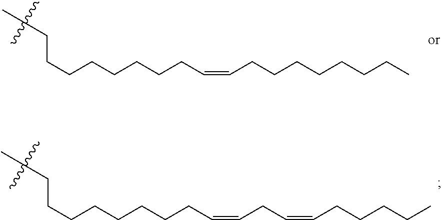 [SMILES: *CCCCCCCC/C=C\CCCCCCCC ; *CCCCCCCC/C=C\C/C=C\CCCCC]

(f) two of R₁, R₂, R₃, and R₄ are independently selected from optionally substituted C₅-C₃₀ alkyl and one of R₁, R₂, R₃, and R₄ is selected from optionally substituted C₅-C₃₀ alkenyl; and/or
(g) one or more of R₁, R₂, R₃, and R₄, or one of R₁, R₂, R₃, and R₄ is independently selected from optionally substituted C₁₋₅ alkyl which is substituted with an optionally substituted heterocyclyl moiety, optionally wherein said optionally substituted C₁₋₅ alkyl which is substituted with an optionally substituted heterocyclyl moiety is

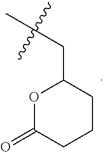 [SMILES: *CC1CCCC(=O)O1]

103. The compound of any one of embodiments 99-102, or a pharmaceutically acceptable salt thereof, wherein one or more of R₁, R₂, R₃, and R₄ is independently selected from

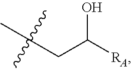

wherein each R_{A} is independently selected from optionally substituted C₃-C₂₈ alkyl, optionally substituted C₃-C₂₈ alkenyl, and —W¹—C(O)—O—X¹, wherein each W¹ is independently selected from optionally substituted C₃-C₅ alkylene and optionally substituted C₃-C₅ alkenylene, and each X¹ is independently selected from optionally substituted C₅-C₃₀ alkyl and optionally substituted C₅-C₃₀ alkenyl, optionally wherein:
(a) one or more R_{A} is independently selected from optionally substituted C₃-C₂₈ alkyl, optionally wherein each R_{A} is independently selected from optionally substituted C₃-C₂₈ alkyl;
(b) one or more R_{A} is independently selected from optionally substituted C₃-C₁₀ alkyl, for example

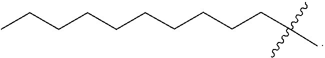

optionally wherein each R_{A} is independently selected from optionally substituted C₃-C₁₀ alkyl, for example

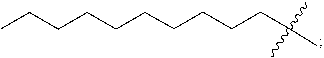

(c) one or more R_{A} is independently selected from optionally substituted C₃-C₂₈ alkenyl, for example optionally substituted C₃-C₁₆ alkenyl, optionally wherein each R_{A} is independently selected from optionally substituted C₃-C₂₈ alkenyl, for example optionally substituted C₃-C₁₆ alkenyl; and/or
(d) one or more R_{A} is independently selected from —W¹—C(O)—O—X¹, optionally wherein each R_{A} is independently selected from —W¹—C(O)—O—X¹.

104. The compound of any one of embodiments 99-103, or a pharmaceutically acceptable salt thereof, wherein one or more of R₁, R₂, R₃, and R₄ is independently selected from

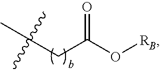

wherein each R_{B} is independently selected from optionally substituted C₅-C₃₀ alkyl and optionally substituted C₅-C₃₀ alkenyl, optionally wherein:
(a) one or more R_{B} is independently selected from optionally substituted C₅-C₃₀ alkyl, optionally wherein each R_{B} is independently selected from optionally substituted C₅-C₃₀ alkyl;
(b) one or more R_{B} is independently selected from optionally substituted C₆-C₁₇ alkyl, for example optionally substituted branched C₆-C₁₇ alkyl, optionally wherein each R_{B} is independently selected from optionally substituted C₆-C₁₇ alkyl, for example optionally substituted branched C₆-C₁₇ alkyl;
(c) one or more R_{B} is independently selected from
(i)

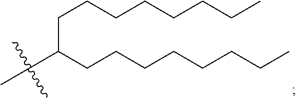 [SMILES: *C(CCCCCCCC)CCCCCCCC]

(ii)

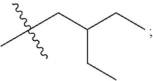

and
(iii)

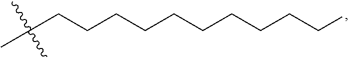

optionally wherein each R_{B} is independently selected from options (i), (ii), and (iii) above;
(d) one or more R_{B} is independently selected from optionally substituted C₅-C₃₀ alkenyl, optionally wherein each R_{B} is independently selected from optionally substituted C₅-C₃₀ alkenyl; and/or
(e) one or more R_{B} is independently selected from optionally substituted C₅-C₉ alkenyl, for example

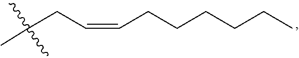

optionally wherein each R_{B} is independently selected from optionally substituted C₅—C, alkenyl, for example

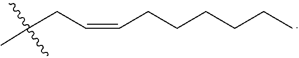

105. The compound of any one of embodiments 99-104, or a pharmaceutically acceptable salt thereof, wherein one or more of R₁, R₂, R₃, and R₄ are independently selected from

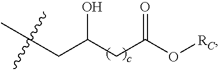

wherein each R_{C} independently selected from optionally substituted C₅-C₃₀ alkyl and optionally substituted C₅-C₃₀ alkenyl, optionally wherein:
(a) one or more c is selected from 3 or 5, optionally wherein each c is selected from 3 or 5;
(b) one or more c is 4, optionally wherein each c is 4;
(c) one or more R_{C} is independently selected from optionally substituted C₅-C₃₀ alkyl, optionally wherein each R_{C} is independently selected from optionally substituted C₅-C₃₀ alkyl;
(d) one or more R_{C} is independently selected from optionally substituted C₆-C₁₇ alkyl, for example optionally substituted branched C₆-C₁₇ alkyl, optionally wherein each R_{C} is independently selected from optionally substituted C₆-C₁₇ alkyl, for example optionally substituted branched C₆-C₁₇ alkyl;
(e) one or more R_{C} is independently selected from
   (i)

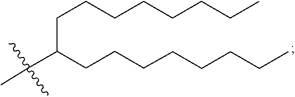

(ii)

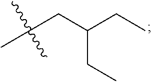

and
(iii)

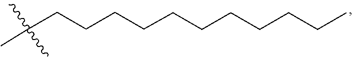

optionally wherein each R_{C} is independently selected from options (i), (ii) and (iii) above;
(f) one or more R_{C} is independently selected from optionally substituted C₅-C₃₀ alkenyl, optionally wherein each R_{C} is independently selected from optionally substituted C₅-C₃₀ alkenyl; and/or
(g) one or more R_{C} are independently selected from optionally substituted C₅-C₉ alkenyl, for example

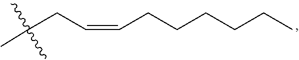

optionally wherein each R_{C} are independently selected from optionally substituted C₅-C₉ alkenyl, for example

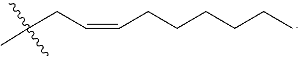

106. The compound of any one of embodiments 99-105, or a pharmaceutically acceptable salt thereof, wherein the compound has a structure according to Formula (II′), optionally Formula (II), Formula (III′), optionally Formula (III), Formula (IV′), optionally Formula (IV); or Formula (V′), optionally Formula (V):

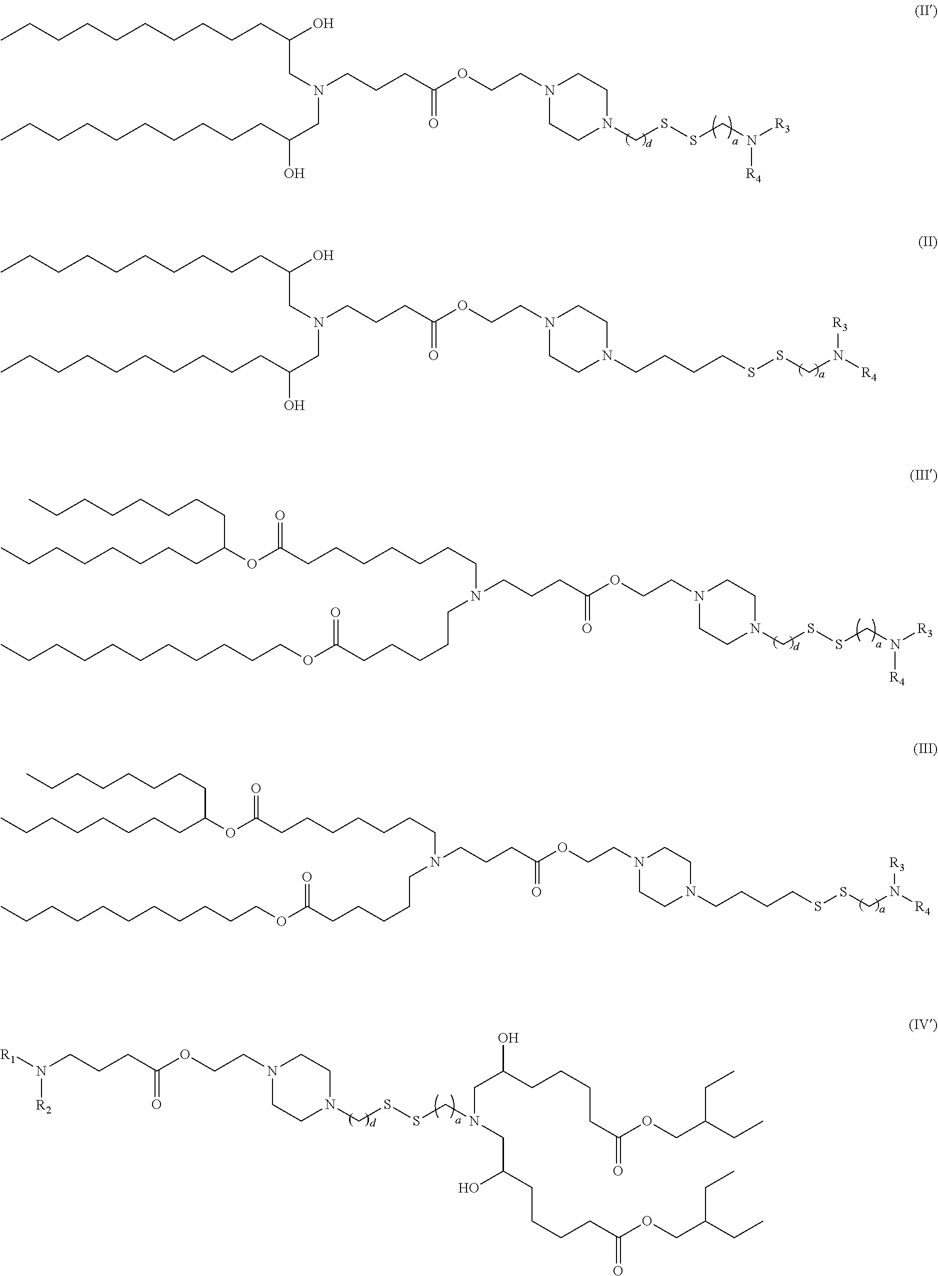

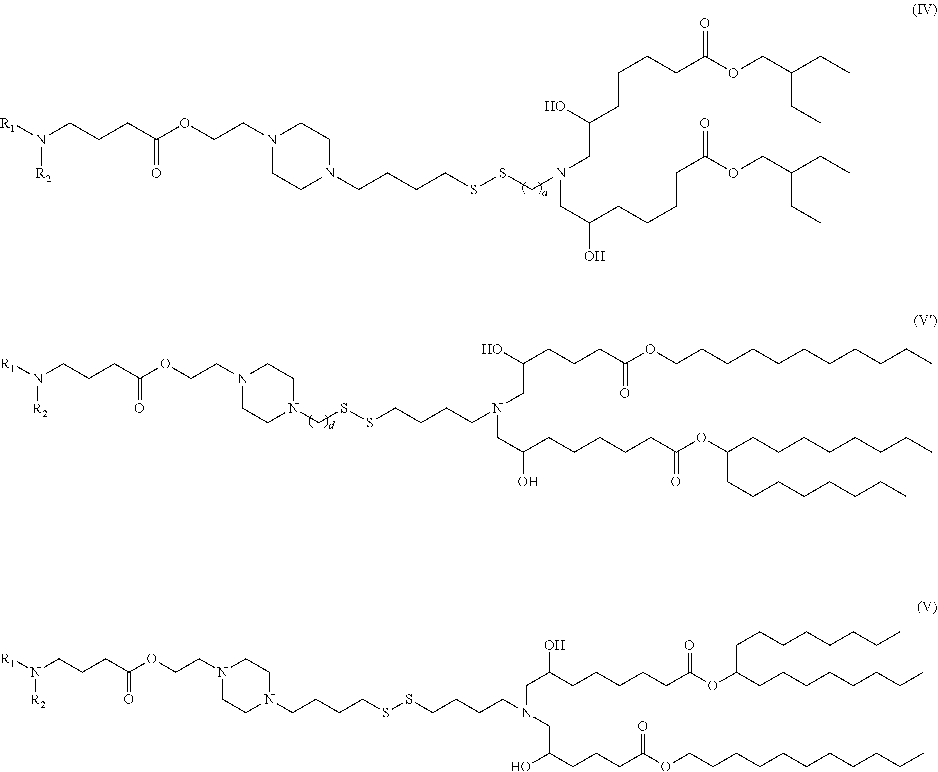

or a pharmaceutically acceptable salt thereof.

107. A compound having a structure according to Formula (VI′). optionally Formula (VI):

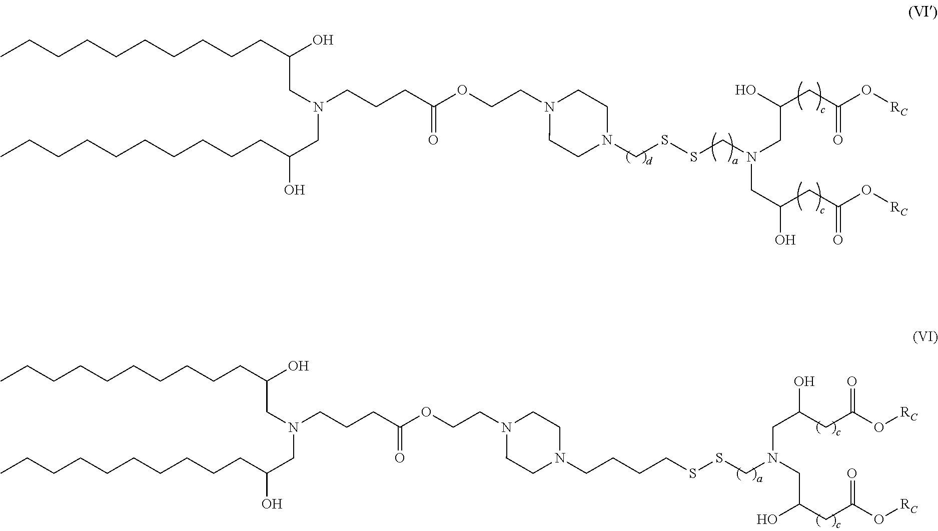

or a pharmaceutically acceptable salt thereof, wherein:
a is 3 or 4;
d is 2, 3, or 4;
each c is independently selected from 3 and 4; and
each R_{C} is independently selected from

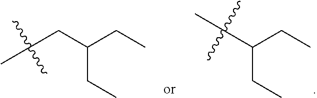

108. The compound of embodiment 107, or a pharmaceutically acceptable salt thereof, wherein:
(a) a is 3; or
(b) a is 4; and/or
(c) each c is 3; or
(d) each c is 4; and/or
(e) each R_{C} is

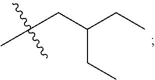 [SMILES: *CC(CC)CC]

or
(f) each R_{C} is

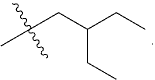 [SMILES: *CC(CC)CC]

109. The compound of any one of embodiments 99-108, or a pharmaceutically acceptable salt thereof, wherein:
(a) in the compound of Formula (I′), (II′), (III′), (IV′), (V′), or (VI′):
   (i) d is 2;
   (ii) d is 3; or
   (iii) d is 4; or
(b) the compound is selected from those listed in Tables 1 or 2, or a pharmaceutically acceptable salt thereof.

110. A composition comprising the compound of any one of embodiments 99-109 or a pharmaceutically acceptable salt thereof, one or more non-cationic lipids, one or more cholesterol-based lipids and one or more PEG-modified lipids, optionally wherein:
(a) the composition is a lipid nanoparticle, optionally a liposome;
(b) the composition is a lipid nanoparticle, optionally a liposome and the one or more cationic lipid(s) constitute(s) about 30 mol %-60 mol % of the lipid nanoparticle;
(c) the composition is a lipid nanoparticle, optionally a liposome and the one or more non-cationic lipid(s) constitute(s) 10 mol %-50 mol % of the lipid nanoparticle;
(d) the composition is a lipid nanoparticle, optionally a liposome and the one or more PEG-modified lipid(s) constitute(s) 1 mol %-10 mol % of the lipid nanoparticle; and/or
(e) the composition is a lipid nanoparticle, optionally a liposome and the cholesterol-based lipid constitutes 10 mol %-50 mol % of the lipid nanoparticle.

111. The composition of embodiment 110, wherein the composition is a lipid nanoparticle, optionally a liposome, and wherein said lipid nanoparticle encapsulates:
(a) a nucleic acid;
(b) a mRNA encoding a peptide or protein;
(c) a mRNA encoding an antigen;
(d) a mRNA encoding an influenza antigen, for example an antigen of influenza A virus or an antigen of influenza B virus;
(e) a mRNA encoding an antigen of Chlamydia sp. bacteria, for example an antigen of C. trachomatis;
(f) a mRNA encoding an antigen of P. gingivalis; or
(g) a mRNA encoding an acne antigen, for example an antigen of C. acnes.

112. The composition of embodiment 111 encapsulating a mRNA encoding a peptide, protein or antigen for use in therapy optionally wherein said composition is for use in:
(a) a method of treating or preventing a disease or disorder amenable to treatment or prevention by the peptide, protein or antigen encoded by the mRNA;
(b) a method of treating or preventing a disease or disorder amenable to treatment or prevention by the peptide, protein or antigen encoded by the mRNA, wherein the disease or disorder is caused by a viral infection;
(c) a method of treating or preventing a disease or disorder amenable to treatment or prevention by the peptide, protein or antigen encoded by the mRNA, wherein the disease or disorder is influenza;
(d) a method of treating or preventing a disease or disorder amenable to treatment or prevention by the peptide, protein or antigen encoded by the mRNA, wherein the disease or disorder is caused by a bacterial infection;
(e) a method of treating or preventing a disease or disorder amenable to treatment or prevention by the peptide, protein or antigen encoded by the mRNA, wherein the disease or disorder is chlamydia;
(f) a method of treating or preventing a disease or disorder amenable to treatment or prevention by the peptide, protein or antigen encoded by the mRNA, wherein the disease or disorder is induced by infection with P. gingivalis; or
(g) a method of treating or preventing a disease or disorder amenable to treatment or prevention by the peptide, protein or antigen encoded by the mRNA, wherein the disease or disorder is acne.

113. The composition for use according to embodiment 112, wherein the composition is administered intravenously, intrathecally or intramuscularly, or by pulmonary delivery, optionally through nebulization, optionally wherein the composition is administered intramuscularly.

1. A compound having a structure according to Formula (I): 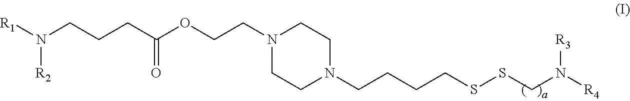
or a pharmaceutically acceptable salt thereof, wherein:
a is 3 or 4;
each of R₁, R₂, R₃, and R₄ is independently selected from:
(i) optionally substituted C₅-C₃₀ alkyl, and optionally substituted C₅-C₃₀ alkenyl;
(ii) optionally substituted C₁₋₅ alkyl which is substituted with an optionally substituted heterocyclyl moiety;
(iii) 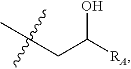
wherein each R_{A} is independently selected from optionally substituted C₃-C₂₈ alkyl, optionally substituted C₃-C₂₈ alkenyl, and —W¹—C(O)—O—X¹, wherein each W¹ is independently selected from optionally substituted C₃-C₅ alkylene and optionally substituted C₃-C₅ alkenylene, and each X¹ is independently selected from optionally substituted C₅-C₃₀ alkyl and optionally substituted C₅-C₃₀ alkenyl;
(iv) 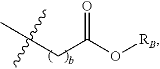
wherein each R_{B} is independently selected from optionally substituted C₅-C₃₀ alkyl, optionally substituted C₅-C₃₀ alkenyl; and
(v) 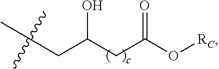
wherein each R_{C} is independently selected from optionally substituted C₅-C₃₀ alkyl and optionally substituted C₅-C₃₀ alkenyl;
each b is independently selected from 5, 6, and 7;
each c is independently selected from 3, 4, and 5;
d is 2, 3, or 4;
and wherein:
R₁ and R₂ are not identical to one another and/or R₃ and R₄ are not identical to one another; and wherein:
at least one of R₁, R₂, R₃, and R₄, is independently selected from option (iv) 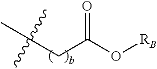
or option (v)

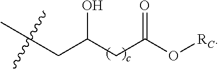

2. The compound of claim 1, or a pharmaceutically acceptable salt thereof, wherein:
(a) if R₁ and R₂ are both 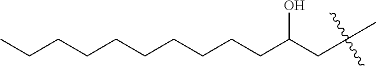 [SMILES: *CC(O)CCCCCCCCCC]
and one of R₃ and R₄ is 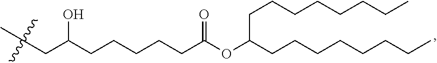 [SMILES: *CC(O)CCCCCC(=O)OC(CCCCCCCC)CCCCCCCC]
then the other of R₃ and R₄ is not selected from 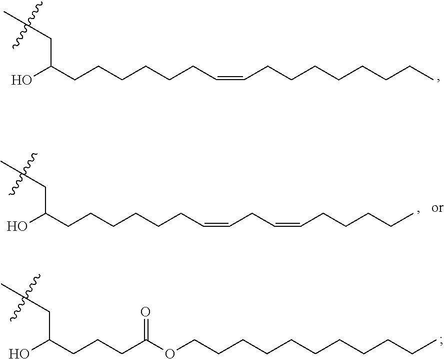 [SMILES: *CC(O)CCCCCC/C=C\CCCCCCCC ; *CC(O)CCCCCC/C=C\C/C=C\CCCCC ; *CC(O)CCCC(=O)OCCCCCCCCCCC]
or
(b) if R₁ and R₂ are both 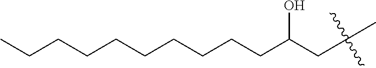 [SMILES: *CC(O)CCCCCCCCCC]
and one of R₃ and R₄ is 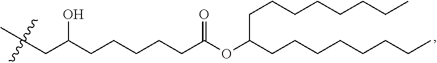 [SMILES: *CC(O)CCCCCC(=O)OC(CCCCCCCC)CCCCCCCC]
and a is 4 then the other of R₃ and R₄ is not selected from 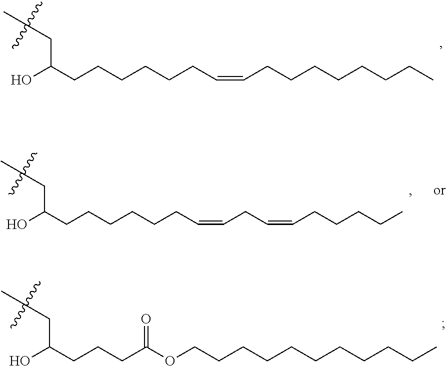 [SMILES: *CC(O)CCCCCC/C=C\CCCCCCCC ; *CC(O)CCCCCC/C=C\C/C=C\CCCCC ; *CC(O)CCCC(=O)OCCCCCCCCCCC]
or
(c) the compound is not one of the following structures:

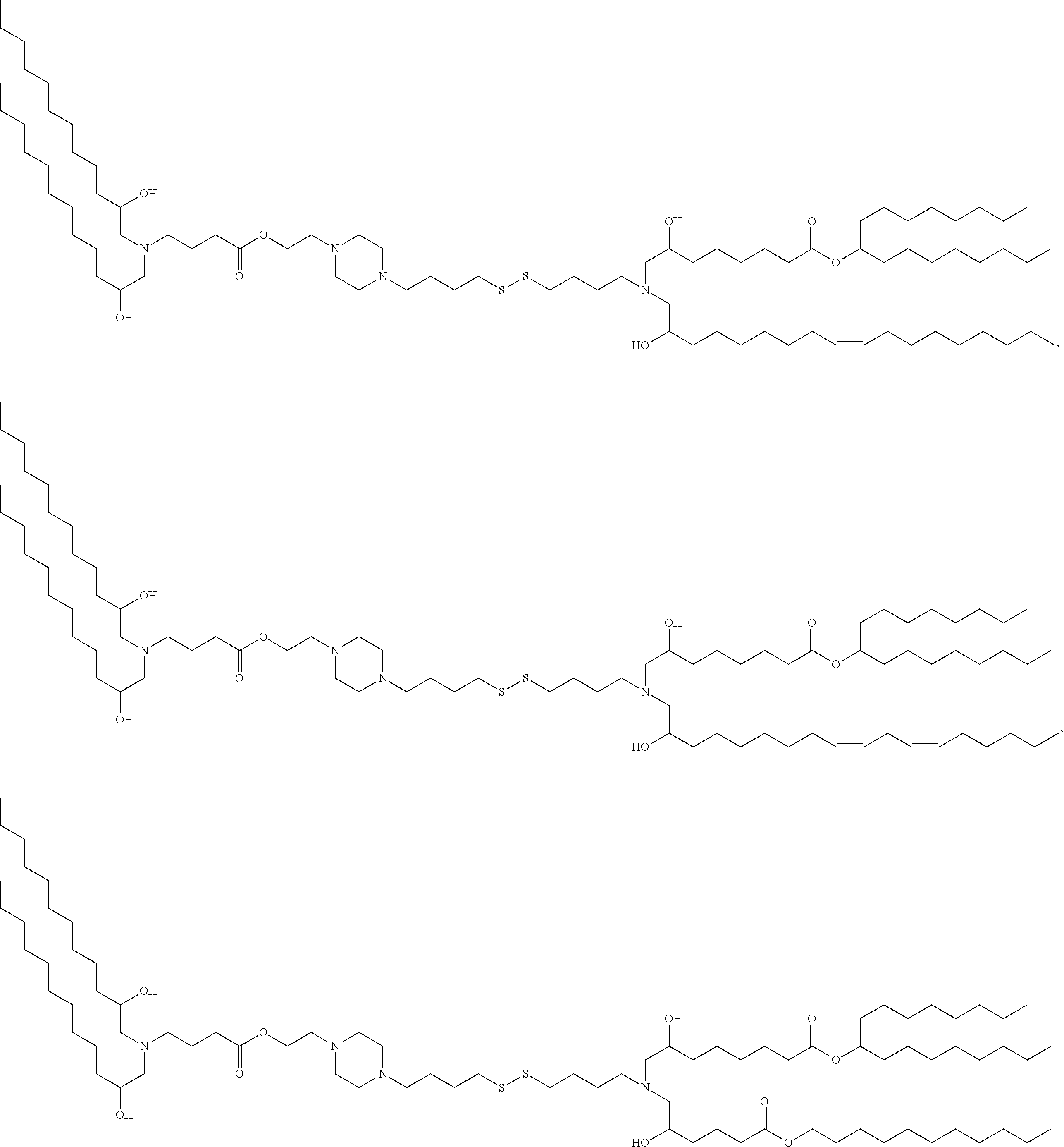 [SMILES: CCCCCCCC/C=C\CCCCCCC(O)CN(CCCCSSCCCCN1CCN(CCOC(=O)CCCN(CC(O)CCCCCCCCCC)CC(O)CCCCCCCCCC)CC1)CC(O)CCCCCC(=O)OC(CCCCCCCC)CCCCCCCC ; CCCCC/C=C\C/C=C\CCCCCCC(O)CN(CCCCSSCCCCN1CCN(CCOC(=O)CCCN(CC(O)CCCCCCCCCC)CC(O)CCCCCCCCCC)CC1)CC(O)CCCCCC(=O)OC(CCCCCCCC)CCCCCCCC ; CCCCCCCCCCCOC(=O)CCCC(O)CN(CCCCSSCCCCN1CCN(CCOC(=O)CCCN(CC(O)CCCCCCCCCC)CC(O)CCCCCCCCCC)CC1)CC(O)CCCCCC(=O)OC(CCCCCCCC)CCCCCCCC]

3. The compound of claim 1, or a pharmaceutically acceptable salt thereof, wherein:
R₁ and R₂ are not identical to one another and R₃ and R₄ are identical to one another; or
R₁ and R₂ are identical to one another and R₃ and R₄ are not identical to one another.

4. The compound of claim 1, or a pharmaceutically acceptable salt thereof, wherein:
one or more of R₁, R₂, R₃, and R₄ is independently selected from optionally substituted C₅-C₃₀ alkyl or optionally substituted C₅-C₃₀ alkenyl; or
one or more of R₁, R₂, R₃, and R₄, is independently selected from optionally substituted C₁₋₅ alkyl which is further substituted with an optionally substituted heterocyclyl moiety.

5. The compound of claim 1, or a pharmaceutically acceptable salt thereof, wherein one or more of R₁, R₂, R₃, and R₄ is independently selected from 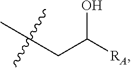
wherein each R_{A} is independently selected from optionally substituted C₃-C₂₈ alkyl, optionally substituted C₃-C₂₈ alkenyl, and —W¹—C(O)—O—X¹, wherein each W¹ is independently selected from optionally substituted C₃-C₅ alkylene and optionally substituted C₃-C₅ alkenylene, and each X¹ is independently selected from optionally substituted C₅-C₃₀ alkyl and optionally substituted C₅-C₃₀ alkenyl.

6. The compound of claim 1, or a pharmaceutically acceptable salt thereof, wherein one or more of R₁, R₂, R₃, and R₄ is independently selected from 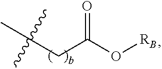
wherein each R_{B} is independently selected from optionally substituted C₅-C₃₀ alkyl and optionally substituted C₅-C₃₀ alkenyl.

7. The compound of claim 1, or a pharmaceutically acceptable salt thereof, wherein one or more of R₁, R₂, R₃, and R₄ are independently selected from 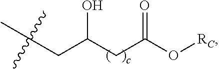
wherein each R_{C} independently selected from optionally substituted C₅-C₃₀ alkyl and optionally substituted C₅-C₃₀ alkenyl.

8. The compound of claim 1, or a pharmaceutically acceptable salt thereof, wherein the compound has a structure according to Formula (II), Formula (III), Formula (IV), or Formula (V): 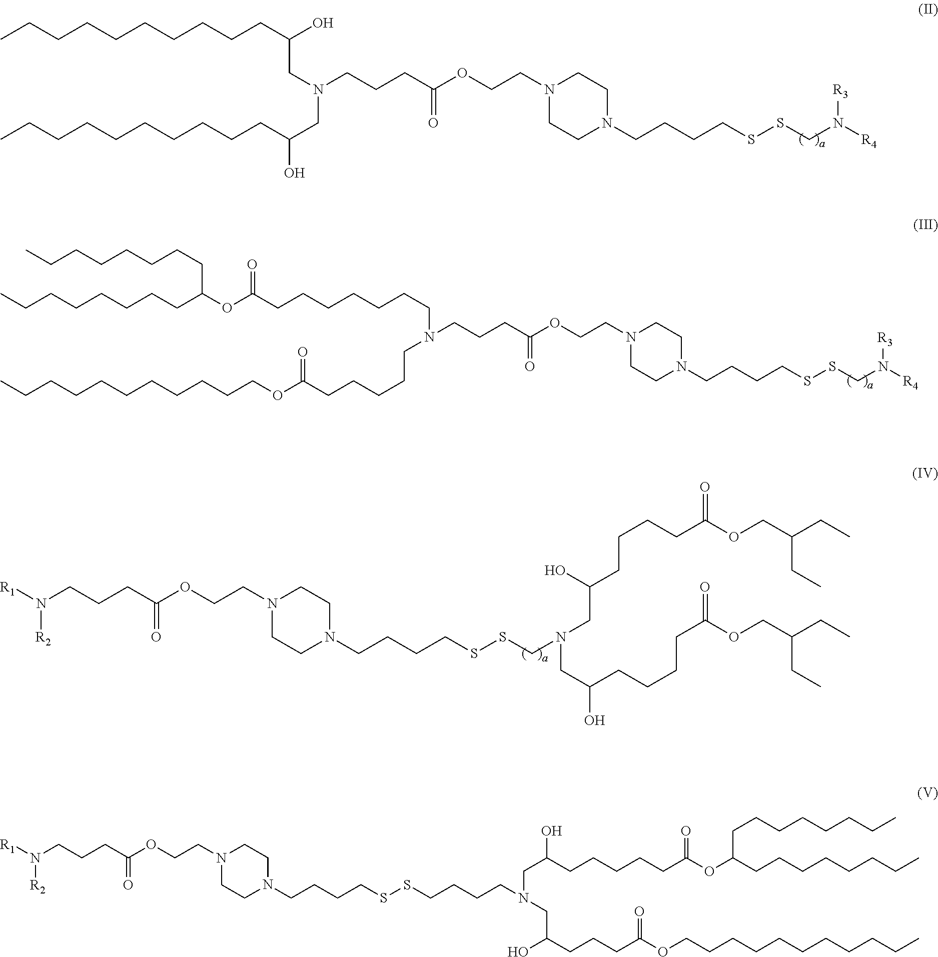
or a pharmaceutically acceptable salt thereof.

9. A compound having a structure according to Formula (VI): 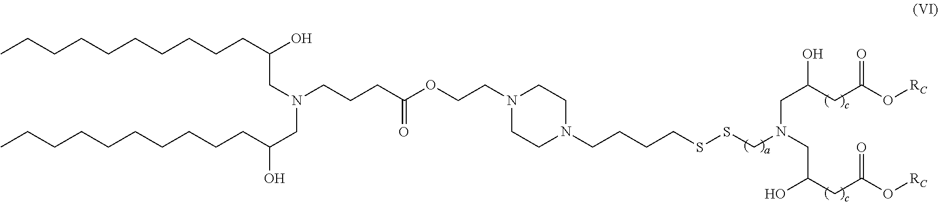
or a pharmaceutically acceptable salt thereof, wherein:
a is 3 or 4;
d is 2, 3, or 4;
each c is independently selected from 3 and 4; and
each R_{C} is independently selected from 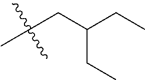 [SMILES: *CC(CC)CC]
and

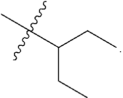 [SMILES: *C(CC)CC]

10. The compound of claim 9, or a pharmaceutically acceptable salt thereof, wherein:
(a) a is 3; or
(b) a is 4; and/or
(c) each c is 3; or
(d) each c is 4; and/or
(e) each R_{C} is 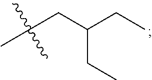 [SMILES: *CC(CC)CC]
or
(f) each R_{C} is

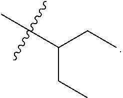 [SMILES: *C(CC)CC]

11. The compound of claim 1, or a pharmaceutically acceptable salt thereof, wherein the compound is selected from those listed in Table 1, or a pharmaceutically acceptable salt thereof.

12. A composition comprising a compound of having a structure according to Formula (I): 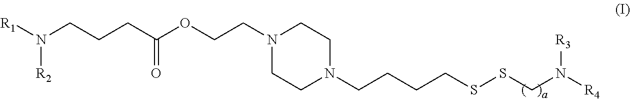
or a pharmaceutically acceptable salt thereof, wherein:
a is 3 or 4;
each of R₁, R₂, R₃, and R₄ is independently selected from:
(i) optionally substituted C₅-C₃₀ alkyl, and optionally substituted C₅-C₃₀ alkenyl;
(ii) optionally substituted C₁₋₅ alkyl which is substituted with an optionally substituted heterocyclyl moiety;
(iii) 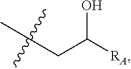
wherein each R_{A} is independently selected from optionally substituted C₃-C₂₈ alkyl, optionally substituted C₃-C₂₈ alkenyl, and —W¹—C(O)—O—X¹, wherein each W¹ is independently selected from optionally substituted C₃-C₅ alkylene and optionally substituted C₃-C₅ alkenylene, and each X¹ is independently selected from optionally substituted C₅-C₃₀ alkyl and optionally substituted C₅-C₃₀ alkenyl;
(iv) 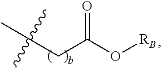
wherein each R_{B} is independently selected from optionally substituted C₅-C₃₀ alkyl, optionally substituted C₅-C₃₀ alkenyl; and
(v) 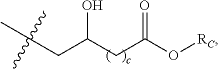
wherein each R_{C} is independently selected from optionally substituted C₅-C₃₀ alkyl and optionally substituted C₅-C₃₀ alkenyl;
each b is independently selected from 5, 6, and 7;
each c is independently selected from 3, 4, and 5;
d is 2, 3, or 4;
and wherein:
R₁ and R₂ are not identical to one another and/or R₃ and R₄ are not identical to one another; and wherein:
at least one of R₁, R₂, R₃, and R₄, is independently selected from option (iv) 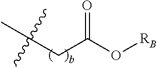
or
option (v) 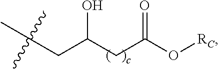
or a pharmaceutically acceptable salt thereof, one or more non-cationic lipids, one or more cholesterol-based lipids and one or more PEG-modified lipids.

13. The composition of claim 12, wherein the composition is a lipid nanoparticle, optionally a liposome, and wherein said lipid nanoparticle encapsulates a nucleic acid.

14. A method of treating or preventing a disease or disorder amenable to treatment or prevention by a peptide, protein, or antigen encoded by a mRNA; wherein the method comprises administering to a subject in need thereof the composition of claim 13, wherein the disease or disorder is: (a) viral infection; (b) influenza; (c) bacterial infection; (d) chlamydia; (e) infection with P. gingivalis; or (f) acne.

15. The method of claim 14, wherein the composition is administered intravenously, intrathecally or intramuscularly, or by pulmonary delivery, optionally through nebulization, optionally wherein the composition is administered intramuscularly.

16. The compound of claim 1, or a pharmaceutically acceptable salt thereof, wherein R₁ and R₂ are not identical to one another and R₃ and R₄ are not identical to one another.

17. The compound of claim 1, or a pharmaceutically acceptable salt thereof, wherein none of R₁, R₂, R₃, and R₄ are identical.

18. The compound of claim 9, or a pharmaceutically acceptable salt thereof, wherein the compound is selected from those listed in Table 2, or a pharmaceutically acceptable salt thereof.

19. A composition comprising the compound of claim 9, or a pharmaceutically acceptable salt thereof, one or more non-cationic lipids, one or more cholesterol-based lipids and one or more PEG-modified lipids.

20. A method of treating or preventing a disease or disorder amenable to treatment or prevention by a peptide, protein, or antigen encoded by a mRNA, wherein the method comprises administering to a subject in need thereof the composition of claim 19, wherein the composition encapsulates a mRNA encoding a peptide, protein, or antigen, wherein the disease or disorder is: (a) caused by a viral infection; (b) influenza; (c) caused by a bacterial infection; (d) chlamydia; (e) induced by infection with P. gingivalis; or (f) acne
